(19) United States
(12) Patent Application Publication
(10) Pub. No.: US 2023/0107173 A1
(43) Pub. Date: Apr. 6, 2023

(54) Methods Of Treating Chronic Kidney Disease (CKD) With Inhibitors Of Protective Loss-Of-Function Genes

(71) Applicant: Regeneron Pharmaceuticals, Inc., Tarrytown, NY (US)

(72) Inventors: Mary E. Haas, Tarrytown, NY (US); Luca Andrea Lotta, Tarrytown, NY (US); Aris Baras, Tarrytown, NY (US); Manuel Allen Revez Ferreira, Tarrytown, NY (US); Adam E. Locke, Tarrytown, NY (US); Joshua Backman, Tarrytown, NY (US)

(21) Appl. No.: 17/934,277

(22) Filed: Sep. 22, 2022

Related U.S. Application Data
(60) Provisional application No. 63/247,507, filed on Sep. 23, 2021.

(51) Int. Cl. C12N 15/113

(52) U.S. Cl. CPC C12N 15/1138; C12N 15/1137; C12N 2310/14

(57) ABSTRACT

The present disclosure provides methods of treating a subject having a kidney disease or preventing a subject from developing a kidney disease, and methods of identifying subjects having an increased risk of developing a kidney disease.

### REFERENCE TO SEQUENCE LISTING

This application includes a Sequence Listing filed electronically as an XML file named 381203594SEQ, created on Sep. 21, 2022, with a size of 1,302 kilobytes. The Sequence Listing is incorporated herein by reference.

### FIELD

The present disclosure relates generally to the treatment of subjects having kidney disease with Aldehyde Dehydrogenase 1 Family Member L1 (ALDH1L1) inhibitors, Fructose-Bisphosphate Aldolase B (ALDOB) inhibitors, Glucose-6-Phosphatase Catalytic Subunit 1 (G6PC) inhibitors, LDL Receptor Related Protein 2 (LRP2), Ribosomal Protein L3 Like (RPL3L) inhibitors, Solute Carrier Family 25, Member 45 (SLC25A45) inhibitors, Solute Carrier Family 7 Member 9 (SLC7A9) inhibitors, or any combination thereof, and methods of identifying subjects having an increased risk of developing kidney disease.

### BACKGROUND

In the United States, based on data from the 1999-2006 National Health and Nutrition Examination Survey (NHANES) study, an estimated 11.1 percent (22.4 million) of adults aged 20 or older have chronic kidney disease (CKD) stages 1-3. An additional 0.8 million U.S. adults aged 20 or older have CKD stage 4, and more than 0.3 million have stage 5 CKD and receive hemodialysis. Analyses of NHANES data between 1988-1994 and 1999-2004 suggest that the prevalence of CKD is rising for every CKD stage, but with a particular increase in the prevalence of individuals classified with CKD stage 3. The number of patients with stage 5 CKD requiring dialysis also has increased. It has been estimated that more than 700,000 individuals will have End Stage Renal Disease (ESRD) by 2015. Although CKD can be caused by primary kidney disease (e.g., glomerular diseases, tubulointerstitial diseases, obstruction, and polycystic kidney disease), in the vast majority of patients with CKD, the kidney damage is associated with other medical conditions such as diabetes and hypertension. In 2008, excluding those with ESRD, 48 percent of Medicare patients with CKD had diabetes, 91 percent had hypertension, and 46 percent had atherosclerotic heart disease. Other risk factors for CKD include age, obesity, family history, and ethnicity. CKD has been associated with numerous adverse health outcomes.

A Glomerular Filtration Rate (GFR) of 90 mL/min or higher (Stage 1) is normal in most healthy people. Usually few symptoms are present at this stage of CKD. A GFR of 60-89 mL/min (Stage 2) may for some patients, such as the elderly or infants, be normal if no kidney damage is present. A GFR between 60-89 mL/min for three months or longer along with kidney damage is a sign of early CKD. Usually few symptoms are present at this stage. A GFR between 30-59 mL/min (Stage 3) for a patient is indicative of moderate CKD, and are more likely to develop anemia, early bone disease or high blood pressure, and may desire to see a nephrologist. A GFR between 15-29 mL/min (Stage 4) indicates that the patient has severe CKD, and will likely need dialysis or a kidney transplant in the future. A GFR of 15 mL/min or less (Stage 5) indicates that the patient has chronic CKD, and have ESRD. The kidneys have lost almost all ability to function effectively at this stage. They will need dialysis or a kidney transplant to live.

Aldehyde Dehydrogenase 1 Family Member L1 (ALDH1L1) is a member of aldehyde dehydrogenase family, and catalyzes the conversion of 10-formyltetrahydrofolate, nicotinamide adenine dinucleotide phosphate (NADP⁺), and water to tetrahydrofolate, NADPH, and carbon dioxide.

Fructose-Bisphosphate Aldolase B (ALDOB) is a tetrameric glycolytic enzyme that catalyzes the reversible conversion of fructose-1,6-bisphosphate to glyceraldehyde 3-phosphate and dihydroxyacetone phosphate. Vertebrates have 3 aldolase isozymes which are distinguished by their electrophoretic and catalytic properties. Differences indicate that aldolases A, B, and C are distinct proteins, the products of a family of related ‘housekeeping’ genes exhibiting developmentally regulated expression of the different isozymes. The developing embryo produces aldolase A, which is produced in even greater amounts in adult muscle where it can be as much as 5% of total cellular protein. In adult liver, kidney and intestine, aldolase A expression is repressed and aldolase B is produced. In brain and other nervous tissue, aldolase A and C are expressed about equally. There is a high degree of homology between aldolase A and C. Defects in ALDOB cause hereditary fructose intolerance.

Glucose-6-Phosphatase Catalytic Subunit 1 (G6PC) is a multi-subunit integral membrane protein of the endoplasmic reticulum that is composed of a catalytic subunit and transporters for G6P, inorganic phosphate, and glucose. Glucose-6-phosphatase catalyzes the hydrolysis of D-glucose 6-phosphate to D-glucose and orthophosphate and is a key enzyme in glucose homeostasis, functioning in gluconeogenesis and glycogenolysis. Mutations in this gene cause glycogen storage disease type I (GSD1). This disease, also known as von Gierke disease, is a metabolic disorder characterized by severe hypoglycemia associated with the accumulation of glycogen and fat in the liver and kidneys.

LDL Receptor Related Protein 2 (LRP2) is a multi-ligand endocytic receptor that is expressed in many different tissues but primarily in absorptive epithelial tissues such as the kidney. This glycoprotein has a large amino-terminal extracellular domain, a single transmembrane domain, and a short carboxy-terminal cytoplasmic tail. The extracellular ligand-binding-domains bind diverse macromolecules including albumin, apolipoproteins B and E, and lipoprotein lipase. The LRP2 protein is critical for the reuptake of numerous ligands, including lipoproteins, sterols, vitamin-binding proteins, and hormones. This protein also has a role in cell-signaling; extracellular ligands include parathyroid hormones and the morphogen sonic hedgehog while cytosolic ligands include MAP kinase scaffold proteins and JNK interacting proteins.

Ribosomal Protein L3 Like (RPL3L) gene encodes a protein that shares sequence similarity with ribosomal protein L3. The protein belongs to the L3P family of ribosomal proteins. Unlike the ubiquitous expression of ribosomal protein genes, this gene has a tissue-specific pattern of expression, with the highest levels of expression in skeletal muscle and heart. It is not currently known whether the encoded protein is a functional ribosomal protein or whether it has evolved a function that is independent of the ribosome.

Solute Carrier Family 25, Member 45 (SLC25A45) belongs to the SLC25 family of mitochondrial carrier proteins, a large family of nuclear-encoded transporters embedded in the inner mitochondrial membrane and in a few cases other organelle membranes. The members of this superfamily are widespread in eukaryotes and involved in numerous metabolic pathways and cell functions. SLC25 members vary greatly in the nature and size of their transported substrates, modes of transport (i.e., uniport, symport or antiport) and driving forces, although the molecular mechanism of substrate translocation may be basically the same.

Solute Carrier Family 7 Member 9 (SLC7A9) is a member of a family of light subunits of amino acid transporters. This protein plays a role in the high-affinity and sodium-independent transport of cystine and neutral and dibasic amino acids, and appears to function in the reabsorption of cystine in the kidney tubule. Mutations in this gene cause non-type I cystinuria, a disease that leads to cystine stones in the urinary system due to impaired transport of cystine and dibasic amino acids.

### SUMMARY

The present disclosure provides methods of treating a subject having a kidney disease or preventing a subject from developing a kidney disease, the methods comprising administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject.

The present disclosure also provides methods of treating a subject having chronic kidney disease or preventing a subject from developing chronic kidney disease, the methods comprising administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject.

The present disclosure also provides methods of treating a subject having a kidney stone or preventing a subject from developing a kidney stone, the methods comprising administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject.

The present disclosure also provides methods of treating a subject having chronic glomerulonephritis or preventing a subject from developing chronic glomerulonephritis, the methods comprising administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject.

The present disclosure also provides methods of treating a subject having nephrosis or preventing a subject from developing nephrosis, the methods comprising administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject.

The present disclosure also provides methods of treating a subject having nephronophthisis or preventing a subject from developing nephronophthisis, the methods comprising administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject.

The present disclosure also provides methods of treating a subject having chronic interstitial nephritis or preventing a subject from developing chronic interstitial nephritis, the methods comprising administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject.

The present disclosure also provides methods of treating a subject having nephrosclerosis or preventing a subject from developing nephrosclerosis, the methods comprising administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject.

The present disclosure also provides methods of treating a subject with a therapeutic agent that treats or inhibits a kidney disease wherein the subject has a kidney disease, or preventing a subject from developing a kidney disease by administering a therapeutic agent that prevents a kidney disease, the methods comprising the steps of: determining whether the subject has any one or more variant nucleic acid molecules encoding an ALDH1L1, an ALDOB, a G6PC, an LRP2, an RPL3L, an SLC25A45, or an SLC7A9 predicted loss-of-function polypeptide by: obtaining or having obtained a biological sample from the subject; and performing or having performed a sequence analysis on the biological sample to determine if the subject has a genotype comprising any one or more of ALDH1L1, ALDOB, G6PC, LRP2, RPL3L, SLC25A45, or SLC7A9 variant nucleic acid molecules encoding a predicted loss-of-function polypeptide; and administering or continuing to administer the therapeutic agent that treats, prevents, or inhibits the kidney disease in an amount that is the same as or less than a standard dosage amount to a subject that is ALDH1L1, ALDOB, G6PC, LRP2, RPL3L, SLC25A45, and SLC7A9 reference, and/or administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject; administering or continuing to administer the therapeutic agent that treats, prevents, or inhibits the kidney disease in an amount that is the same as or less than a standard dosage amount to a subject that is heterozygous for any one or more of ALDH1L1, ALDOB, G6PC, LRP2, RPL3L, SLC25A45, or SLC7A9 variant nucleic acid molecules, and/or administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject; or administering or continuing to administer the therapeutic agent that treats, prevents, or inhibits the kidney disease in a standard dosage amount to a subject that is homozygous for any one or more of ALDH1L1, ALDOB, G6PC, LRP2, RPL3L, SLC25A45, or SLC7A9 variant nucleic acid molecules; wherein the presence of a genotype having one or more of ALDH1L1, ALDOB, G6PC, LRP2, RPL3L, SLC25A45, or SLC7A9 variant nucleic acid molecules indicates the subject has a decreased risk of developing the kidney disease.

The present disclosure also provides methods of identifying a subject having an increased risk of developing a kidney disease, the methods comprising: determining or having determined the presence or absence of any one or more variant nucleic acid molecules encoding an ALDH1L1, an ALDOB, a G6PC, an LRP2, an RPL3L, an SLC25A45, or an SLC7A9 predicted loss-of-function polypeptide in a biological sample obtained from the subject; wherein: when the subject is ALDH1L1, ALDOB, G6PC, LRP2, RPL3L, SLC25A45, and SLC7A9 reference, then the subject has an increased risk of developing the kidney disease; and when the subject is heterozygous or homozygous for any one or more of ALDH1L1, ALDOB, G6PC, LRP2, RPL3L, SLC25A45, or SLC7A9 variant nucleic acid molecules encoding a predicted loss-of-function polypeptide, then the subject has a decreased risk of developing the kidney disease.

The present disclosure also provides therapeutic agents that treat, prevent, or inhibit a kidney disease for use in the treatment and/or prevention of a kidney disease in a subject having: one or more of a variant genomic nucleic acid molecule encoding an ALDH1L1, an ALDOB, a G6PC, an LRP2, an RPL3L, an SLC25A45, or an SLC7A9 predicted loss-of-function polypeptide; one or more of a variant mRNA molecule encoding an ALDH1L1, ALDOB, G6PC, LRP2, RPL3L, SLC25A45, or SLC7A9 predicted loss-of-function polypeptide; or one or more of a variant cDNA molecule encoding an ALDH1L1, ALDOB, G6PC, LRP2, RPL3L, SLC25A45, or SLC7A9 predicted loss-of-function polypeptide.

The present disclosure also provides ALDH1L1 inhibitors for use in the treatment and/or prevention of a kidney disease in a subject that is: a) reference for ALDH1L1; or b) heterozygous for one or more of: i) a variant genomic nucleic acid molecule encoding ALDH1L1 predicted loss-of-function polypeptide; ii) a variant mRNA molecule encoding an ALDH1L1 predicted loss-of-function polypeptide; or iii) a variant cDNA molecule encoding an ALDH1L1 predicted loss-of-function polypeptide.

The present disclosure also provides ALDOB inhibitors for use in the treatment and/or prevention of a kidney disease in a subject that is: a) reference for ALDOB; or b) heterozygous for one or more of: i) a variant genomic nucleic acid molecule encoding an ALDOB predicted loss-of-function polypeptide; ii) a variant mRNA molecule encoding an ALDOB predicted loss-of-function polypeptide; or iii) a variant cDNA molecule encoding an ALDOB predicted loss-of-function polypeptide.

The present disclosure also provides G6PC inhibitors for use in the treatment and/or prevention of a kidney disease in a subject that is: a) reference for G6PC; or b) heterozygous for one or more of: i) a variant genomic nucleic acid molecule encoding a G6PC predicted loss-of-function polypeptide; ii) a variant mRNA molecule encoding a G6PC predicted loss-of-function polypeptide; or iii) a variant cDNA molecule encoding a G6PC predicted loss-of-function polypeptide.

The present disclosure also provides LRP2 inhibitors for use in the treatment and/or prevention of a kidney disease in a subject that is: a) reference for LRP2; or b) heterozygous for one or more of: i) a variant genomic nucleic acid molecule encoding an LRP2 predicted loss-of-function polypeptide; ii) a variant mRNA molecule encoding an LRP2 predicted loss-of-function polypeptide; or iii) a variant cDNA molecule encoding an LRP2 predicted loss-of-function polypeptide.

The present disclosure also provides RPL3L inhibitors for use in the treatment and/or prevention of a kidney disease in a subject that is: a) reference for RPL3L; or b) heterozygous for one or more of: i) a variant genomic nucleic acid molecule encoding an RPL3L predicted loss-of-function polypeptide; ii) a variant mRNA molecule encoding an RPL3L predicted loss-of-function polypeptide; or iii) a variant cDNA molecule encoding an RPL3L predicted loss-of-function polypeptide.

The present disclosure also provides SLC25A45 inhibitors for use in the treatment and/or prevention of a kidney disease in a subject that is: a) reference for SLC25A45; or b) heterozygous for one or more of: i) a variant genomic nucleic acid molecule encoding an SLC25A45 predicted loss-of-function polypeptide; ii) a variant mRNA molecule encoding an SLC25A45 predicted loss-of-function polypeptide; or iii) a variant cDNA molecule encoding an SLC25A45 predicted loss-of-function polypeptide.

The present disclosure also provides SLC7A9 inhibitors for use in the treatment and/or prevention of a kidney disease in a subject that is: a) reference for SLC7A9; or b) heterozygous for one or more of: i) a variant genomic nucleic acid molecule encoding an SLC7A9 predicted loss-of-function polypeptide; ii) a variant mRNA molecule encoding an SLC7A9 predicted loss-of-function polypeptide; or iii) a variant cDNA molecule encoding an SLC7A9 predicted loss-of-function polypeptide.

### BRIEF DESCRIPTION OF THE DRAWINGS

The accompanying FIGURES, which are incorporated in and constitute a part of this specification, illustrate several aspects and together with the description serve to explain the principles of the present disclosure.

FIG. 1 shows a burden of pLOF+missense variants in genes associated with increased eGFR show directionally consistent effects (i.e., increased eGFR) for a burden of pLOF variants in the same gene. Effect of burden of pLOF+missense variants as listed in Table 1 is plotted on the x-axis, and burden of pLOF-only variants with AAF>1% is plotted on the y-axis. pLOF indicates predicted loss of function; AAF indicates alternate allele frequency; eGFR indicates estimated glomerular filtration rate.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that several variant nucleic acid molecules (i.e., ALDH1L1, ALDOB, G6PC, LRP2, RPL3L, SLC25A45, and SLC7A9) encoding a predicted loss-of-function polypeptide (whether these variations are homozygous or heterozygous in a particular subject) associate with a decreased risk of developing a kidney disease. It is believed that these variant nucleic acid molecules encoding predicted loss-of-function polypeptides have not been associated with decreased risk of kidney disease. Moreover, the identification by the present disclosure of the association between additional variants and gene burden masks indicates that these several genes (i.e., ALDH1L1, ALDOB, G6PC, LRP2, RPL3L, SLC25A45, and SLC7A9) themselves (rather than linkage disequilibrium with variants in another gene) is responsible for a protective effect in kidney disease.

Therefore, subjects that are reference or heterozygous for variant nucleic acid molecules encoding (ALDH1L1, ALDOB, G6PC, LRP2, RPL3L, SLC25A45, and/or SLC7A9) predicted loss-of-function polypeptides may be treated with one or more inhibitors (of ALDH1L1, ALDOB, G6PC, LRP2, RPL3L, SLC25A45, and/or SLC7A9) such that the kidney disease is inhibited or prevented, the symptoms thereof are reduced or prevented, and/or development of symptoms is repressed or prevented. It is also believed that such subjects having a kidney disease may further be treated with therapeutic agents that treat or inhibit the kidney disease.

For purposes of the present disclosure, any particular subject, such as a human, can be categorized as having one of three ALDH1L1 genotypes: i) ALDH1L1 reference; ii) heterozygous for an ALDH1L1 variant nucleic acid molecule encoding an ALDH1L1 predicted loss-of-function polypeptide; or iii) homozygous for an ALDH1L1 variant nucleic acid molecule encoding an ALDH1L1 predicted loss-of-function polypeptide. A subject is ALDH1L1 reference when the subject does not have a copy of an ALDH1L1 variant nucleic acid molecule encoding an ALDH1L1 predicted loss-of-function polypeptide. A subject is heterozygous for an ALDH1L1 variant nucleic acid molecule encoding an ALDH1L1 predicted loss-of-function polypeptide when the subject has a single copy of an ALDH1L1 variant nucleic acid molecule encoding an ALDH1L1 predicted loss-of-function polypeptide. An ALDH1L1 variant nucleic acid molecule encoding an ALDH1L1 predicted loss-of-function polypeptide is any nucleic acid molecule (such as, a genomic nucleic acid molecule, an mRNA molecule, or a cDNA molecule) encoding a ALDH1L1 polypeptide having a partial loss-of-function, a complete loss-of-function, a predicted partial loss-of-function, or a predicted complete loss-of-function. A subject who has an ALDH1L1 polypeptide having a partial loss-of-function (or predicted partial loss-of-function) is hypomorphic for ALDH1L1. A subject is homozygous for an ALDH1L1 variant nucleic acid molecule encoding an ALDH1L1 predicted loss-of-function polypeptide when the subject has two copies (same or different) of an ALDH1L1 variant nucleic acid molecule encoding an ALDH1L1 predicted loss-of-function polypeptide.

For purposes of the present disclosure, any particular subject, such as a human, can be categorized as having one of three ALDOB genotypes: i) ALDOB reference; ii) heterozygous for an ALDOB variant nucleic acid molecule encoding an ALDOB predicted loss-of-function polypeptide; or iii) homozygous for an ALDOB variant nucleic acid molecule encoding an ALDOB predicted loss-of-function polypeptide. A subject is ALDOB reference when the subject does not have a copy of an ALDOB variant nucleic acid molecule encoding an ALDOB predicted loss-of-function polypeptide. A subject is heterozygous for an ALDOB variant nucleic acid molecule encoding an ALDOB predicted loss-of-function polypeptide when the subject has a single copy of an ALDOB variant nucleic acid molecule encoding an ALDOB predicted loss-of-function polypeptide. An ALDOB variant nucleic acid molecule encoding an ALDOB predicted loss-of-function polypeptide is any nucleic acid molecule (such as, a genomic nucleic acid molecule, an mRNA molecule, or a cDNA molecule) encoding a variant ALDOB polypeptide having a partial loss-of-function, a complete loss-of-function, a predicted partial loss-of-function, or a predicted complete loss-of-function. A subject who has an ALDOB polypeptide having a partial loss-of-function (or predicted partial loss-of-function) is hypomorphic for ALDOB. A subject is homozygous for an ALDOB variant nucleic acid molecule encoding an ALDOB predicted loss-of-function polypeptide when the subject has two copies (same or different) of an ALDOB variant nucleic acid molecule encoding an ALDOB predicted loss-of-function polypeptide.

For purposes of the present disclosure, any particular subject, such as a human, can be categorized as having one of three G6PC genotypes: i) G6PC reference; ii) heterozygous for a G6PC variant nucleic acid molecule encoding a G6PC predicted loss-of-function polypeptide; or iii) homozygous for a G6PC variant nucleic acid molecule encoding a G6PC predicted loss-of-function polypeptide. A subject is G6PC reference when the subject does not have a copy of a G6PC variant nucleic acid molecule encoding a G6PC predicted loss-of-function polypeptide. A subject is heterozygous for a G6PC variant nucleic acid molecule encoding a G6PC predicted loss-of-function polypeptide when the subject has a single copy of a G6PC variant nucleic acid molecule encoding a G6PC predicted loss-of-function polypeptide. A G6PC variant nucleic acid molecule encoding a G6PC predicted loss-of-function polypeptide is any nucleic acid molecule (such as, a genomic nucleic acid molecule, an mRNA molecule, or a cDNA molecule) encoding a G6PC polypeptide having a partial loss-of-function, a complete loss-of-function, a predicted partial loss-of-function, or a predicted complete loss-of-function. A subject who has a G6PC polypeptide having a partial loss-of-function (or predicted partial loss-of-function) is hypomorphic for G6PC. A subject is homozygous for a G6PC variant nucleic acid molecule encoding a G6PC predicted loss-of-function polypeptide when the subject has two copies (same or different) of a G6PC variant nucleic acid molecule encoding a G6PC predicted loss-of-function polypeptide.

For purposes of the present disclosure, any particular subject, such as a human, can be categorized as having one of three LRP2 genotypes: i) LRP2 reference; ii) heterozygous for an LRP2 variant nucleic acid molecule encoding an LRP2 predicted loss-of-function polypeptide; or iii) homozygous for an LRP2 variant nucleic acid molecule encoding an LRP2 predicted loss-of-function polypeptide. A subject is LRP2 reference when the subject does not have a copy of an LRP2 variant nucleic acid molecule encoding an LRP2 predicted loss-of-function polypeptide. A subject is heterozygous for an LRP2 variant nucleic acid molecule encoding an LRP2 predicted loss-of-function polypeptide when the subject has a single copy of an LRP2 variant nucleic acid molecule encoding an LRP2 predicted loss-of-function polypeptide. An LRP2 variant nucleic acid molecule encoding an LRP2 predicted loss-of-function polypeptide is any nucleic acid molecule (such as, a genomic nucleic acid molecule, an mRNA molecule, or a cDNA molecule) encoding an LRP2 polypeptide having a partial loss-of-function, a complete loss-of-function, a predicted partial loss-of-function, or a predicted complete loss-of-function. A subject who has an LRP2 polypeptide having a partial loss-of-function (or predicted partial loss-of-function) is hypomorphic for LRP2. A subject is homozygous for an LRP2 variant nucleic acid molecule encoding an LRP2 predicted loss-of-function polypeptide when the subject has two copies (same or different) of an LRP2 variant nucleic acid molecule encoding an LRP2 predicted loss-of-function polypeptide.

For purposes of the present disclosure, any particular subject, such as a human, can be categorized as having one of three RPL3L genotypes: i) RPL3L reference; ii) heterozygous for an RPL3L variant nucleic acid molecule encoding an RPL3L predicted loss-of-function polypeptide; or iii) homozygous for an RPL3L variant nucleic acid molecule encoding an RPL3L predicted loss-of-function polypeptide. A subject is RPL3L reference when the subject does not have a copy of an RPL3L variant nucleic acid molecule encoding an RPL3L predicted loss-of-function polypeptide. A subject is heterozygous for an RPL3L variant nucleic acid molecule encoding an RPL3L predicted loss-of-function polypeptide when the subject has a single copy of an RPL3L variant nucleic acid molecule encoding an RPL3L predicted loss-of-function polypeptide. An RPL3L variant nucleic acid molecule encoding an RPL3L predicted loss-of-function polypeptide is any nucleic acid molecule (such as, a genomic nucleic acid molecule, an mRNA molecule, or a cDNA molecule) encoding a RPL3L polypeptide having a partial loss-of-function, a complete loss-of-function, a predicted partial loss-of-function, or a predicted complete loss-of-function. A subject who has an RPL3L polypeptide having a partial loss-of-function (or predicted partial loss-of-function) is hypomorphic for RPL3L. A subject is homozygous for an RPL3L variant nucleic acid molecule encoding an RPL3L predicted loss-of-function polypeptide when the subject has two copies (same or different) of an RPL3L variant nucleic acid molecule encoding an RPL3L predicted loss-of-function polypeptide.

For purposes of the present disclosure, any particular subject, such as a human, can be categorized as having one of three SLC25A45 genotypes: i) SLC25A45 reference; ii) heterozygous for an SLC25A45 variant nucleic acid molecule encoding an SLC25A45 predicted loss-of-function polypeptide; or iii) homozygous for an SLC25A45 variant nucleic acid molecule encoding an SLC25A45 predicted loss-of-function polypeptide. A subject is SLC25A45 reference when the subject does not have a copy of an SLC25A45 variant nucleic acid molecule encoding an SLC25A45 predicted loss-of-function polypeptide. A subject is heterozygous for an SLC25A45 variant nucleic acid molecule encoding an SLC25A45 predicted loss-of-function polypeptide when the subject has a single copy of an SLC25A45 variant nucleic acid molecule encoding an SLC25A45 predicted loss-of-function polypeptide. An SLC25A45 variant nucleic acid molecule encoding an SLC25A45 predicted loss-of-function polypeptide is any nucleic acid molecule (such as, a genomic nucleic acid molecule, an mRNA molecule, or a cDNA molecule) encoding an SLC25A45 polypeptide having a partial loss-of-function, a complete loss-of-function, a predicted partial loss-of-function, or a predicted complete loss-of-function. A subject who has an SLC25A45 polypeptide having a partial loss-of-function (or predicted partial loss-of-function) is hypomorphic for SLC25A45. A subject is homozygous for an SLC25A45 variant nucleic acid molecule encoding an SLC25A45 predicted loss-of-function polypeptide when the subject has two copies (same or different) of an SLC25A45 variant nucleic acid molecules encoding an SLC25A45 predicted loss-of-function polypeptide.

For purposes of the present disclosure, any particular subject, such as a human, can be categorized as having one of three SLC7A9 genotypes: i) SLC7A9 reference; ii) heterozygous for an SLC7A9 variant nucleic acid molecule encoding an SLC7A9 predicted loss-of-function polypeptide; or iii) homozygous for an SLC7A9 variant nucleic acid molecule encoding an SLC7A9 predicted loss-of-function polypeptide. A subject is SLC7A9 reference when the subject does not have a copy of an SLC7A9 variant nucleic acid molecule encoding an SLC7A9 predicted loss-of-function polypeptide. A subject is heterozygous for an SLC7A9 variant nucleic acid molecule encoding an SLC7A9 predicted loss-of-function polypeptide when the subject has a single copy of an SLC7A9 variant nucleic acid molecule encoding an SLC7A9 predicted loss-of-function polypeptide. An SLC7A9 variant nucleic acid molecule encoding an SLC7A9 predicted loss-of-function polypeptide is any nucleic acid molecule (such as, a genomic nucleic acid molecule, an mRNA molecule, or a cDNA molecule) encoding a SLC7A9 polypeptide having a partial loss-of-function, a complete loss-of-function, a predicted partial loss-of-function, or a predicted complete loss-of-function. A subject who has an SLC7A9 polypeptide having a partial loss-of-function (or predicted partial loss-of-function) is hypomorphic for SLC7A9. A subject is homozygous for an SLC7A9 variant nucleic acid molecule encoding an SLC7A9 predicted loss-of-function polypeptide when the subject has two copies (same or different) of an SLC7A9 variant nucleic acid molecule encoding an SLC7A9 predicted loss-of-function polypeptide.

For subjects that are genotyped or determined to be ALDH1L1 reference, such subjects have an increased risk of developing a kidney disease, such as chronic kidney disease, a kidney stone, chronic glomerulonephritis, nephrosis, nephronophthisis, chronic interstitial nephritis, and/or nephrosclerosis. For subjects that are genotyped or determined to be either ALDH1L1 reference or heterozygous for an ALDH1L1 variant nucleic acid molecule encoding an ALDH1L1 predicted loss-of-function polypeptide, such subjects or subjects can be treated with an ALDH1L1 inhibitor.

For subjects that are genotyped or determined to be ALDOB reference, such subjects have an increased risk of developing a kidney disease, such as chronic kidney disease, a kidney stone, chronic glomerulonephritis, nephrosis, nephronophthisis, chronic interstitial nephritis, and/or nephrosclerosis. For subjects that are genotyped or determined to be either ALDOB reference or heterozygous for an ALDOB variant nucleic acid molecule encoding an ALDOB predicted loss-of-function polypeptide, such subjects or subjects can be treated with an ALDOB inhibitor.

For subjects that are genotyped or determined to be G6PC reference, such subjects have an increased risk of developing a kidney disease, such as chronic kidney disease, a kidney stone, chronic glomerulonephritis, nephrosis, nephronophthisis, chronic interstitial nephritis, and/or nephrosclerosis. For subjects that are genotyped or determined to be either G6PC reference or heterozygous for a G6PC variant nucleic acid molecule encoding a G6PC predicted loss-of-function polypeptide, such subjects or subjects can be treated with a G6PC inhibitor.

For subjects that are genotyped or determined to be LRP2 reference, such subjects have an increased risk of developing a kidney disease, such as chronic kidney disease, a kidney stone, chronic glomerulonephritis, nephrosis, nephronophthisis, chronic interstitial nephritis, and/or nephrosclerosis. For subjects that are genotyped or determined to be either LRP2 reference or heterozygous for an LRP2 variant nucleic acid molecule encoding an LRP2 predicted loss-of-function polypeptide, such subjects or subjects can be treated with an LRP2 inhibitor.

For subjects that are genotyped or determined to be SLC25A45 reference, such subjects have an increased risk of developing a kidney disease, such as chronic kidney disease, a kidney stone, chronic glomerulonephritis, nephrosis, nephronophthisis, chronic interstitial nephritis, and/or nephrosclerosis. For subjects that are genotyped or determined to be either SLC25A45 reference or heterozygous for an SLC25A45 variant nucleic acid molecule encoding an SLC25A45 predicted loss-of-function polypeptide, such subjects or subjects can be treated with an SLC25A45 inhibitor.

For subjects that are genotyped or determined to be SLC7A9 reference, such subjects have an increased risk of developing a kidney disease, such as chronic kidney disease, a kidney stone, chronic glomerulonephritis, nephrosis, nephronophthisis, chronic interstitial nephritis, and/or nephrosclerosis. For subjects that are genotyped or determined to be either SLC7A9 reference or heterozygous for an SLC7A9 variant nucleic acid molecule encoding an SLC7A9 predicted loss-of-function polypeptide, such subjects or subjects can be treated with an SLC7A9 inhibitor.

For subjects who are determined to be reference for any combination of ALDH1L1, ALDOB, G6PC, LRP2, RPL3L, SLC7A9, and/or SLC25A45, such subjects have an increased risk of developing a kidney disease, such as chronic kidney disease, a kidney stone, chronic glomerulonephritis, nephrosis, nephronophthisis, chronic interstitial nephritis, and/or nephrosclerosis. For subjects that are genotyped or determined to be either reference for any combination of ALDH1L1, ALDOB, G6PC, LRP2, RPL3L, SLC7A9, and/or SLC25A45 or heterozygous for any combination of ALDH1L1, ALDOB, G6PC, LRP2, RPL3L, SLC7A9, and/or SLC25A45 variant nucleic acid molecules encoding corresponding predicted loss-of-function polypeptides, such subjects or subjects can be treated with a corresponding combination of inhibitors of ALDH1L1, ALDOB, G6PC, LRP2, RPL3L, SLC7A9, and/or SLC25A45.

In any of the embodiments described herein, the subject in whom a kidney disease is prevented by administering one or more of the ALDH1L1 inhibitors, ALDOB inhibitors, G6PC inhibitors, LRP2 inhibitors, RPL3L inhibitors, SLC25A45 inhibitors, and/or SLC7A9 inhibitors can be anyone at risk for developing a kidney disease including, but not limited to, chronic kidney disease, a kidney stone, chronic glomerulonephritis, nephrosis, nephronophthisis, chronic interstitial nephritis, and/or nephrosclerosis. In addition, in some embodiments, any subject can be at risk of developing a kidney disease. In some embodiments, administering one or more of the ALDH1L1 inhibitors, ALDOB inhibitors, G6PC inhibitors, LRP2 inhibitors, RPL3L inhibitors, SLC25A45 inhibitors, and/or SLC7A9 inhibitors may be carried out to prevent development of an additional kidney disease in a subject who has already had a kidney disease.

In any of the embodiments described herein, the ALDH1L1 variant nucleic acid molecules encoding an ALDH1L1 predicted loss-of-function polypeptide can be any nucleic acid molecule (such as, for example, genomic nucleic acid molecule, mRNA molecule, or cDNA molecule) encoding an ALDH1L1 polypeptide having a partial loss-of-function, a complete loss-of-function, a predicted partial loss-of-function, or a predicted complete loss-of-function. In some embodiments, the ALDH1L1 variant nucleic acid molecule encoding an ALDH1L1 predicted loss-of-function polypeptide is associated with a reduced in vitro response to ALDH1L1 ligands compared with reference ALDH1L1. In some embodiments, the ALDH1L1 variant nucleic acid molecule encoding an ALDH1L1 predicted loss-of-function polypeptide is an ALDH1L1 variant that results or is predicted to result in a premature truncation of an ALDH1L1 polypeptide compared to the human reference genome sequence. In some embodiments, the ALDH1L1 variant nucleic acid molecule encoding an ALDH1L1 predicted loss-of-function polypeptide is a variant that is predicted to be damaging by in vitro prediction algorithms such as Polyphen, SIFT, or similar algorithms. In some embodiments, the ALDH1L1 variant nucleic acid molecule encoding an ALDH1L1 predicted loss-of-function polypeptide is a variant that causes or is predicted to cause a nonsynonymous amino-acid substitution in ALDH1L1 and whose allele frequency is less than 1/100 alleles in the population from which the subject is selected. In some embodiments, the ALDH1L1 variant nucleic acid molecule encoding an ALDH1L1 predicted loss-of-function polypeptide is any rare missense variant (allele frequency <0.1%; or 1 in 1,000 alleles), or any splice-site, stop-gain, start-loss, stop-loss, frameshift, or in-frame indel, or other frameshift ALDH1L1 variant.

In any of the embodiments described herein, the ALDH1L1 predicted loss-of-function polypeptide can be any ALDH1L1 polypeptide having a partial loss-of-function, a complete loss-of-function, a predicted partial loss-of-function, or a predicted complete loss-of-function.

In any of the embodiments described herein, the ALDH1L1 variant nucleic acid molecule encoding an ALDH1L1 predicted loss-of-function polypeptide can include variations at positions of chromosome 3 using the nucleotide sequence of the ALDH1L1 reference genomic nucleic acid molecule (SEQ ID NO:1; chr3:126,103,562-126,180,802 in the GRCh38/hg38 human genome assembly) as a reference sequence.

Numerous genetic variants in ALDH1L1 exist which cause subsequent changes in the ALDH1L1 polypeptide sequence including, but not limited to, rs143122118 (3:126160912:C:T) and rs775766256 (3:126110055:G:C) (according to the GRCh38/hg38 human genome assembly).

In any of the embodiments described herein, the ALDOB variant nucleic acid molecule encoding an ALDOB predicted loss-of-function polypeptide can be any nucleic acid molecule (such as, for example, genomic nucleic acid molecule, mRNA molecule, or cDNA molecule) encoding an ALDOB variant polypeptide having a partial loss-of-function, a complete loss-of-function, a predicted partial loss-of-function, or a predicted complete loss-of-function. In some embodiments, the ALDOB variant nucleic acid molecule encoding an ALDOB predicted loss-of-function polypeptide is associated with a reduced in vitro response to ALDOB ligands compared with reference ALDOB. In some embodiments, the ALDOB variant nucleic acid molecule encoding an ALDOB predicted loss-of-function polypeptide is an ALDOB variant that results or is predicted to result in a premature truncation of an ALDOB polypeptide compared to the human reference genome sequence. In some embodiments, the ALDOB variant nucleic acid molecule encoding an ALDOB predicted loss-of-function polypeptide is a variant that is predicted to be damaging by in vitro prediction algorithms such as Polyphen, SIFT, or similar algorithms. In some embodiments, the ALDOB variant nucleic acid molecule encoding an ALDOB predicted loss-of-function polypeptide is a variant that causes or is predicted to cause a nonsynonymous amino-acid substitution in ALDOB and whose allele frequency is less than 1/100 alleles in the population from which the subject is selected. In some embodiments, the ALDOB variant nucleic acid molecule encoding an ALDOB predicted loss-of-function polypeptide is any rare missense variant (allele frequency <0.1%; or 1 in 1,000 alleles), or any splice-site, stop-gain, start-loss, stop-loss, frameshift, or in-frame indel, or other frameshift ALDOB variant.

In any of the embodiments described herein, the ALDOB predicted loss-of-function polypeptide can be any ALDOB polypeptide having a partial loss-of-function, a complete loss-of-function, a predicted partial loss-of-function, or a predicted complete loss-of-function.

In any of the embodiments described herein, the ALDOB variant nucleic acid molecule encoding an ALDOB predicted loss-of-function polypeptide can include variations at positions of chromosome 9 using the nucleotide sequence of the ALDOB reference genomic nucleic acid molecule (SEQ ID NO:73; chr9:101,421,439-101,449,664 in the GRCh38/hg38 human genome assembly) as a reference sequence.

Numerous genetic variants in ALDOB exist which cause subsequent changes in the ALDOB polypeptide sequence including, but not limited to, rs1800546 (9:101427574:C:G), rs201397971 (9:101429913:G:A), and 9:101426570:C:A (according to the GRCh38/hg38 human genome assembly).

In any of the embodiments described herein, the G6PC variant nucleic acid molecule encoding a G6PC predicted loss-of-function polypeptide can be any nucleic acid molecule (such as, for example, genomic nucleic acid molecule, mRNA molecule, or cDNA molecule) encoding a G6PC variant polypeptide having a partial loss-of-function, a complete loss-of-function, a predicted partial loss-of-function, or a predicted complete loss-of-function. In some embodiments, the G6PC variant nucleic acid molecule encoding a G6PC predicted loss-of-function polypeptide is associated with a reduced in vitro response to G6PC ligands compared with reference G6PC. In some embodiments, the G6PC variant nucleic acid molecule encoding a G6PC predicted loss-of-function polypeptide is a G6PC variant that results or is predicted to result in a premature truncation of a G6PC polypeptide compared to the human reference genome sequence. In some embodiments, the G6PC variant nucleic acid molecule encoding a G6PC predicted loss-of-function polypeptide is a variant that is predicted to be damaging by in vitro prediction algorithms such as Polyphen, SIFT, or similar algorithms. In some embodiments, the G6PC variant nucleic acid molecule encoding a G6PC predicted loss-of-function polypeptide is a variant that causes or is predicted to cause a nonsynonymous amino-acid substitution in G6PC and whose allele frequency is less than 1/100 alleles in the population from which the subject is selected. In some embodiments, the G6PC variant nucleic acid molecule encoding a G6PC predicted loss-of-function polypeptide is any rare missense variant (allele frequency <0.1%; or 1 in 1,000 alleles), or any splice-site, stop-gain, start-loss, stop-loss, frameshift, or in-frame indel, or other frameshift G6PC variant.

In any of the embodiments described herein, the G6PC predicted loss-of-function polypeptide can be any G6PC polypeptide having a partial loss-of-function, a complete loss-of-function, a predicted partial loss-of-function, or a predicted complete loss-of-function.

In any of the embodiments described herein, the G6PC variant nucleic acid molecule encoding a G6PC predicted loss-of-function polypeptide can include variations at positions of chromosome 17 using the nucleotide sequence of the G6PC reference genomic nucleic acid molecule (SEQ ID NO:102; chr17:42,900,799-42,914,438 in the GRCh38/hg38 human genome assembly) as a reference sequence.

Numerous genetic variants in G6PC exist which cause subsequent changes in the G6PC polypeptide sequence including, but not limited to, rs1251265849 (17:42900952:TC:T), rs746978011 (17:42911417:G:T), rs1801175 (17:42903947:C:T), rs1157674386 (17:42903998:C:T), rs80356487 (17:42911391:C:T), and 17:42900910:G:C (according to the GRCh38/hg38 human genome assembly).

In any of the embodiments described herein, the LRP2 variant nucleic acid molecule encoding an LRP2 predicted loss-of-function polypeptide can be any nucleic acid molecule (such as, for example, genomic nucleic acid molecule, mRNA molecule, or cDNA molecule) encoding an LRP2 variant polypeptide having a partial loss-of-function, a complete loss-of-function, a predicted partial loss-of-function, or a predicted complete loss-of-function. In some embodiments, the LRP2 variant nucleic acid molecule encoding an LRP2 predicted loss-of-function polypeptide is associated with a reduced in vitro response to LRP2 ligands compared with reference LRP2. In some embodiments, the LRP2 variant nucleic acid molecule encoding an LRP2 predicted loss-of-function polypeptide is an LRP2 variant that results or is predicted to result in a premature truncation of an LRP2 polypeptide compared to the human reference genome sequence. In some embodiments, the LRP2 variant nucleic acid molecule encoding an LRP2 predicted loss-of-function polypeptide is a variant that is predicted to be damaging by in vitro prediction algorithms such as Polyphen, SIFT, or similar algorithms. In some embodiments, the LRP2 variant nucleic acid molecule encoding an LRP2 predicted loss-of-function polypeptide is a variant that causes or is predicted to cause a nonsynonymous amino-acid substitution in LRP2 and whose allele frequency is less than 1/100 alleles in the population from which the subject is selected. In some embodiments, the LRP2 variant nucleic acid molecule encoding an LRP2 predicted loss-of-function polypeptide is any rare missense variant (allele frequency <0.1%; or 1 in 1,000 alleles), or any splice-site, stop-gain, start-loss, stop-loss, frameshift, or in-frame indel, or other frameshift LRP2 variant.

In any of the embodiments described herein, the LRP2 predicted loss-of-function polypeptide can be any LRP2 polypeptide having a partial loss-of-function, a complete loss-of-function, a predicted partial loss-of-function, or a predicted complete loss-of-function.

In any of the embodiments described herein, the LRP2 variant nucleic acid molecule encoding an LRP2 predicted loss-of-function polypeptide can include variations at positions of chromosome 2 using the nucleotide sequence of the LRP2 reference genomic nucleic acid molecule (SEQ ID NO:120; chr2:169,127,109-169,362,534 in the GRCh38/hg38 human genome assembly) as a reference sequence.

Numerous genetic variants in LRP2 exist which cause subsequent changes in the LRP2 polypeptide sequence including, but not limited to, rs200475391 (2:169170643:T:A), rs34355135 (2:169173147:C:T), rs200475391 (2:169170643:T:A), 2:169174132:A:G, and 2:169174132:A:G (according to the GRCh38/hg38 human genome assembly).

In any of the embodiments described herein, the RPL3L variant nucleic acid molecule encoding an RPL3L predicted loss-of-function polypeptide can be any nucleic acid molecule (such as, for example, genomic nucleic acid molecule, mRNA molecule, or cDNA molecule) encoding an RPL3L variant polypeptide having a partial loss-of-function, a complete loss-of-function, a predicted partial loss-of-function, or a predicted complete loss-of-function. In some embodiments, the RPL3L variant nucleic acid molecule encoding an RPL3L predicted loss-of-function polypeptide is associated with a reduced in vitro response to RPL3L ligands compared with reference RPL3L. In some embodiments, the RPL3L variant nucleic acid molecule encoding an RPL3L predicted loss-of-function polypeptide is an RPL3L variant that results or is predicted to result in a premature truncation of an RPL3L polypeptide compared to the human reference genome sequence. In some embodiments, the RPL3L variant nucleic acid molecule encoding an RPL3L predicted loss-of-function polypeptide is a variant that is predicted to be damaging by in vitro prediction algorithms such as Polyphen, SIFT, or similar algorithms. In some embodiments, the RPL3L variant nucleic acid molecule encoding an RPL3L predicted loss-of-function polypeptide is a variant that causes or is predicted to cause a nonsynonymous amino-acid substitution in RPL3L and whose allele frequency is less than 1/100 alleles in the population from which the subject is selected. In some embodiments, the RPL3L variant nucleic acid molecule encoding an RPL3L predicted loss-of-function polypeptide is any rare missense variant (allele frequency <0.1%; or 1 in 1,000 alleles), or any splice-site, stop-gain, start-loss, stop-loss, frameshift, or in-frame indel, or other frameshift RPL3L variant.

In any of the embodiments described herein, the RPL3L predicted loss-of-function polypeptide can be any RPL3L polypeptide having a partial loss-of-function, a complete loss-of-function, a predicted partial loss-of-function, or a predicted complete loss-of-function.

In any of the embodiments described herein, the RPL3L variant nucleic acid molecule encoding an RPL3L predicted loss-of-function polypeptide can include variations at positions of chromosome 16 using the nucleotide sequence of the RPL3L reference genomic nucleic acid molecule (SEQ ID NO:144; chr16:1,943,974-1,954,689 in the GRCh38/hg38 human genome assembly) as a reference sequence.

Numerous genetic variants in RPL3L exist which cause subsequent changes in the RPL3L polypeptide sequence including, but not limited to, rs113956264 (16:1947003:C:T) and rs140185678 (16:1953015:G:A) (according to the GRCh38/hg38 human genome assembly).

In any of the embodiments described herein, the SLC25A45 variant nucleic acid molecule encoding an SLC25A45 predicted loss-of-function polypeptide can be any nucleic acid molecule (such as, for example, genomic nucleic acid molecule, mRNA molecule, or cDNA molecule) encoding an SLC25A45 variant polypeptide having a partial loss-of-function, a complete loss-of-function, a predicted partial loss-of-function, or a predicted complete loss-of-function. In some embodiments, the SLC25A45 variant nucleic acid molecule encoding an SLC25A45 predicted loss-of-function polypeptide is associated with a reduced in vitro response to SLC25A45 ligands compared with reference SLC25A45. In some embodiments, the SLC25A45 variant nucleic acid molecule encoding an SLC25A45 predicted loss-of-function polypeptide is an SLC25A45 variant that results or is predicted to result in a premature truncation of an SLC25A45 polypeptide compared to the human reference genome sequence. In some embodiments, the SLC25A45 variant nucleic acid molecule encoding an SLC25A45 predicted loss-of-function polypeptide is a variant that is predicted to be damaging by in vitro prediction algorithms such as Polyphen, SIFT, or similar algorithms. In some embodiments, the SLC25A45 variant nucleic acid molecule encoding an SLC25A45 predicted loss-of-function polypeptide is a variant that causes or is predicted to cause a nonsynonymous amino-acid substitution in SLC25A45 and whose allele frequency is less than 1/100 alleles in the population from which the subject is selected. In some embodiments, the SLC25A45 variant nucleic acid molecule encoding an SLC25A45 predicted loss-of-function polypeptide is any rare missense variant (allele frequency <0.1%; or 1 in 1,000 alleles), or any splice-site, stop-gain, start-loss, stop-loss, frameshift, or in-frame indel, or other frameshift SLC25A45 variant.

In any of the embodiments described herein, the SLC25A45 predicted loss-of-function polypeptide can be any SLC25A45 polypeptide having a partial loss-of-function, a complete loss-of-function, a predicted partial loss-of-function, or a predicted complete loss-of-function.

In any of the embodiments described herein, the SLC25A45 variant nucleic acid molecule encoding an SLC25A45 predicted loss-of-function polypeptide can include variations at positions of chromosome 11 using the nucleotide sequence of the SLC25A45 reference genomic nucleic acid molecule (SEQ ID NO:159; chr11:65,375,192-65,382,671 in the GRCh38/hg38 human genome assembly) as a reference sequence.

Numerous genetic variants in SLC25A45 exist which cause subsequent changes in the SLC25A45 polypeptide sequence including, but not limited to, rs34400381 (11:65376421:G:A) and rs78829599 (11:65379542:C:T) (according to the GRCh38/hg38 human genome assembly).

In any of the embodiments described herein, the SLC7A9 variant nucleic acid molecule encoding an SLC7A9 predicted loss-of-function polypeptide can be any nucleic acid molecule (such as, for example, genomic nucleic acid molecule, mRNA molecule, or cDNA molecule) encoding an SLC7A9 variant polypeptide having a partial loss-of-function, a complete loss-of-function, a predicted partial loss-of-function, or a predicted complete loss-of-function. In some embodiments, the SLC7A9 variant nucleic acid molecule encoding an SLC7A9 predicted loss-of-function polypeptide is associated with a reduced in vitro response to SLC7A9 ligands compared with reference SLC7A9. In some embodiments, the SLC7A9 variant nucleic acid molecule encoding an SLC7A9 predicted loss-of-function polypeptide is an SLC7A9 variant that results or is predicted to result in a premature truncation of an SLC7A9 polypeptide compared to the human reference genome sequence. In some embodiments, the SLC7A9 variant nucleic acid molecule encoding an SLC7A9 predicted loss-of-function polypeptide is a variant that is predicted to be damaging by in vitro prediction algorithms such as Polyphen, SIFT, or similar algorithms. In some embodiments, the SLC7A9 variant nucleic acid molecule encoding an SLC7A9 predicted loss-of-function polypeptide is a variant that causes or is predicted to cause a nonsynonymous amino-acid substitution in SLC7A9 and whose allele frequency is less than 1/100 alleles in the population from which the subject is selected. In some embodiments, the SLC7A9 variant nucleic acid molecule encoding an SLC7A9 predicted loss-of-function polypeptide is any rare missense variant (allele frequency <0.1%; or 1 in 1,000 alleles), or any splice-site, stop-gain, start-loss, stop-loss, frameshift, or in-frame indel, or other frameshift SLC7A9 variant.

In any of the embodiments described herein, the SLC7A9 predicted loss-of-function polypeptide can be any SLC7A9 polypeptide having a partial loss-of-function, a complete loss-of-function, a predicted partial loss-of-function, or a predicted complete loss-of-function.

In any of the embodiments described herein, the SLC7A9 variant nucleic acid molecule encoding an SLC7A9 predicted loss-of-function polypeptide can include variations at positions of chromosome 19 using the nucleotide sequence of the SLC7A9 reference genomic nucleic acid molecule (SEQ ID NO:205; chr19:32,830,511-32,869,767 in the GRCh38/hg38 human genome assembly) as a reference sequence.

Numerous genetic variants in SLC7A9 exist which cause subsequent changes in the SLC7A9 polypeptide sequence including, but not limited to, rs79389353 (19:32862521:C:T) and rs121908480 (19:32864261:C:T) (according to the GRCh38/hg38 human genome assembly).

Any one or more (i.e., any combination) of the ALDH1L1, ALDOB, G6PC, LRP2, RPL3L, SLC25A45, or SLC7A9 variant nucleic acid molecules encoding ALDH1L1, ALDOB, G6PC, LRP2, RPL3L, SLC25A45, or SLC7A9 predicted loss-of-function polypeptides can be used within any of the methods described herein to determine whether a subject has an increased risk of developing kidney disease. The combinations of particular variants can form a mask used for statistical analysis of the particular correlation of ALDH1L1, ALDOB, G6PC, LRP2, RPL3L, SLC25A45, and/or SLC7A9 and decreased risk of developing kidney disease.

In any of the embodiments described herein, the kidney disease is chronic kidney disease, a kidney stone, chronic glomerulonephritis, nephrosis, nephronophthisis, chronic interstitial nephritis, and/or nephrosclerosis. In some embodiments, the kidney disease is chronic kidney disease. In some embodiments, the kidney disease is a kidney stone. In some embodiments, the kidney disease is chronic glomerulonephritis. In some embodiments, the kidney disease is nephrosis. In some embodiments, the kidney disease is nephronophthisis. In some embodiments, the kidney disease is chronic interstitial nephritis. In some embodiments, the kidney disease is nephrosclerosis.

Other kidney disease include, but are not limited to, acquired cystic disease, acute (postinfectious) glomerulonephritis, acute infectious interstitial nephritis, acute interstitial nephritis, acute pyelonephritis, acute renal failure, acute transplant failure, acute tubular necrosis, adult polycystic kidney disease, AL amyloid, analgesic nephrosis, anti-glomerular basement membrane disease (Goodpasture's Syndrome), asymptomatic hematuria, asymptomatic proteinuria, autosomal dominant polycystic kidney disease, autosomal recessive polycystic kidney disease, Bence Jones cast nephrosis, benign familial hematuria, benign nephrosclerosis and atheromatous embolization, bilateral cortical necrosis, chronic glomerulonephritis, chronic interstitial nephritis, chronic pyelonephritis, chronic renal failure, chronic transplant failure, circulating immune complex nephritis, crescentic glomerulonephritis, cryoglobulinemia, cystic renal dysplasia, diabetic glomerulosclerosis, diabetic nephrosis, dialysis cystic disease, drug induced (allergic) acute interstitial nephritis, ectopic kidney, Fabry's disease, familial juvenile nephronophthisis-medullary cystic disease complex, focal glomerulosclerosis (segmental hyalinosis), glomerulocystic disease, glomerulonephritis, glomerulonephritis associated with bacterial endocarditis, glomerulosclerosis, hemolytic-uremic syndrome, Henoch-Schonlein purpura, hepatitis-associated glomerulonephritis, hereditary nephritis (Alport syndrome), horseshoe kidney, hydronephrosis, IgA nephrosis, infantile polycystic kidney disease, ischemic acute tubular necrosis, light-cahin deposit disease, malignant nephrosclerosis, medullary cystic disease, membranoproliferative (mesangiocapillary) glomerulonephritis, membranous glomerulonephritis, membranous nephrosis, mesangial proliferative glomerulonephritis (includes Berger's Disease), minimal change glomerular disease, minimal change nephrotic syndrome, nephritic syndrome, nephroblastoma (Wilms tumor), nephronophthisis (medullary cystic disease complex), nephrotic syndrome, plasma cell dyscrasias (monoclonal immunoglobulin-induced renal damage), polyarteritis nodosa, proteinuria, pyelonephritis, rapidly progressive (crescentic) glomerulonephritis, renal agenesis, renal amyloidosis, renal cell carcinoma, renal dysgenesis, renal dysplasia, renal hypoplasia, renal infection, renal osteodystrophy, renal stones (urolithiasis), renal tubular acidosis, renal vasculitis, renovascular hypertension, scleroderma (progressive systemic sclerosis), secondary acquired glomerulonephritis, simple renal cysts, systemic lupus erythematosus, thin basement membrane nephrosis, thrombotic microangiopathy, thrombotic thrombocytopenic purpura, toxic acute tubular necrosis, tubular defects, tubulointerstitial disease in multiple myeloma, urate nephrosis, urinary obstruction, and vasculitis.

Symptoms of chronic kidney disease include, but are not limited to, nausea, vomiting, loss of appetite, fatigue and weakness, sleep problems, changes urination volume, decreased mental sharpness, muscle twitches and cramps, swelling of feet and ankles, persistent itching, chest pain, fluid build-up around the lining of the heart, shortness of breath, fluid build-up in the lungs, and high blood pressure (hypertension) that's difficult to control.

Symptoms of a kidney stone include, but are not limited to, severe, sharp pain in the side and back, below the ribs, pain that radiates to the lower abdomen and groin, pain that comes in waves and fluctuates in intensity, pain or burning sensation while urinating, pink, red or brown urine, cloudy or foul-smelling urine, a persistent need to urinate, urinating more often than usual or urinating in small amounts, nausea and vomiting, and fever and chills if an infection is present.

Symptoms of chronic glomerulonephritis include, but are not limited to, pink or cola-colored urine from red blood cells in your urine (hematuria), foamy urine due to excess protein (proteinuria), high blood pressure (hypertension), and fluid retention (edema) with swelling evident in the face, hands, feet and abdomen.

Symptoms of nephrosis include, but are not limited to, severe swelling (edema), particularly around eyes and in ankles and feet, foamy urine as a result of excess protein in the urine, weight gain due to fluid retention, fatigue, loss of appetite.

Symptoms of nephronophthisis include, but are not limited to, increased urine production (polyuria), excessive thirst (polydipsia), general weakness, and extreme tiredness (fatigue).

Symptoms of chronic interstitial nephritis include, but are not limited to, blood in the urine, fever, increased or decreased urine output, mental status changes (drowsiness, confusion, coma), nausea, vomiting, rash, swelling of any area of body, and weight gain (from retaining fluid).

Symptoms of nephrosclerosis include, but are not limited to, impaired vision, blood in the urine, loss of weight, and the accumulation of urea and other nitrogenous waste products in the blood, a condition known as uremia.

The present disclosure provides methods of treating a subject having a kidney disease, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of treating a subject having chronic kidney disease, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of treating a subject having a kidney stone, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of treating a subject having chronic glomerulonephritis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of treating a subject having nephrosis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of treating a subject having nephronophthisis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of treating a subject having chronic interstitial nephritis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of treating a subject having nephrosclerosis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of preventing a subject from developing a kidney disease, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of preventing a subject from developing chronic kidney disease, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of preventing a subject from developing a kidney stone, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of preventing a subject from developing chronic glomerulonephritis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of preventing a subject from developing nephrosis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of preventing a subject from developing nephronophthisis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of preventing a subject from developing chronic interstitial nephritis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

The present disclosure also provides methods of preventing a subject from developing nephrosclerosis, the methods comprising administering one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, to the subject.

In some embodiments, the ALDH1L1 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an ALDH1L1 nucleic acid molecule. In some embodiments, the antisense RNA, siRNA, or shRNA hybridizes to a sequence within an ALDH1L1 genomic nucleic acid molecule or mRNA molecule and decreases expression of the ALDH1L1 polypeptide in a cell in the subject. In some embodiments, the ALDH1L1 inhibitor comprises an antisense molecule that hybridizes to an ALDH1L1 genomic nucleic acid molecule or mRNA molecule and decreases expression of the ALDH1L1 polypeptide in a cell in the subject. In some embodiments, the ALDH1L1 inhibitor comprises an siRNA that hybridizes to an ALDH1L1 genomic nucleic acid molecule or mRNA molecule and decreases expression of the ALDH1L1 polypeptide in a cell in the subject. In some embodiments, the ALDH1L1 inhibitor comprises an shRNA that hybridizes to an ALDH1L1 genomic nucleic acid molecule or mRNA molecule and decreases expression of the ALDH1L1 polypeptide in a cell in the subject.

In some embodiments, the ALDOB inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of an ALDOB nucleic acid molecule. In some embodiments, the antisense RNA, siRNA, or shRNA hybridizes to a sequence within an ALDOB genomic nucleic acid molecule or mRNA molecule and decreases expression of the ALDOB polypeptide in a cell in the subject. In some embodiments, the ALDOB inhibitor comprises an antisense molecule that hybridizes to an ALDOB genomic nucleic acid molecule or mRNA molecule and decreases expression of the ALDOB polypeptide in a cell in the subject. In some embodiments, the ALDOB inhibitor comprises an siRNA that hybridizes to an ALDOB genomic nucleic acid molecule or mRNA molecule and decreases expression of the ALDOB polypeptide in a cell in the subject. In some embodiments, the ALDOB inhibitor comprises an shRNA that hybridizes to an ALDOB genomic nucleic acid molecule or mRNA molecule and decreases expression of the ALDOB polypeptide in a cell in the subject.

In some embodiments, the G6PC inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of a G6PC nucleic acid molecule. In some embodiments, the antisense RNA, siRNA, or shRNA hybridizes to a sequence within a G6PC genomic nucleic acid molecule or mRNA molecule and decreases expression of the G6PC polypeptide in a cell in the subject. In some embodiments, the G6PC inhibitor comprises an antisense molecule that hybridizes to a G6PC genomic nucleic acid molecule or mRNA molecule and decreases expression of the G6PC polypeptide in a cell in the subject. In some embodiments, the G6PC inhibitor comprises an siRNA that hybridizes to a G6PC genomic nucleic acid molecule or mRNA molecule and decreases expression of the G6PC polypeptide in a cell in the subject. In some embodiments, the G6PC inhibitor comprises an shRNA that hybridizes to a G6PC genomic nucleic acid molecule or mRNA molecule and decreases expression of the G6PC polypeptide in a cell in the subject.

In some embodiments, the LRP2 inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of an LRP2 nucleic acid molecule. In some embodiments, the antisense RNA, siRNA, or shRNA hybridizes to a sequence within an LRP2 genomic nucleic acid molecule or mRNA molecule and decreases expression of the LRP2 polypeptide in a cell in the subject. In some embodiments, the LRP2 inhibitor comprises an antisense molecule that hybridizes to an LRP2 genomic nucleic acid molecule or mRNA molecule and decreases expression of the LRP2 polypeptide in a cell in the subject. In some embodiments, the LRP2 inhibitor comprises an siRNA that hybridizes to an LRP2 genomic nucleic acid molecule or mRNA molecule and decreases expression of the LRP2 polypeptide in a cell in the subject. In some embodiments, the LRP2 inhibitor comprises an shRNA that hybridizes to an LRP2 genomic nucleic acid molecule or mRNA molecule and decreases expression of the LRP2 polypeptide in a cell in the subject.

In some embodiments, the RPL3L inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of an RPL3L nucleic acid molecule. In some embodiments, the antisense RNA, siRNA, or shRNA hybridizes to a sequence within an RPL3L genomic nucleic acid molecule or mRNA molecule and decreases expression of the RPL3L polypeptide in a cell in the subject. In some embodiments, the RPL3L inhibitor comprises an antisense molecule that hybridizes to an RPL3L genomic nucleic acid molecule or mRNA molecule and decreases expression of the RPL3L polypeptide in a cell in the subject. In some embodiments, the RPL3L inhibitor comprises an siRNA that hybridizes to an RPL3L genomic nucleic acid molecule or mRNA molecule and decreases expression of the RPL3L polypeptide in a cell in the subject. In some embodiments, the RPL3L inhibitor comprises an shRNA that hybridizes to an RPL3L genomic nucleic acid molecule or mRNA molecule and decreases expression of the RPL3L polypeptide in a cell in the subject.

In some embodiments, the SLC25A45 inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of an SLC25A45 nucleic acid molecule. In some embodiments, the antisense RNA, siRNA, or shRNA hybridizes to a sequence within an SLC25A45 genomic nucleic acid molecule or mRNA molecule and decreases expression of the SLC25A45 polypeptide in a cell in the subject. In some embodiments, the SLC25A45 inhibitor comprises an antisense molecule that hybridizes to an SLC25A45 genomic nucleic acid molecule or mRNA molecule and decreases expression of the SLC25A45 polypeptide in a cell in the subject. In some embodiments, the SLC25A45 inhibitor comprises an siRNA that hybridizes to an SLC25A45 genomic nucleic acid molecule or mRNA molecule and decreases expression of the SLC25A45 polypeptide in a cell in the subject. In some embodiments, the SLC25A45 inhibitor comprises an shRNA that hybridizes to an SLC25A45 genomic nucleic acid molecule or mRNA molecule and decreases expression of the SLC25A45 polypeptide in a cell in the subject.

In some embodiments, the SLC7A9 inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of an SLC7A9 nucleic acid molecule. In some embodiments, the antisense RNA, siRNA, or shRNA hybridizes to a sequence within an SLC7A9 genomic nucleic acid molecule or mRNA molecule and decreases expression of the SLC7A9 polypeptide in a cell in the subject. In some embodiments, the SLC7A9 inhibitor comprises an antisense molecule that hybridizes to an SLC7A9 genomic nucleic acid molecule or mRNA molecule and decreases expression of the SLC7A9 polypeptide in a cell in the subject. In some embodiments, the SLC7A9 inhibitor comprises an siRNA that hybridizes to an SLC7A9 genomic nucleic acid molecule or mRNA molecule and decreases expression of the SLC7A9 polypeptide in a cell in the subject. In some embodiments, the SLC7A9 inhibitor comprises an shRNA that hybridizes to an SLC7A9 genomic nucleic acid molecule or mRNA molecule and decreases expression of the SLC7A9 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (1), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

### Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the ALDH1L1 inhibitor comprises a nuclease agent that induces one or more nicks or double-strand breaks at a recognition sequence(s) or a DNA-binding protein that binds to a recognition sequence within an ALDH1L1 genomic nucleic acid molecule. The recognition sequence can be located within a coding region of the ALDH1L1 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the ALDH1L1 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

In some embodiments, the ALDOB inhibitor comprises a nuclease agent that induces one or more nicks or double-strand breaks at a recognition sequence(s) or a DNA-binding protein that binds to a recognition sequence within an ALDOB genomic nucleic acid molecule. The recognition sequence can be located within a coding region of the ALDOB gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the ALDOB gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

In some embodiments, the G6PC inhibitor comprises a nuclease agent that induces one or more nicks or double-strand breaks at a recognition sequence(s) or a DNA-binding protein that binds to a recognition sequence within a G6PC genomic nucleic acid molecule. The recognition sequence can be located within a coding region of the G6PC gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the G6PC gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

In some embodiments, the LRP2 inhibitor comprises a nuclease agent that induces one or more nicks or double-strand breaks at a recognition sequence(s) or a DNA-binding protein that binds to a recognition sequence within an LRP2 genomic nucleic acid molecule. The recognition sequence can be located within a coding region of the LRP2 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the LRP2 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

In some embodiments, the RPL3L inhibitor comprises a nuclease agent that induces one or more nicks or double-strand breaks at a recognition sequence(s) or a DNA-binding protein that binds to a recognition sequence within an RPL3L genomic nucleic acid molecule. The recognition sequence can be located within a coding region of the RPL3L gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the RPL3L gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

In some embodiments, the SLC25A45 inhibitor comprises a nuclease agent that induces one or more nicks or double-strand breaks at a recognition sequence(s) or a DNA-binding protein that binds to a recognition sequence within an SLC25A45 genomic nucleic acid molecule. The recognition sequence can be located within a coding region of the SLC25A45 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the SLC25A45 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

In some embodiments, the SLC7A9 inhibitor comprises a nuclease agent that induces one or more nicks or double-strand breaks at a recognition sequence(s) or a DNA-binding protein that binds to a recognition sequence within an SLC7A9 genomic nucleic acid molecule. The recognition sequence can be located within a coding region of the SLC7A9 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the SLC7A9 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify one or more of ALDH1L1, ALDOB, G6PC, LRP2, RPL3L, SLC25A45, and/or SLC7A9 genomic nucleic acid molecules within a cell. The methods and compositions disclosed herein can employ CRISPR-Cas systems by utilizing CRISPR complexes (comprising a guide RNA (gRNA) complexed with a Cas protein) for site-directed cleavage of any one of ALDH1L1, ALDOB, G6PC, LRP2, RPL3L, SLC25A45, and/or SLC7A9 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in any one of ALDH1L1, ALDOB, G6PC, LRP2, RPL3L, SLC25A45, or SLC7A9 genomic nucleic acid molecules or it can be a nickase that creates a single-strand break in any one of ALDH1L1, ALDOB, G6PC, LRP2, RPL3L, SLC25A45, or SLC7A9 genomic nucleic acid molecules. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of one or more of ALDH1L1, ALDOB, G6PC, LRP2, RPL3L, SLC25A45, and/or SLC7A9 genomic nucleic acid molecules can be generated by contacting a cell with a Cas protein and one or more gRNAs that hybridize to one or more gRNA recognition sequences within a target genomic locus in the one or more of ALDH1L1, ALDOB, G6PC, LRP2, RPL3L, SLC25A45, and/or SLC7A9 genomic nucleic acid molecules. For example, when targeting ALDH1L1, a gRNA recognition sequence can be located within a region of SEQ ID NO:1. The gRNA recognition sequence can include or be proximate to the start codon of an ALDH1L1 genomic nucleic acid molecule or the stop codon of an ALDH1L1 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon. For example, when targeting ALDOB, a gRNA recognition sequence can be located within a region of SEQ ID NO:73. The gRNA recognition sequence can include or be proximate to the start codon of an ALDOB genomic nucleic acid molecule or the stop codon of an ALDOB genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon. For example, when targeting G6PC, a gRNA recognition sequence can be located within a region of SEQ ID NO:102. The gRNA recognition sequence can include or be proximate to the start codon of a G6PC genomic nucleic acid molecule or the stop codon of a G6PC genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon. For example, when targeting LRP2, a gRNA recognition sequence can be located within a region of SEQ ID NO:120. The gRNA recognition sequence can include or be proximate to the start codon of an LRP2 genomic nucleic acid molecule or the stop codon of an LRP2 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon. For example, when targeting RPL3L, a gRNA recognition sequence can be located within a region of SEQ ID NO:144. The gRNA recognition sequence can include or be proximate to the start codon of an RPL3L genomic nucleic acid molecule or the stop codon of an RPL3L genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon. For example, when targeting SLC25A45, a gRNA recognition sequence can be located within a region of SEQ ID NO:159. The gRNA recognition sequence can include or be proximate to the start codon of an SLC25A45 genomic nucleic acid molecule or the stop codon of an SLC25A45 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon. For example, when targeting SLC7A9, a gRNA recognition sequence can be located within a region of SEQ ID NO:205. The gRNA recognition sequence can include or be proximate to the start codon of an SLC7A9 genomic nucleic acid molecule or the stop codon of an SLC7A9 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in ALDH1L1, ALDOB, G6PC, LRP2, RPL3L, SLC25A45, or SLC7A9 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within ALDH1L1, ALDOB, G6PC, LRP2, RPL3L, SLC25A45, or SLC7A9 genomic nucleic acid molecule. An exemplary gRNA is a gRNA effective to direct a Cas enzyme to bind to or cleave ALDH1L1, ALDOB, G6PC, LRP2, RPL3L, SLC25A45, or SLC7A9 genomic nucleic acid molecule, wherein the gRNA comprises a DNA-targeting segment that hybridizes to a gRNA recognition sequence within the ALDH1L1, ALDOB, G6PC, LRP2, RPL3L, SLC25A45, or SLC7A9 genomic nucleic acid molecule. Exemplary gRNAs comprise a DNA-targeting segment that hybridizes to a gRNA recognition sequence present within ALDH1L1, ALDOB, G6PC, LRP2, RPL3L, SLC25A45, or SLC7A9 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human ALDH1L1 reference gene are set forth in Table 1 as SEQ ID NOs:229-248.

TABLE 1
Guide RNA Recognition Sequences
Near ALDH1L1 Variation(s)

| Strand | gRNA Recognition Sequence | SEQ ID NO: |
|---|---|---|
| + | TCTGGATGGACATGCCCACG | 229 |
| − | CGGTATTCAAGTACTCCCGG | 230 |
| − | CTGGCCCTGAAGACCCACGT | 231 |
| − | ACTGCTCCCTAGGCACCGAG | 232 |
| + | CTGGATGGACATGCCCACGT | 233 |
| + | TCATCAGGGGATAGTTCCAG | 234 |
| + | GGGCACTGATTATCTCCATG | 235 |
| − | ATGCCTTTGAGAATGGACGG | 236 |
| − | CCAAGTCACCGACGTCGACA | 237 |
| + | CCGGCACCTTGTCGTTCCCG | 238 |
| + | GGTGGGATTGATGGTCTCAG | 239 |
| + | CCAGCCAGCAAAGTAGCGGA | 240 |
| − | GGTGGGGGAAGATCAGTGCG | 241 |
| + | CCTCACAACGTCCACAGACG | 242 |
| − | CTGGATCCGCGGGAACGACA | 243 |
| − | AGGAAGGAGCCACCTATGAG | 244 |
| + | GCACTCACACTTCCATCGGT | 245 |
| + | GCCCTCAGCGATCAGCCTCA | 246 |
| + | TGCCCCCTTAAAGAAGTTCG | 247 |
| − | CTGCGAGGGGACGATGAGGA | 248 |

Examples of suitable gRNA recognition sequences located within the human ALDOB reference gene are set forth in Table 2 as SEQ ID NOs:249-268.

TABLE 2
Guide RNA Recognition Sequences
Near ALDOB Variation(s)

| Strand | gRNA Recognition Sequence | SEQ ID NO: |
|---|---|---|
| + | GCACCTCCTTGGTCTAACTG | 249 |
| − | TTGTATCCACAGTTAGACCA | 250 |
| − | TCCATCAACCAGAGCATCGG | 251 |
| − | CTCCCACCATAGGTACCATG | 252 |
| − | TATCCACAGTTAGACCAAGG | 253 |
| − | GGTACCTATTGTTGAACCAG | 254 |
| − | AAGGAAAAGGGGATCGTGGT | 255 |
| − | AAAACACTGAAGAGAACCGC | 256 |
| + | TAGCGAGGCTGGATGGACAC | 257 |
| − | TTCCATCAACCAGAGCATCG | 258 |
| − | CCACGAGACCCTCTACCAGA | 259 |
| + | GAGGCTGGATGGACACTGGT | 260 |
| − | CTCTACCAGAAGGACAGCCA | 261 |
| + | AATATCCTTACCTTGAATGG | 262 |
| + | GGATTTCTCGGAACTGCCGG | 263 |
| + | ACTGAGCACAGCGCTCTGAG | 264 |
| − | CCTCCCACCATAGGTACCAT | 265 |
| − | CACCCTGCTAAAGCCCAACA | 266 |
| − | CTGCCAGTATGTTACTGAGA | 267 |
| + | GGGCCTTGTAGACAGCAGCC | 268 |

Examples of suitable gRNA recognition sequences located within the human G6PC reference gene are set forth in Table 3 as SEQ ID NOs:269-288.

TABLE 3
Guide RNA Recognition Sequences
Near G6PC Variation(s)

| Strand | gRNA Recognition Sequence | SEQ ID NO: |
|---|---|---|
| − | CTCCAATCACAGCTACCCAA | 269 |
| − | GTTCTTACCACTTAAAGACG | 270 |
| + | AGCAGGTGTATACTACGTGA | 271 |
| − | ACAGGGAACTGCTTTATCAG | 272 |
| − | ACTCCAGCAACAACTTGATG | 273 |
| + | TGGACAGCGTCCATACTGGT | 274 |
| − | GAGCAGCAGATAAAATCCGA | 275 |
| − | GAGTTCTTACCGAAATCTGT | 276 |
| + | CCAGTCAACACATTACCTCC | 277 |
| + | GTAACCTGTGAGACTGGACC | 278 |
| − | TCTTACCGAAATCTGTAGGT | 279 |
| − | GGACGTAGAAGGCATTCCTG | 280 |
| + | ATTACCAAGACTCCCAGGAC | 281 |
| − | CAGATCAGCCCATACCTGAC | 282 |
| + | TTGGACAGCGTCCATACTGG | 283 |
| − | GCTGTCCAAAGAGAATCCTA | 284 |
| − | GTATCCAAAACCCACCAGTA | 285 |
| + | TTCCTGTTCAGCTTCGCCAT | 286 |
| + | TCTTTGGACAGCGTCCATAC | 287 |
| − | GGCTGGCATTATAGATGCTG | 288 |

Examples of suitable gRNA recognition sequences located within the human LRP2 reference gene are set forth in Table 4 as SEQ ID NOs:289-308.

TABLE 4
Guide RNA Recognition Sequences
Near LRP2 Variation(s)

| Strand | gRNA Recognition Sequence | SEQ ID NO: |
|---|---|---|
| − | GGTGACTATAGCGACGAGAG | 289 |
| + | GCATAACCCGACGAGTAGCA | 290 |
| + | GATGAACTACCACCGACCGT | 291 |
| + | CCATTGCAGATAAACTCACG | 292 |
| + | CCGTCACACAAATAAACGCG | 293 |
| + | CACCATCACAGACAAACGAT | 294 |
| − | GCATCCTAGAGGAATTGCCG | 295 |
| − | GAAATGACAGTTATCCGAAG | 296 |
| + | CCATCACAGACCCAAGACTG | 297 |
| − | TCCTAGAACTCAGTCAATCG | 298 |
| − | TGACAAACGCAACGACTGTG | 299 |
| + | AATAAACGTAGCCATCACTT | 300 |
| − | AAAAACCTAACAAATCCAAG | 301 |
| + | CTGTTCGATTGGTACAGTCA | 302 |
| − | CAGCACTGTTACAATATGAG | 303 |
| − | GGGTTATGCGAGCCAACAAG | 304 |
| + | ATTGCACATAAACTCCGTGG | 305 |
| + | GAACATTTATGAACATCATG | 306 |
| + | CATGGACTGCAATTCCCCAG | 307 |
| + | GAACGCGGGCTATCACAGTG | 308 |

Examples of suitable gRNA recognition sequences located within the human RPL3L reference gene are set forth in Table 5 as SEQ ID NOs:309-328.

TABLE 5
Guide RNA Recognition Sequences
Near RPL3L Variation(s)

| Strand | gRNA Recognition Sequence | SEQ ID NO: |
|---|---|---|
| − | TGTAGAAACGCCGCCCCTAG | 309 |
| + | GGCGCCGGCACTCATCACTG | 310 |
| − | AGAAACGCCGCCCCTAGTGG | 311 |
| + | AGGCCCTTATGGGTCTTCCG | 312 |
| − | TCCAGAAATTTCCAAACGGG | 313 |
| − | ATGGAGATCCAGCTGAACGG | 314 |
| − | CCATAAGGGCCTGCGCAAGG | 315 |
| + | AGTCCTTGTAGAATCGGCGC | 316 |
| + | ACCACGCCCACCACCACTAG | 317 |
| + | GCAGCTTCTTGGTATGCCAG | 318 |
| + | AACTGTGACTCACTGAGCCC | 319 |
| − | GGCGCCGATTCTACAAGGAC | 320 |
| − | GTGCCGGCGCCGATTCTACA | 321 |
| − | GGCATACCAAGAAGCTGCCG | 322 |
| + | TGAAGCTCCGGAGACCTCGA | 323 |
| − | ACCAAGGGTCGAGGCGTCAA | 324 |
| − | AAGAGGTGGCGGGACACAGA | 325 |
| + | TGACATCAATGACCTCACTC | 326 |
| + | TGTAGCCCAGGAAGGCCGTG | 327 |
| − | TGCCCCATAAGAGGAGCCAC | 328 |

Examples of suitable gRNA recognition sequences located within the human SLC25A45 reference gene are set forth in Table 6 as SEQ ID NOs:329-348.

TABLE 6
Guide RNA Recognition Sequences
Near SLC25A45 Variation(s)

| Strand | gRNA Recognition Sequence | SEQ ID NO: |
|---|---|---|
| − | CAGACCCAGACCACCTACCG | 329 |
| + | TGAAGTAGATCCCCACCGTG | 330 |
| + | GGCCACTCACGGACTCATGG | 331 |
| + | ATGAAGTAGATCCCCACCGT | 332 |
| + | ATCAACGATGCCCCGGTAGG | 333 |
| + | GCAATCAACGATGCCCCGGT | 334 |
| + | GATGAAGTAGATCCCCACCG | 335 |
| + | TGTTGCTATAGACCCCAAAC | 336 |
| + | GTGGGCCACTCACGGACTCA | 337 |
| + | GAAGTAGATCCCCACCGTGG | 338 |
| − | TACATGCACATCTTCCTAGC | 339 |
| + | TTTGTAGCCGGACTTTGATG | 340 |
| − | TGAGGGACACCCCCACGGTG | 341 |
| − | TCTACTTCATCACCTATGAA | 342 |
| − | ACACCCGTTTGACACTGTAA | 343 |
| + | CAGTGTCAAACGGGTGTCCC | 344 |
| + | GCTCCTCGGAACAGCCCCCG | 345 |
| − | GCCCCCCACCCCGGTACCAG | 346 |
| + | TCATTCCCTTGAAGAAGCCC | 347 |
| + | GGCCCGCCGCTCCTGGTGGG | 348 |

Examples of suitable gRNA recognition sequences located within the human SLC7A9 reference gene are set forth in Table 6 as SEQ ID NOs:349-368.

TABLE 7
Guide RNA Recognition Sequences
Near SLC7A9 Variation(s)

| Strand | gRNA Recognition Sequence | SEQ ID NO: |
|---|---|---|
| + | ATGTAGCACGCCGTCACCAG | 349 |
| + | TGAAGTAGGACACGTTCATG | 350 |
| + | GAAGGGCGCACACACATACT | 351 |
| − | GCCCTGCCTCATCATATGGG | 352 |
| − | GCACAATGATCACCAAGTCA | 353 |
| + | CATTTCACAACGATTTGAGG | 354 |
| + | CGCAAGCCGCCCATATGATG | 355 |
| + | TTCACTGTCGAGATGAACAC | 356 |
| − | CATTATCATCGGGATCCCCC | 357 |
| + | GGTCCCGTTAGCAGCACCGA | 358 |
| − | TCAGCAACACGGAAGCTGTG | 359 |
| − | CAGTGAACTCACTGAGCGTG | 360 |
| − | AGAGTATCCCTACCTGATGG | 361 |
| − | ACAATGATCACCAAGTCAGG | 362 |
| + | AGAGTCCATTGTAAAACGCC | 363 |
| + | AGTGGAACGATCCAAGAAGC | 364 |
| + | TGTTGCTGAGCACAGACTTG | 365 |
| − | AGTGGCATCTCCATCATCGT | 366 |
| − | GTGGCCATCATCATCATCAG | 367 |
| + | ACTCACCACAGCCACCGCCT | 368 |

The Cas protein and the gRNA form a complex, and the Cas protein cleaves the target ALDH1L1, ALDOB, G6PC, LRP2, RPL3L, SLC25A45, or SLC7A9 genomic nucleic acid molecule. The Cas protein can cleave the nucleic acid molecule at a site within or outside of the nucleic acid sequence present in the target ALDH1L1, ALDOB, G6PC, LRP2, RPL3L, SLC25A45, or SLC7A9 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the ALDH1L1, ALDOB, G6PC, LRP2, RPL3L, SLC25A45, or SLC7A9 genomic nucleic acid molecule to which a DNA-targeting segment of a gRNA will bind.

Such methods can result, for example, in an ALDH1L1 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the ALDH1L1 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

Such methods can also result, for example, in an ALDOB genomic nucleic acid molecule in which a region of SEQ ID NO:73 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the ALDOB genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

Such methods can also result, for example, in a G6PC genomic nucleic acid molecule in which a region of SEQ ID NO:102 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the G6PC genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

Such methods can also result, for example, in an LRP2 genomic nucleic acid molecule in which a region of SEQ ID NO:120 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the LRP2 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

Such methods can also result, for example, in an RPL3L genomic nucleic acid molecule in which a region of SEQ ID NO:144 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the RPL3L genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

Such methods can also result, for example, in an SLC25A45 genomic nucleic acid molecule in which a region of SEQ ID NO:159 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the SLC25A45 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

Such methods can also result, for example, in an SLC7A9 genomic nucleic acid molecule in which a region of SEQ ID NO:205 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the SLC7A9 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods of treatment and/or prevention further comprise detecting the presence or absence of one or more of ALDH1L1, ALDOB, G6PC, LRP2, RPL3L, SLC25A45, or SLC7A9 variant nucleic acid molecule encoding one or more ALDH1L1, ALDOB, G6PC, LRP2, RPL3L, SLC25A45, or SLC7A9 predicted loss-of-function polypeptides in a biological sample from the subject. As used throughout the present disclosure, an “ALDH1L1 variant nucleic acid molecule encoding an ALDH1L1 predicted loss-of-function polypeptide” is any ALDH1L1 nucleic acid molecule (such as, for example, genomic nucleic acid molecule, mRNA molecule, or cDNA molecule) encoding an ALDH1L1 polypeptide having a partial loss-of-function, a complete loss-of-function, a predicted partial loss-of-function, or a predicted complete loss-of-function. As used throughout the present disclosure, an “ALDOB variant nucleic acid molecule encoding an ALDOB predicted loss-of-function polypeptide” is any ALDOB nucleic acid molecule (such as, for example, genomic nucleic acid molecule, mRNA molecule, or cDNA molecule) encoding an ALDOB polypeptide having a partial loss-of-function, a complete loss-of-function, a predicted partial loss-of-function, or a predicted complete loss-of-function. As used throughout the present disclosure, a “G6PC variant nucleic acid molecule encoding a G6PC predicted loss-of-function polypeptide” is any G6PC nucleic acid molecule (such as, for example, genomic nucleic acid molecule, mRNA molecule, or cDNA molecule) encoding a G6PC polypeptide having a partial loss-of-function, a complete loss-of-function, a predicted partial loss-of-function, or a predicted complete loss-of-function. As used throughout the present disclosure, an “LRP2 variant nucleic acid molecule encoding an LRP2 predicted loss-of-function polypeptide” is any LRP2 nucleic acid molecule (such as, for example, genomic nucleic acid molecule, mRNA molecule, or cDNA molecule) encoding an LRP2 polypeptide having a partial loss-of-function, a complete loss-of-function, a predicted partial loss-of-function, or a predicted complete loss-of-function. As used throughout the present disclosure, an “RPL3L variant nucleic acid molecule encoding an RPL3L predicted loss-of-function polypeptide” is any RPL3L nucleic acid molecule (such as, for example, genomic nucleic acid molecule, mRNA molecule, or cDNA molecule) encoding an RPL3L polypeptide having a partial loss-of-function, a complete loss-of-function, a predicted partial loss-of-function, or a predicted complete loss-of-function. As used throughout the present disclosure, an “SLC25A45 variant nucleic acid molecule encoding an SLC25A45 predicted loss-of-function polypeptide” is any SLC25A45 nucleic acid molecule (such as, for example, genomic nucleic acid molecule, mRNA molecule, or cDNA molecule) encoding an SLC25A45 polypeptide having a partial loss-of-function, a complete loss-of-function, a predicted partial loss-of-function, or a predicted complete loss-of-function. As used throughout the present disclosure, an “SLC7A9 variant nucleic acid molecule encoding an SLC7A9 predicted loss-of-function polypeptide” is any SLC7A9 nucleic acid molecule (such as, for example, genomic nucleic acid molecule, mRNA molecule, or cDNA molecule) encoding an SLC7A9 polypeptide having a partial loss-of-function, a complete loss-of-function, a predicted partial loss-of-function, or a predicted complete loss-of-function.

The present disclosure also provides methods of treating a subject with a therapeutic agent that treats or inhibits a kidney disease, wherein the subject has a kidney disease. The present disclosure also provides methods of preventing a subject from developing a kidney disease by administering a therapeutic agent that prevents a kidney disease. In some embodiments, the methods comprise determining whether the subject has an ALDH1L1 variant nucleic acid molecule encoding an ALDH1L1 predicted loss-of-function polypeptide by obtaining or having obtained a biological sample from the subject, and performing or having performed a sequence analysis on the biological sample to determine if the subject has a genotype comprising the ALDH1L1 variant nucleic acid molecule encoding an ALDH1L1 predicted loss-of-function polypeptide. In some embodiments, the methods further comprise administering or continuing to administer the therapeutic agent that treats, prevents, or inhibits the kidney disease in an amount that is the same as or less than a standard dosage amount to a subject that is ALDH1L1 reference, and/or administering an ALDH1L1 inhibitor to the subject. In some embodiments, the methods further comprise administering or continuing to administer the therapeutic agent that treats, prevents, or inhibits the kidney disease in an amount that is the same as or less than a standard dosage amount to a subject that is heterozygous for the ALDH1L1 variant nucleic acid molecule, and/or administering an ALDH1L1 inhibitor to the subject. In some embodiments, the methods further comprise administering or continuing to administer the therapeutic agent that treats, prevents, or inhibits the kidney disease in a standard dosage amount to a subject that is homozygous for the ALDH1L1 variant nucleic acid molecule. The presence of a genotype having the ALDH1L1 variant nucleic acid molecule encoding the ALDH1L1 predicted loss-of-function polypeptide indicates the subject has a decreased risk of developing a kidney disease. In some embodiments, the subject is ALDH1L1 reference. In some embodiments, the subject is heterozygous for an ALDH1L1 variant nucleic acid molecule encoding an ALDH1L1 predicted loss-of-function polypeptide.

For subjects that are genotyped or determined to be either ALDH1L1 reference or heterozygous for an ALDH1L1 variant nucleic acid molecule encoding an ALDH1L1 predicted loss-of-function polypeptide, such subjects can be administered an ALDH1L1 inhibitor, as described herein.

The present disclosure also provides methods of treating a subject with a therapeutic agent that treats or inhibits a kidney disease, wherein the subject has a kidney disease. The present disclosure also provides methods of preventing a subject from developing a kidney disease by administering a therapeutic agent that prevents the kidney disease. In some embodiments, the methods comprise determining whether the subject has an ALDOB variant nucleic acid molecule encoding an ALDOB predicted loss-of-function polypeptide by obtaining or having obtained a biological sample from the subject, and performing or having performed a sequence analysis on the biological sample to determine if the subject has a genotype comprising the ALDOB variant nucleic acid molecule encoding an ALDOB predicted loss-of-function polypeptide. In some embodiments, the methods further comprise administering or continuing to administer the therapeutic agent that treats, prevents, or inhibits the kidney disease in an amount that is the same as or less than a standard dosage amount to a subject that is ALDOB reference, and/or administering an ALDOB inhibitor to the subject. In some embodiments, the methods further comprise administering or continuing to administer the therapeutic agent that treats, prevents, or inhibits the kidney disease in an amount that is the same as or less than a standard dosage amount to a subject that is heterozygous for the ALDOB variant nucleic acid molecule, and/or administering an ALDOB inhibitor to the subject. In some embodiments, the methods further comprise administering or continuing to administer the therapeutic agent that treats, prevents, or inhibits the kidney disease in a standard dosage amount to a subject that is homozygous for the ALDOB variant nucleic acid molecule. The presence of a genotype having the ALDOB variant nucleic acid molecule encoding the ALDOB predicted loss-of-function polypeptide indicates the subject has a decreased risk of developing a kidney disease. In some embodiments, the subject is ALDOB reference. In some embodiments, the subject is heterozygous for an ALDOB variant nucleic acid molecule encoding an ALDOB predicted loss-of-function polypeptide.

For subjects that are genotyped or determined to be either ALDOB reference or heterozygous for an ALDOB variant nucleic acid molecule encoding an ALDOB predicted loss-of-function polypeptide, such subjects can be administered an ALDOB inhibitor, as described herein.

The present disclosure also provides methods of treating a subject with a therapeutic agent that treats or inhibits a kidney disease, wherein the subject has a kidney disease. The present disclosure also provides methods of preventing a subject from developing a kidney disease by administering a therapeutic agent that prevents the kidney disease. In some embodiments, the methods comprise determining whether the subject has an LRP2 variant nucleic acid molecule encoding an LRP2 predicted loss-of-function polypeptide by obtaining or having obtained a biological sample from the subject, and performing or having performed a sequence analysis on the biological sample to determine if the subject has a genotype comprising the LRP2 variant nucleic acid molecule encoding an LRP2 predicted loss-of-function polypeptide. In some embodiments, the methods further comprise administering or continuing to administer the therapeutic agent that treats, prevents, or inhibits the kidney disease in an amount that is the same as or less than a standard dosage amount to a subject that is LRP2 reference, and/or administering an LRP2 inhibitor to the subject. In some embodiments, the methods further comprise administering or continuing to administer the therapeutic agent that treats, prevents, or inhibits the kidney disease in an amount that is the same as or less than a standard dosage amount to a subject that is heterozygous for the LRP2 variant nucleic acid molecule, and/or administering an LRP2 inhibitor to the subject. In some embodiments, the methods further comprise administering or continuing to administer the therapeutic agent that treats, prevents, or inhibits the kidney disease in a standard dosage amount to a subject that is homozygous for the LRP2 variant nucleic acid molecule. The presence of a genotype having the LRP2 variant nucleic acid molecule encoding the LRP2 predicted loss-of-function polypeptide indicates the subject has a decreased risk of developing a kidney disease. In some embodiments, the subject is LRP2 reference. In some embodiments, the subject is heterozygous for an LRP2 variant nucleic acid molecule encoding an LRP2 predicted loss-of-function polypeptide.

For subjects that are genotyped or determined to be either LRP2 reference or heterozygous for an LRP2 variant nucleic acid molecule encoding an LRP2 predicted loss-of-function polypeptide, such subjects can be administered an LRP2 inhibitor, as described herein.

The present disclosure also provides methods of treating a subject with a therapeutic agent that treats or inhibits a kidney disease, wherein the subject has a kidney disease. The present disclosure also provides methods of preventing a subject from developing a kidney disease by administering a therapeutic agent that prevents the kidney disease. In some embodiments, the methods comprise determining whether the subject has an RPL3L variant nucleic acid molecule encoding an RPL3L predicted loss-of-function polypeptide by obtaining or having obtained a biological sample from the subject, and performing or having performed a sequence analysis on the biological sample to determine if the subject has a genotype comprising the RPL3L variant nucleic acid molecule encoding an RPL3L predicted loss-of-function polypeptide. In some embodiments, the methods further comprise administering or continuing to administer the therapeutic agent that treats, prevents, or inhibits the kidney disease in an amount that is the same as or less than a standard dosage amount to a subject that is RPL3L reference, and/or administering an RPL3L inhibitor to the subject. In some embodiments, the methods further comprise administering or continuing to administer the therapeutic agent that treats, prevents, or inhibits the kidney disease in an amount that is the same as or less than a standard dosage amount to a subject that is heterozygous for the RPL3L variant nucleic acid molecule, and/or administering an RPL3L inhibitor to the subject. In some embodiments, the methods further comprise administering or continuing to administer the therapeutic agent that treats, prevents, or inhibits the kidney disease in a standard dosage amount to a subject that is homozygous for the RPL3L variant nucleic acid molecule. The presence of a genotype having the RPL3L variant nucleic acid molecule encoding the RPL3L predicted loss-of-function polypeptide indicates the subject has a decreased risk of developing a kidney disease. In some embodiments, the subject is RPL3L reference. In some embodiments, the subject is heterozygous for an RPL3L variant nucleic acid molecule encoding an RPL3L predicted loss-of-function polypeptide.

For subjects that are genotyped or determined to be either RPL3L reference or heterozygous for an RPL3L variant nucleic acid molecule encoding an RPL3L predicted loss-of-function polypeptide, such subjects can be administered an RPL3L inhibitor, as described herein.

The present disclosure also provides methods of treating a subject with a therapeutic agent that treats or inhibits a kidney disease, wherein the subject has a kidney disease. The present disclosure also provides methods of preventing a subject from developing a kidney disease by administering a therapeutic agent that prevents the kidney disease. In some embodiments, the methods comprise determining whether the subject has an SLC25A45 variant nucleic acid molecule encoding an SLC25A45 predicted loss-of-function polypeptide by obtaining or having obtained a biological sample from the subject, and performing or having performed a sequence analysis on the biological sample to determine if the subject has a genotype comprising the SLC25A45 variant nucleic acid molecule encoding an SLC25A45 predicted loss-of-function polypeptide. In some embodiments, the methods further comprise administering or continuing to administer the therapeutic agent that treats, prevents, or inhibits the kidney disease in an amount that is the same as or less than a standard dosage amount to a subject that is SLC25A45 reference, and/or administering an SLC25A45 inhibitor to the subject. In some embodiments, the methods further comprise administering or continuing to administer the therapeutic agent that treats, prevents, or inhibits the kidney disease in an amount that is the same as or less than a standard dosage amount to a subject that is heterozygous for the SLC25A45 variant nucleic acid molecule, and/or administering an SLC25A45 inhibitor to the subject. In some embodiments, the methods further comprise administering or continuing to administer the therapeutic agent that treats, prevents, or inhibits the kidney disease in a standard dosage amount to a subject that is homozygous for the SLC25A45 variant nucleic acid molecule. The presence of a genotype having the SLC25A45 variant nucleic acid molecule encoding the SLC25A45 predicted loss-of-function polypeptide indicates the subject has a decreased risk of developing a kidney disease. In some embodiments, the subject is SLC25A45 reference. In some embodiments, the subject is heterozygous for an SLC25A45 variant nucleic acid molecule encoding an SLC25A45 predicted loss-of-function polypeptide.

For subjects that are genotyped or determined to be either SLC25A45 reference or heterozygous for an SLC25A45 variant nucleic acid molecule encoding an SLC25A45 predicted loss-of-function polypeptide, such subjects can be administered an SLC25A45 inhibitor, as described herein.

The present disclosure also provides methods of treating a subject with a therapeutic agent that treats or inhibits a kidney disease, wherein the subject has a kidney disease. The present disclosure also provides methods of preventing a subject from developing a kidney disease by administering a therapeutic agent that prevents the kidney disease. In some embodiments, the methods comprise determining whether the subject has an SLC7A9 variant nucleic acid molecule encoding an SLC7A9 predicted loss-of-function polypeptide by obtaining or having obtained a biological sample from the subject, and performing or having performed a sequence analysis on the biological sample to determine if the subject has a genotype comprising the SLC7A9 variant nucleic acid molecule encoding an SLC7A9 predicted loss-of-function polypeptide. In some embodiments, the methods further comprise administering or continuing to administer the therapeutic agent that treats, prevents, or inhibits the kidney disease in an amount that is the same as or less than a standard dosage amount to a subject that is SLC7A9 reference, and/or administering an SLC7A9 inhibitor to the subject. In some embodiments, the methods further comprise administering or continuing to administer the therapeutic agent that treats, prevents, or inhibits the kidney disease in an amount that is the same as or less than a standard dosage amount to a subject that is heterozygous for the SLC7A9 variant nucleic acid molecule, and/or administering an SLC7A9 inhibitor to the subject. In some embodiments, the methods further comprise administering or continuing to administer the therapeutic agent that treats, prevents, or inhibits the kidney disease in a standard dosage amount to a subject that is homozygous for the SLC7A9 variant nucleic acid molecule. The presence of a genotype having the SLC7A9 variant nucleic acid molecule encoding the SLC7A9 predicted loss-of-function polypeptide indicates the subject has a decreased risk of developing a kidney disease. In some embodiments, the subject is SLC7A9 reference. In some embodiments, the subject is heterozygous for an SLC7A9 variant nucleic acid molecule encoding an SLC7A9 predicted loss-of-function polypeptide.

For subjects that are genotyped or determined to be either SLC7A9 reference or heterozygous for an SLC7A9 variant nucleic acid molecule encoding an SLC7A9 predicted loss-of-function polypeptide, such subjects can be administered an SLC7A9 inhibitor, as described herein.

Detecting the presence or absence of one or more ALDH1L1, ALDOB, G6PC, LRP2, RPL3L, SLC25A45, and/or SLC7A9 variant nucleic acid molecules encoding one or more ALDH1L1, ALDOB, G6PC, LRP2, RPL3L, SLC25A45, or SLC7A9 predicted loss-of-function polypeptides in a biological sample from a subject and/or determining whether a subject has one or more ALDH1L1, ALDOB, G6PC, LRP2, RPL3L, SLC25A45, or SLC7A9 variant nucleic acid molecules encoding one or more ALDH1L1, ALDOB, G6PC, LRP2, RPL3L, SLC25A45, or SLC7A9 predicted loss-of-function polypeptides can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is ALDH1L1 reference, the subject is administered a therapeutic agent that treats, prevents, or inhibits a kidney disease in an amount that is the same as or less than a standard dosage amount. In some embodiments, when the subject is heterozygous for an ALDH1L1 variant nucleic acid molecule encoding an ALDH1L1 predicted loss-of-function polypeptide, the subject is administered a therapeutic agent that treats, prevents, or inhibits a kidney disease in a dosage amount that is the same as or less than a standard dosage amount.

In some embodiments, when the subject is ALDOB reference, the subject is administered a therapeutic agent that treats, prevents, or inhibits a kidney disease in an amount that is the same as or less than a standard dosage amount. In some embodiments, when the subject is heterozygous for an ALDOB variant nucleic acid molecule encoding an ALDOB predicted loss-of-function polypeptide, the subject is administered a therapeutic agent that treats, prevents, or inhibits a kidney disease in a dosage amount that is the same as or less than a standard dosage amount.

In some embodiments, when the subject is G6PC reference, the subject is administered a therapeutic agent that treats, prevents, or inhibits a kidney disease in an amount that is the same as or less than a standard dosage amount. In some embodiments, when the subject is heterozygous for a G6PC variant nucleic acid molecule encoding a G6PC predicted loss-of-function polypeptide, the subject is administered a therapeutic agent that treats, prevents, or inhibits a kidney disease in a dosage amount that is the same as or less than a standard dosage amount.

In some embodiments, when the subject is LRP2 reference, the subject is administered a therapeutic agent that treats, prevents, or inhibits a kidney disease in an amount that is the same as or less than a standard dosage amount. In some embodiments, when the subject is heterozygous for an LRP2 variant nucleic acid molecule encoding an LRP2 predicted loss-of-function polypeptide, the subject is administered a therapeutic agent that treats, prevents, or inhibits a kidney disease in a dosage amount that is the same as or less than a standard dosage amount.

In some embodiments, when the subject is RPL3L reference, the subject is administered a therapeutic agent that treats, prevents, or inhibits a kidney disease in an amount that is the same as or less than a standard dosage amount. In some embodiments, when the subject is heterozygous for an RPL3L variant nucleic acid molecule encoding an RPL3L predicted loss-of-function polypeptide, the subject is administered a therapeutic agent that treats, prevents, or inhibits a kidney disease in a dosage amount that is the same as or less than a standard dosage amount.

In some embodiments, when the subject is SLC25A45 reference, the subject is administered a therapeutic agent that treats, prevents, or inhibits a kidney disease in an amount that is the same as or less than a standard dosage amount. In some embodiments, when the subject is heterozygous for an SLC25A45 variant nucleic acid molecule encoding an SLC25A45 predicted loss-of-function polypeptide, the subject is administered a therapeutic agent that treats, prevents, or inhibits a kidney disease in a dosage amount that is the same as or less than a standard dosage amount.

In some embodiments, when the subject is SLC7A9 reference, the subject is administered a therapeutic agent that treats, prevents, or inhibits a kidney disease in an amount that is the same as or less than a standard dosage amount. In some embodiments, when the subject is heterozygous for an SLC7A9 variant nucleic acid molecule encoding an SLC7A9 predicted loss-of-function polypeptide, the subject is administered a therapeutic agent that treats, prevents, or inhibits a kidney disease in a dosage amount that is the same as or less than a standard dosage amount.

In any of the embodiments described herein, when the subject is reference for any combination of two or more of ALDH1L1, ALDOB, G6PC, LRP2, RPL3L, SLC25A45, and SLC7A9, the subject is administered a therapeutic agent that treats, prevents, or inhibits a kidney disease in an amount that is the same as or less than a standard dosage amount. In any of the embodiments described herein, when the subject is is heterozygous for any combination of two or more of ALDH1L1, ALDOB, G6PC, LRP2, RPL3L, SLC25A45, and SLC7A9 variant nucleic acid molecules encoding any combination of two or more of ALDH1L1, ALDOB, G6PC, LRP2, RPL3L, SLC25A45, and SLC7A9 predicted loss-of-function polypeptides, the subject is administered a therapeutic agent that treats, prevents, or inhibits a kidney disease in a dosage amount that is the same as or less than a standard dosage amount.

In some embodiments, the treatment and/or prevention methods further comprise detecting the presence or absence of an ALDH1L1 predicted loss-of-function polypeptide in a biological sample from the subject. In some embodiments, when the subject does not have an ALDH1L1 predicted loss-of-function polypeptide, the subject is also administered a therapeutic agent that treats, prevents, or inhibits a kidney disease in an amount that is the same as or less than a standard dosage amount. In some embodiments, when the subject has an ALDH1L1 predicted loss-of-function polypeptide, the subject is also administered a therapeutic agent that treats, prevents, or inhibits a kidney disease in amount that is the same as or less than a standard dosage amount.

In some embodiments, the treatment and/or prevention methods further comprise detecting the presence or absence of an ALDOB predicted loss-of-function polypeptide in a biological sample from the subject. In some embodiments, when the subject does not have an ALDOB predicted loss-of-function polypeptide, the subject is also administered a therapeutic agent that treats, prevents, or inhibits a kidney disease in an amount that is the same as or less than a standard dosage amount. In some embodiments, when the subject has an ALDOB predicted loss-of-function polypeptide, the subject is also administered a therapeutic agent that treats, prevents, or inhibits a kidney disease in a dosage amount that is the same as or less than a standard dosage amount.

In some embodiments, the treatment and/or prevention methods further comprise detecting the presence or absence of a G6PC predicted loss-of-function polypeptide in a biological sample from the subject. In some embodiments, when the subject does not have a G6PC predicted loss-of-function polypeptide, the subject is also administered a therapeutic agent that treats, prevents, or inhibits a kidney disease in an amount that is the same as or less than a standard dosage amount. In some embodiments, when the subject has a G6PC predicted loss-of-function polypeptide, the subject is also administered a therapeutic agent that treats, prevents, or inhibits a kidney disease in a dosage amount that is the same as or less than a standard dosage amount.

In some embodiments, the treatment and/or prevention methods further comprise detecting the presence or absence of an LRP2 predicted loss-of-function polypeptide in a biological sample from the subject. In some embodiments, when the subject does not have an LRP2 predicted loss-of-function polypeptide, the subject is also administered a therapeutic agent that treats, prevents, or inhibits a kidney disease in an amount that is the same as or less than a standard dosage amount. In some embodiments, when the subject has an LRP2 predicted loss-of-function polypeptide, the subject is also administered a therapeutic agent that treats, prevents, or inhibits a kidney disease in a dosage amount that is the same as or less than a standard dosage amount.

In some embodiments, the treatment and/or prevention methods further comprise detecting the presence or absence of an RPL3L predicted loss-of-function polypeptide in a biological sample from the subject. In some embodiments, when the subject does not have an RPL3L predicted loss-of-function polypeptide, the subject is also administered a therapeutic agent that treats, prevents, or inhibits a kidney disease in an amount that is the same as or less than a standard dosage amount. In some embodiments, when the subject has an RPL3L predicted loss-of-function polypeptide, the subject is also administered a therapeutic agent that treats, prevents, or inhibits a kidney disease in a dosage amount that is the same as or less than a standard dosage amount.

In some embodiments, the treatment and/or prevention methods further comprise detecting the presence or absence of an SLC25A45 predicted loss-of-function polypeptide in a biological sample from the subject. In some embodiments, when the subject does not have an SLC25A45 predicted loss-of-function polypeptide, the subject is also administered a therapeutic agent that treats, prevents, or inhibits a kidney disease in an amount that is the same as or less than a standard dosage amount. In some embodiments, when the subject has an SLC25A45 predicted loss-of-function polypeptide, the subject is also administered a therapeutic agent that treats, prevents, or inhibits a kidney disease in a dosage amount that is the same as or less than a standard dosage amount.

In some embodiments, the treatment and/or prevention methods further comprise detecting the presence or absence of an SLC7A9 predicted loss-of-function polypeptide in a biological sample from the subject. In some embodiments, when the subject does not have an SLC7A9 predicted loss-of-function polypeptide, the subject is also administered a therapeutic agent that treats, prevents, or inhibits a kidney disease in an amount that is the same as or less than a standard dosage amount. In some embodiments, when the subject has an SLC7A9 predicted loss-of-function polypeptide, the subject is also administered a therapeutic agent that treats, prevents, or inhibits a kidney disease in a dosage amount that is the same as or less than a standard dosage amount.

The present disclosure also provides methods of treating a subject with a therapeutic agent that treats or inhibits a kidney disease, wherein the subject has a kidney disease. In some embodiments, the method comprises determining whether the subject has an ALDH1L1 predicted loss-of-function polypeptide by obtaining or having obtained a biological sample from the subject, and performing or having performed an assay on the biological sample to determine if the subject has an ALDH1L1 predicted loss-of-function polypeptide. When the subject does not have an ALDH1L1 predicted loss-of-function polypeptide, the therapeutic agent that treats or inhibits the kidney disease is administered or continued to be administered to the subject in an amount that is the same as or less than a standard dosage amount, and/or an ALDH1L1 inhibitor is administered to the subject. When the subject has an ALDH1L1 predicted loss-of-function polypeptide, the therapeutic agent that treats or inhibits the kidney disease is administered or continued to be administered to the subject in an amount that is the same as or less than a standard dosage amount, and/or an ALDH1L1 inhibitor is administered to the subject. The presence of an ALDH1L1 predicted loss-of-function polypeptide indicates the subject has a decreased risk of developing a kidney disease. In some embodiments, the subject has an ALDH1L1 predicted loss-of-function polypeptide. In some embodiments, the subject does not have an ALDH1L1 predicted loss-of-function polypeptide.

The present disclosure also provides methods of preventing a subject from developing a kidney disease by administering a therapeutic agent that prevents the kidney disease. In some embodiments, the method comprises determining whether the subject has an ALDH1L1 predicted loss-of-function polypeptide by obtaining or having obtained a biological sample from the subject, and performing or having performed an assay on the biological sample to determine if the subject has an ALDH1L1 predicted loss-of-function polypeptide. When the subject does not have an ALDH1L1 predicted loss-of-function polypeptide, the therapeutic agent that prevents the kidney disease is administered or continued to be administered to the subject in an amount that is the same as or less than a standard dosage amount, and/or an ALDH1L1 inhibitor is administered to the subject. When the subject has an ALDH1L1 predicted loss-of-function polypeptide, the therapeutic agent that prevents the kidney disease is administered or continued to be administered to the subject in an amount that is the same as or less than a standard dosage amount, and/or an ALDH1L1 inhibitor is administered to the subject. The presence of an ALDH1L1 predicted loss-of-function polypeptide indicates the subject has a decreased risk of developing a kidney disease. In some embodiments, the subject has an ALDH1L1 predicted loss-of-function polypeptide. In some embodiments, the subject does not have an ALDH1L1 predicted loss-of-function polypeptide.

Detecting the presence or absence of an ALDH1L1 predicted loss-of-function polypeptide in a biological sample from a subject and/or determining whether a subject has an ALDH1L1 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the ALDH1L1 inhibitor is a small molecule. In some embodiments, the ALDH1L1 inhibitor is gossypol. In some embodiments, the ALDH1L1 inhibitor is N,N-diethylaminobenzaldehyde (DEAB). Additional inhibitors are disclosed in, for example, Koppaka et al., Pharmacol. Rev., 2012, 64, 520-539.

The present disclosure also provides methods of treating a subject with a therapeutic agent that treats or inhibits a kidney disease, wherein the subject has a kidney disease. In some embodiments, the method comprises determining whether the subject has an ALDOB predicted loss-of-function polypeptide by obtaining or having obtained a biological sample from the subject, and performing or having performed an assay on the biological sample to determine if the subject has an ALDOB predicted loss-of-function polypeptide. When the subject does not have an ALDOB predicted loss-of-function polypeptide, the therapeutic agent that treats or inhibits the kidney disease is administered or continued to be administered to the subject in an amount that is the same as or less than a standard dosage amount, and/or an ALDOB inhibitor is administered to the subject. When the subject has an ALDOB predicted loss-of-function polypeptide, the therapeutic agent that treats or inhibits the kidney disease is administered or continued to be administered to the subject in an amount that is the same as or less than a standard dosage amount, and/or an ALDOB inhibitor is administered to the subject. The presence of an ALDOB predicted loss-of-function polypeptide indicates the subject has a decreased risk of developing a kidney disease. In some embodiments, the subject has an ALDOB predicted loss-of-function polypeptide. In some embodiments, the subject does not have an ALDOB predicted loss-of-function polypeptide.

The present disclosure also provides methods of preventing a subject from developing a kidney disease by administering a therapeutic agent that prevents the kidney disease. In some embodiments, the method comprises determining whether the subject has an ALDOB predicted loss-of-function polypeptide by obtaining or having obtained a biological sample from the subject, and performing or having performed an assay on the biological sample to determine if the subject has an ALDOB predicted loss-of-function polypeptide. When the subject does not have an ALDOB predicted loss-of-function polypeptide, the therapeutic agent that prevents the kidney disease is administered or continued to be administered to the subject in an amount that is the same as or less than a standard dosage amount, and/or an ALDOB inhibitor is administered to the subject. When the subject has an ALDOB predicted loss-of-function polypeptide, the therapeutic agent that prevents the kidney disease is administered or continued to be administered to the subject in an amount that is the same as or less than a standard dosage amount, and/or an ALDOB inhibitor is administered to the subject. The presence of an ALDOB predicted loss-of-function polypeptide indicates the subject has a decreased risk of developing a kidney disease. In some embodiments, the subject has an ALDOB predicted loss-of-function polypeptide. In some embodiments, the subject does not have an ALDOB predicted loss-of-function polypeptide.

Detecting the presence or absence of an ALDOB predicted loss-of-function polypeptide in a biological sample from a subject and/or determining whether a subject has an ALDOB predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the ALDOB inhibitor is a small molecule. In some embodiments, the ALDOB inhibitor is TDZD-8. Small molecule inhibitors of aldolase include, but are not limited to, phosphorylated α-dicarbonyl compounds (e.g., phosphoric acid mono-(2,3-dioxo-butyl) ester. Additional inhibitors are disclosed in, for example, U.S. Patent Application Publication No. 2019/0231761, Charbot et al., J. Enzyme Inhibition Med. Chem., 2008, 23, 21-27, and Daher et al., ACS Med. Chem. Lett., 2010, 1, 101-104.

The present disclosure also provides methods of treating a subject with a therapeutic agent that treats or inhibits a kidney disease, wherein the subject has a kidney disease. In some embodiments, the method comprises determining whether the subject has a G6PC predicted loss-of-function polypeptide by obtaining or having obtained a biological sample from the subject, and performing or having performed an assay on the biological sample to determine if the subject has a G6PC predicted loss-of-function polypeptide. When the subject does not have a G6PC predicted loss-of-function polypeptide, the therapeutic agent that treats or inhibits the kidney disease is administered or continued to be administered to the subject in an amount that is the same as or less than a standard dosage amount, and/or a G6PC inhibitor is administered to the subject. When the subject has a G6PC predicted loss-of-function polypeptide, the therapeutic agent that treats or inhibits the kidney disease is administered or continued to be administered to the subject in an amount that is the same as or less than a standard dosage amount, and/or a G6PC inhibitor is administered to the subject. The presence of a G6PC predicted loss-of-function polypeptide indicates the subject has a decreased risk of developing a kidney disease. In some embodiments, the subject has a G6PC predicted loss-of-function polypeptide. In some embodiments, the subject does not have a G6PC predicted loss-of-function polypeptide.

The present disclosure also provides methods of preventing a subject from developing a kidney disease by administering a therapeutic agent that prevents the kidney disease. In some embodiments, the method comprises determining whether the subject has a G6PC predicted loss-of-function polypeptide by obtaining or having obtained a biological sample from the subject, and performing or having performed an assay on the biological sample to determine if the subject has a G6PC predicted loss-of-function polypeptide. When the subject does not have a G6PC predicted loss-of-function polypeptide, the therapeutic agent that prevents the kidney disease is administered or continued to be administered to the subject in an amount that is the same as or less than a standard dosage amount, and/or a G6PC inhibitor is administered to the subject. When the subject has a G6PC predicted loss-of-function polypeptide, the therapeutic agent that prevents the kidney disease is administered or continued to be administered to the subject in an amount that is the same as or less than a standard dosage amount, and/or a G6PC inhibitor is administered to the subject. The presence of a G6PC predicted loss-of-function polypeptide indicates the subject has a decreased risk of developing a kidney disease. In some embodiments, the subject has a G6PC predicted loss-of-function polypeptide. In some embodiments, the subject does not have a G6PC predicted loss-of-function polypeptide.

Detecting the presence or absence of a G6PC predicted loss-of-function polypeptide in a biological sample from a subject and/or determining whether a subject has a G6PC predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the G6PC inhibitor is a small molecule. In some embodiments, the G6PC inhibitor is 4,5,6,7-tetrahydrothieno[3,2-c]-and-[2,3-c]pyridine. In some embodiments, the G6PC inhibitor is a chlorogenic acid derivative, for example S-4048. Numerous chlorogenic acid derivative that inhibit G6PC are described in, for example, Arion et al., Arch. Biochem. Biophys., 1997, 339, 315-322, Herling et al., Eur. J. Pharmacol., 1999, 386, 75-82; and Herling et al., Am. J. Physiol., 1998, 274, G1087-G1093. In some embodiments, the G6PC inhibitor is 3-mercaptopicolinate. In some embodiments, the G6PC inhibitor is 1-cyclohexyl-3-(2-morpholinoethyl)carbodiimide. In some embodiments, the G6PC inhibitor is an alkanonyl glycoside. In some embodiments, the G6PC inhibitor is a (3-pyridin-2-yl-thiouriedo)alkanoic acid ester. In some embodiments, the G6PC inhibitor is a bicyclic biaryl. In some embodiments, the G6PC inhibitor is a N,N-dibenzyl-N′-benzylidenehydrazine. In some embodiments, the G6PC inhibitor is a peroxyvanadium or vandate compound. In some embodiments, the G6PC inhibitor is tungstate. In some embodiments, the G6PC inhibitor is an unsaturated aliphatic aldehydes and ketone. In some embodiments, the G6PC inhibitor is pentamidine. In some embodiments, the G6PC inhibitor is amiloride. In some embodiments, the G6PC inhibitor is methylthioadenosine sulfoxide. In some embodiments, the G6PC inhibitor is diethyl pyrocarbonate (DEPC). In some embodiments, the G6PC inhibitor is 4,4′-diisothiocyanostilbene 2,2′-disulfonic acid (DIDS). In some embodiments, the G6PC inhibitor is a N-alkylmaleimide. In some embodiments, the G6PC inhibitor is p-chloromercuribenzenesulphonate. In some embodiments, the G6PC inhibitor is an orthophosphate ester. In some embodiments, the G6PC inhibitor is 4-Hydroxynonenal. In some embodiments, the G6PC inhibitor is p-aminophenol. In some embodiments, the G6PC inhibitor is saccharin. In some embodiments, the G6PC inhibitor is phlorizin. In some embodiments, the G6PC inhibitor is cyclamate. In some embodiments, the G6PC inhibitor is a sulfhydryl compound. In some embodiments, the G6PC inhibitor is an α-bromo-, α,α-dibromo-, or α-bromo-α,β-unsaturated phosphonate. In some embodiments, the G6PC inhibitor is 4-methoxyphenyl-[4-(4-methoxyphenyl)-4,5,6,7-tetrahydrothieno[3,2-c]pyridin-5-yl]methanone or 4-methoxyphenyl-[4-(4-trifluoromethoxyphenyl)-4,5,6,7-tetrahydrothieno[3,2-c]pyridin-5-yl]methanone.

The present disclosure also provides methods of treating a subject with a therapeutic agent that treats or inhibits a kidney disease, wherein the subject has a kidney disease. In some embodiments, the method comprises determining whether the subject has an LRP2 predicted loss-of-function polypeptide by obtaining or having obtained a biological sample from the subject, and performing or having performed an assay on the biological sample to determine if the subject has an LRP2 predicted loss-of-function polypeptide. When the subject does not have an LRP2 predicted loss-of-function polypeptide, the therapeutic agent that treats or inhibits the kidney disease is administered or continued to be administered to the subject in an amount that is the same as or less than a standard dosage amount, and/or an LRP2 inhibitor is administered to the subject. When the subject has an LRP2 predicted loss-of-function polypeptide, the therapeutic agent that treats or inhibits the kidney disease is administered or continued to be administered to the subject in an amount that is the same as or less than a standard dosage amount, and/or an LRP2 inhibitor is administered to the subject. The presence of an LRP2 predicted loss-of-function polypeptide indicates the subject has a decreased risk of developing a kidney disease. In some embodiments, the subject has an LRP2 predicted loss-of-function polypeptide. In some embodiments, the subject does not have an LRP2 predicted loss-of-function polypeptide.

The present disclosure also provides methods of preventing a subject from developing a kidney disease by administering a therapeutic agent that prevents the kidney disease. In some embodiments, the method comprises determining whether the subject has an LRP2 predicted loss-of-function polypeptide by obtaining or having obtained a biological sample from the subject, and performing or having performed an assay on the biological sample to determine if the subject has an LRP2 predicted loss-of-function polypeptide. When the subject does not have an LRP2 predicted loss-of-function polypeptide, the therapeutic agent that prevents the kidney disease is administered or continued to be administered to the subject in an amount that is the same as or less than a standard dosage amount, and/or an LRP2 inhibitor is administered to the subject. When the subject has an LRP2 predicted loss-of-function polypeptide, the therapeutic agent that prevents the kidney disease is administered or continued to be administered to the subject in an amount that is the same as or less than a standard dosage amount, and/or an LRP2 inhibitor is administered to the subject. The presence of an LRP2 predicted loss-of-function polypeptide indicates the subject has a decreased risk of developing a kidney disease. In some embodiments, the subject has an LRP2 predicted loss-of-function polypeptide. In some embodiments, the subject does not have an LRP2 predicted loss-of-function polypeptide.

Detecting the presence or absence of an LRP2 predicted loss-of-function polypeptide in a biological sample from a subject and/or determining whether a subject has an LRP2 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the LRP2 inhibitor is a small molecule. In some embodiments, the LRP2 inhibitor is an aminoglycoside, such as gentamicin. In some embodiments, the LRP2 inhibitor is polymyxin, such as polymyxin B or polymyxin E (colistin). In some embodiments, the LRP2 inhibitor is aprotinin. In some embodiments, the LRP2 inhibitor is cilastatin.

The present disclosure also provides methods of treating a subject with a therapeutic agent that treats or inhibits a kidney disease, wherein the subject has a kidney disease. In some embodiments, the method comprises determining whether the subject has an RPL3L predicted loss-of-function polypeptide by obtaining or having obtained a biological sample from the subject, and performing or having performed an assay on the biological sample to determine if the subject has an RPL3L predicted loss-of-function polypeptide. When the subject does not have an RPL3L predicted loss-of-function polypeptide, the therapeutic agent that treats or inhibits the kidney disease is administered or continued to be administered to the subject in an amount that is the same as or less than a standard dosage amount, and/or an RPL3L inhibitor is administered to the subject. When the subject has an RPL3L predicted loss-of-function polypeptide, the therapeutic agent that treats or inhibits the kidney disease is administered or continued to be administered to the subject in an amount that is the same as or less than a standard dosage amount, and/or an RPL3L inhibitor is administered to the subject. The presence of an RPL3L predicted loss-of-function polypeptide indicates the subject has a decreased risk of developing a kidney disease. In some embodiments, the subject has an RPL3L predicted loss-of-function polypeptide. In some embodiments, the subject does not have an RPL3L predicted loss-of-function polypeptide.

The present disclosure also provides methods of preventing a subject from developing a kidney disease by administering a therapeutic agent that prevents the kidney disease. In some embodiments, the method comprises determining whether the subject has an RPL3L predicted loss-of-function polypeptide by obtaining or having obtained a biological sample from the subject, and performing or having performed an assay on the biological sample to determine if the subject has an RPL3L predicted loss-of-function polypeptide. When the subject does not have an RPL3L predicted loss-of-function polypeptide, the therapeutic agent that prevents the kidney disease is administered or continued to be administered to the subject in an amount that is the same as or less than a standard dosage amount, and/or an RPL3L inhibitor is administered to the subject. When the subject has an RPL3L predicted loss-of-function polypeptide, the therapeutic agent that prevents the kidney disease is administered or continued to be administered to the subject in an amount that is the same as or less than a standard dosage amount, and/or an RPL3L inhibitor is administered to the subject. The presence of an RPL3L predicted loss-of-function polypeptide indicates the subject has a decreased risk of developing a kidney disease. In some embodiments, the subject has an RPL3L predicted loss-of-function polypeptide. In some embodiments, the subject does not have an RPL3L predicted loss-of-function polypeptide.

Detecting the presence or absence of an RPL3L predicted loss-of-function polypeptide in a biological sample from a subject and/or determining whether a subject has an RPL3L predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the RPL3L inhibitor is a small molecule. In some embodiments, the RPL3L inhibitor is an inhibitory nucleic acid molecule, such as an antisense molecule or an siRNA molecule.

The present disclosure also provides methods of treating a subject with a therapeutic agent that treats or inhibits a kidney disease, wherein the subject has a kidney disease. In some embodiments, the method comprises determining whether the subject has an SLC25A45 predicted loss-of-function polypeptide by obtaining or having obtained a biological sample from the subject, and performing or having performed an assay on the biological sample to determine if the subject has an SLC25A45 predicted loss-of-function polypeptide. When the subject does not have an SLC25A45 predicted loss-of-function polypeptide, the therapeutic agent that treats or inhibits the kidney disease is administered or continued to be administered to the subject in an amount that is the same as or less than a standard dosage amount, and/or an SLC25A45 inhibitor is administered to the subject. When the subject has an SLC25A45 predicted loss-of-function polypeptide, the therapeutic agent that treats or inhibits the kidney disease is administered or continued to be administered to the subject in an amount that is the same as or less than a standard dosage amount, and/or an SLC25A45 inhibitor is administered to the subject. The presence of an SLC25A45 predicted loss-of-function polypeptide indicates the subject has a decreased risk of developing a kidney disease. In some embodiments, the subject has an SLC25A45 predicted loss-of-function polypeptide. In some embodiments, the subject does not have an SLC25A45 predicted loss-of-function polypeptide.

The present disclosure also provides methods of preventing a subject from developing a kidney disease by administering a therapeutic agent that prevents the kidney disease. In some embodiments, the method comprises determining whether the subject has an SLC25A45 predicted loss-of-function polypeptide by obtaining or having obtained a biological sample from the subject, and performing or having performed an assay on the biological sample to determine if the subject has an SLC25A45 predicted loss-of-function polypeptide. When the subject does not have an SLC25A45 predicted loss-of-function polypeptide, the therapeutic agent that prevents the kidney disease is administered or continued to be administered to the subject in an amount that is the same as or less than a standard dosage amount, and/or an SLC25A45 inhibitor is administered to the subject. When the subject has an SLC25A45 predicted loss-of-function polypeptide, the therapeutic agent that prevents the kidney disease is administered or continued to be administered to the subject in an amount that is the same as or less than a standard dosage amount, and/or an SLC25A45 inhibitor is administered to the subject. The presence of an SLC25A45 predicted loss-of-function polypeptide indicates the subject has a decreased risk of developing a kidney disease. In some embodiments, the subject has an SLC25A45 predicted loss-of-function polypeptide. In some embodiments, the subject does not have an SLC25A45 predicted loss-of-function polypeptide.

Detecting the presence or absence of an SLC25A45 predicted loss-of-function polypeptide in a biological sample from a subject and/or determining whether a subject has an SLC25A45 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the SLC25A45 inhibitor is a small molecule. In some embodiments, the SLC25A45 inhibitor is an inhibitory nucleic acid molecule, such as an antisense molecule or an siRNA molecule.

The present disclosure also provides methods of treating a subject with a therapeutic agent that treats or inhibits a kidney disease, wherein the subject has a kidney disease. In some embodiments, the method comprises determining whether the subject has an SLC7A9 predicted loss-of-function polypeptide by obtaining or having obtained a biological sample from the subject, and performing or having performed an assay on the biological sample to determine if the subject has an SLC7A9 predicted loss-of-function polypeptide. When the subject does not have an SLC7A9 predicted loss-of-function polypeptide, the therapeutic agent that treats or inhibits the kidney disease is administered or continued to be administered to the subject in an amount that is the same as or less than a standard dosage amount, and/or an SLC7A9 inhibitor is administered to the subject. When the subject has an SLC7A9 predicted loss-of-function polypeptide, the therapeutic agent that treats or inhibits the kidney disease is administered or continued to be administered to the subject in an amount that is the same as or less than a standard dosage amount, and/or an SLC7A9 inhibitor is administered to the subject. The presence of an SLC7A9 predicted loss-of-function polypeptide indicates the subject has a decreased risk of developing a kidney disease. In some embodiments, the subject has an SLC7A9 predicted loss-of-function polypeptide. In some embodiments, the subject does not have an SLC7A9 predicted loss-of-function polypeptide.

The present disclosure also provides methods of preventing a subject from developing a kidney disease by administering a therapeutic agent that prevents the kidney disease. In some embodiments, the method comprises determining whether the subject has an SLC7A9 predicted loss-of-function polypeptide by obtaining or having obtained a biological sample from the subject, and performing or having performed an assay on the biological sample to determine if the subject has an SLC7A9 predicted loss-of-function polypeptide. When the subject does not have an SLC7A9 predicted loss-of-function polypeptide, the therapeutic agent that prevents the kidney disease is administered or continued to be administered to the subject in an amount that is the same as or less than a standard dosage amount, and/or an SLC7A9 inhibitor is administered to the subject. When the subject has an SLC7A9 predicted loss-of-function polypeptide, the therapeutic agent that prevents the kidney disease is administered or continued to be administered to the subject in an amount that is the same as or less than a standard dosage amount, and/or an SLC7A9 inhibitor is administered to the subject. The presence of an SLC7A9 predicted loss-of-function polypeptide indicates the subject has a decreased risk of developing a kidney disease. In some embodiments, the subject has an SLC7A9 predicted loss-of-function polypeptide. In some embodiments, the subject does not have an SLC7A9 predicted loss-of-function polypeptide.

Detecting the presence or absence of an SLC7A9 predicted loss-of-function polypeptide in a biological sample from a subject and/or determining whether a subject has an SLC7A9 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the SLC7A9 inhibitor is a small molecule. In some embodiments, the SLC7A9 inhibitor is dibutylyl adenosine 3′,5′-cyclic monophosphate (db-cAMP).

In any of the embodiments described herein, when the subject does not have any of ALDH1L1, ALDOB, G6PC, LRP2, RPL3L, SLC25A45, or SLC7A9 predicted loss-of-function polypeptides, the therapeutic agent that treats or inhibits a kidney disease is administered or continued to be administered to the subject in an amount that is the same as or less than a standard dosage amount, and/or a combination of one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, is administered to the subject. When the subject has one or more of ALDH1L1, ALDOB, G6PC, LRP2, RPL3L, SLC25A45, or SLC7A9 predicted loss-of-function polypeptides, the therapeutic agent that prevents a kidney disease is administered or continued to be administered to the subject in an amount that is the same as or less than a standard dosage amount. In addition, the subject may be administered a combination of one or more of ALDH1L1 inhibitors, ALDOB inhibitors, G6PC inhibitors, LRP2 inhibitors, RPL3L inhibitors, SLC25A45 inhibitors, or SLC7A9 inhibitors, wherein the combination comprises the inhibitors for the targets for which the subject is reference, i.e., lacks predicted loss-of-function polypeptides. For example, when the subject is determined to be ALDOB, G6PC, RPL3L, and SLC7A9 reference (i.e., the subject does not have ALDOB, G6PC, RPL3L, and SLC7A9 loss-of-function polypeptides) the subject is administered or continued to be administered the therapeutic agent that treats or inhibits the kidney disease in an amount that is the same as or less than a standard dosage amount and is administered a combination of an ALDOB inhibitor, a G6PC inhibitor, a RPL3L inhibitor, and an SLC7A9 inhibitor. In some embodiments, the subject is administered the combination of inhibitors that corresponds to any subset of ALDH1L1, ALDOB, G6PC, LRP2, RPL3L, SLC25A45, and SLC7A9 for which the subject was found to be reference or heterozygous for a variant nucleic acid molecule encoding corresponding loss-of-function polypeptides. The presence of one or more of ALDH1L1, ALDOB, G6PC, LRP2, RPL3L, SLC25A45, and/or SLC7A9 predicted loss-of-function polypeptides indicates the subject has a decreased risk of developing a kidney disease. The presence of a greater number of any of ALDH1L1, ALDOB, G6PC, LRP2, RPL3L, SLC25A45, and/or SLC7A9 predicted loss-of-function polypeptides is considered more protective of a kidney disease. Thus, having any four of ALDH1L1, ALDOB, G6PC, LRP2, RPL3L, SLC25A45, and SLC7A9 predicted loss-of-function polypeptides is more protective than having any three of ALDH1L1, ALDOB, G6PC, LRP2, RPL3L, SLC25A45, and SLC7A9 predicted loss-of-function polypeptides, which is, in turn, more protective than having any two of ALDH1L1, ALDOB, G6PC, LRP2, RPL3L, SLC25A45, and SLC7A9 predicted loss-of-function polypeptides. In some embodiments, the subject has one or more of ALDH1L1, ALDOB, G6PC, LRP2, RPL3L, SLC25A45, and/or SLC7A9 predicted loss-of-function polypeptides. In some embodiments, the subject does not have one or more of ALDH1L1, ALDOB, G6PC, LRP2, RPL3L, SLC25A45, and/or SLC7A9 predicted loss-of-function polypeptides.

Examples of therapeutic agents that treat or inhibit chronic kidney disease include, but are not limited to, erythropoietin, a diuretic (such as, for example, furosemide, bumetanide, ethacrynic acid, metolazone, and hydrochlorothiazide), a blood pressure medication, a phosphate binder, sodium bicarbonate, a cholesterol medication, and a gliflozin, or any combination thereof.

Examples of therapeutic agents that treat or inhibit a kidney stone include, but are not limited to, potassium citrate, a diuretic (such as, for example, furosemide, bumetanide, ethacrynic acid, metolazone, and hydrochlorothiazide), allopurinol, acetohydroxamic acid, tamsulosin, nifedipine, d-penicillamine, tiopronin, and mercaptopropionyl glycine, or any combination thereof.

Examples of therapeutic agents that treat or inhibit chronic glomerulonephritis include, but are not limited to, an angiotensin-converting enzyme (ACE) inhibitor (such as, for example, lisinopril, enalapril, captopril, benazepril, fosinopril, and quinapril), a diuretic (such as, for example, furosemide, bumetanide, ethacrynic acid, metolazone, and hydrochlorothiazide), a calcium channel blocker (such as, for example, amlodipine, nifedipine, felodipine, isradipine, verapamil, and diltiazem), a beta-adrenergic blocker (such as, for example, metoprolol, bisoprolol, esmolol, atenolol, propranolol, sotalol, labetalol, pindolol, and penbutolol), an alpha-adrenergic agonist (such as, for example, clonidine, tizanidine, and dexmedetomidine), a corticosteroid (such as, for example, prednisone), and an immunosuppressant (such as, for example, cyclosphosphamide), or any combination thereof.

Examples of therapeutic agents that treat or inhibit nephrosis include, but are not limited to, an ACE inhibitor (such as, for example, lisinopril, enalapril, captopril, benazepril, fosinopril, and quinapril), a diuretic (such as, for example, furosemide, bumetanide, ethacrynic acid, metolazone, and hydrochlorothiazide), a statin (such as, for example, atorvastatin, fluvastatin, lovastatin, pravastatin, rosuvastatin, and simvastatin), an angiotensin II receptor blockers (ARBs) (such as, for example, losartan and valsartan), an anticoagulant (such as, for example, heparin, warfarin, dabigatran, apixaban, and rivaroxaban), and an anti-inflammatory immunosuppressant (such as, for example, rituximab, cyclosporine, and cyclophosphamide), or any combination thereof.

Examples of therapeutic agents that treat or inhibit nephronophthisis include, but are not limited to, erythropoietin and a blood pressure medication, or any combination thereof.

Examples of therapeutic agents that treat or inhibit chronic interstitial nephritis include, but are not limited to, a corticosteroid, erythropoietin, a blood pressure medication, a statin, and a chelating agent (such as, for example, succimer and edetate calcium disodium), or a combination thereof.

Examples of therapeutic agents that treat or inhibit nephrosclerosis include, but are not limited to, a diuretic (such as, for example, furosemide, bumetanide, ethacrynic acid, metolazone, and hydrochlorothiazide), an ACE inhibitor (such as, for example, lisinopril, enalapril, captopril, benazepril, fosinopril, and quinapril), an ARB (such as, for example, losartan, and valsartan), a calcium channel blocker (such as, for example, amlodipine, nifedipine, felodipine, isradipine, verapamil, and diltiazem), a beta-adrenergic blocker (such as, for example, metoprolol, bisoprolol, esmolol, atenolol, propranolol, sotalol, labetalol, pindolol, and penbutolol), an alpha-adrenergic agonist (such as, for example, clonidine, tizanidine, and dexmedetomidine), a renin inhibitor (such as, for example, aliskiren), a vasodilator (such as, for example, minoxidil and hydralazine), and an alpha-1 blocker (such as, for example, doxazosin), or any combination thereof.

In some embodiments, the dose of the therapeutic agents that treat, prevent, or inhibit a kidney disease can be decreased by about 10%, by about 20%, by about 30%, by about 40%, by about 50%, by about 60%, by about 70%, by about 80%, or by about 90% for subjects that are heterozygous for one or more of ALDH1L1, ALDOB, G6PC, LRP2, RPL3L, SLC25A45, and/or SLC7A9 variant nucleic acid molecules encoding one or more ALDH1L1, ALDOB, G6PC, LRP2, RPL3L, SLC25A45, and/or SLC7A9 predicted loss-of-function polypeptides (i.e., a less than the standard dosage amount) compared to subjects that are reference for one or more of ALDH1L1, ALDOB, G6PC, LRP2, RPL3L, SLC25A45, and/or SLC7A9. In some embodiments, the dose of the therapeutic agents that treat, prevent, or inhibit a kidney disease can be decreased by about 10%, by about 20%, by about 30%, by about 40%, or by about 50%. In addition, the subjects that are heterozygous for one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides can be administered less frequently compared to subjects that are ALDH1L1, ALDOB, G6PC, LRP2, RPL3L, SLC25A45, and SLC7A9 reference.

In some embodiments, the dose of the therapeutic agents that treat, prevent, or inhibit a kidney disease can be decreased by about 10%, by about 20%, by about 30%, by about 40%, by about 50%, for subjects that are homozygous for one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides compared to subjects that are heterozygous for one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides. In some embodiments, the dose of the therapeutic agents that treat, prevent, or inhibit a kidney disease can be decreased by about 10%, by about 20%, by about 30%, by about 40%, or by about 50%. In addition, the dose of therapeutic agents that treat, prevent, or inhibit a kidney disease in subjects that are homozygous for one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides can be administered less frequently compared to subjects that are heterozygous for one or more of ALDH1L1, ALDOB, G6PC, LRP2, RPL3L, SLC25A45, and/or SLC7A9 variant nucleic acid molecule encoding one or more of ALDH1L1, ALDOB, G6PC, LRP2, RPL3L, SLC25A45, and/or SLC7A9 predicted loss-of-function polypeptides.

Administration of the therapeutic agents that treat, prevent, or inhibit a kidney disease and/or one or more of ALDH1L1, ALDOB, G6PC, LRP2, RPL3L, SLC25A45, and/or SLC7A9 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat, prevent, or inhibit a kidney disease and/or one or more of ALDH1L1, ALDOB, G6PC, LRP2, RPL3L, SLC25A45, and/or SLC7A9 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in kidney disease, a decrease/reduction in the severity of kidney disease (such as, for example, a reduction or inhibition of development of kidney disease), a decrease/reduction in symptoms and kidney disease-related effects, delaying the onset of symptoms and kidney disease-related effects, reducing the severity of symptoms of kidney disease-related effects, reducing the number of symptoms and kidney disease-related effects, reducing the latency of symptoms and kidney disease-related effects, an amelioration of symptoms and kidney disease-related effects, reducing secondary symptoms, reducing secondary infections, preventing relapse to kidney disease, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of kidney disease development/progression (such as, for example, a complete or partial avoidance/inhibition or a delay), and an increased survival time of the affected host animal, following administration of a therapeutic protocol. Treatment of kidney disease encompasses the treatment of a subject already diagnosed as having any form of kidney disease at any clinical stage or manifestation, the delay of the onset or evolution or aggravation or deterioration of the symptoms or signs of kidney disease, and/or preventing and/or reducing the severity of kidney disease.

The present disclosure also provides methods of identifying a subject having an increased risk of developing a kidney disease. In some embodiments, the method comprises determining or having determined in a biological sample obtained from the subject the presence or absence of one or more of ALDH1L1, ALDOB, G6PC, LRP2, RPL3L, SLC25A45, and/or SLC7A9 variant nucleic acid molecules (such as a genomic nucleic acid molecule, mRNA molecule, and/or cDNA molecule) encoding one or more of ALDH1L1, ALDOB, G6PC, LRP2, RPL3L, SLC25A45, and/or SLC7A9 predicted loss-of-function polypeptides. When the subject lacks ALDH1L1, ALDOB, G6PC, LRP2, RPL3L, SLC25A45, and SLC7A9 variant nucleic acid molecules encoding ALDH1L1, ALDOB, G6PC, LRP2, RPL3L, SLC25A45, and SLC7A9 predicted loss-of-function polypeptides (i.e., the subject is genotypically categorized as ALDH1L1, ALDOB, G6PC, LRP2, RPL3L, SLC25A45, and SLC7A9 reference), then the subject has an increased risk of developing a kidney disease. When the subject has one or more of ALDH1L1, ALDOB, G6PC, LRP2, RPL3L, SLC25A45, and SLC7A9 variant nucleic acid molecules encoding one or more of ALDH1L1, ALDOB, G6PC, LRP2, RPL3L, SLC25A45, and/or SLC7A9 predicted loss-of-function polypeptides (i.e., the subject is heterozygous or homozygous for one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides), then the subject has a decreased risk of developing a kidney disease.

Having a single copy of any one of ALDH1L1, ALDOB, G6PC, LRP2, RPL3L, SLC25A45, and SLC7A9 variant nucleic acid molecules encoding any one of ALDH1L1, ALDOB, G6PC, LRP2, RPL3L, SLC25A45, and SLC7A9 predicted loss-of-function polypeptides is more protective of a subject from developing a kidney disease than having no copies of an ALDH1L1, ALDOB, G6PC, LRP2, RPL3L, SLC25A45, and/or SLC7A9 variant nucleic acid molecules encoding ALDH1L1, ALDOB, G6PC, LRP2, RPL3L, SLC25A45, and/or SLC7A9 predicted loss-of-function polypeptides. Without intending to be limited to any particular theory or mechanism of action, it is believed that a single copy of any one of ALDH1L1, ALDOB, G6PC, LRP2, RPL3L, SLC25A45, and/or SLC7A9 variant nucleic acid molecules encoding ALDH1L1, ALDOB, G6PC, LRP2, RPL3L, SLC25A45, and/or SLC7A9 predicted loss-of-function polypeptides (i.e., heterozygous for one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides) is protective of a subject from developing a kidney disease, and it is also believed that having two copies of any one of ALDH1L1, ALDOB, G6PC, LRP2, RPL3L, SLC25A45, and/or SLC7A9 variant nucleic acid molecules encoding ALDH1L1, ALDOB, G6PC, LRP2, RPL3L, SLC25A45, and/or SLC7A9 predicted loss-of-function polypeptides (i.e., homozygous for one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides) may be more protective of a subject from developing a kidney disease, relative to a subject with a single copy of the corresponding gene. Thus, in some embodiments, a single copy of any one of ALDH1L1, ALDOB, G6PC, LRP2, RPL3L, SLC25A45, and SLC7A9 variant nucleic acid molecules encoding any one of ALDH1L1, ALDOB, G6PC, LRP2, RPL3L, SLC25A45, and SLC7A9 predicted loss-of-function polypeptides may not be completely protective, but instead, may be partially or incompletely protective of a subject from developing a kidney disease. While not desiring to be bound by any particular theory, there may be additional factors or molecules involved in the development of a kidney disease that are still present in a subject having a single copy of any one of ALDH1L1, ALDOB, G6PC, LRP2, RPL3L, SLC25A45, and/or SLC7A9 variant nucleic acid molecule encoding any one of ALDH1L1, ALDOB, G6PC, LRP2, RPL3L, SLC25A45, and SLC7A9 predicted loss-of-function polypeptide, thus resulting in less than complete protection from the development of a kidney disease.

Determining whether a subject has one or more of ALDH1L1, ALDOB, G6PC, LRP2, RPL3L, SLC25A45, and/or SLC7A9 variant nucleic acid molecule encoding one or more of ALDH1L1, ALDOB, G6PC, LRP2, RPL3L, SLC25A45, and/or SLC7A9 predicted loss-of-function polypeptides in a biological sample from a subject and/or determining whether a subject has one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing a kidney disease, the subject is administered a therapeutic agent that treats, prevents, or inhibits a kidney disease, and/or one or more ALDH1L1 inhibitors, ALDOB inhibitors, G6PC inhibitors, LRP2 inhibitors, RPL3L inhibitors, SLC25A45 inhibitors, and/or SLC7A9 inhibitors, or any combination thereof, as described herein.

For example, when the subject is ALDH1L1 reference, and therefore has an increased risk of developing a kidney disease, the subject is administered an ALDH1L1 inhibitor. In some embodiments, such a subject is also administered a therapeutic agent that treats, prevents, or inhibits a kidney disease. In some embodiments, when the subject is heterozygous for an ALDH1L1 variant nucleic acid molecule encoding an ALDH1L1 predicted loss-of-function polypeptide, the subject is administered the therapeutic agent that treats, prevents, or inhibits a kidney disease in a dosage amount that is the same as or less than a standard dosage amount, and is also administered an ALDH1L1 inhibitor. In some embodiments, such a subject is also administered a therapeutic agent that treats, prevents, or inhibits a kidney disease. In some embodiments, when the subject is homozygous for an ALDH1L1 variant nucleic acid molecule encoding an ALDH1L1 predicted loss-of-function polypeptide, the subject is administered the therapeutic agent that treats, prevents, or inhibits a kidney disease in a standard dosage amount. In some embodiments, the subject is ALDH1L1 reference. In some embodiments, the subject is heterozygous for an ALDH1L1 variant nucleic acid molecule encoding an ALDH1L1 predicted loss-of-function polypeptide. In some embodiments, the subject is homozygous for an ALDH1L1 variant nucleic acid molecule encoding an ALDH1L1 predicted loss-of-function polypeptide.

In addition, when the subject is ALDOB reference, and therefore has an increased risk of developing a kidney disease, the subject is administered an ALDOB inhibitor. In some embodiments, such a subject is also administered a therapeutic agent that treats, prevents, or inhibits a kidney disease. In some embodiments, when the subject is heterozygous for an ALDOB variant nucleic acid molecule encoding an ALDOB predicted loss-of-function polypeptide, the subject is administered the therapeutic agent that treats, prevents, or inhibits a kidney disease in a dosage amount that is the same as or less than a standard dosage amount, and is also administered an ALDOB inhibitor. In some embodiments, such a subject is also administered a therapeutic agent that treats, prevents, or inhibits a kidney disease. In some embodiments, when the subject is homozygous for an ALDOB variant nucleic acid molecule encoding an ALDOB predicted loss-of-function polypeptide, the subject is administered the therapeutic agent that treats, prevents, or inhibits a kidney disease in a standard dosage amount. In some embodiments, the subject is ALDOB reference. In some embodiments, the subject is heterozygous for an ALDOB variant nucleic acid molecule encoding an ALDOB predicted loss-of-function polypeptide. In some embodiments, the subject is homozygous for an ALDOB variant nucleic acid molecule encoding an ALDOB predicted loss-of-function polypeptide.

In addition, when the subject is ALDH1L1 reference, and therefore has an increased risk of developing a kidney disease, the subject is administered an ALDH1L1 inhibitor. In some embodiments, such a subject is also administered a therapeutic agent that treats, prevents, or inhibits a kidney disease. In some embodiments, when the subject is heterozygous for an ALDH1L1 variant nucleic acid molecule encoding an ALDH1L1 predicted loss-of-function polypeptide, the subject is administered the therapeutic agent that treats, prevents, or inhibits a kidney disease in a dosage amount that is the same as or less than a standard dosage amount, and is also administered an ALDH1L1 inhibitor. In some embodiments, such a subject is also administered a therapeutic agent that treats, prevents, or inhibits a kidney disease. In some embodiments, when the subject is homozygous for an ALDH1L1 variant nucleic acid molecule encoding an ALDH1L1 predicted loss-of-function polypeptide, the subject is administered the therapeutic agent that treats, prevents, or inhibits a kidney disease in a standard dosage amount. In some embodiments, the subject is ALDH1L1 reference. In some embodiments, the subject is heterozygous for an ALDH1L1 variant nucleic acid molecule encoding an ALDH1L1 predicted loss-of-function polypeptide. In some embodiments, the subject is homozygous for an ALDH1L1 variant nucleic acid molecule encoding an ALDH1L1 predicted loss-of-function polypeptide.

In addition, when the subject is LRP2 reference, and therefore has an increased risk of developing a kidney disease, the subject is administered an LRP2 inhibitor. In some embodiments, such a subject is also administered a therapeutic agent that treats, prevents, or inhibits a kidney disease. In some embodiments, when the subject is heterozygous for an LRP2 variant nucleic acid molecule encoding an LRP2 predicted loss-of-function polypeptide, the subject is administered the therapeutic agent that treats, prevents, or inhibits a kidney disease in a dosage amount that is the same as or less than a standard dosage amount, and is also administered an LRP2 inhibitor. In some embodiments, such a subject is also administered a therapeutic agent that treats, prevents, or inhibits a kidney disease. In some embodiments, when the subject is homozygous for an LRP2 variant nucleic acid molecule encoding an LRP2 predicted loss-of-function polypeptide, the subject is administered the therapeutic agent that treats, prevents, or inhibits a kidney disease in a standard dosage amount. In some embodiments, the subject is LRP2 reference. In some embodiments, the subject is heterozygous for an LRP2 variant nucleic acid molecule encoding an LRP2 predicted loss-of-function polypeptide. In some embodiments, the subject is homozygous for an LRP2 variant nucleic acid molecule encoding an LRP2 predicted loss-of-function polypeptide.

In addition, when the subject is RPL3L reference, and therefore has an increased risk of developing a kidney disease, the subject is administered an RPL3L inhibitor. In some embodiments, such a subject is also administered a therapeutic agent that treats, prevents, or inhibits a kidney disease. In some embodiments, when the subject is heterozygous for an RPL3L variant nucleic acid molecule encoding an RPL3L predicted loss-of-function polypeptide, the subject is administered the therapeutic agent that treats, prevents, or inhibits a kidney disease in a dosage amount that is the same as or less than a standard dosage amount, and is also administered an RPL3L inhibitor. In some embodiments, such a subject is also administered a therapeutic agent that treats, prevents, or inhibits a kidney disease. In some embodiments, when the subject is homozygous for an RPL3L variant nucleic acid molecule encoding an RPL3L predicted loss-of-function polypeptide, the subject is administered the therapeutic agent that treats, prevents, or inhibits a kidney disease in a standard dosage amount. In some embodiments, the subject is RPL3L reference. In some embodiments, the subject is heterozygous for an RPL3L variant nucleic acid molecule encoding an RPL3L predicted loss-of-function polypeptide. In some embodiments, the subject is homozygous for an RPL3L variant nucleic acid molecule encoding an RPL3L predicted loss-of-function polypeptide.

In addition, when the subject is SLC25A45 reference, and therefore has an increased risk of developing a kidney disease, the subject is administered an SLC25A45 inhibitor. In some embodiments, such a subject is also administered a therapeutic agent that treats, prevents, or inhibits a kidney disease. In some embodiments, when the subject is heterozygous for an SLC25A45 variant nucleic acid molecule encoding an SLC25A45 predicted loss-of-function polypeptide, the subject is administered the therapeutic agent that treats, prevents, or inhibits a kidney disease in a dosage amount that is the same as or less than a standard dosage amount, and is also administered an SLC25A45 inhibitor. In some embodiments, such a subject is also administered a therapeutic agent that treats, prevents, or inhibits a kidney disease. In some embodiments, when the subject is homozygous for an SLC25A45 variant nucleic acid molecule encoding an SLC25A45 predicted loss-of-function polypeptide, the subject is administered the therapeutic agent that treats, prevents, or inhibits a kidney disease in a standard dosage amount. In some embodiments, the subject is SLC25A45 reference. In some embodiments, the subject is heterozygous for an SLC25A45 variant nucleic acid molecule encoding an SLC25A45 predicted loss-of-function polypeptide. In some embodiments, the subject is homozygous for an SLC25A45 variant nucleic acid molecule encoding an SLC25A45 predicted loss-of-function polypeptide.

In addition, when the subject is SLC7A9 reference, and therefore has an increased risk of developing a kidney disease, the subject is administered an SLC7A9 inhibitor. In some embodiments, such a subject is also administered a therapeutic agent that treats, prevents, or inhibits a kidney disease. In some embodiments, when the subject is heterozygous for an SLC7A9 variant nucleic acid molecule encoding an SLC7A9 predicted loss-of-function polypeptide, the subject is administered the therapeutic agent that treats, prevents, or inhibits a kidney disease in a dosage amount that is the same as or less than a standard dosage amount, and is also administered an SLC7A9 inhibitor. In some embodiments, such a subject is also administered a therapeutic agent that treats, prevents, or inhibits a kidney disease. In some embodiments, when the subject is homozygous for an SLC7A9 variant nucleic acid molecule encoding an SLC7A9 predicted loss-of-function polypeptide, the subject is administered the therapeutic agent that treats, prevents, or inhibits a kidney disease in a standard dosage amount. In some embodiments, the subject is SLC7A9 reference. In some embodiments, the subject is heterozygous for an SLC7A9 variant nucleic acid molecule encoding an SLC7A9 predicted loss-of-function polypeptide. In some embodiments, the subject is homozygous for an SLC7A9 variant nucleic acid molecule encoding an SLC7A9 predicted loss-of-function polypeptide.

In some embodiments, any of the methods described herein can further comprise determining the subject's aggregate gene burden of having ALDH1L1 variant nucleic acid molecules encoding ALDH1L1 predicted loss-of-function polypeptides associated with a decreased risk of developing a kidney disease. The aggregate gene burden is the sum of all variants in the ALDH1L1 gene, which can be carried out in an association analysis with a kidney disease. In some embodiments, the subject is homozygous for one or more ALDH1L1 variant nucleic acid molecules encoding ALDH1L1 predicted loss-of-function polypeptides associated with a decreased risk of developing a kidney disease. In some embodiments, the subject is heterozygous for one or more ALDH1L1 variant nucleic acid molecules encoding ALDH1L1 predicted loss-of-function polypeptides associated with a decreased risk of developing a kidney disease. The result of the association analysis suggests that ALDH1L1 variant nucleic acid molecules encoding ALDH1L1 predicted loss-of-function polypeptides are associated with decreased risk of developing a kidney disease. When the subject has a lower aggregate gene burden, the subject is at a higher risk of developing a kidney disease and the subject is administered or continued to be administered a therapeutic agent that treats, prevents, or inhibits a kidney disease in an amount that is the same as or less than a standard dosage amount, and/or an ALDH1L1 inhibitor. When the subject has a greater aggregate gene burden, the subject is at a lower risk of developing a kidney disease and the subject is administered or continued to be administered a therapeutic agent that treats, prevents, or inhibits a kidney disease in a standard dosage amount. The greater the aggregate gene burden, the lower the risk of developing a kidney disease.

ALDH1L1 pLOF variants include, but are not limited to: 3:126107153:CGA:C, 3:126112779:T:TA, 3:126112781:C:A, 3:126112817:C:A, 3:126114556:C:A, 3:126114556:C:G, 3:126118050:AT:A, 3:126118099:C:T, 3:126118100:T:C, 3:126130224:CA:C, 3:126136803:GC:G, 3:126136822:TC:T, 3:126137812:C:T, 3:126137851:G:A, 3:126150513:G:A, 3:126153567:GA:G, 3:126154620:C:T, 3:126154635:C:T, 3:126155464:TG:T, 3:126157473:GA:G, 3:126157482:CCT:C, 3:126157508:C:T, 3:126158403:AC:A, 3:126158415:C:A, 3:126158427:G:A, 3:126105750:G:A, 3:126112829:G:A, 3:126114599:GA:G, 3:126114658:T:A, 3:126118009:T:A, 3:126125615:C:A, 3:126136822:T:TC, 3:126137961:C:A, 3:126154553:C:T, 3:126157463:C:T, 3:126158477:GC:G, 3:126158641:T:C, 3:126160978:A:T, 3:126180961:C:A, 3:126180967:A:T, 3:126103829:C:A, 3:126105744:CA:C, 3:126105773:GC:G, 3:126105870:CA:C, 3:126105927:T:C, 3:126110004:G:A, 3:126112805:GA:G, 3:126112853:CT:C, 3:126112881:C:G, 3:126114556:C:T, 3:126118045:AC:A, 3:126118074:G:C, 3:126118075:AG:A, 3:126118099:CT:C, 3:126124453:T:C, 3:126125719:A:AC, 3:126125722:C:G, 3:126130244:A:T, 3:126130272:G:A, 3:126130295:T:C, 3:126131536:T:C, 3:126135534:C:T, 3:126135572:T:TC, 3:126136763:C:T, 3:126136763:C:G, 3:126136823:C:A, 3:126136848:C:CA, 3:126136885:T:C, 3:126137872:G:A, 3:126146834:C:T, 3:126146915:CT:C, 3:126146917:G:A, 3:126150403:T:TA, 3:126153495:TG:T, 3:126153510:T:TG, 3:126154645:T:G, 3:126155427:CCCTCATA:C, 3:126155446:C:A, 3:126155464:T:TG, 3:126155500:G:A, 3:126157341:A:G, 3:126157426:G:A, 3:126158403:A:G, 3:126158404:C:T, 3:126158471:C:CG, 3:126158523:C:CGTTG, 3:126158560:CA:C, 3:126158590:C:T, 3:126158617:TC:T, 3:126160852:C:T, 3:126160855:AG:A, 3:126160866:C:CT, 3:126161003:C:G, 3:126161004:T:A, 3:126180474:A:G, and 3:126180967:A:G (according to the GRCh38/hg38 human genome assembly).

ALDH1L1 pLOF or missense variants predicted to be deleterious by 5 out of 5, respectively, in silico prediction algorithms AAF<1% include, but are not limited to: 3:126103820:G:A, 3:126105751:T:A, 3:126105752:T:C, 3:126105758:C:A, 3:126105777:C:T, 3:126105787:G:T, 3:126105791:T:A, 3:126105816:C:T, 3:126105822:G:A, 3:126105831:T:C, 3:126105869:C:A, 3:126105876:C:A, 3:126105896:T:C, 3:126105924:C:A, 3:126107153:CGA:C, 3:126107154:G:A, 3:126107172:G:A, 3:126107180:G:A, 3:126107201:A:T, 3:126107214:C:T, 3:126107222:A:G, 3:126107241:A:G, 3:126109965:C:T, 3:126109982:G:A, 3:126109985:C:T, 3:126110031:G:A, 3:126110055:G:C, 3:126112779:T:TA, 3:126112781:C:A, 3:126112807:A:G, 3:126112817:C:A, 3:126114556:C:G, 3:126114556:C:A, 3:126114567:G:A, 3:126118043:G:C, 3:126118047:C:T, 3:126118048:C:T, 3:126118050:AT:A, 3:126118069:G:T, 3:126118087:C:A, 3:126118099:C:T, 3:126118100:T:C, 3:126124372:G:A, 3:126124372:G:T, 3:126124403:C:T, 3:126124438:G:A, 3:126124447:G:T, 3:126124451:C:T, 3:126125623:G:A, 3:126125624:G:T, 3:126125645:C:T, 3:126125650:G:C, 3:126125656:C:T, 3:126125678:G:C, 3:126130224:CA:C, 3:126130257:G:A, 3:126131415:C:G, 3:126131416:G:A, 3:126131421:G:A, 3:126131422:T:A, 3:126131434:T:C, 3:126131436:C:A, 3:126131460:G:A, 3:126131475:G:A, 3:126131490:A:G, 3:126131491:T:C, 3:126131527:C:T, 3:126135551:C:T, 3:126135553:C:T, 3:126135554:G:A, 3:126135559:C:T, 3:126135560:G:A, 3:126135562:G:A, 3:126135563:C:T, 3:126135577:C:G, 3:126135617:C:T, 3:126136803:GC:G, 3:126136822:TC:T, 3:126136831:A:G, 3:126136883:C:T, 3:126137812:C:T, 3:126137851:G:A, 3:126137920:A:T, 3:126146842:C:T, 3:126146844:T:C, 3:126146845:C:T, 3:126146865:T:C, 3:126146880:G:A, 3:126146913:C:T, 3:126146914:G:A, 3:126150511:C:A, 3:126150513:G:A, 3:126153567:GA:G, 3:126154582:G:A, 3:126154592:C:T, 3:126154600:C:T, 3:126154601:G:A, 3:126154620:C:T, 3:126154635:C:T, 3:126155440:C:T, 3:126155464:TG:T, 3:126155496:G:T, 3:126157400:G:T, 3:126157416:C:G, 3:126157441:C:T, 3:126157473:GA:G, 3:126157482:CCT:C, 3:126157495:C:T, 3:126157500:A:T, 3:126157508:C:T, 3:126158403:AC:A, 3:126158412:T:C, 3:126158415:C:A, 3:126158417:G:A, 3:126158421:C:T, 3:126158423:C:T, 3:126158426:C:T, 3:126158427:G:A, 3:126158447:G:A, 3:126158523:C:T, 3:126158529:G:A, 3:126158532:C:T, 3:126158535:C:T, 3:126158538:C:T, 3:126158588:C:T, 3:126158589:G:A, 3:126158595:G:A, 3:126158597:G:C, 3:126158602:C:A, 3:126158621:T:C, 3:126158623:C:A, 3:126160860:G:T, 3:126160862:C:T, 3:126160883:G:A, 3:126160901:C:T, 3:126160912:C:T, 3:126160945:C:T, 3:126160954:C:T, 3:126160970:C:T, 3:126103799:C:G, 3:126103829:C:G, 3:126105750:G:A, 3:126105765:A:G, 3:126105768:G:C, 3:126105771:C:T, 3:126105780:C:T, 3:126105786:T:G, 3:126105794:G:A, 3:126105794:G:T, 3:126105861:T:A, 3:126105895:A:T, 3:126105902:C:G, 3:126105903:G:A, 3:126107163:T:A, 3:126107175:C:T, 3:126107228:G:T, 3:126107237:A:G, 3:126109950:G:A, 3:126109968:C:T, 3:126109968:C:G, 3:126110020:C:G, 3:126110060:G:A, 3:126112798:T:C, 3:126112829:G:A, 3:126112870:G:A, 3:126114579:T:A, 3:126114582:C:A, 3:126114599:GA:G, 3:126114641:G:T, 3:126114658:T:A, 3:126118009:T:A, 3:126118063:C:T, 3:126118083:T:A, 3:126124421:C:T, 3:126124424:C:A, 3:126125615:C:A, 3:126125636:C:T, 3:126125638:G:A, 3:126130257:G:C, 3:126131390:C:G, 3:126131394:T:C, 3:126131437:C:T, 3:126131463:T:C, 3:126131484:G:A, 3:126131487:T:A, 3:126131488:C:T, 3:126135535:C:A, 3:126135557:C:T, 3:126135568:A:T, 3:126135596:C:T, 3:126135629:C:T, 3:126136810:G:T, 3:126136822:T:TC, 3:126136847:G:A, 3:126137881:C:T, 3:126137928:C:A, 3:126137940:T:C, 3:126137952:T:G, 3:126137961:C:A, 3:126146839:C:T, 3:126146854:C:T, 3:126150408:G:A, 3:126150443:A:C, 3:126150443:A:T, 3:126150503:T:A, 3:126153484:G:A, 3:126153496:G:A, 3:126153500:G:A, 3:126154553:C:T, 3:126154577:C:T, 3:126154598:C:T, 3:126155413:C:T, 3:126155433:T:C, 3:126155436:G:A, 3:126155439:G:T, 3:126155475:C:T, 3:126155476:C:T, 3:126155503:C:T, 3:126157371:A:G, 3:126157375:G:T, 3:126157375:G:A, 3:126157380:T:C, 3:126157395:G:A, 3:126157424:C:G, 3:126157438:C:T, 3:126157458:T:A, 3:126157459:C:T, 3:126157463:C:T, 3:126157464:C:G, 3:126157483:C:G, 3:126157494:C:G, 3:126158475:G:T, 3:126158477:GC:G, 3:126158491:C:T, 3:126158492:A:G, 3:126158496:G:T, 3:126158517:G:A, 3:126158526:T:G, 3:126158639:C:A, 3:126158641:T:C, 3:126160898:C:G, 3:126160951:A:G, 3:126160954:C:A, 3:126160957:T:C, 3:126160978:A:T, 3:126180961:C:A, 3:126180967:A:T, 3:126103810:G:A, 3:126103820:G:C, 3:126103829:C:A, 3:126103831:T:C, 3:126103846:C:T, 3:126105744:CA:C, 3:126105744:C:G, 3:126105748:C:A, 3:126105762:C:T, 3:126105773:GC:G, 3:126105785:T:C, 3:126105787:G:C, 3:126105795:T:C, 3:126105796:G:C, 3:126105798:T:G, 3:126105802:A:C, 3:126105806:A:T, 3:126105821:A:G, 3:126105855:C:G, 3:126105855:C:A, 3:126105870:CA:C, 3:126105872:G:T, 3:126105887:T:G, 3:126105890:G:A, 3:126105918:C:T, 3:126105927:T:C, 3:126107142:C:G, 3:126107165:A:G, 3:126107175:C:G, 3:126107202:T:C, 3:126107204:T:C, 3:126107231:G:C, 3:126107231:G:A, 3:126107246:C:G, 3:126107246:C:T, 3:126109944:C:T, 3:126109949:C:A, 3:126109949:C:G, 3:126109989:C:T, 3:126110004:G:A, 3:126110041:A:C, 3:126110051:C:T, 3:126110052:C:T, 3:126110054:T:C, 3:126110055:G:A, 3:126110067:T:C, 3:126110075:G:A, 3:126112783:A:C, 3:126112784:C:T, 3:126112793:C:T, 3:126112805:GA:G, 3:126112831:C:G, 3:126112849:T:G, 3:126112852:C:T, 3:126112853:CT:C, 3:126112859:T:A, 3:126112881:C:G, 3:126114556:C:T, 3:126114577:G:A, 3:126114594:A:T, 3:126114604:G:A, 3:126114615:C:T, 3:126114616:C:T, 3:126114624:A:G, 3:126114627:G:C, 3:126114632:C:A, 3:126114633:T:G, 3:126114633:T:A, 3:126114634:T:C, 3:126118011:A:C, 3:126118024:C:T, 3:126118035:G:A, 3:126118038:C:G, 3:126118045:AC:A, 3:126118056:C:T, 3:126118074:G:C, 3:126118075:AG:A, 3:126118083:T:G, 3:126118087:C:T, 3:126118098:C:G, 3:126118099:CT:C, 3:126124369:C:A, 3:126124369:C:T, 3:126124375:A:G, 3:126124378:A:T, 3:126124381:T:G, 3:126124382:T:G, 3:126124390:C:T, 3:126124391:C:T, 3:126124415:T:G, 3:126124426:A:G, 3:126124442:G:A, 3:126124447:G:A, 3:126124453:T:C, 3:126125626:T:A, 3:126125639:T:C, 3:126125639:T:G, 3:126125648:C:A, 3:126125654:G:C, 3:126125657:A:T, 3:126125677:A:G, 3:126125702:G:A, 3:126125716:C:G, 3:126125719:A:AC, 3:126125720:C:A, 3:126125722:C:G, 3:126130226:A:G, 3:126130230:G:T, 3:126130244:A:T, 3:126130251:G:C, 3:126130257:G:T, 3:126130272:G:A, 3:126130292:C:A, 3:126130295:T:C, 3:126131385:T:C, 3:126131391:T:G, 3:126131397:C:T, 3:126131404:C:T, 3:126131416:G:C, 3:126131416:G:T, 3:126131421:G:T, 3:126131433:A:T, 3:126131441:G:C, 3:126131442:T:A, 3:126131472:C:T, 3:126131493:G:A, 3:126131499:A:C, 3:126131517:T:C, 3:126131534:C:A, 3:126131536:T:C, 3:126135534:C:T, 3:126135548:G:A, 3:126135550:C:A, 3:126135551:C:A, 3:126135562:G:T, 3:126135568:A:C, 3:126135572:T:TC, 3:126135577:C:A, 3:126135578:A:G, 3:126135595:G:C, 3:126135595:G:A, 3:126135604:G:A, 3:126135625:A:T, 3:126136763:C:T, 3:126136763:C:G, 3:126136781:T:G, 3:126136823:C:A, 3:126136832:T:A, 3:126136848:C:CA, 3:126136885:T:C, 3:126137814:T:C, 3:126137815:A:C, 3:126137842:C:A, 3:126137847:C:T, 3:126137853:A:G, 3:126137871:T:C, 3:126137872:G:A, 3:126137886:A:C, 3:126137886:A:G, 3:126137895:G:A, 3:126137904:A:G, 3:126137907:T:C, 3:126137952:T:C, 3:126146834:C:T, 3:126146835:C:A, 3:126146836:T:C, 3:126146838:A:C, 3:126146847:A:C, 3:126146853:G:A, 3:126146857:C:A, 3:126146857:C:T, 3:126146869:A:G, 3:126146875:C:T, 3:126146889:A:G, 3:126146908:G:T, 3:126146911:T:C, 3:126146915:CT:C, 3:126146917:G:A, 3:126150403:T:TA, 3:126150450:G:A, 3:126150474:A:T, 3:126150493:C:A, 3:126150500:C:T, 3:126150525:C:T, 3:126153454:T:G, 3:126153455:C:A, 3:126153461:C:T, 3:126153473:G:A, 3:126153487:A:C, 3:126153493:C:T, 3:126153494:C:T, 3:126153494:C:A, 3:126153495:TG:T, 3:126153510:T:TG, 3:126153520:A:G, 3:126153564:G:T, 3:126153568:A:G, 3:126153577:A:T, 3:126154555:T:C, 3:126154600:C:G, 3:126154601:G:T, 3:126154610:T:A, 3:126154625:C:T, 3:126154637:A:G, 3:126154638:G:T, 3:126154641:G:C, 3:126154645:T:G, 3:126155427:CCCTCATA:C, 3:126155431:C:T, 3:126155434:A:G, 3:126155446:C:A, 3:126155464:T:TG, 3:126155464:T:C, 3:126155482:C:G, 3:126155494:C:T, 3:126155498:C:G, 3:126155500:G:A, 3:126157341:A:G, 3:126157347:C:T, 3:126157348:C:T, 3:126157351:T:C, 3:126157356:C:T, 3:126157371:A:C, 3:126157374:C:T, 3:126157374:C:A, 3:126157393:C:T, 3:126157399:C:T, 3:126157411:C:T, 3:126157426:G:A, 3:126157428:A:G, 3:126157450:G:C, 3:126157452:C:T, 3:126157456:C:T, 3:126157461:G:T, 3:126157461:G:A, 3:126157462:C:T, 3:126157465:A:G, 3:126157468:A:G, 3:126157473:G:A, 3:126157477:A:C, 3:126157477:A:T, 3:126157482:C:A, 3:126157482:C:T, 3:126158403:A:G, 3:126158404:C:T, 3:126158406:A:T, 3:126158411:A:T, 3:126158421:C:A, 3:126158433:T:C, 3:126158442:G:C, 3:126158457:T:C, 3:126158458:G:C, 3:126158469:G:A, 3:126158471:C:CG, 3:126158475:G:C, 3:126158493:T:C, 3:126158499:T:C, 3:126158502:A:T, 3:126158516:G:T, 3:126158523:C:CGTTG, 3:126158529:G:T, 3:126158534:G:A, 3:126158560:CA:C, 3:126158567:G:A, 3:126158573:G:T, 3:126158580:C:G, 3:126158590:C:T, 3:126158592:A:T, 3:126158594:C:T, 3:126158617:TC:T, 3:126158618:C:T, 3:126158619:C:T, 3:126160852:C:T, 3:126160853:C:G, 3:126160855:AG:A, 3:126160865:C:A, 3:126160866:C:CT, 3:126160874:C:A, 3:126160882:G:A, 3:126160892:A:G, 3:126160897:C:T, 3:126160937:C:T, 3:126160956:C:A, 3:126160962:A:C, 3:126160972:A:G, 3:126161003:C:G, 3:126161004:T:A, 3:126180474:A:G, and 3:126180967:A:G (according to the GRCh38/hg38 human genome assembly).

In some embodiments, the subject's aggregate gene burden of having any one or more ALDH1L1 variant nucleic acid molecules encoding ALDH1L1 predicted loss-of-function polypeptides represents a weighted sum of a plurality of any of the ALDH1L1 variant nucleic acid molecules encoding ALDH1L1 predicted loss-of-function polypeptides. In some embodiments, the aggregate gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the ALDH1L1 gene where the gene burden is the number of alleles multiplied by the association estimate with kidney disease or related outcome for each allele (e.g., a weighted burden score). This can include any genetic variants, regardless of their genomic annotation, in proximity to the ALDH1L1 gene (up to 10 Mb around the gene) that show a non-zero association with kidney disease-related traits in a genetic association analysis. In some embodiments, when the subject has an aggregate gene burden above a desired threshold score, the subject has a decreased risk of developing a kidney disease. In some embodiments, when the subject has an aggregate gene burden below a desired threshold score, the subject has an increased risk of developing a kidney disease.

In some embodiments, any of the methods described herein can further comprise determining the subject's aggregate gene burden of having ALDOB variant nucleic acid molecules encoding ALDOB predicted loss-of-function polypeptides associated with a decreased risk of developing a kidney disease. The aggregate gene burden is the sum of all variants in the ALDOB gene, which can be carried out in an association analysis with a kidney disease. In some embodiments, the subject is homozygous for one or more ALDOB variant nucleic acid molecules encoding ALDOB predicted loss-of-function polypeptides associated with a decreased risk of developing a kidney disease. In some embodiments, the subject is heterozygous for one or more ALDOB variant nucleic acid molecules encoding ALDOB predicted loss-of-function polypeptides associated with a decreased risk of developing a kidney disease. The result of the association analysis suggests that ALDOB variant nucleic acid molecules encoding ALDOB predicted loss-of-function polypeptides are associated with decreased risk of developing a kidney disease. When the subject has a lower aggregate gene burden, the subject is at a higher risk of developing a kidney disease and the subject is administered or continued to be administered a therapeutic agent that treats, prevents, or inhibits a kidney disease in an amount that is the same as or less than a standard dosage amount, and/or an ALDOB inhibitor. When the subject has a greater aggregate gene burden, the subject is at a lower risk of developing a kidney disease and the subject is administered or continued to be administered a therapeutic agent that treats, prevents, or inhibits a kidney disease in a standard dosage amount. The greater the aggregate gene burden, the lower the risk of developing a kidney disease.

ALDOB predicted loss of function variants include, but are not limited to: 9:101421809:C:G, 9:101421837:G:T, 9:101424886:G:A, 9:101424936:A:G, 9:101424947:TTA:T, 9:101425044:T:G, 9:101426567:A:C, 9:101426567:A:T, 9:101426572:G:A, 9:101427500:G:C, 9:101428468:C:A, 9:101428484:CTTTG:C, 9:101429753:A:T, 9:101429776:CT:C, 9:101429828:AG:A, 9:101429901:G:A, 9:101430878:G:A, 9:101430886:A:G, 9:101430887:T:C, 9:101424878:C:A, 9:101425629:T:C, 9:101426589:TG:T, 9:101426611:CT:C, 9:101429963:GTACC:G, 9:101421841:G:A, 9:101421863:AC:A, 9:101421875:A:C, 9:101421905:C:T, 9:101421905:C:A, 9:101424846:GGCCC:G, 9:101424855:C:A, 9:101424894:GC:G, 9:101424923:G:A, 9:101424976:AG:A, 9:101425007:CCTCTTCA:C, 9:101425028:AC:A, 9:101425452:C:G, 9:101425558:TG:T, 9:101425610:G:T, 9:101425628:C:A, 9:101426554:C:T, 9:101426623:TA:T, 9:101427481:C:T, 9:101427481:C:G, 9:101427607:TG:T, 9:101427624:TC:T, 9:101427644:T:G, 9:101428487:TG:T, 9:101428488:GT:G, 9:101428517:G:A, 9:101428520:CT:C, 9:101429754:C:T, 9:101429839:GA:G, 9:101429880:CCA:C, 9:101429884:AG:A, 9:101429937:T:A, 9:101429967:C:T, 9:101429968:T:A, and 9:101430836:C:A (according to the GRCh38/hg38 human genome assembly).

ALDO pLOF or missense variants predicted to be deleterious by at least 1 out of 5 in silico prediction algorithms with AAF<1% include, but are not limited to: 9:101421809:C:G, 9:101421816:G:A, 9:101421837:G:T, 9:101421837:G:A, 9:101421855:C:G, 9:101421867:G:A, 9:101421871:G:A, 9:101421876:T:C, 9:101421877:A:G, 9:101421891:G:A, 9:101421899:G:C, 9:101424850:C:T, 9:101424851:G:A, 9:101424862:G:A, 9:101424876:C:T, 9:101424886:G:A, 9:101424887:C:T, 9:101424895:C:T, 9:101424915:A:C, 9:101424931:C:T, 9:101424932:G:A, 9:101424936:A:G, 9:101424947:TTA:T, 9:101424959:G:A, 9:101424964:G:C, 9:101424973:C:A, 9:101424976:A:G, 9:101424977:G:T, 9:101424983:T:C, 9:101424985:G:A, 9:101424994:T:C, 9:101425003:G:A, 9:101425024:C:T, 9:101425042:C:T, 9:101425044:T:G, 9:101425477:G:C, 9:101425477:G:A, 9:101425480:G:A, 9:101425492:C:T, 9:101425501:T:C, 9:101425504:C:A, 9:101425537:C:A, 9:101425554:A:T, 9:101425577:C:A, 9:101425589:A:T, 9:101425591:G:T, 9:101425614:A:T, 9:101425614:A:G, 9:101426560:C:G, 9:101426567:A:C, 9:101426567:A:T, 9:101426572:G:A, 9:101426589:T:C, 9:101426598:T:C, 9:101426602:G:T, 9:101426608:C:T, 9:101426619:A:G, 9:101426622:A:G, 9:101426623:T:C, 9:101426636:A:C, 9:101427493:T:G, 9:101427493:T:C, 9:101427498:G:T, 9:101427499:C:T, 9:101427500:G:C, 9:101427505:G:A, 9:101427520:C:T, 9:101427534:G:A, 9:101427535:C:T, 9:101427556:C:T, 9:101427574:C:G, 9:101427576:C:T, 9:101427577:G:A, 9:101427580:A:G, 9:101427621:C:T, 9:101427622:G:T, 9:101427622:G:A, 9:101428468:C:A, 9:101428483:T:C, 9:101428484:CTTTG:C, 9:101428496:C:T, 9:101428502:G:A, 9:101428504:G:A, 9:101428510:C:T, 9:101429753:A:T, 9:101429765:A:C, 9:101429769:C:T, 9:101429776:CT:C, 9:101429797:G:C, 9:101429803:C:A, 9:101429813:C:T, 9:101429816:T:C, 9:101429828:AG:A, 9:101429829:G:A, 9:101429835:C:T, 9:101429900:C:T, 9:101429901:G:A, 9:101429909:C:T, 9:101429910:G:A, 9:101429912:C:A, 9:101429913:G:T, 9:101429913:G:A, 9:101429943:T:A, 9:101429951:C:T, 9:101429952:G:A, 9:101429960:A:G, 9:101430878:G:A, 9:101430886:A:G, 9:101430887:T:C, 9:101421813:T:C, 9:101421819:T:C, 9:101421838:A:G, 9:101421840:T:C, 9:101421847:A:G, 9:101421874:C:T, 9:101424860:A:T, 9:101424878:C:A, 9:101424879:C:A, 9:101424888:A:G, 9:101424890:C:A, 9:101424916:C:A, 9:101424920:C:A, 9:101424967:A:G, 9:101424972:G:C, 9:101424974:A:G, 9:101424977:G:A, 9:101424989:T:G, 9:101425015:C:T, 9:101425016:T:G, 9:101425017:C:G, 9:101425470:A:G, 9:101425471:C:G, 9:101425476:C:T, 9:101425482:A:G, 9:101425486:C:A, 9:101425492:C:A, 9:101425537:C:G, 9:101425588:C:A, 9:101425629:T:C, 9:101426555:C:G, 9:101426571:T:G, 9:101426589:TG:T, 9:101426596:C:T, 9:101426599:C:G, 9:101426611:C:T, 9:101426611:CT:C, 9:101427483:T:A, 9:101427504:C:T, 9:101427526:C:G, 9:101427544:A:G, 9:101427546:G:A, 9:101427550:A:G, 9:101427556:C:G, 9:101427586:C:T, 9:101427617:A:C, 9:101427618:C:T, 9:101428507:G:A, 9:101428514:C:T, 9:101429775:C:T, 9:101429787:G:A, 9:101429815:G:T, 9:101429831:G:A, 9:101429859:T:A, 9:101429876:C:A, 9:101429909:C:A, 9:101429912:C:T, 9:101429944:C:A, 9:101429951:C:A, 9:101429952:G:T, 9:101429963:GTACC:G, 9:101430779:C:T, 9:101430799:A:T, 9:101430805:T:G, 9:101430811:T:C, 9:101430815:C:T, 9:101430829:G:A, 9:101430877:C:G, 9:101421817:T:C, 9:101421820:A:G, 9:101421823:A:G, 9:101421841:G:A, 9:101421841:G:T, 9:101421849:G:A, 9:101421850:C:A, 9:101421855:C:A, 9:101421863:AC:A, 9:101421865:C:T, 9:101421870:T:G, 9:101421875:A:C, 9:101421878:C:G, 9:101421879:T:C, 9:101421880:G:C, 9:101421891:G:T, 9:101421892:C:T, 9:101421898:A:G, 9:101421901:T:A, 9:101421901:T:C, 9:101421903:G:A, 9:101421905:C:A, 9:101421905:C:T, 9:101424844:A:G, 9:101424845:T:G, 9:101424846:GGCCC:G, 9:101424848:C:T, 9:101424850:C:A, 9:101424855:C:A, 9:101424857:T:C, 9:101424874:G:T, 9:101424879:C:G, 9:101424879:C:T, 9:101424881:T:C, 9:101424889:G:T, 9:101424892:T:G, 9:101424893:T:C, 9:101424894:GC:G, 9:101424905:C:A, 9:101424913:G:A, 9:101424917:T:G, 9:101424919:G:C, 9:101424922:T:C, 9:101424922:T:G, 9:101424923:G:A, 9:101424923:G:T, 9:101424929:C:T, 9:101424934:C:A, 9:101424941:A:G, 9:101424946:C:G, 9:101424956:A:G, 9:101424958:G:A, 9:101424968:G:T, 9:101424976:AG:A, 9:101424978:G:C, 9:101424982:A:T, 9:101424991:A:G, 9:101424992:G:A, 9:101424995:T:C, 9:101425000:G:A, 9:101425003:G:C, 9:101425007:CCTCTTCA:C, 9:101425008:C:G, 9:101425013:C:T, 9:101425015:C:G, 9:101425016:T:C, 9:101425017:C:T, 9:101425018:A:C, 9:101425019:T:C, 9:101425021:C:T, 9:101425027:G:A, 9:101425028:AC:A, 9:101425029:C:G, 9:101425030:A:G, 9:101425036:C:G, 9:101425452:C:G, 9:101425458:A:T, 9:101425465:C:T, 9:101425471:C:T, 9:101425480:G:C, 9:101425480:G:T, 9:101425494:G:A, 9:101425495:T:G, 9:101425501:T:G, 9:101425513:C:T, 9:101425518:G:A, 9:101425522:A:G, 9:101425523:C:A, 9:101425525:T:G, 9:101425526:C:G, 9:101425530:G:A, 9:101425538:A:T, 9:101425539:T:G, 9:101425542:C:G, 9:101425542:C:T, 9:101425546:C:T, 9:101425546:C:A, 9:101425557:T:C, 9:101425557:T:G, 9:101425558:TG:T, 9:101425564:T:C, 9:101425570:G:T, 9:101425570:G:C, 9:101425576:C:T, 9:101425578:T:C, 9:101425581:A:G, 9:101425590:T:C, 9:101425592:A:T, 9:101425595:G:T, 9:101425607:C:G, 9:101425608:T:C, 9:101425609:T:G, 9:101425610:G:T, 9:101425612:A:C, 9:101425628:C:A, 9:101426554:C:T, 9:101426562:G:A, 9:101426565:A:G, 9:101426566:C:T, 9:101426568:T:C, 9:101426570:C:A, 9:101426574:C:T, 9:101426581:C:G, 9:101426583:A:G, 9:101426587:C:G, 9:101426593:C:T, 9:101426623:TA:T, 9:101426626:G:C, 9:101426628:A:G, 9:101426632:G:T, 9:101426632:G:C, 9:101426634:C:T, 9:101426637:T:C, 9:101427481:C:T, 9:101427481:C:G, 9:101427484:G:T, 9:101427489:C:T, 9:101427496:T:C, 9:101427499:C:G, 9:101427504:C:G, 9:101427514:C:A, 9:101427516:T:C, 9:101427519:G:A, 9:101427521:G:T, 9:101427525:T:A, 9:101427532:T:C, 9:101427537:A:G, 9:101427538:G:A, 9:101427544:A:T, 9:101427547:G:C, 9:101427547:G:T, 9:101427549:C:T, 9:101427550:A:T, 9:101427554:G:T, 9:101427565:T:C, 9:101427571:C:G, 9:101427571:C:T, 9:101427574:C:T, 9:101427576:C:A, 9:101427580:A:C, 9:101427589:A:G, 9:101427601:C:T, 9:101427607:TG:T, 9:101427612:T:C, 9:101427615:G:C, 9:101427615:G:A, 9:101427621:C:A, 9:101427624:TC:T, 9:101427634:C:T, 9:101427642:C:T, 9:101427644:T:G, 9:101428474:A:T, 9:101428475:T:C, 9:101428484:C:T, 9:101428487:TG:T, 9:101428488:GT:G, 9:101428488:G:T, 9:101428498:G:A, 9:101428499:C:T, 9:101428508:C:G, 9:101428511:C:G, 9:101428513:C:T, 9:101428517:G:A, 9:101428519:T:A, 9:101428520:CT:C, 9:101429754:C:T, 9:101429755:C:G, 9:101429763:C:G, 9:101429765:A:G, 9:101429766:C:T, 9:101429771:A:T, 9:101429772:T:A, 9:101429776:C:A, 9:101429780:T:C, 9:101429782:C:A, 9:101429784:T:C, 9:101429787:G:C, 9:101429789:A:T, 9:101429804:T:A, 9:101429807:C:A, 9:101429809:C:G, 9:101429814:T:C, 9:101429834:T:A, 9:101429839:GA:G, 9:101429846:A:T, 9:101429849:A:G, 9:101429850:C:T, 9:101429852:C:A, 9:101429853:C:T, 9:101429855:C:T, 9:101429856:C:T, 9:101429856:C:G, 9:101429858:A:G, 9:101429858:A:C, 9:101429859:T:C, 9:101429861:C:G, 9:101429866:G:T, 9:101429867:T:C, 9:101429871:T:G, 9:101429871:T:A, 9:101429880:CCA:C, 9:101429883:C:A, 9:101429883:C:T, 9:101429884:AG:A, 9:101429888:A:G, 9:101429898:C:G, 9:101429917:C:G, 9:101429925:T:C, 9:101429936:T:A, 9:101429937:T:A, 9:101429939:A:C, 9:101429946:G:T, 9:101429948:A:G, 9:101429948:A:C, 9:101429949:G:C, 9:101429953:G:T, 9:101429958:C:A, 9:101429958:C:G, 9:101429959:C:G, 9:101429961:T:C, 9:101429967:C:T, 9:101429968:T:A, 9:101430776:C:G, 9:101430776:C:A, 9:101430782:A:G, 9:101430794:C:G, 9:101430796:A:G, 9:101430802:C:A, 9:101430803:C:G, 9:101430808:C:T, 9:101430811:T:A, 9:101430812:T:C, 9:101430814:G:A, 9:101430823:C:T, 9:101430830:C:A, 9:101430831:A:C, 9:101430836:C:T, 9:101430836:C:A, 9:101430845:C:T, 9:101430847:T:C, 9:101430848:T:C, 9:101430854:G:T, 9:101430855:C:A, 9:101430857:C:G, 9:101430862:G:T, 9:101430872:G:A, and 9:101430883:G:A (according to the GRCh38/hg38 human genome assembly).

In some embodiments, the subject's aggregate gene burden of having any one or more ALDOB variant nucleic acid molecules encoding ALDOB predicted loss-of-function polypeptides represents a weighted sum of a plurality of any of the ALDOB variant nucleic acid molecules encoding ALDOB predicted loss-of-function polypeptides. In some embodiments, the aggregate gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the ALDOB gene where the gene burden is the number of alleles multiplied by the association estimate with kidney disease or related outcome for each allele (e.g., a weighted burden score). This can include any genetic variants, regardless of their genomic annotation, in proximity to the ALDOB gene (up to 10 Mb around the gene) that show a non-zero association with kidney disease-related traits in a genetic association analysis. In some embodiments, when the subject has an aggregate gene burden above a desired threshold score, the subject has a decreased risk of developing a kidney disease. In some embodiments, when the subject has an aggregate gene burden below a desired threshold score, the subject has an increased risk of developing a kidney disease.

In some embodiments, any of the methods described herein can further comprise determining the subject's aggregate gene burden of having G6PC variant nucleic acid molecules encoding G6PC predicted loss-of-function polypeptides associated with a decreased risk of developing a kidney disease. The aggregate gene burden is the sum of all variants in the G6PC gene, which can be carried out in an association analysis with a kidney disease. In some embodiments, the subject is homozygous for one or more G6PC variant nucleic acid molecules encoding G6PC predicted loss-of-function polypeptides associated with a decreased risk of developing a kidney disease. In some embodiments, the subject is heterozygous for one or more G6PC variant nucleic acid molecules encoding G6PC predicted loss-of-function polypeptides associated with a decreased risk of developing a kidney disease. The result of the association analysis suggests that G6PC variant nucleic acid molecules encoding G6PC predicted loss-of-function polypeptides are associated with decreased risk of developing a kidney disease. When the subject has a lower aggregate gene burden, the subject is at a higher risk of developing a kidney disease and the subject is administered or continued to be administered a therapeutic agent that treats, prevents, or inhibits a kidney disease in an amount that is the same as or less than a standard dosage amount, and/or a G6PC inhibitor. When the subject has a greater aggregate gene burden, the subject is at a lower risk of developing a kidney disease and the subject is administered or continued to be administered a therapeutic agent that treats, prevents, or inhibits a kidney disease in a standard dosage amount. The greater the aggregate gene burden, the lower the risk of developing a kidney disease.

G6PC predicted loss of function variants include, but are not limited to: 17:42900952:TC:T, 17:42901065:G:A, 17:42901085:G:A, 17:42909364:C:T, 17:42910958:T:C, 17:42911076:C:T, 17:42911084:G:A, 17:42911221:AG:A, 7:42911321:C:A, 17:42911391:C:T, 17:42900878:T:A, 17:42903956:TG:T, 17:42907558:G:GTA, 17:42909302:G:A, 17:42909412:CTG:C, 17:42910914:G:T, 17:42911087:C:A, 17:42900920:C:G, 17:42901010:TC:T, 17:42901025:GGT:G, 17:42901063:TG:T, 17:42901107:G:C, 17:42903929:AGGATT:A, 17:42903930:G:A, 17:42903948:G:GTCCA, 17:42903955:C:A, 17:42903958:G:A, 17:42903959:TG:T, 17:42903962:GT:G, 17:42903992:TC:T, 17:42904024:CTG:C, 17:42907530:CT:C, 17:42907563:C:G, 17:42907620:C:T, 17:42907629:G:C, 17:42909350:ATGTC:A, 17:42909354:C:CT, 17:42909403:GC:G, 17:42909416:C:G, 17:42911138:CA:C, 17:42911149:GC:G, 17:42911213:ACT:A, 17:42911336:C:A, 17:42911355:CT:C, and 17:42911403:C:T (according to the GRCh38/hg38 human genome assembly).

G6PC pLOF or missense variants predicted to be deleterious by at least 1 out of 5 or 5 out of 5, respectively, in silico prediction algorithms with AAF<1% include, but are not limited to: 17:42900935:A:G, 17:42900952:TC:T, 17:42900989:A:T, 17:42900994:A:G, 17:42900999:T:G, 17:42901065:G:A, 17:42901067:T:G, 17:42901069:G:T, 17:42901085:G:A, 17:42901105:T:C, 17:42903939:T:C, 17:42903947:C:T, 17:42903948:G:A, 17:42903968:G:T, 17:42904028:G:A, 17:42904034:G:A, 17:42907552:G:A, 17:42909356:G:A, 17:42909364:C:T, 17:42909365:G:A, 17:42910924:T:C, 17:42910958:T:C, 17:42911017:G:C, 17:42911076:C:T, 17:42911084:G:A, 17:42911085:T:C, 17:42911098:A:T, 17:42911121:C:A, 17:42911124:T:C, 17:42911144:C:A, 17:42911221:AG:A, 17:42911235:C:T, 17:42911236:G:A, 17:42911295:C:G, 17:42911296:C:T, 17:42911321:C:A, 17:42911329:C:G, 17:42911391:C:T, 17:42900878:T:A, 17:42900890:T:G, 17:42900891:G:A, 17:42900910:G:C, 17:42900985:G:A, 17:42901093:C:A, 17:42903956:TG:T, 17:42903959:T:A, 17:42904035:G:A, 17:42907534:G:A, 17:42907537:C:T, 17:42907558:G:GTA, 17:42909302:G:A, 17:42909412:CTG:C, 17:42910914:G:T, 17:42911087:C:A, 17:42911116:C:T, 17:42911127:G:T, 17:42911161:G:T, 17:42911292:C:T, 17:42911318:C:A, 17:42900902:A:G, 17:42900920:C:G, 17:42900931:C:T, 17:42900935:A:C, 17:42900977:C:G, 17:42900988:G:T, 17:42901010:TC:T, 17:42901013:T:C, 17:42901016:T:C, 17:42901016:T:G, 17:42901018:C:G, 17:42901025:GGT:G, 17:42901026:G:T, 17:42901034:T:C, 17:42901048:G:T, 17:42901057:C:T, 17:42901063:TG:T, 17:42901066:G:A, 17:42901067:T:C, 17:42901070:C:T, 17:42901075:A:T, 17:42901084:T:G, 17:42901097:T:C, 17:42901107:G:C, 17:42903929:AGGATT:A, 17:42903930:G:A, 17:42903935:C:A, 17:42903939:T:A, 17:42903947:C:G, 17:42903948:G:GTCCA, 17:42903950:C:T, 17:42903955:C:A, 17:42903957:G:T, 17:42903958:G:A, 17:42903959:TG:T, 17:42903962:GT:G, 17:42903968:G:A, 17:42903969:A:G, 17:42903992:TC:T, 17:42903998:C:T, 17:42904004:A:G, 17:42904005:T:C, 17:42904024:CTG:C, 17:42904026:G:A, 17:42904032:C:T, 17:42904035:G:C, 17:42904037:C:A, 17:42907523:G:A, 17:42907530:CT:C, 17:42907538:A:G, 17:42907543:A:G, 17:42907545:G:A, 17:42907547:G:A, 17:42907563:C:G, 17:42907564:T:C, 17:42907567:G:A, 17:42907577:C:T, 17:42907620:C:T, 17:42907629:G:C, 17:42909322:T:G, 17:42909350:ATGTC:A, 17:42909354:C:CT, 17:42909403:GC:G, 17:42909416:C:G, 17:42910927:C:T, 17:42910957:A:C, 17:42910977:T:C, 17:42911008:T:G, 17:42911016:G:A, 17:42911029:T:C, 17:42911074:C:T, 17:42911102:G:T, 17:42911119:C:G, 17:42911127:G:A, 17:42911128:C:T, 17:42911132:C:A, 17:42911138:CA:C, 17:42911149:GC:G, 17:42911160:G:A, 17:42911182:C:T, 17:42911190:T:A, 17:42911213:ACT:A, 17:42911316:T:G, 17:42911329:C:T, 17:42911336:C:A, 17:42911336:C:G, 17:42911343:G:A, 17:42911344:C:G, 17:42911355:CT:C, 17:42911403:C:T, and 17:42911417:G:T (according to the GRCh38/hg38 human genome assembly).

In some embodiments, the subject's aggregate gene burden of having any one or more G6PC variant nucleic acid molecules encoding G6PC predicted loss-of-function polypeptides represents a weighted sum of a plurality of any of the G6PC variant nucleic acid molecules encoding G6PC predicted loss-of-function polypeptides. In some embodiments, the aggregate gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the G6PC gene where the gene burden is the number of alleles multiplied by the association estimate with kidney disease or related outcome for each allele (e.g., a weighted burden score). This can include any genetic variants, regardless of their genomic annotation, in proximity to the G6PC gene (up to 10 Mb around the gene) that show a non-zero association with kidney disease-related traits in a genetic association analysis. In some embodiments, when the subject has an aggregate gene burden above a desired threshold score, the subject has a decreased risk of developing a kidney disease. In some embodiments, when the subject has an aggregate gene burden below a desired threshold score, the subject has an increased risk of developing a kidney disease.

In some embodiments, any of the methods described herein can further comprise determining the subject's aggregate gene burden of having LRP2 variant nucleic acid molecules encoding LRP2 predicted loss-of-function polypeptides associated with a decreased risk of developing a kidney disease. The aggregate gene burden is the sum of all variants in the LRP2 gene, which can be carried out in an association analysis with a kidney disease. In some embodiments, the subject is homozygous for one or more LRP2 variant nucleic acid molecules encoding LRP2 predicted loss-of-function polypeptides associated with a decreased risk of developing a kidney disease. In some embodiments, the subject is heterozygous for one or more LRP2 variant nucleic acid molecules encoding LRP2 predicted loss-of-function polypeptides associated with a decreased risk of developing a kidney disease. The result of the association analysis suggests that LRP2 variant nucleic acid molecules encoding LRP2 predicted loss-of-function polypeptides are associated with decreased risk of developing a kidney disease. When the subject has a lower aggregate gene burden, the subject is at a higher risk of developing a kidney disease and the subject is administered or continued to be administered a therapeutic agent that treats, prevents, or inhibits a kidney disease in an amount that is the same as or less than a standard dosage amount, and/or an LRP2 inhibitor. When the subject has a greater aggregate gene burden, the subject is at a lower risk of developing a kidney disease and the subject is administered or continued to be administered a therapeutic agent that treats, prevents, or inhibits a kidney disease in a standard dosage amount. The greater the aggregate gene burden, the lower the risk of developing a kidney disease.

LRP2 predicted loss-of-function variants include, but are not limited to: 2:169128827:C:A, 2:169137390:A:G, 2:169137391:C:A, 2:169140514:T:TG, 2:169145826:TC:T, 2:169156407:T:C, 2:169170608:G:A, 2:169174130:G:T, 2:169175301:G:A, 2:169181609:G:T, 2:169186020:C:A, 2:169188266:C:T, 2:169193760:C:T, 2:169201627:C:T, 2:169206561:C:A, 2:169206842:AC:A, 2:169206993:G:A, 2:169209643:T:C, 2:169213822:G:A, 2:169220565:T:C, 2:169238089:A:G, 2:169240944:T:C, 2:169240951:G:T, 2:169241019:G:T, 2:169242989:C:CA, 2:169256152:GC:G, 2:169257126:C:T, 2:169257205:CT:C, 2:169275035:C:T, 2:169279371:C:T, 2:169292325:G:A, 2:169294711:C:A, 2:169128741:G:GTCTC, 2:169129060:G:A, 2:169132661:CA:C, 2:169132664:AT:A, 2:169137405:AC:A, 2:169139250:C:T, 2:169140514:TG:T, 2:169145840:T:A, 2:169145924:C:T, 2:169152840:G:T, 2:169154604:C:G, 2:169156289:G:A, 2:169157370:C:T, 2:169162559:T:A, 2:169162560:A:C, 2:169172041:CCA:C, 2:169173916:T:TA, 2:169173917:A:T, 2:169174099:G:A, 2:169186011:C:A, 2:169188011:TG:T, 2:169198791:TA:T, 2:169198834:C:A, 2:169201806:G:T, 2:169204175:A:T, 2:169206368:AG:A, 2:169209639:G:A, 2:169216431:C:G, 2:169233510:G:A, 2:169236053:GA:G, 2:169242955:C:T, 2:169257123:C:T, 2:169270925:T:TA, 2:169275228:CTACAGTCT:C, 2:169275239:CTGT:C, 2:169292251:AC:A, 2:169294598:A:T, 2:169294708:ACT:A, 2:169307280:C:T, 2:169307386:AT:A, 2:169307398:C:T, 2:169307399:T:C, 2:169362374:GC:G, 2:169128751:G:T, 2:169128778:G:T, 2:169129011:A:T, 2:169129012:C:T, 2:169129033:C:A, 2:169129085:C:T, 2:169132655:AT:A, 2:169137403:G:A, 2:169138576:C:T, 2:169139249:A:T, 2:169139538:CTTACTTG:C, 2:169140513:A:AT, 2:169142729:A:T, 2:169142735:G:T, 2:169142757:CAG:C, 2:169142784:AG:A, 2:169145780:G:A, 2:169145851:C:T, 2:169146737:A:C, 2:169146823:G:A, 2:169152798:C:A, 2:169152944:G:A, 2:169154511:C:CAA, 2:169154513:TC:T, 2:169154533:GA:G, 2:169154562:G:A, 2:169154568:G:A, 2:169156295:C:A, 2:169157426:GATAA:G, 2:169157434:C:CGTGG, 2:169157491:C:A, 2:169162592:GT:G, 2:169166003:CA:C, 2:169168633:GA:G, 2:169169701:C:T, 2:169169701:C:G, 2:169169726:CCAAA:C, 2:169169760:AC:A, 2:169172011:TCA:T, 2:169172121:G:T, 2:169173152:C:T, 2:169173978:C:T, 2:169174076:AC:A, 2:169174162:TC:T, 2:169175192:C:A, 2:169177802:C:A, 2:169177811:TG:T, 2:169177866:GTGTC:G, 2:169177890:ATT:A, 2:169177922:C:T, 2:169177934:T:TA, 2:169177986:C:CG, 2:169178001:G:A, 2:169181492:GGT:G, 2:169181615:G:T, 2:169185618:TC:T, 2:169185808:TG:T, 2:169185888:C:CAATA, 2:169187969:C:T, 2:169188033:C:A, 2:169193822:C:T, 2:169193881:G:A, 2:169198804:C:A, 2:169198912:C:A, 2:169201761:T:TG, 2:169201774:C:CATTGTAGT, 2:169201779:AC:A, 2:169201794:G:C, 2:169201826:G:A, 2:169202755:C:T, 2:169202870:G:A, 2:169202889:G:T, 2:169204116:G:C, 2:169204123:CAT:C, 2:169204138:G:A, 2:169204207:G:GGA, 2:169205534:TG:T, 2:169205638:C:T, 2:169206022:C:T, 2:169206065:C:CG, 2:169206596:AG:A, 2:169206844:C:T, 2:169207047:G:A, 2:169209451:A:G, 2:169209532:ATCTG:A, 2:169211966:A:G, 2:169211968:C:A, 2:169212132:C:CCAAAAGCATT, 2:169213735:T:A, 2:169213840:G:A, 2:169216252:C:G, 2:169216360:CA:C, 2:169216411:G:A, 2:169226434:C:T, 2:169226437:AT:A, 2:169226563:TAC:T, 2:169235839:C:A, 2:169235986:T:TG, 2:169236019:G:A, 2:169236026:G:GC, 2:169236042:C:T, 2:169237144:G:GT, 2:169237210:A:C, 2:169237222:G:T, 2:169237276:ACTGTCAAATAC:A, 2:169239525:A:G, 2:169239564:G:C, 2:169239681:TA:T, 2:169239682:A:C, 2:169240946:A:G, 2:169241126:T:A, 2:169241145:G:T, 2:169241224:GT:G, 2:169241239:G:GCATTGTGC, 2:169242954:A:T, 2:169243460:AT:A, 2:169243473:TC:T, 2:169243475:G:A, 2:169244692:C:T, 2:169244920:GA:G, 2:169246848:TC:T, 2:169246864:AG:A, 2:169246889:A:C, 2:169246909:G:A, 2:169246912:G:A, 2:169247474:G:A, 2:169247501:T:TA, 2:169256206:A:T, 2:169256229:G:A, 2:169257251:T:A, 2:169259024:C:A, 2:169259049:G:T, 2:169259064:A:C, 2:169259188:C:A, 2:169270902:A:T, 2:169270902:A:C, 2:169270902:A:G, 2:169270973:G:A, 2:169272970:G:T, 2:169273005:TTC:T, 2:169275045:G:A, 2:169275154:C:T, 2:169277843:C:T, 2:169279514:C:CCA, 2:169279583:CA:C, 2:169279588:G:T, 2:169282951:G:A, 2:169282996:CA:C, 2:169289025:C:A, 2:169289146:C:A, 2:169290870:G:GT, 2:169290935:G:A, 2:169292314:A:T, 2:169292320:G:T, 2:169294147:C:A, 2:169294159:TC:T, 2:169294255:ATC:A, 2:169294597:T:TAA, 2:169294601:G:T, 2:169294706:G:T, 2:169294712:T:A, 2:169307279:A:C, 2:169307279:AC:A, 2:169307382:G:C, 2:169307397:G:T, 2:169318760:A:AC, 2:169318761:C:T, 2:169318761:C:G, and 2:169320778:G:T (according to the GRCh38/hg38 human genome assembly).

LRP2 pLOF or missense variants with AAF<0.1%+pLOF or missense variants predicted to be deleterious by at least 1 out of 5 in silico prediction algorithms with AAF<1%: 2:169128668:C:G, 2:169128689:G:A, 2:169128690:A:T, 2:169128695:C:T, 2:169128710:T:A, 2:169128736:G:A, 2:169128740:G:C, 2:169128751:G:C, 2:169128776:G:A, 2:169128778:G:A, 2:169128781:G:A, 2:169128787:G:A, 2:169128799:G:A, 2:169128811:T:A, 2:169128818:G:T, 2:169128824:T:C, 2:169128827:C:A, 2:169129014:T:C, 2:169129020:T:A, 2:169129056:T:C, 2:169129062:T:G, 2:169132594:G:A, 2:169132604:G:T, 2:169132622:C:A, 2:169132650:T:C, 2:169132652:C:A, 2:169132656:T:C, 2:169132659:T:A, 2:169137390:A:G, 2:169137391:C:A, 2:169137402:T:G, 2:169137411:T:G, 2:169137464:C:G, 2:169137470:C:T, 2:169137472:T:C, 2:169137477:A:G, 2:169138578:A:G, 2:169138585:T:C, 2:169138597:T:C, 2:169138617:C:A, 2:169138636:T:C, 2:169138645:C:G, 2:169138693:C:G, 2:169138693:C:T, 2:169138699:T:C, 2:169138701:C:T, 2:169139259:C:G, 2:169139296:T:A, 2:169139333:C:T, 2:169139336:C:T, 2:169139338:A:G, 2:169139345:G:A, 2:169139348:T:C, 2:169139366:C:T, 2:169139579:C:T, 2:169139600:C:T, 2:169139603:T:C, 2:169140475:C:G, 2:169140480:C:T, 2:169140489:A:C, 2:169140498:C:T, 2:169140502:C:T, 2:169140504:T:C, 2:169140510:T:A, 2:169140514:T:TG, 2:169140516:G:C, 2:169140519:G:C, 2:169140521:G:A, 2:169140524:A:G, 2:169140531:T:C, 2:169142691:G:A, 2:169142692:T:C, 2:169142697:C:T, 2:169142719:G:T, 2:169142776:G:C, 2:169145777:T:C, 2:169145812:G:A, 2:169145826:TC:T, 2:169145834:C:T, 2:169145877:C:G, 2:169145920:A:T, 2:169146798:T:C, 2:169146874:C:T, 2:169146896:C:G, 2:169146898:C:G, 2:169146953:C:T, 2:169150910:T:C, 2:169150980:T:C, 2:169150987:C:G, 2:169150995:G:T, 2:169150995:G:A, 2:169152839:C:T, 2:169152875:T:C, 2:169152881:G:A, 2:169152884:C:T, 2:169152910:C:T, 2:169152911:G:A, 2:169152917:T:C, 2:169152943:C:T, 2:169154468:A:G, 2:169154469:T:C, 2:169154473:C:G, 2:169154528:T:G, 2:169154561:C:T, 2:169154567:C:T, 2:169154570:A:G, 2:169154573:T:C, 2:169154594:G:A, 2:169156300:C:T, 2:169156306:C:T, 2:169156307:T:A, 2:169156407:T:C, 2:169157379:G:A, 2:169157400:G:T, 2:169157404:C:G, 2:169157405:G:T, 2:169157413:C:T, 2:169157487:C:T, 2:169162489:G:A, 2:169162493:A:G, 2:169162508:C:T, 2:169162527:G:T, 2:169162549:T:A, 2:169162549:T:C, 2:169165973:A:C, 2:169165976:C:A, 2:169166007:G:A, 2:169166021:C:T, 2:169166022:G:A, 2:169166027:C:T, 2:169166053:C:G, 2:169168551:G:A, 2:169168569:C:T, 2:169168602:A:G, 2:169168613:C:T, 2:169168620:G:C, 2:169168632:C:T, 2:169168643:G:A, 2:169168653:A:C, 2:169168670:C:T, 2:169168671:G:A, 2:169169722:G:A, 2:169169723:C:T, 2:169169734:T:C, 2:169169738:C:T, 2:169169756:G:T, 2:169169760:A:C, 2:169169778:A:T, 2:169169783:T:C, 2:169169816:T:A, 2:169170560:G:A, 2:169170585:G:C, 2:169170596:C:T, 2:169170603:C:A, 2:169170608:G:A, 2:169170610:G:A, 2:169170643:T:A, 2:169170644:C:T, 2:169170652:G:C, 2:169170652:G:T, 2:169170661:C:A, 2:169172067:C:A, 2:169172071:C:T, 2:169172075:G:A, 2:169172081:T:A, 2:169172081:T:C, 2:169172101:C:T, 2:169172102:G:A, 2:169172120:G:A, 2:169172128:C:G, 2:169172134:T:A, 2:169173113:C:T, 2:169173117:T:C, 2:169173156:T:G, 2:169173167:C:T, 2:169173168:G:A, 2:169173179:T:C, 2:169173224:A:G, 2:169173930:A:G, 2:169173948:T:G, 2:169173951:C:T, 2:169173982:C:T, 2:169173987:G:A, 2:169173991:T:C, 2:169174011:C:T, 2:169174012:G:A, 2:169174023:G:A, 2:169174026:C:T, 2:169174027:G:A, 2:169174039:T:C, 2:169174050:G:A, 2:169174081:C:T, 2:169174086:A:T, 2:169174119:C:T, 2:169174120:G:A, 2:169174128:G:A, 2:169174129:C:T, 2:169174130:G:T, 2:169174143:T:C, 2:169175204:C:T, 2:169175205:G:A, 2:169175250:A:T, 2:169175258:G:A, 2:169175264:G:A, 2:169175270:T:C, 2:169175280:T:C, 2:169175300:C:T, 2:169175301:G:A, 2:169175310:G:A, 2:169175315:G:A, 2:169175328:G:T, 2:169176423:G:A, 2:169176434:C:A, 2:169176434:C:G, 2:169176435:T:C, 2:169176436:G:T, 2:169176439:T:G, 2:169176451:T:G, 2:169176456:A:G, 2:169176477:A:C, 2:169176490:G:A, 2:169176496:C:T, 2:169176508:C:T, 2:169176520:C:G, 2:169176540:A:T, 2:169176579:G:A, 2:169176584:G:T, 2:169177866:G:T, 2:169177884:C:T, 2:169177889:T:A, 2:169177913:C:T, 2:169177914:T:C, 2:169177957:A:C, 2:169177959:T:C, 2:169177962:C:T, 2:169177970:T:C, 2:169177982:C:T, 2:169177985:T:A, 2:169177994:G:A, 2:169177994:G:C, 2:169181452:T:C, 2:169181460:A:G, 2:169181473:C:T, 2:169181487:T:G, 2:169181490:T:C, 2:169181581:T:C, 2:169181587:C:T, 2:169181589:C:T, 2:169181590:G:A, 2:169181592:T:A, 2:169181595:C:T, 2:169181597:C:G, 2:169181609:G:T, 2:169181614:G:A, 2:169182173:T:C, 2:169182218:T:C, 2:169182221:T:C, 2:169182251:C:T, 2:169182252:G:A, 2:169182254:C:T, 2:169182255:G:A, 2:169182264:C:T, 2:169182266:T:C, 2:169182270:G:A, 2:169182276:A:T, 2:169182296:C:T, 2:169182297:G:T, 2:169182299:G:T, 2:169182307:C:A, 2:169182315:G:A, 2:169182315:G:T, 2:169185504:T:C, 2:169185525:G:T, 2:169185536:G:A, 2:169185537:C:G, 2:169185539:G:A, 2:169185545:C:T, 2:169185549:G:A, 2:169185561:T:C, 2:169185572:G:T, 2:169185583:A:T, 2:169185590:C:G, 2:169185611:G:A, 2:169185617:A:G, 2:169185641:C:T, 2:169185666:T:C, 2:169185687:G:A, 2:169185707:A:G, 2:169185725:T:A, 2:169185731:C:T, 2:169185734:T:C, 2:169185756:C:T, 2:169185773:C:T, 2:169185774:G:A, 2:169185809:G:A, 2:169185812:G:A, 2:169185825:T:C, 2:169185856:G:T, 2:169185858:T:C, 2:169185870:G:T, 2:169185871:C:T, 2:169185890:A:G, 2:169185905:C:G, 2:169185906:G:A, 2:169185916:C:T, 2:169185922:C:A, 2:169185935:C:A, 2:169185935:C:T, 2:169185981:C:T, 2:169186012:A:C, 2:169186019:C:A, 2:169186020:C:A, 2:169187987:C:T, 2:169188005:T:A, 2:169188008:A:G, 2:169188014:T:C, 2:169188042:G:A, 2:169188062:T:C, 2:169188074:C:A, 2:169188086:G:C, 2:169188120:C:T, 2:169188134:T:C, 2:169188150:C:T, 2:169188165:T:C, 2:169188170:C:T, 2:169188171:G:A, 2:169188174:C:T, 2:169188185:G:A, 2:169188207:T:C, 2:169188221:C:G, 2:169188224:C:A, 2:169188234:T:C, 2:169188248:T:C, 2:169188258:G:A, 2:169188266:C:T, 2:169191836:A:G, 2:169191857:C:T, 2:169191874:T:C, 2:169191890:T:G, 2:169191903:A:C, 2:169191923:C:T, 2:169191982:G:A, 2:169192010:C:T, 2:169192016:C:A, 2:169192018:G:A, 2:169193760:C:T, 2:169193782:C:T, 2:169193787:C:A, 2:169193799:C:T, 2:169193806:T:C, 2:169193811:C:G, 2:169193812:C:T, 2:169193827:C:T, 2:169193847:C:A, 2:169193849:A:C, 2:169193889:T:C, 2:169193890:G:A, 2:169196923:G:T, 2:169196970:T:C, 2:169196980:T:C, 2:169196985:C:T, 2:169196986:G:A, 2:169197000:T:C, 2:169197006:T:G, 2:169197015:C:A, 2:169197018:C:A, 2:169197021:G:A, 2:169197028:T:C, 2:169197030:G:A, 2:169198801:T:G, 2:169198818:C:T, 2:169198845:T:C, 2:169198849:C:T, 2:169198849:C:A, 2:169198851:C:T, 2:169198852:G:A, 2:169198855:G:A, 2:169198894:A:G, 2:169198906:G:T, 2:169198906:G:A, 2:169201627:C:T, 2:169201669:T:C, 2:169201685:T:C, 2:169201694:G:A, 2:169201708:C:T, 2:169201738:T:C, 2:169201746:C:A, 2:169201780:C:A, 2:169201780:C:T, 2:169201798:T:C, 2:169201805:G:A, 2:169201825:C:A, 2:169201831:T:C, 2:169201835:C:A, 2:169201852:G:A, 2:169201868:G:A, 2:169202761:A:G, 2:169202770:A:G, 2:169202783:C:G, 2:169202789:C:T, 2:169202833:C:T, 2:169202834:G:A, 2:169202842:G:A, 2:169202863:C:A, 2:169202869:C:G, 2:169202869:C:T, 2:169202878:T:C, 2:169202904:G:T, 2:169202910:C:G, 2:169202932:G:A, 2:169202945:C:T, 2:169202950:C:T, 2:169203987:G:A, 2:169204036:A:T, 2:169204062:G:C, 2:169204062:G:T, 2:169204069:T:C, 2:169204074:C:T, 2:169204093:T:G, 2:169204095:G:A, 2:169204098:G:A, 2:169204113:C:T, 2:169204149:T:C, 2:169204160:G:T, 2:169204164:G:A, 2:169204165:T:C, 2:169204185:T:C, 2:169204186:A:G, 2:169204206:T:C, 2:169204207:G:A, 2:169204210:C:T, 2:169204218:T:C, 2:169204225:T:A, 2:169204233:C:T, 2:169204245:G:A, 2:169204245:G:T, 2:169204253:G:T, 2:169204266:C:G, 2:169204268:C:A, 2:169204270:G:T, 2:169205507:C:T, 2:169205533:C:T, 2:169205538:C:T, 2:169205540:T:C, 2:169205543:C:G, 2:169205567:C:T, 2:169205569:C:T, 2:169205572:A:G, 2:169205608:T:C, 2:169205609:G:A, 2:169205625:C:A, 2:169206028:G:C, 2:169206034:A:T, 2:169206036:C:G, 2:169206054:G:C, 2:169206063:C:T, 2:169206065:C:T, 2:169206066:G:A, 2:169206075:C:T, 2:169206080:C:T, 2:169206081:G:A, 2:169206107:T:A, 2:169206113:A:G, 2:169206126:A:G, 2:169206171:C:T, 2:169206173:T:C, 2:169206428:T:C, 2:169206432:T:C, 2:169206436:A:C, 2:169206450:G:C, 2:169206459:C:T, 2:169206477:T:C, 2:169206480:T:C, 2:169206482:T:G, 2:169206494:C:T, 2:169206507:G:C, 2:169206531:A:T, 2:169206561:C:A, 2:169206564:T:C, 2:169206570:T:C, 2:169206626:G:A, 2:169206631:C:A, 2:169206656:T:C, 2:169206660:A:G, 2:169206666:C:A, 2:169206713:C:T, 2:169206714:G:A, 2:169206737:A:G, 2:169206749:C:T, 2:169206773:A:G, 2:169206782:G:A, 2:169206782:G:C, 2:169206785:G:T, 2:169206800:G:T, 2:169206805:C:G, 2:169206812:G:T, 2:169206822:T:C, 2:169206829:C:A, 2:169206842:AC:A, 2:169206843:C:G, 2:169206843:C:T, 2:169206852:T:C, 2:169206875:C:T, 2:169206881:G:A, 2:169206888:G:T, 2:169206893:C:T, 2:169206905:C:T, 2:169206906:G:A, 2:169206935:C:T, 2:169206936:G:A, 2:169206938:A:G, 2:169206950:A:G, 2:169206954:C:G, 2:169206954:C:A, 2:169206963:C:T, 2:169206992:C:T, 2:169206993:G:A, 2:169207010:C:T, 2:169207022:A:G, 2:169207031:G:A, 2:169207046:C:T, 2:169207050:T:C, 2:169207056:A:C, 2:169207070:C:T, 2:169207124:G:A, 2:169207145:G:C, 2:169207167:G:A, 2:169207175:C:T, 2:169207176:G:A, 2:169207178:C:T, 2:169207189:A:T, 2:169207196:C:T, 2:169207197:G:A, 2:169207209:T:C, 2:169207247:T:G, 2:169207250:C:T, 2:169209453:C:T, 2:169209455:G:C, 2:169209503:T:C, 2:169209509:A:G, 2:169209512:A:G, 2:169209534:C:G, 2:169209548:C:T, 2:169209549:G:A, 2:169209567:C:T, 2:169209584:T:C, 2:169209627:G:A, 2:169209638:C:T, 2:169209643:T:C, 2:169211989:T:C, 2:169211992:T:A, 2:169212054:A:G, 2:169212058:A:T, 2:169212072:G:A, 2:169212082:G:A, 2:169212111:G:T, 2:169212112:C:T, 2:169212118:C:T, 2:169212127:A:G, 2:169212135:C:T, 2:169212136:C:G, 2:169212142:C:T, 2:169212154:T:A, 2:169212166:C:T, 2:169212176:G:T, 2:169212179:G:T, 2:169212187:C:T, 2:169212202:C:T, 2:169213659:C:T, 2:169213660:G:A, 2:169213680:C:A, 2:169213681:C:T, 2:169213686:A:G, 2:169213693:G:A, 2:169213693:G:T, 2:169213712:T:C, 2:169213718:G:T, 2:169213760:C:A, 2:169213821:T:C, 2:169213822:G:A, 2:169213827:A:C, 2:169213841:A:C, 2:169213852:C:T, 2:169213855:C:T, 2:169216294:C:T, 2:169216298:G:C, 2:169216303:G:C, 2:169216336:T:G, 2:169216347:T:G, 2:169216360:C:T, 2:169216377:G:C, 2:169216378:C:T, 2:169216393:C:T, 2:169216412:G:C, 2:169220476:T:C, 2:169220481:A:G, 2:169220485:G:C, 2:169220541:A:G, 2:169220565:T:C, 2:169225325:T:A, 2:169225330:G:A, 2:169225338:G:T, 2:169225384:G:T, 2:169225390:C:T, 2:169225420:T:C, 2:169225433:C:A, 2:169225441:T:C, 2:169226447:T:A, 2:169226454:C:A, 2:169226454:C:T, 2:169226456:T:C, 2:169226502:C:A, 2:169226518:C:A, 2:169226523:C:T, 2:169226525:T:C, 2:169226575:G:C, 2:169226583:G:T, 2:169231774:A:G, 2:169231809:T:C, 2:169231818:C:T, 2:169231819:G:A, 2:169231834:G:A, 2:169231840:C:T, 2:169233413:T:C, 2:169233414:T:C, 2:169233437:G:A, 2:169233443:A:G, 2:169233461:A:G, 2:169233470:A:T, 2:169233504:T:C, 2:169233518:C:T, 2:169233519:G:A, 2:169233521:C:T, 2:169233530:C:T, 2:169233531:G:A, 2:169233581:C:T, 2:169233582:G:A, 2:169233583:T:C, 2:169233585:T:C, 2:169235847:C:T, 2:169235865:C:T, 2:169235866:G:A, 2:169235871:T:C, 2:169235896:T:A, 2:169235905:G:T, 2:169235911:A:T, 2:169235916:A:G, 2:169235946:A:G, 2:169235947:T:C, 2:169235956:C:T, 2:169235963:C:G, 2:169235965:A:G, 2:169235994:C:T, 2:169235997:A:G, 2:169236036:T:C, 2:169236049:A:C, 2:169237105:C:T, 2:169237113:G:C, 2:169237151:C:A, 2:169237153:A:C, 2:169237167:T:C, 2:169237169:T:C, 2:169237181:A:G, 2:169237184:T:C, 2:169237190:A:G, 2:169237197:T:C, 2:169237232:C:T, 2:169237233:G:A, 2:169237245:C:T, 2:169237247:A:G, 2:169237250:G:A, 2:169238089:A:G, 2:169238095:C:G, 2:169238099:T:C, 2:169238104:G:A, 2:169238113:T:C, 2:169238119:G:A, 2:169238122:C:T, 2:169238161:C:T, 2:169238166:A:C, 2:169238203:C:A, 2:169238252:C:T, 2:169238258:T:G, 2:169238278:A:G, 2:169239571:G:A, 2:169239586:C:T, 2:169239587:G:A, 2:169239623:A:G, 2:169239634:A:C, 2:169239640:C:T, 2:169239644:C:T, 2:169239646:T:G, 2:169239673:T:C, 2:169239680:G:T, 2:169239700:A:T, 2:169239701:G:T, 2:169239760:G:A, 2:169240944:T:C, 2:169240951:G:T, 2:169240963:A:G, 2:169240976:C:T, 2:169240976:C:G, 2:169240990:C:A, 2:169241000:A:T, 2:169241009:T:G, 2:169241014:T:C, 2:169241018:G:T, 2:169241019:G:T, 2:169241022:G:T, 2:169241075:G:A, 2:169241101:C:T, 2:169241101:C:G, 2:169241102:G:A, 2:169241108:G:T, 2:169241124:C:G, 2:169241129:C:T, 2:169241143:C:A, 2:169241144:C:T, 2:169241156:C:T, 2:169241158:C:T, 2:169241230:G:A, 2:169241239:G:A, 2:169241260:T:C, 2:169241263:A:T, 2:169241279:C:T, 2:169241281:T:C, 2:169241289:C:A, 2:169241296:T:C, 2:169241299:G:T, 2:169241309:T:C, 2:169241311:C:A, 2:169241312:C:A, 2:169241325:C:A, 2:169241326:T:C, 2:169241342:G:A, 2:169241359:C:T, 2:169241360:T:C, 2:169242956:G:C, 2:169242956:G:A, 2:169242961:C:A, 2:169242964:G:C, 2:169242964:G:A, 2:169242973:G:A, 2:169242989:C:CA, 2:169243016:C:T, 2:169243033:C:T, 2:169243040:C:A, 2:169243047:G:C, 2:169243060:G:T, 2:169243447:T:A, 2:169243459:G:A, 2:169243465:T:C, 2:169243472:A:G, 2:169243474:C:T, 2:169243478:G:A, 2:169243480:T:C, 2:169243489:T:A, 2:169243503:T:A, 2:169243514:C:G, 2:169243519:G:A, 2:169244716:T:C, 2:169244744:C:T, 2:169244759:T:C, 2:169244768:G:T, 2:169244783:A:T, 2:169244810:C:T, 2:169244812:T:G, 2:169244812:T:C, 2:169244828:G:A, 2:169244846:C:A, 2:169244857:C:T, 2:169244858:G:A, 2:169244869:C:T, 2:169244870:G:T, 2:169244874:G:T, 2:169244890:T:G, 2:169244911:G:A, 2:169244914:G:A, 2:169244932:T:A, 2:169246705:T:C, 2:169246713:C:G, 2:169246741:G:C, 2:169246750:C:T, 2:169246767:T:C, 2:169246773:T:C, 2:169246795:T:C, 2:169246815:C:G, 2:169246815:C:A, 2:169246820:C:G, 2:169246827:G:A, 2:169246855:C:T, 2:169246882:T:C, 2:169246893:G:C, 2:169246899:C:G, 2:169246902:C:A, 2:169246906:C:T, 2:169246908:C:A, 2:169246926:G:A, 2:169246947:T:C, 2:169246951:C:T, 2:169246960:G:A, 2:169246965:G:T, 2:169246971:T:C, 2:169246978:C:T, 2:169247382:C:A, 2:169247389:T:C, 2:169247395:T:C, 2:169247401:G:A, 2:169247426:T:C, 2:169247446:A:T, 2:169247452:C:A, 2:169247473:C:T, 2:169247479:A:G, 2:169247501:T:C, 2:169247504:A:C, 2:169247512:T:C, 2:169256129:G:A, 2:169256132:T:G, 2:169256142:G:T, 2:169256150:T:C, 2:169256152:GC:G, 2:169256169:C:T, 2:169256187:T:C, 2:169256188:G:C, 2:169257124:G:A, 2:169257126:C:T, 2:169257131:C:T, 2:169257137:C:A, 2:169257142:C:T, 2:169257160:G:C, 2:169257164:T:C, 2:169257191:C:T, 2:169257199:C:A, 2:169257205:CT:C, 2:169257223:C:T, 2:169257224:G:A, 2:169257225:G:C, 2:169257229:C:A, 2:169257233:C:T, 2:169259052:C:T, 2:169259053:G:A, 2:169259062:T:A, 2:169259082:C:T, 2:169259082:C:A, 2:169259083:G:A, 2:169259088:G:A, 2:169259093:A:T, 2:169259098:C:G, 2:169259103:C:T, 2:169259116:T:C, 2:169259134:C:G, 2:169259140:A:C, 2:169259162:A:C, 2:169259178:T:C, 2:169259179:C:T, 2:169259196:T:C, 2:169259203:C:T, 2:169270923:C:G, 2:169270948:T:C, 2:169270949:C:T, 2:169270949:C:G, 2:169270976:C:A, 2:169270976:C:T, 2:169270991:C:T, 2:169271014:G:A, 2:169271018:C:T, 2:169271021:G:A, 2:169271024:C:A, 2:169271026:A:G, 2:169271042:T:C, 2:169271050:G:A, 2:169271056:G:A, 2:169271065:C:T, 2:169271066:G:A, 2:169271069:T:C, 2:169271069:T:G, 2:169271080:G:A, 2:169272930:T:C, 2:169272938:C:T, 2:169272939:G:T, 2:169272939:G:A, 2:169272980:C:T, 2:169272981:G:A, 2:169273005:T:C, 2:169273008:T:C, 2:169273019:A:G, 2:169273045:G:T, 2:169273058:G:A, 2:169275035:C:T, 2:169275038:T:C, 2:169275038:T:A, 2:169275048:T:C, 2:169275074:T:C, 2:169275078:G:A, 2:169275092:T:C, 2:169275103:T:A, 2:169275105:G:C, 2:169275107:G:T, 2:169275110:T:C, 2:169275125:T:C, 2:169275172:C:G, 2:169275209:A:G, 2:169275219:C:T, 2:169275221:C:T, 2:169275230:A:G, 2:169275233:G:A, 2:169275233:G:T, 2:169277751:A:G, 2:169277757:T:C, 2:169277766:A:G, 2:169277775:A:G, 2:169277788:G:A, 2:169277808:C:T, 2:169277814:G:A, 2:169277816:T:C, 2:169277823:T:C, 2:169277869:C:A, 2:169277875:G:A, 2:169277876:G:T, 2:169277878:T:C, 2:169277910:G:C, 2:169277917:C:G, 2:169277924:G:T, 2:169277924:G:C, 2:169277938:A:G, 2:169279371:C:T, 2:169279382:G:A, 2:169279388:C:T, 2:169279400:T:C, 2:169279420:G:C, 2:169279435:C:T, 2:169279436:G:A, 2:169279459:A:G, 2:169279466:T:C, 2:169279468:C:T, 2:169279469:G:A, 2:169279502:T:G, 2:169279504:T:G, 2:169279510:C:A, 2:169279531:G:A, 2:169279536:T:G, 2:169279541:C:T, 2:169279543:G:A, 2:169279549:T:C, 2:169279550:T:C, 2:169279563:G:C, 2:169279591:A:T, 2:169280381:C:A, 2:169280411:A:C, 2:169280412:C:T, 2:169280423:C:T, 2:169280448:G:A, 2:169280457:T:A, 2:169280472:C:A, 2:169280486:C:T, 2:169280502:T:C, 2:169280511:C:G, 2:169280514:C:G, 2:169282876:A:C, 2:169282877:A:C, 2:169282878:T:A, 2:169282893:T:C, 2:169282904:C:G, 2:169282929:T:C, 2:169282941:C:T, 2:169282950:C:T, 2:169282998:A:T, 2:169289037:C:T, 2:169289046:T:C, 2:169289052:T:C, 2:169289082:G:A, 2:169289083:C:T, 2:169289092:A:G, 2:169289094:G:A, 2:169289094:G:T, 2:169289097:G:A, 2:169289139:G:A, 2:169289145:C:T, 2:169290857:C:T, 2:169290859:G:A, 2:169290873:T:G, 2:169290932:A:G, 2:169290940:G:A, 2:169290985:T:C, 2:169290986:G:A, 2:169290992:C:T, 2:169292258:C:A, 2:169292267:T:C, 2:169292271:C:T, 2:169292277:C:A, 2:169292294:C:T, 2:169292324:C:T, 2:169292325:G:A, 2:169292329:A:T, 2:169292331:T:C, 2:169292334:G:A, 2:169292355:C:T, 2:169292363:G:A, 2:169294166:T:C, 2:169294183:T:A, 2:169294204:G:C, 2:169294207:C:T, 2:169294216:C:T, 2:169294220:C:T, 2:169294234:G:A, 2:169294241:C:T, 2:169294255:A:G, 2:169294603:A:G, 2:169294620:G:A, 2:169294635:A:T, 2:169294645:C:G, 2:169294647:C:G, 2:169294650:T:G, 2:169294659:G:A, 2:169294665:T:C, 2:169294671:G:T, 2:169294671:G:A, 2:169294679:G:T, 2:169294711:C:A, 2:169307305:C:T, 2:169307312:G:C, 2:169307315:T:G, 2:169307329:A:G, 2:169307346:A:G, 2:169307361:G:A, 2:169307373:T:G, 2:169307376:T:C, 2:169318773:C:T, 2:169318789:C:G, 2:169318825:T:C, 2:169318839:C:G, 2:169318842:T:A, 2:169318851:C:A, 2:169318879:C:A, 2:169318884:G:A, 2:169320794:G:A, 2:169320795:C:T, 2:169320795:C:A, 2:169320818:C:T, 2:169320826:C:A, 2:169320827:C:T, 2:169320849:G:A, 2:169320863:C:T, 2:169320864:G:A, 2:169320868:A:C, 2:169320872:G:A, 2:169320872:G:T, 2:169320875:C:T, 2:169362338:G:T, 2:169362348:C:G, 2:169362374:G:A, 2:169362390:C:A, 2:169128668:C:G, 2:169128689:G:A, 2:169128690:A:T, 2:169128695:C:T, 2:169128710:T:A, 2:169128778:G:A, 2:169128781:G:A, 2:169128787:G:A, 2:169128827:C:A, 2:169128828:C:T, 2:169129014:T:C, 2:169129020:T:A, 2:169129056:T:C, 2:169129062:T:G, 2:169132622:C:A, 2:169132650:T:C, 2:169132659:T:A, 2:169137390:A:G, 2:169137391:C:A, 2:169137464:C:G, 2:169137477:A:G, 2:169138578:A:G, 2:169138585:T:C, 2:169138597:T:C, 2:169138617:C:A, 2:169138636:T:C, 2:169138645:C:G, 2:169138693:C:T, 2:169138693:C:G, 2:169138699:T:C, 2:169138701:C:T, 2:169139259:C:G, 2:169139296:T:A, 2:169139338:A:G, 2:169139345:G:A, 2:169139366:C:T, 2:169139579:C:T, 2:169139600:C:T, 2:169140475:C:G, 2:169140480:C:T, 2:169140489:A:C, 2:169140498:C:T, 2:169140502:C:T, 2:169140504:T:C, 2:169140510:T:A, 2:169140514:T:TG, 2:169140516:G:C, 2:169140521:G:A, 2:169140524:A:G, 2:169142697:C:T, 2:169142719:G:T, 2:169145777:T:C, 2:169145826:TC:T, 2:169145834:C:T, 2:169145877:C:G, 2:169146798:T:C, 2:169146825:T:C, 2:169146874:C:T, 2:169146896:C:G, 2:169146898:C:G, 2:169146953:C:T, 2:169150910:T:C, 2:169150980:T:C, 2:169150987:C:G, 2:169150995:G:T, 2:169150995:G:A, 2:169152839:C:T, 2:169152875:T:C, 2:169152880:C:T, 2:169152881:G:A, 2:169152910:C:T, 2:169152911:G:A, 2:169152917:T:C, 2:169154473:C:G, 2:169154528:T:G, 2:169154561:C:T, 2:169154567:C:T, 2:169154570:A:G, 2:169154573:T:C, 2:169154594:G:A, 2:169156306:C:T, 2:169156307:T:A, 2:169156407:T:C, 2:169157379:G:A, 2:169157400:G:T, 2:169157404:C:G, 2:169157405:G:T, 2:169157413:C:T, 2:169157487:C:T, 2:169162489:G:A, 2:169162508:C:T, 2:169162549:T:A, 2:169162549:T:C, 2:169165973:A:C, 2:169165976:C:A, 2:169166007:G:A, 2:169166021:C:T, 2:169166022:G:A, 2:169166027:C:T, 2:169166053:C:G, 2:169168551:G:A, 2:169168569:C:T, 2:169168613:C:T, 2:169168620:G:C, 2:169168632:C:T, 2:169168643:G:A, 2:169168653:A:C, 2:169168670:C:T, 2:169168671:G:A, 2:169169723:C:T, 2:169169734:T:C, 2:169169738:C:T, 2:169169778:A:T, 2:169170560:G:A, 2:169170585:G:C, 2:169170596:C:T, 2:169170603:C:A, 2:169170608:G:A, 2:169170610:G:A, 2:169170643:T:A, 2:169170644:C:T, 2:169170652:G:C, 2:169170652:G:T, 2:169170661:C:A, 2:169172067:C:A, 2:169172071:C:T, 2:169172075:G:A, 2:169172081:T:C, 2:169172081:T:A, 2:169172101:C:T, 2:169172102:G:A, 2:169172120:G:A, 2:169172128:C:G, 2:169172134:T:A, 2:169173113:C:T, 2:169173117:T:C, 2:169173147:C:T, 2:169173167:C:T, 2:169173168:G:A, 2:169173224:A:G, 2:169173930:A:G, 2:169173948:T:G, 2:169173951:C:T, 2:169173987:G:A, 2:169173991:T:C, 2:169173996:C:T, 2:169174011:C:T, 2:169174012:G:A, 2:169174023:G:A, 2:169174027:G:A, 2:169174039:T:C, 2:169174050:G:A, 2:169174081:C:T, 2:169174086:A:T, 2:169174119:C:T, 2:169174120:G:A, 2:169174128:G:A, 2:169174129:C:T, 2:169174130:G:T, 2:169174143:T:C, 2:169175205:G:A, 2:169175250:A:T, 2:169175258:G:A, 2:169175264:G:A, 2:169175270:T:C, 2:169175300:C:T, 2:169175301:G:A, 2:169175310:G:A, 2:169175315:G:A, 2:169175328:G:T, 2:169176423:G:A, 2:169176434:C:G, 2:169176434:C:A, 2:169176435:T:C, 2:169176436:G:T, 2:169176439:T:G, 2:169176451:T:G, 2:169176490:G:A, 2:169176508:C:T, 2:169176520:C:G, 2:169176540:A:T, 2:169176579:G:A, 2:169177866:G:T, 2:169177884:C:T, 2:169177889:T:A, 2:169177913:C:T, 2:169177914:T:C, 2:169177957:A:C, 2:169177959:T:C, 2:169177962:C:T, 2:169177970:T:C, 2:169177982:C:T, 2:169177985:T:A, 2:169177994:G:A, 2:169177994:G:C, 2:169181452:T:C, 2:169181460:A:G, 2:169181473:C:T, 2:169181487:T:G, 2:169181490:T:C, 2:169181581:T:C, 2:169181587:C:T, 2:169181590:G:A, 2:169181595:C:T, 2:169181597:C:G, 2:169181609:G:T, 2:169181614:G:A, 2:169182218:T:C, 2:169182221:T:C, 2:169182251:C:T, 2:169182252:G:A, 2:169182254:C:T, 2:169182255:G:A, 2:169182264:C:T, 2:169182266:T:C, 2:169182270:G:A, 2:169182276:A:T, 2:169182299:G:T, 2:169182307:C:A, 2:169182315:G:A, 2:169182315:G:T, 2:169185504:T:C, 2:169185525:G:T, 2:169185536:G:A, 2:169185537:C:G, 2:169185539:G:A, 2:169185561:T:C, 2:169185572:G:T, 2:169185583:A:T, 2:169185590:C:G, 2:169185617:A:G, 2:169185641:C:T, 2:169185666:T:C, 2:169185687:G:A, 2:169185698:T:C, 2:169185725:T:A, 2:169185731:C:T, 2:169185734:T:C, 2:169185735:T:C, 2:169185756:C:T, 2:169185773:C:T, 2:169185774:G:A, 2:169185809:G:A, 2:169185812:G:A, 2:169185825:T:C, 2:169185856:G:T, 2:169185858:T:C, 2:169185870:G:T, 2:169185871:C:T, 2:169185890:A:G, 2:169185905:C:G, 2:169185906:G:A, 2:169185916:C:T, 2:169185922:C:A, 2:169185981:C:T, 2:169186012:A:C, 2:169186019:C:A, 2:169186020:C:A, 2:169187987:C:T, 2:169188005:T:A, 2:169188008:A:G, 2:169188014:T:C, 2:169188042:G:A, 2:169188062:T:C, 2:169188074:C:A, 2:169188086:G:C, 2:169188134:T:C, 2:169188165:T:C, 2:169188170:C:T, 2:169188171:G:A, 2:169188174:C:T, 2:169188185:G:A, 2:169188207:T:C, 2:169188221:C:G, 2:169188224:C:A, 2:169188234:T:C, 2:169188248:T:C, 2:169188258:G:A, 2:169188266:C:T, 2:169191836:A:G, 2:169191857:C:T, 2:169191874:T:C, 2:169191903:A:C, 2:169191923:C:T, 2:169191982:G:A, 2:169192010:C:T, 2:169192016:C:A, 2:169192018:G:A, 2:169193760:C:T, 2:169193782:C:T, 2:169193787:C:A, 2:169193799:C:T, 2:169193806:T:C, 2:169193811:C:G, 2:169193812:C:T, 2:169193827:C:T, 2:169193847:C:A, 2:169193849:A:C, 2:169196923:G:T, 2:169196980:T:C, 2:169196985:C:T, 2:169196986:G:A, 2:169197006:T:G, 2:169197015:C:A, 2:169197018:C:A, 2:169197021:G:A, 2:169198801:T:G, 2:169198818:C:T, 2:169198845:T:C, 2:169198851:C:T, 2:169198852:G:A, 2:169198855:G:A, 2:169198906:G:T, 2:169198906:G:A, 2:169201627:C:T, 2:169201669:T:C, 2:169201685:T:C, 2:169201694:G:A, 2:169201708:C:T, 2:169201738:T:C, 2:169201746:C:A, 2:169201780:C:A, 2:169201780:C:T, 2:169201798:T:C, 2:169201805:G:A, 2:169201825:C:A, 2:169201831:T:C, 2:169201852:G:A, 2:169202761:A:G, 2:169202783:C:G, 2:169202789:C:T, 2:169202833:C:T, 2:169202834:G:A, 2:169202869:C:G, 2:169202869:C:T, 2:169202878:T:C, 2:169202904:G:T, 2:169202910:C:G, 2:169202932:G:A, 2:169202945:C:T, 2:169202950:C:T, 2:169203987:G:A, 2:169204036:A:T, 2:169204062:G:C, 2:169204062:G:T, 2:169204069:T:C, 2:169204093:T:G, 2:169204113:C:T, 2:169204160:G:T, 2:169204164:G:A, 2:169204165:T:C, 2:169204185:T:C, 2:169204186:A:G, 2:169204225:T:A, 2:169204233:C:T, 2:169204245:G:A, 2:169204245:G:T, 2:169204253:G:T, 2:169204266:C:G, 2:169204268:C:A, 2:169204270:G:T, 2:169205538:C:T, 2:169205540:T:C, 2:169205543:C:G, 2:169205569:C:T, 2:169205572:A:G, 2:169205625:C:A, 2:169206028:G:C, 2:169206034:A:T, 2:169206036:C:G, 2:169206054:G:C, 2:169206063:C:T, 2:169206065:C:T, 2:169206066:G:A, 2:169206075:C:T, 2:169206080:C:T, 2:169206081:G:A, 2:169206107:T:A, 2:169206126:A:G, 2:169206171:C:T, 2:169206173:T:C, 2:169206428:T:C, 2:169206450:G:C, 2:169206477:T:C, 2:169206494:C:T, 2:169206507:G:C, 2:169206531:A:T, 2:169206561:C:A, 2:169206570:T:C, 2:169206626:G:A, 2:169206631:C:A, 2:169206656:T:C, 2:169206660:A:G, 2:169206666:C:A, 2:169206713:C:T, 2:169206714:G:A, 2:169206737:A:G, 2:169206749:C:T, 2:169206773:A:G, 2:169206782:G:C, 2:169206782:G:A, 2:169206800:G:T, 2:169206805:C:G, 2:169206812:G:T, 2:169206829:C:A, 2:169206842:AC:A, 2:169206843:C:T, 2:169206843:C:G, 2:169206852:T:C, 2:169206862:A:T, 2:169206870:T:C, 2:169206875:C:T, 2:169206881:G:A, 2:169206888:G:T, 2:169206893:C:T, 2:169206905:C:T, 2:169206906:G:A, 2:169206936:G:A, 2:169206938:A:G, 2:169206950:A:G, 2:169206954:C:A, 2:169206954:C:G, 2:169206963:C:T, 2:169206992:C:T, 2:169206993:G:A, 2:169207010:C:T, 2:169207022:A:G, 2:169207031:G:A, 2:169207046:C:T, 2:169207050:T:C, 2:169207056:A:C, 2:169207070:C:T, 2:169207124:G:A, 2:169207145:G:C, 2:169207167:G:A, 2:169207175:C:T, 2:169207176:G:A, 2:169207178:C:T, 2:169207189:A:T, 2:169207196:C:T, 2:169207197:G:A, 2:169207209:T:C, 2:169207247:T:G, 2:169207250:C:T, 2:169209453:C:T, 2:169209455:G:C, 2:169209509:A:G, 2:169209512:A:G, 2:169209534:C:G, 2:169209548:C:T, 2:169209549:G:A, 2:169209584:T:C, 2:169209627:G:A, 2:169209638:C:T, 2:169209643:T:C, 2:169211989:T:C, 2:169211992:T:A, 2:169212054:A:G, 2:169212058:A:T, 2:169212072:G:A, 2:169212082:G:A, 2:169212088:C:T, 2:169212111:G:T, 2:169212112:C:T, 2:169212118:C:T, 2:169212127:A:G, 2:169212135:C:T, 2:169212136:C:G, 2:169212154:T:A, 2:169212166:C:T, 2:169212176:G:T, 2:169212179:G:T, 2:169212187:C:T, 2:169213660:G:A, 2:169213681:C:T, 2:169213686:A:G, 2:169213693:G:A, 2:169213693:G:T, 2:169213712:T:C, 2:169213718:G:T, 2:169213822:G:A, 2:169213827:A:C, 2:169213852:C:T, 2:169213855:C:T, 2:169216298:G:C, 2:169216303:G:C, 2:169216336:T:G, 2:169216360:C:T, 2:169216377:G:C, 2:169216378:C:T, 2:169216412:G:C, 2:169220476:T:C, 2:169220481:A:G, 2:169220485:G:C, 2:169220565:T:C, 2:169225330:G:A, 2:169225420:T:C, 2:169225433:C:A, 2:169225441:T:C, 2:169226426:T:C, 2:169226447:T:A, 2:169226456:T:C, 2:169226502:C:A, 2:169226518:C:A, 2:169226523:C:T, 2:169226525:T:C, 2:169226575:G:C, 2:169231774:A:G, 2:169231809:T:C, 2:169231819:G:A, 2:169231834:G:A, 2:169233437:G:A, 2:169233443:A:G, 2:169233461:A:G, 2:169233470:A:T, 2:169233504:T:C, 2:169233519:G:A, 2:169233530:C:T, 2:169233531:G:A, 2:169233582:G:A, 2:169233583:T:C, 2:169235847:C:T, 2:169235865:C:T, 2:169235866:G:A, 2:169235905:G:T, 2:169235911:A:T, 2:169235946:A:G, 2:169235947:T:C, 2:169235956:C:T, 2:169235963:C:G, 2:169235965:A:G, 2:169235994:C:T, 2:169236049:A:C, 2:169237113:G:C, 2:169237151:C:A, 2:169237153:A:C, 2:169237167:T:C, 2:169237169:T:C, 2:169237181:A:G, 2:169237184:T:C, 2:169237190:A:G, 2:169237197:T:C, 2:169237232:C:T, 2:169237233:G:A, 2:169237245:C:T, 2:169237247:A:G, 2:169237250:G:A, 2:169238089:A:G, 2:169238095:C:G, 2:169238099:T:C, 2:169238104:G:A, 2:169238113:T:C, 2:169238119:G:A, 2:169238122:C:T, 2:169238161:C:T, 2:169238166:A:C, 2:169238203:C:A, 2:169238246:C:A, 2:169238252:C:T, 2:169238258:T:G, 2:169238278:A:G, 2:169239571:G:A, 2:169239586:C:T, 2:169239587:G:A, 2:169239623:A:G, 2:169239640:C:T, 2:169239644:C:T, 2:169239646:T:G, 2:169239673:T:C, 2:169239680:G:T, 2:169239760:G:A, 2:169240944:T:C, 2:169240951:G:T, 2:169240963:A:G, 2:169240976:C:G, 2:169240976:C:T, 2:169240990:C:A, 2:169241000:A:T, 2:169241009:T:G, 2:169241014:T:C, 2:169241018:G:T, 2:169241019:G:T, 2:169241022:G:T, 2:169241075:G:A, 2:169241101:C:T, 2:169241101:C:G, 2:169241102:G:A, 2:169241108:G:T, 2:169241124:C:G, 2:169241129:C:T, 2:169241143:C:A, 2:169241156:C:T, 2:169241158:C:T, 2:169241230:G:A, 2:169241239:G:A, 2:169241260:T:C, 2:169241263:A:T, 2:169241279:C:T, 2:169241281:T:C, 2:169241289:C:A, 2:169241299:G:T, 2:169241311:C:A, 2:169241312:C:A, 2:169241325:C:A, 2:169241326:T:C, 2:169241342:G:A, 2:169241359:C:T, 2:169241360:T:C, 2:169242956:G:A, 2:169242956:G:C, 2:169242961:C:A, 2:169242964:G:C, 2:169242973:G:A, 2:169242989:C:CA, 2:169243033:C:T, 2:169243040:C:A, 2:169243047:G:C, 2:169243060:G:T, 2:169243447:T:A, 2:169243465:T:C, 2:169243472:A:G, 2:169243474:C:T, 2:169243478:G:A, 2:169243480:T:C, 2:169243489:T:A, 2:169243501:G:A, 2:169244716:T:C, 2:169244744:C:T, 2:169244759:T:C, 2:169244783:A:T, 2:169244858:G:A, 2:169244869:C:T, 2:169244870:G:T, 2:169244890:T:G, 2:169244911:G:A, 2:169244932:T:A, 2:169246705:T:C, 2:169246713:C:G, 2:169246741:G:C, 2:169246750:C:T, 2:169246767:T:C, 2:169246815:C:A, 2:169246815:C:G, 2:169246820:C:G, 2:169246855:C:T, 2:169246882:T:C, 2:169246893:G:C, 2:169246899:C:G, 2:169246902:C:A, 2:169246906:C:T, 2:169246908:C:A, 2:169246926:G:A, 2:169246947:T:C, 2:169246951:C:T, 2:169246960:G:A, 2:169246962:G:A, 2:169246965:G:T, 2:169246971:T:C, 2:169246978:C:T, 2:169247382:C:A, 2:169247395:T:C, 2:169247401:G:A, 2:169247426:T:C, 2:169247446:A:T, 2:169247452:C:A, 2:169247473:C:T, 2:169247479:A:G, 2:169247501:T:C, 2:169247504:A:C, 2:169256129:G:A, 2:169256132:T:G, 2:169256142:G:T, 2:169256152:GC:G, 2:169256169:C:T, 2:169256187:T:C, 2:169256188:G:C, 2:169257124:G:A, 2:169257126:C:T, 2:169257131:C:T, 2:169257137:C:A, 2:169257142:C:T, 2:169257160:G:C, 2:169257164:T:C, 2:169257191:C:T, 2:169257199:C:A, 2:169257205:CT:C, 2:169257223:C:T, 2:169257224:G:A, 2:169257225:G:C, 2:169257229:C:A, 2:169257233:C:T, 2:169259052:C:T, 2:169259053:G:A, 2:169259062:T:A, 2:169259082:C:T, 2:169259082:C:A, 2:169259083:G:A, 2:169259088:G:A, 2:169259093:A:T, 2:169259098:C:G, 2:169259116:T:C, 2:169259134:C:G, 2:169259140:A:C, 2:169259162:A:C, 2:169259178:T:C, 2:169259179:C:T, 2:169259196:T:C, 2:169270923:C:G, 2:169270948:T:C, 2:169270949:C:G, 2:169270949:C:T, 2:169270976:C:T, 2:169270976:C:A, 2:169270991:C:T, 2:169271014:G:A, 2:169271018:C:T, 2:169271021:G:A, 2:169271024:C:A, 2:169271026:A:G, 2:169271046:C:A, 2:169271056:G:A, 2:169271065:C:T, 2:169271066:G:A, 2:169271069:T:G, 2:169271069:T:C, 2:169271080:G:A, 2:169272939:G:T, 2:169272939:G:A, 2:169272980:C:T, 2:169272981:G:A, 2:169273005:T:C, 2:169273008:T:C, 2:169273019:A:G, 2:169273045:G:T, 2:169273058:G:A, 2:169275035:C:T, 2:169275038:T:C, 2:169275048:T:C, 2:169275074:T:C, 2:169275078:G:A, 2:169275103:T:A, 2:169275107:G:T, 2:169275110:T:C, 2:169275125:T:C, 2:169275209:A:G, 2:169275219:C:T, 2:169275221:C:T, 2:169275230:A:G, 2:169275233:G:A, 2:169275233:G:T, 2:169277751:A:G, 2:169277757:T:C, 2:169277766:A:G, 2:169277775:A:G, 2:169277788:G:A, 2:169277808:C:T, 2:169277814:G:A, 2:169277823:T:C, 2:169277869:C:A, 2:169277875:G:A, 2:169277876:G:T, 2:169277878:T:C, 2:169277910:G:C, 2:169277917:C:G, 2:169277924:G:T, 2:169277924:G:C, 2:169277938:A:G, 2:169279371:C:T, 2:169279382:G:A, 2:169279388:C:T, 2:169279400:T:C, 2:169279420:G:C, 2:169279435:C:T, 2:169279436:G:A, 2:169279459:A:G, 2:169279466:T:C, 2:169279468:C:T, 2:169279469:G:A, 2:169279502:T:G, 2:169279504:T:G, 2:169279510:C:A, 2:169279531:G:A, 2:169279541:C:T, 2:169279543:G:A, 2:169279549:T:C, 2:169279550:T:C, 2:169279563:G:C, 2:169279591:A:T, 2:169280381:C:A, 2:169280411:A:C, 2:169280412:C:T, 2:169280423:C:T, 2:169280448:G:A, 2:169280457:T:A, 2:169280472:C:A, 2:169280486:C:T, 2:169280502:T:C, 2:169282876:A:C, 2:169282893:T:C, 2:169282904:C:G, 2:169282950:C:T, 2:169282998:A:T, 2:169289037:C:T, 2:169289046:T:C, 2:169289052:T:C, 2:169289094:G:A, 2:169289094:G:T, 2:169289097:G:A, 2:169289139:G:A, 2:169289145:C:T, 2:169290857:C:T, 2:169290859:G:A, 2:169290873:T:G, 2:169290932:A:G, 2:169290940:G:A, 2:169290986:G:A, 2:169292258:C:A, 2:169292267:T:C, 2:169292271:C:T, 2:169292277:C:A, 2:169292294:C:T, 2:169292324:C:T, 2:169292325:G:A, 2:169292329:A:T, 2:169292334:G:A, 2:169292363:G:A, 2:169294166:T:C, 2:169294216:C:T, 2:169294220:C:T, 2:169294234:G:A, 2:169294241:C:T, 2:169294603:A:G, 2:169294635:A:T, 2:169294645:C:G, 2:169294647:C:G, 2:169294650:T:G, 2:169294665:T:C, 2:169294671:G:A, 2:169294671:G:T, 2:169294711:C:A, 2:169307305:C:T, 2:169307312:G:C, 2:169307329:A:G, 2:169307346:A:G, 2:169307361:G:A, 2:169307373:T:G, 2:169318789:C:G, 2:169318839:C:G, 2:169318842:T:A, 2:169318851:C:A, 2:169318884:G:A, 2:169320794:G:A, 2:169320815:G:C, 2:169320818:C:T, 2:169320826:C:A, 2:169320827:C:T, 2:169320863:C:T, 2:169320864:G:A, 2:169320875:C:T, 2:169362348:C:G, 2:169362390:C:A, 2:169128692:T:C, 2:169128725:A:T, 2:169128741:G:GTCTC, 2:169128751:G:A, 2:169128758:G:A, 2:169128784:G:A, 2:169128811:T:C, 2:169128819:C:G, 2:169128823:T:A, 2:169129017:G:A, 2:169129027:G:T, 2:169129048:G:T, 2:169129051:T:C, 2:169129059:C:T, 2:169129060:G:A, 2:169129070:A:T, 2:169129071:T:C, 2:169129075:A:G, 2:169132608:G:T, 2:169132630:G:A, 2:169132638:G:T, 2:169132638:G:A, 2:169132641:C:T, 2:169132647:T:C, 2:169132648:A:G, 2:169132652:C:G, 2:169132660:C:T, 2:169132661:CA:C, 2:169132664:AT:A, 2:169132672:A:G, 2:169137397:T:C, 2:169137405:AC:A, 2:169137429:G:A, 2:169137457:T:C, 2:169137482:A:T, 2:169137489:T:A, 2:169138581:G:T, 2:169138583:C:T, 2:169138587:G:A, 2:169138590:C:A, 2:169138611:C:A, 2:169138635:A:G, 2:169138639:T:C, 2:169138642:G:C, 2:169138655:T:A, 2:169138687:G:A, 2:169139250:C:T, 2:169139273:G:A, 2:169139296:T:C, 2:169139369:C:T, 2:169139545:G:T, 2:169139552:G:A, 2:169139572:G:A, 2:169139602:C:A, 2:169140464:G:A, 2:169140500:T:C, 2:169140509:C:T, 2:169140514:TG:T, 2:169140516:G:A, 2:169140528:T:C, 2:169142776:G:T, 2:169145797:G:C, 2:169145840:T:A, 2:169145889:G:T, 2:169145907:G:C, 2:169145924:C:T, 2:169146779:T:C, 2:169146793:C:T, 2:169146801:T:G, 2:169146829:T:C, 2:169146883:G:C, 2:169146888:T:C, 2:169146889:T:C, 2:169146908:C:A, 2:169146941:G:T, 2:169146947:C:G, 2:169146955:T:A, 2:169150901:A:T, 2:169150933:G:C, 2:169150939:G:T, 2:169150965:A:G, 2:169150994:C:A, 2:169150994:C:T, 2:169151007:C:T, 2:169151012:G:A, 2:169151022:T:C, 2:169152821:T:C, 2:169152827:C:T, 2:169152839:C:G, 2:169152840:G:T, 2:169152904:T:A, 2:169152959:C:T, 2:169154496:C:G, 2:169154496:C:T, 2:169154499:C:A, 2:169154509:T:G, 2:169154550:G:A, 2:169154575:G:T, 2:169154590:C:G, 2:169154600:C:G, 2:169154604:C:G, 2:169156289:G:A, 2:169156298:G:A, 2:169156300:C:A, 2:169156301:G:T, 2:169156318:A:T, 2:169156354:G:C, 2:169156384:A:G, 2:169156403:T:C, 2:169157370:C:T, 2:169157382:G:T, 2:169157383:T:A, 2:169157415:G:T, 2:169157433:C:G, 2:169157434:C:G, 2:169157436:C:G, 2:169157452:T:C, 2:169157469:A:G, 2:169162510:G:A, 2:169162514:C:T, 2:169162517:C:T, 2:169162538:T:C, 2:169162558:T:C, 2:169162559:T:A, 2:169162560:A:C, 2:169162561:T:C, 2:169162569:T:A, 2:169162576:C:G, 2:169162591:G:A, 2:169162594:T:C, 2:169165994:C:A, 2:169166006:C:T, 2:169168568:T:A, 2:169168583:T:A, 2:169168584:C:T, 2:169168607:G:A, 2:169168632:C:G, 2:169168661:T:C, 2:169168665:G:A, 2:169168671:G:C, 2:169169702:G:A, 2:169169747:C:T, 2:169169761:C:G, 2:169169762:T:C, 2:169169774:G:A, 2:169169780:T:C, 2:169169800:G:C, 2:169170559:C:T, 2:169170607:C:T, 2:169170619:C:T, 2:169170637:C:T, 2:169170645:G:T, 2:169170661:C:T, 2:169170662:G:A, 2:169170665:G:C, 2:169172039:C:A, 2:169172041:CCA:C, 2:169172072:G:A, 2:169172101:C:A, 2:169173108:C:T, 2:169173138:C:G, 2:169173140:T:C, 2:169173141:T:C, 2:169173176:G:A, 2:169173916:T:TA, 2:169173917:A:T, 2:169173919:T:A, 2:169173942:G:A, 2:169174000:C:T, 2:169174016:C:G, 2:169174017:T:C, 2:169174032:G:A, 2:169174046:G:C, 2:169174053:C:A, 2:169174054:T:C, 2:169174055:G:C, 2:169174099:G:A, 2:169174147:A:C, 2:169174152:C:T, 2:169175199:G:A, 2:169175223:C:T, 2:169175264:G:C, 2:169175265:T:C, 2:169175285:T:C, 2:169175292:G:C, 2:169175309:C:T, 2:169175309:C:A, 2:169175354:T:C, 2:169175376:A:C, 2:169175378:A:G, 2:169176424:C:T, 2:169176484:G:C, 2:169176489:C:G, 2:169176508:C:G, 2:169176537:A:G, 2:169176549:T:G, 2:169176552:G:A, 2:169176567:T:C, 2:169176574:C:T, 2:169177850:T:C, 2:169177968:G:C, 2:169177983:C:T, 2:169177994:G:T, 2:169178010:C:T, 2:169181458:C:A, 2:169181488:C:T, 2:169181499:T:C, 2:169181503:T:C, 2:169181509:T:C, 2:169181522:C:G, 2:169182178:G:C, 2:169182209:C:A, 2:169182220:G:T, 2:169182228:C:A, 2:169182255:G:T, 2:169182317:T:C, 2:169185527:C:T, 2:169185533:G:A, 2:169185534:C:A, 2:169185555:T:A, 2:169185572:G:A, 2:169185581:A:G, 2:169185585:A:G, 2:169185615:C:A, 2:169185626:A:C, 2:169185633:T:C, 2:169185663:C:T, 2:169185665:T:C, 2:169185677:T:C, 2:169185743:A:G, 2:169185744:T:G, 2:169185779:G:T, 2:169185813:C:A, 2:169185852:T:G, 2:169185905:C:T, 2:169185945:A:G, 2:169185947:T:C, 2:169185948:A:G, 2:169185956:G:A, 2:169185962:G:A, 2:169186002:C:T, 2:169186011:C:A, 2:169186014:T:G, 2:169187983:A:C, 2:169187983:A:T, 2:169188011:TG:T, 2:169188030:T:C, 2:169188033:C:T, 2:169188041:C:T, 2:169188047:T:C, 2:169188077:G:A, 2:169188101:T:A, 2:169188105:T:A, 2:169188200:T:C, 2:169188221:C:A, 2:169188231:C:T, 2:169188253:G:T, 2:169188257:C:T, 2:169191833:T:C, 2:169191899:T:G, 2:169191915:A:C, 2:169191917:C:T, 2:169191941:C:T, 2:169191946:T:C, 2:169191972:C:A, 2:169192007:A:G, 2:169192010:C:G, 2:169193789:C:T, 2:169193790:A:G, 2:169193811:C:T, 2:169193850:T:C, 2:169196971:G:A, 2:169196976:G:A, 2:169197010:T:C, 2:169198791:TA:T, 2:169198800:T:A, 2:169198809:C:T, 2:169198834:C:A, 2:169198840:A:G, 2:169198854:G:A, 2:169198858:T:C, 2:169198905:C:T, 2:169198909:C:T, 2:169201628:G:A, 2:169201648:G:A, 2:169201660:T:G, 2:169201719:C:T, 2:169201781:C:T, 2:169201789:T:C, 2:169201793:A:C, 2:169201805:G:T, 2:169201806:G:T, 2:169201825:C:T, 2:169202775:A:C, 2:169202788:C:G, 2:169202795:C:T, 2:169202824:G:A, 2:169202834:G:T, 2:169202836:C:T, 2:169202839:T:C, 2:169202884:A:G, 2:169202920:T:A, 2:169202928:A:T, 2:169203985:G:C, 2:169203999:C:G, 2:169204021:C:G, 2:169204030:T:A, 2:169204049:G:C, 2:169204054:T:C, 2:169204120:C:A, 2:169204132:T:C, 2:169204137:C:T, 2:169204143:A:G, 2:169204157:C:G, 2:169204175:A:T, 2:169204189:G:A, 2:169204225:T:C, 2:169204229:T:G, 2:169204258:G:A, 2:169205492:C:T, 2:169205539:A:C, 2:169205570:G:A, 2:169205574:G:T, 2:169205590:G:A, 2:169205606:C:T, 2:169206047:G:C, 2:169206048:C:T, 2:169206089:C:T, 2:169206101:A:C, 2:169206111:T:G, 2:169206129:C:T, 2:169206143:C:T, 2:169206350:G:C, 2:169206368:AG:A, 2:169206380:G:A, 2:169206389:C:G, 2:169206403:T:G, 2:169206413:G:A, 2:169206414:T:C, 2:169206476:A:G, 2:169206493:G:T, 2:169206543:C:T, 2:169206563:G:T, 2:169206564:T:G, 2:169206589:A:T, 2:169206599:G:T, 2:169206624:G:T, 2:169206639:G:T, 2:169206687:G:A, 2:169206689:T:C, 2:169206691:G:T, 2:169206694:G:T, 2:169206699:C:A, 2:169206734:A:G, 2:169206765:T:C, 2:169206772:T:C, 2:169206782:G:T, 2:169206825:T:G, 2:169206911:A:C, 2:169206935:C:G, 2:169206936:G:C, 2:169206969:C:T, 2:169206978:C:T, 2:169207007:C:T, 2:169207023:T:A, 2:169207038:G:A, 2:169207049:T:C, 2:169207052:G:A, 2:169207056:A:T, 2:169207056:A:G, 2:169207059:C:A, 2:169207071:G:A, 2:169207074:C:T, 2:169207149:T:C, 2:169207155:C:T, 2:169207179:G:A, 2:169207181:T:G, 2:169207185:T:G, 2:169207194:T:C, 2:169209453:C:G, 2:169209490:T:A, 2:169209490:T:G, 2:169209566:G:A, 2:169209583:A:C, 2:169209639:G:A, 2:169211969:T:G, 2:169212012:T:A, 2:169212022:A:C, 2:169212028:T:C, 2:169212039:A:G, 2:169212066:T:C, 2:169212078:G:A, 2:169212179:G:C, 2:169212195:G:A, 2:169212199:C:T, 2:169213713:A:G, 2:169213732:C:G, 2:169213738:C:T, 2:169213747:C:G, 2:169213756:C:T, 2:169213763:T:A, 2:169213770:G:A, 2:169213794:A:G, 2:169213819:G:T, 2:169213830:A:C, 2:169213834:T:C, 2:169213835:C:T, 2:169216254:A:T, 2:169216260:C:T, 2:169216266:G:A, 2:169216272:G:T, 2:169216287:T:C, 2:169216297:T:C, 2:169216305:G:T, 2:169216328:G:T, 2:169216342:G:C, 2:169216377:G:A, 2:169216390:G:T, 2:169216398:C:T, 2:169216399:T:C, 2:169216431:C:G, 2:169220457:C:T, 2:169220458:G:A, 2:169220465:A:T, 2:169220472:G:C, 2:169220481:A:T, 2:169220493:A:G, 2:169220497:C:T, 2:169220502:G:C, 2:169220506:T:C, 2:169220521:T:C, 2:169220526:G:A, 2:169225360:A:G, 2:169225389:A:G, 2:169225392:A:G, 2:169225397:T:C, 2:169225404:G:A, 2:169225419:T:C, 2:169225452:C:T, 2:169226432:A:C, 2:169226442:T:C, 2:169226453:G:A, 2:169226469:C:T, 2:169226480:T:C, 2:169226489:C:T, 2:169226499:G:T, 2:169226501:A:C, 2:169226511:C:T, 2:169226513:T:C, 2:169226516:C:T, 2:169226565:C:T, 2:169226579:G:T, 2:169226580:G:T, 2:169226581:T:A, 2:169231717:T:C, 2:169231740:G:A, 2:169231752:C:T, 2:169231759:G:A, 2:169231783:G:A, 2:169231785:C:T, 2:169231786:C:G, 2:169231789:G:C, 2:169231821:G:A, 2:169231828:C:G, 2:169233437:G:T, 2:169233441:C:T, 2:169233464:T:G, 2:169233509:C:T, 2:169233510:G:A, 2:169233522:G:A, 2:169233548:G:C, 2:169233582:G:C, 2:169235891:G:C, 2:169235916:A:C, 2:169235917:T:C, 2:169235926:G:A, 2:169235967:A:G, 2:169235978:C:A, 2:169235978:C:G, 2:169235994:C:A, 2:169236005:G:T, 2:169236015:G:A, 2:169236022:C:T, 2:169236027:C:A, 2:169236028:G:A, 2:169236028:G:C, 2:169236048:G:T, 2:169236053:GA:G, 2:169236054:A:T, 2:169237121:G:A, 2:169237131:T:C, 2:169237140:T:A, 2:169237149:T:C, 2:169237170:G:T, 2:169237196:A:G, 2:169237206:G:C, 2:169237239:C:T, 2:169237254:T:C, 2:169237265:A:C, 2:169238101:T:C, 2:169238102:C:G, 2:169238104:G:T, 2:169238162:G:A, 2:169238167:C:T, 2:169238221:T:C, 2:169238227:T:C, 2:169238267:T:A, 2:169238271:A:C, 2:169238293:C:G, 2:169239532:A:G, 2:169239542:T:A, 2:169239544:C:G, 2:169239554:T:G, 2:169239574:T:C, 2:169239601:A:G, 2:169239648:T:C, 2:169239718:G:A, 2:169240978:G:A, 2:169241020:C:T, 2:169241116:G:A, 2:169241159:G:A, 2:169241171:A:G, 2:169241177:T:A, 2:169241192:C:T, 2:169241239:G:T, 2:169241285:A:G, 2:169241299:G:A, 2:169242955:C:T, 2:169242976:T:C, 2:169242983:T:G, 2:169242997:C:G, 2:169243006:C:T, 2:169243021:A:G, 2:169243022:T:C, 2:169243025:A:G, 2:169243030:T:A, 2:169243054:G:A, 2:169243057:G:A, 2:169243068:T:G, 2:169243421:A:T, 2:169243431:A:C, 2:169243455:A:C, 2:169243468:A:G, 2:169243486:C:T, 2:169243502:G:C, 2:169243513:T:C, 2:169244702:T:G, 2:169244782:T:G, 2:169244810:C:G, 2:169244819:G:A, 2:169244821:C:A, 2:169244833:T:C, 2:169244843:C:T, 2:169244860:T:C, 2:169244873:A:G, 2:169244899:C:T, 2:169244921:A:G, 2:169246738:C:G, 2:169246785:C:A, 2:169246800:G:A, 2:169246800:G:C, 2:169246831:G:A, 2:169246843:T:C, 2:169246875:G:A, 2:169246942:T:C, 2:169246978:C:A, 2:169247378:C:T, 2:169247384:G:T, 2:169247408:A:T, 2:169247417:T:C, 2:169256142:G:C, 2:169256147:A:G, 2:169256184:T:C, 2:169256197:T:G, 2:169256235:C:T, 2:169257123:C:T, 2:169257127:C:G, 2:169257134:T:C, 2:169257145:T:C, 2:169257161:T:A, 2:169257207:C:T, 2:169257215:T:C, 2:169259026:C:T, 2:169259028:G:A, 2:169259031:A:G, 2:169259049:G:A, 2:169259052:C:A, 2:169259053:G:C, 2:169259067:T:C, 2:169259074:C:T, 2:169259076:A:T, 2:169259079:G:A, 2:169259085:C:G, 2:169259086:T:C, 2:169259102:C:A, 2:169259106:A:C, 2:169259112:C:G, 2:169259112:C:T, 2:169259193:C:T, 2:169259194:T:C, 2:169270925:T:TA, 2:169270928:A:G, 2:169270963:G:C, 2:169270966:C:G, 2:169270966:C:T, 2:169271011:C:G, 2:169271075:C:G, 2:169271098:T:C, 2:169272963:C:T, 2:169272968:G:A, 2:169272969:T:C, 2:169273016:A:G, 2:169273038:C:T, 2:169273044:T:C, 2:169273050:C:T, 2:169273065:T:C, 2:169273067:G:A, 2:169275053:G:A, 2:169275090:C:T, 2:169275099:A:G, 2:169275154:C:A, 2:169275224:T:A, 2:169275228:CTACAGTCT:C, 2:169275239:CTGT:C, 2:169277758:C:T, 2:169277817:A:G, 2:169277848:C:T, 2:169277864:C:A, 2:169277893:A:G, 2:169277905:G:C, 2:169277911:G:A, 2:169277930:C:A, 2:169279376:C:G, 2:169279379:T:C, 2:169279402:G:A, 2:169279420:G:A, 2:169279441:C:T, 2:169279448:C:A, 2:169279478:T:C, 2:169279519:G:T, 2:169279540:A:G, 2:169279556:C:A, 2:169279579:A:C, 2:169279592:A:G, 2:169280379:C:T, 2:169280406:C:T, 2:169280471:C:G, 2:169280487:G:A, 2:169280492:T:C, 2:169280504:A:C, 2:169280514:C:T, 2:169282903:G:A, 2:169282917:C:T, 2:169282942:G:A, 2:169282953:C:T, 2:169282953:C:A, 2:169282968:T:C, 2:169282978:C:T, 2:169283001:T:A, 2:169289038:G:A, 2:169289047:T:C, 2:169289050:G:A, 2:169289065:C:A, 2:169289088:C:G, 2:169289104:G:A, 2:169289112:T:C, 2:169289121:T:A, 2:169289143:T:C, 2:169290865:G:A, 2:169290866:T:C, 2:169290868:T:G, 2:169290878:C:T, 2:169290902:T:C, 2:169290923:T:C, 2:169290929:T:C, 2:169290961:C:G, 2:169290964:G:A, 2:169290971:A:G, 2:169290993:G:T, 2:169292251:AC:A, 2:169292312:T:C, 2:169292334:G:C, 2:169292354:C:T, 2:169292361:T:C, 2:169294169:C:T, 2:169294208:G:A, 2:169294208:G:C, 2:169294232:A:G, 2:169294247:G:C, 2:169294254:T:C, 2:169294598:A:T, 2:169294626:C:G, 2:169294636:C:T, 2:169294659:G:C, 2:169294660:T:C, 2:169294686:G:T, 2:169294690:G:A, 2:169294697:A:C, 2:169294701:G:T, 2:169294704:G:A, 2:169294708:ACT:A, 2:169294709:C:A, 2:169307280:C:T, 2:169307326:C:T, 2:169307350:A:G, 2:169307358:T:C, 2:169307371:T:A, 2:169307386:AT:A, 2:169307397:G:A, 2:169307398:C:T, 2:169307399:T:C, 2:169318774:G:A, 2:169318774:G:T, 2:169318853:C:G, 2:169318863:C:T, 2:169318881:A:T, 2:169320777:C:T, 2:169320785:A:G, 2:169320787:T:G, 2:169320831:A:G, 2:169320840:G:T, 2:169320846:A:G, 2:169320884:T:A, 2:169362321:C:G, 2:169362374:GC:G, 2:169362390:C:T, 2:169362392:C:T, 2:169128692:T:C, 2:169128741:G:GTCTC, 2:169128784:G:A, 2:169128819:C:G, 2:169128823:T:A, 2:169129017:G:A, 2:169129027:G:T, 2:169129048:G:T, 2:169129051:T:C, 2:169129059:C:T, 2:169129060:G:A, 2:169129070:A:T, 2:169129075:A:G, 2:169132638:G:T, 2:169132638:G:A, 2:169132641:C:T, 2:169132647:T:C, 2:169132648:A:G, 2:169132661:CA:C, 2:169132664:AT:A, 2:169137405:AC:A, 2:169137457:T:C, 2:169137482:A:T, 2:169137489:T:A, 2:169138583:C:T, 2:169138587:G:A, 2:169138590:C:A, 2:169138611:C:A, 2:169138635:A:G, 2:169138639:T:C, 2:169138655:T:A, 2:169138687:G:A, 2:169139250:C:T, 2:169139273:G:A, 2:169139296:T:C, 2:169139369:C:T, 2:169139545:G:T, 2:169139552:G:A, 2:169139572:G:A, 2:169140464:G:A, 2:169140500:T:C, 2:169140509:C:T, 2:169140514:TG:T, 2:169140516:G:A, 2:169145797:G:C, 2:169145840:T:A, 2:169145889:G:T, 2:169145907:G:C, 2:169145924:C:T, 2:169146779:T:C, 2:169146801:T:G, 2:169146883:G:C, 2:169146908:C:A, 2:169146941:G:T, 2:169146947:C:G, 2:169146955:T:A, 2:169150901:A:T, 2:169150933:G:C, 2:169150965:A:G, 2:169150994:C:A, 2:169150994:C:T, 2:169151007:C:T, 2:169151012:G:A, 2:169151022:T:C, 2:169152821:T:C, 2:169152827:C:T, 2:169152839:C:G, 2:169152840:G:T, 2:169152904:T:A, 2:169152959:C:T, 2:169154499:C:A, 2:169154509:T:G, 2:169154550:G:A, 2:169154575:G:T, 2:169154590:C:G, 2:169154604:C:G, 2:169156289:G:A, 2:169156318:A:T, 2:169156354:G:C, 2:169156384:A:G, 2:169156403:T:C, 2:169157370:C:T, 2:169157383:T:A, 2:169157433:C:G, 2:169157434:C:G, 2:169157436:C:G, 2:169157452:T:C, 2:169157469:A:G, 2:169162514:C:T, 2:169162517:C:T, 2:169162538:T:C, 2:169162558:T:C, 2:169162559:T:A, 2:169162560:A:C, 2:169162561:T:C, 2:169162576:C:G, 2:169162591:G:A, 2:169166006:C:T, 2:169168568:T:A, 2:169168583:T:A, 2:169168584:C:T, 2:169168607:G:A, 2:169168632:C:G, 2:169168661:T:C, 2:169168665:G:A, 2:169168671:G:C, 2:169169702:G:A, 2:169169747:C:T, 2:169169774:G:A, 2:169169780:T:C, 2:169169800:G:C, 2:169170559:C:T, 2:169170607:C:T, 2:169170619:C:T, 2:169170637:C:T, 2:169170645:G:T, 2:169170661:C:T, 2:169170662:G:A, 2:169170665:G:C, 2:169172039:C:A, 2:169172041:CCA:C, 2:169172072:G:A, 2:169172101:C:A, 2:169173108:C:T, 2:169173138:C:G, 2:169173140:T:C, 2:169173141:T:C, 2:169173176:G:A, 2:169173916:T:TA, 2:169173917:A:T, 2:169173919:T:A, 2:169173942:G:A, 2:169174000:C:T, 2:169174016:C:G, 2:169174017:T:C, 2:169174032:G:A, 2:169174046:G:C, 2:169174053:C:A, 2:169174054:T:C, 2:169174055:G:C, 2:169174099:G:A, 2:169174152:C:T, 2:169175199:G:A, 2:169175223:C:T, 2:169175264:G:C, 2:169175265:T:C, 2:169175285:T:C, 2:169175292:G:C, 2:169175309:C:T, 2:169175309:C:A, 2:169175376:A:C, 2:169175378:A:G, 2:169176424:C:T, 2:169176508:C:G, 2:169176537:A:G, 2:169176549:T:G, 2:169176552:G:A, 2:169176567:T:C, 2:169176574:C:T, 2:169177850:T:C, 2:169177968:G:C, 2:169177983:C:T, 2:169177994:G:T, 2:169178010:C:T, 2:169181458:C:A, 2:169181488:C:T, 2:169181499:T:C, 2:169181503:T:C, 2:169181509:T:C, 2:169181522:C:G, 2:169182178:G:C, 2:169182209:C:A, 2:169182220:G:T, 2:169182228:C:A, 2:169182255:G:T, 2:169182317:T:C, 2:169185527:C:T, 2:169185555:T:A, 2:169185572:G:A, 2:169185581:A:G, 2:169185585:A:G, 2:169185615:C:A, 2:169185626:A:C, 2:169185633:T:C, 2:169185663:C:T, 2:169185665:T:C, 2:169185677:T:C, 2:169185743:A:G, 2:169185744:T:G, 2:169185779:G:T, 2:169185813:C:A, 2:169185852:T:G, 2:169185905:C:T, 2:169185945:A:G, 2:169185947:T:C, 2:169185948:A:G, 2:169185956:G:A, 2:169185962:G:A, 2:169186002:C:T, 2:169186011:C:A, 2:169186014:T:G, 2:169187983:A:C, 2:169187983:A:T, 2:169188011:TG:T, 2:169188030:T:C, 2:169188033:C:T, 2:169188047:T:C, 2:169188077:G:A, 2:169188101:T:A, 2:169188221:C:A, 2:169188231:C:T, 2:169188257:C:T, 2:169191833:T:C, 2:169191899:T:G, 2:169191915:A:C, 2:169191917:C:T, 2:169191941:C:T, 2:169191946:T:C, 2:169191972:C:A, 2:169192007:A:G, 2:169192010:C:G, 2:169193789:C:T, 2:169193790:A:G, 2:169193811:C:T, 2:169193850:T:C, 2:169196976:G:A, 2:169197010:T:C, 2:169198791:TA:T, 2:169198800:T:A, 2:169198809:C:T, 2:169198834:C:A, 2:169198840:A:G, 2:169198854:G:A, 2:169198858:T:C, 2:169198905:C:T, 2:169198909:C:T, 2:169201628:G:A, 2:169201648:G:A, 2:169201660:T:G, 2:169201719:C:T, 2:169201781:C:T, 2:169201789:T:C, 2:169201793:A:C, 2:169201805:G:T, 2:169201806:G:T, 2:169201825:C:T, 2:169202775:A:C, 2:169202788:C:G, 2:169202795:C:T, 2:169202834:G:T, 2:169202836:C:T, 2:169202839:T:C, 2:169202920:T:A, 2:169203985:G:C, 2:169203999:C:G, 2:169204030:T:A, 2:169204054:T:C, 2:169204120:C:A, 2:169204132:T:C, 2:169204137:C:T, 2:169204143:A:G, 2:169204175:A:T, 2:169204189:G:A, 2:169204229:T:G, 2:169204258:G:A, 2:169205492:C:T, 2:169205539:A:C, 2:169205570:G:A, 2:169205574:G:T, 2:169205590:G:A, 2:169205606:C:T, 2:169206047:G:C, 2:169206048:C:T, 2:169206089:C:T, 2:169206101:A:C, 2:169206111:T:G, 2:169206129:C:T, 2:169206143:C:T, 2:169206368:AG:A, 2:169206380:G:A, 2:169206389:C:G, 2:169206413:G:A, 2:169206414:T:C, 2:169206476:A:G, 2:169206493:G:T, 2:169206543:C:T, 2:169206564:T:G, 2:169206624:G:T, 2:169206639:G:T, 2:169206687:G:A, 2:169206689:T:C, 2:169206691:G:T, 2:169206694:G:T, 2:169206699:C:A, 2:169206734:A:G, 2:169206765:T:C, 2:169206772:T:C, 2:169206782:G:T, 2:169206825:T:G, 2:169206911:A:C, 2:169206969:C:T, 2:169207007:C:T, 2:169207023:T:A, 2:169207038:G:A, 2:169207049:T:C, 2:169207052:G:A, 2:169207056:A:G, 2:169207056:A:T, 2:169207059:C:A, 2:169207071:G:A, 2:169207074:C:T, 2:169207149:T:C, 2:169207155:C:T, 2:169207179:G:A, 2:169207181:T:G, 2:169207185:T:G, 2:169207194:T:C, 2:169209453:C:G, 2:169209583:A:C, 2:169209639:G:A, 2:169211969:T:G, 2:169212012:T:A, 2:169212022:A:C, 2:169212028:T:C, 2:169212039:A:G, 2:169212066:T:C, 2:169212078:G:A, 2:169212179:G:C, 2:169212195:G:A, 2:169212199:C:T, 2:169213713:A:G, 2:169213732:C:G, 2:169213738:C:T, 2:169213747:C:G, 2:169213756:C:T, 2:169213763:T:A, 2:169213794:A:G, 2:169213819:G:T, 2:169213830:A:C, 2:169216254:A:T, 2:169216260:C:T, 2:169216266:G:A, 2:169216272:G:T, 2:169216287:T:C, 2:169216297:T:C, 2:169216305:G:T, 2:169216328:G:T, 2:169216342:G:C, 2:169216377:G:A, 2:169216390:G:T, 2:169216398:C:T, 2:169216399:T:C, 2:169216431:C:G, 2:169220457:C:T, 2:169220458:G:A, 2:169220465:A:T, 2:169220472:G:C, 2:169220481:A:T, 2:169220497:C:T, 2:169220506:T:C, 2:169220521:T:C, 2:169220526:G:A, 2:169225360:A:G, 2:169225392:A:G, 2:169225404:G:A, 2:169225419:T:C, 2:169225452:C:T, 2:169226432:A:C, 2:169226442:T:C, 2:169226469:C:T, 2:169226480:T:C, 2:169226489:C:T, 2:169226499:G:T, 2:169226501:A:C, 2:169226511:C:T, 2:169226513:T:C, 2:169226516:C:T, 2:169226565:C:T, 2:169226579:G:T, 2:169226580:G:T, 2:169231717:T:C, 2:169231752:C:T, 2:169231759:G:A, 2:169231783:G:A, 2:169231785:C:T, 2:169231786:C:G, 2:169231821:G:A, 2:169231828:C:G, 2:169233437:G:T, 2:169233441:C:T, 2:169233464:T:G, 2:169233509:C:T, 2:169233510:G:A, 2:169233522:G:A, 2:169233548:G:C, 2:169233582:G:C, 2:169235891:G:C, 2:169235916:A:C, 2:169235926:G:A, 2:169235967:A:G, 2:169235978:C:A, 2:169235978:C:G, 2:169235994:C:A, 2:169236005:G:T, 2:169236015:G:A, 2:169236022:C:T, 2:169236027:C:A, 2:169236028:G:A, 2:169236028:G:C, 2:169236048:G:T, 2:169236053:GA:G, 2:169236054:A:T, 2:169237121:G:A, 2:169237131:T:C, 2:169237140:T:A, 2:169237170:G:T, 2:169237196:A:G, 2:169237206:G:C, 2:169237239:C:T, 2:169237254:T:C, 2:169237265:A:C, 2:169238101:T:C, 2:169238102:C:G, 2:169238104:G:T, 2:169238162:G:A, 2:169238167:C:T, 2:169238221:T:C, 2:169238267:T:A, 2:169238271:A:C, 2:169239532:A:G, 2:169239542:T:A, 2:169239544:C:G, 2:169239554:T:G, 2:169239574:T:C, 2:169239648:T:C, 2:169239718:G:A, 2:169240978:G:A, 2:169241020:C:T, 2:169241116:G:A, 2:169241159:G:A, 2:169241171:A:G, 2:169241177:T:A, 2:169241192:C:T, 2:169241239:G:T, 2:169241285:A:G, 2:169241299:G:A, 2:169242955:C:T, 2:169242976:T:C, 2:169242997:C:G, 2:169243021:A:G, 2:169243022:T:C, 2:169243025:A:G, 2:169243030:T:A, 2:169243057:G:A, 2:169243068:T:G, 2:169243421:A:T, 2:169243431:A:C, 2:169243455:A:C, 2:169243468:A:G, 2:169243486:C:T, 2:169243502:G:C, 2:169244702:T:G, 2:169244782:T:G, 2:169244819:G:A, 2:169244821:C:A, 2:169244833:T:C, 2:169244843:C:T, 2:169244873:A:G, 2:169244921:A:G, 2:169246738:C:G, 2:169246785:C:A, 2:169246800:G:A, 2:169246800:G:C, 2:169246831:G:A, 2:169246942:T:C, 2:169246978:C:A, 2:169247378:C:T, 2:169247384:G:T, 2:169247408:A:T, 2:169256142:G:C, 2:169256147:A:G, 2:169256184:T:C, 2:169256197:T:G, 2:169256235:C:T, 2:169257123:C:T, 2:169257127:C:G, 2:169257134:T:C, 2:169257145:T:C, 2:169257161:T:A, 2:169257207:C:T, 2:169257215:T:C, 2:169259026:C:T, 2:169259049:G:A, 2:169259052:C:A, 2:169259053:G:C, 2:169259067:T:C, 2:169259076:A:T, 2:169259079:G:A, 2:169259085:C:G, 2:169259086:T:C, 2:169259102:C:A, 2:169259106:A:C, 2:169259112:C:G, 2:169259112:C:T, 2:169259193:C:T, 2:169259194:T:C, 2:169270925:T:TA, 2:169270928:A:G, 2:169271011:C:G, 2:169272963:C:T, 2:169273016:A:G, 2:169273038:C:T, 2:169273044:T:C, 2:169275053:G:A, 2:169275099:A:G, 2:169275154:C:A, 2:169275224:T:A, 2:169275228:CTACAGTCT:C, 2:169275239:CTGT:C, 2:169277758:C:T, 2:169277848:C:T, 2:169277864:C:A, 2:169277893:A:G, 2:169277905:G:C, 2:169277911:G:A, 2:169277930:C:A, 2:169279376:C:G, 2:169279379:T:C, 2:169279402:G:A, 2:169279420:G:A, 2:169279448:C:A, 2:169279478:T:C, 2:169279519:G:T, 2:169279540:A:G, 2:169279556:C:A, 2:169279579:A:C, 2:169279592:A:G, 2:169280379:C:T, 2:169280406:C:T, 2:169280471:C:G, 2:169280487:G:A, 2:169280492:T:C, 2:169280504:A:C, 2:169280514:C:T, 2:169282903:G:A, 2:169282917:C:T, 2:169282942:G:A, 2:169282953:C:A, 2:169282968:T:C, 2:169282978:C:T, 2:169283001:T:A, 2:169289038:G:A, 2:169289047:T:C, 2:169289050:G:A, 2:169289065:C:A, 2:169289088:C:G, 2:169289104:G:A, 2:169289112:T:C, 2:169289121:T:A, 2:169290868:T:G, 2:169290878:C:T, 2:169290929:T:C, 2:169290961:C:G, 2:169290964:G:A, 2:169290971:A:G, 2:169290993:G:T, 2:169292251:AC:A, 2:169292312:T:C, 2:169292334:G:C, 2:169292361:T:C, 2:169294169:C:T, 2:169294208:G:A, 2:169294232:A:G, 2:169294598:A:T, 2:169294626:C:G, 2:169294686:G:T, 2:169294690:G:A, 2:169294697:A:C, 2:169294704:G:A, 2:169294708:ACT:A, 2:169307280:C:T, 2:169307326:C:T, 2:169307350:A:G, 2:169307386:AT:A, 2:169307397:G:A, 2:169307398:C:T, 2:169307399:T:C, 2:169318774:G:T, 2:169318774:G:A, 2:169320777:C:T, 2:169320787:T:G, 2:169320831:A:G, 2:169320840:G:T, 2:169320846:A:G, 2:169320884:T:A, 2:169362321:C:G, 2:169362374:GC:G, 2:169362392:C:T, 2:169128674:A:C, 2:169128677:C:T, 2:169128686:C:T, 2:169128691:T:C, 2:169128692:T:A, 2:169128694:G:A, 2:169128697:G:A, 2:169128700:T:G, 2:169128709:G:C, 2:169128719:T:C, 2:169128727:T:C, 2:169128737:T:G, 2:169128743:C:G, 2:169128743:C:T, 2:169128751:G:T, 2:169128757:G:A, 2:169128760:T:C, 2:169128764:G:T, 2:169128767:T:G, 2:169128778:G:T, 2:169128785:A:C, 2:169128791:G:A, 2:169128797:T:G, 2:169128802:G:A, 2:169128807:A:T, 2:169128808:C:T, 2:169128808:C:A, 2:169128812:C:T, 2:169128820:T:C, 2:169128821:C:T, 2:169128829:A:G, 2:169128829:A:T, 2:169129011:A:T, 2:169129012:C:T, 2:169129015:G:T, 2:169129022:G:C, 2:169129023:A:G, 2:169129026:G:A, 2:169129033:C:A, 2:169129056:T:A, 2:169129073:C:A, 2:169129080:G:A, 2:169129085:C:T, 2:169132578:G:T, 2:169132581:C:T, 2:169132591:C:T, 2:169132599:G:C, 2:169132599:G:A, 2:169132602:G:C, 2:169132609:T:C, 2:169132615:G:T, 2:169132617:A:T, 2:169132618:C:A, 2:169132630:G:T, 2:169132639:G:A, 2:169132653:T:C, 2:169132655:AT:A, 2:169132657:T:A, 2:169132663:C:T, 2:169132667:T:A, 2:169132678:T:A, 2:169137396:A:T, 2:169137399:G:A, 2:169137399:G:C, 2:169137403:G:A, 2:169137406:C:G, 2:169137406:C:T, 2:169137411:T:C, 2:169137415:C:T, 2:169137420:C:T, 2:169137421:T:C, 2:169137424:C:A, 2:169137432:G:A, 2:169137439:T:C, 2:169137442:G:A, 2:169137450:A:T, 2:169137453:A:T, 2:169137459:G:T, 2:169137463:G:C, 2:169137469:C:A, 2:169137469:C:T, 2:169137475:C:T, 2:169137476:C:T, 2:169137478:T:C, 2:169137481:C:T, 2:169137487:C:T, 2:169137489:T:C, 2:169137493:T:A, 2:169138576:C:T, 2:169138582:C:T, 2:169138582:C:G, 2:169138584:A:G, 2:169138624:C:G, 2:169138624:C:T, 2:169138630:T:C, 2:169138653:G:A, 2:169138660:A:C, 2:169138665:A:G, 2:169138666:C:A, 2:169138681:C:T, 2:169138688:C:G, 2:169138693:C:A, 2:169138696:G:C, 2:169138698:C:T, 2:169138701:C:G, 2:169138702:T:C, 2:169138705:A:T, 2:169139249:A:T, 2:169139251:C:T, 2:169139255:G:A, 2:169139260:T:C, 2:169139263:G:A, 2:169139269:G:A, 2:169139281:G:A, 2:169139285:C:T, 2:169139290:C:G, 2:169139299:T:C, 2:169139314:A:G, 2:169139317:G:A, 2:169139318:C:A, 2:169139326:C:G, 2:169139327:C:T, 2:169139329:A:G, 2:169139330:T:C, 2:169139332:A:T, 2:169139371:G:C, 2:169139538:CTTACTTG:C, 2:169139543:T:C, 2:169139548:C:T, 2:169139573:C:T, 2:169139574:C:T, 2:169139587:T:A, 2:169139591:C:T, 2:169139594:T:C, 2:169139594:T:A, 2:169139597:A:T, 2:169139609:A:G, 2:169140456:T:C, 2:169140461:T:G, 2:169140461:T:C, 2:169140468:G:C, 2:169140473:G:T, 2:169140474:T:G, 2:169140475:C:A, 2:169140476:T:C, 2:169140479:T:A, 2:169140485:T:C, 2:169140489:A:T, 2:169140499:G:T, 2:169140503:A:T, 2:169140503:A:C, 2:169140513:A:AT, 2:169140513:A:T, 2:169140515:G:T, 2:169140516:G:T, 2:169140519:G:A, 2:169140521:G:C, 2:169140521:G:T, 2:169140522:G:A, 2:169140525:G:C, 2:169140526:G:T, 2:169140527:T:C, 2:169140533:G:A, 2:169140539:T:C, 2:169140540:C:T, 2:169140545:G:C, 2:169142679:T:A, 2:169142683:A:C, 2:169142685:T:C, 2:169142691:G:T, 2:169142703:A:G, 2:169142710:T:C, 2:169142715:C:T, 2:169142727:G:T, 2:169142729:A:T, 2:169142735:G:T, 2:169142743:C:T, 2:169142757:CAG:C, 2:169142772:A:T, 2:169142773:T:C, 2:169142778:T:C, 2:169142779:T:C, 2:169142781:C:T, 2:169142784:A:C, 2:169142784:AG:A, 2:169142785:G:A, 2:169142785:G:T, 2:169142786:G:T, 2:169142787:T:C, 2:169142790:G:A, 2:169142793:A:G, 2:169145755:T:A, 2:169145758:T:C, 2:169145759:A:G, 2:169145760:T:G, 2:169145779:C:T, 2:169145780:G:A, 2:169145786:G:C, 2:169145812:G:T, 2:169145829:T:A, 2:169145851:C:T, 2:169145855:C:T, 2:169145861:C:T, 2:169145863:T:G, 2:169145872:G:T, 2:169145875:A:G, 2:169145876:T:C, 2:169145888:G:T, 2:169145890:T:C, 2:169145908:C:T, 2:169145914:G:C, 2:169145915:G:T, 2:169145916:G:T, 2:169145918:T:C, 2:169146737:A:C, 2:169146739:C:T, 2:169146741:T:A, 2:169146741:T:C, 2:169146742:C:T, 2:169146745:T:G, 2:169146750:A:G, 2:169146751:T:C, 2:169146754:C:G, 2:169146774:T:C, 2:169146777:T:G, 2:169146790:T:C, 2:169146796:C:T, 2:169146808:C:T, 2:169146819:A:G, 2:169146819:A:C, 2:169146822:C:T, 2:169146823:G:A, 2:169146838:A:G, 2:169146843:A:G, 2:169146852:C:G, 2:169146855:G:A, 2:169146869:G:C, 2:169146871:C:T, 2:169146885:A:G, 2:169146891:C:A, 2:169146891:C:T, 2:169146892:G:T, 2:169146892:G:A, 2:169146897:T:C, 2:169146905:C:T, 2:169146915:G:A, 2:169146919:C:G, 2:169146937:C:G, 2:169146938:C:G, 2:169146945:G:A, 2:169146952:A:T, 2:169150898:C:G, 2:169150920:T:C, 2:169150923:C:T, 2:169150925:G:A, 2:169150937:A:G, 2:169150941:C:T, 2:169150967:C:T, 2:169150976:A:G, 2:169150977:G:C, 2:169150982:G:A, 2:169150983:C:A, 2:169150986:C:A, 2:169150986:C:G, 2:169150988:T:G, 2:169151004:T:C, 2:169152798:C:A, 2:169152800:T:C, 2:169152805:A:G, 2:169152835:A:G, 2:169152835:A:C, 2:169152838:A:G, 2:169152839:C:A, 2:169152845:T:G, 2:169152855:G:T, 2:169152869:C:T, 2:169152901:A:T, 2:169152902:T:C, 2:169152917:T:G, 2:169152923:C:A, 2:169152927:C:A, 2:169152937:T:G, 2:169152944:G:A, 2:169154466:C:T, 2:169154487:C:A, 2:169154501:T:C, 2:169154511:C:G, 2:169154511:C:CAA, 2:169154512:C:G, 2:169154513:TC:T, 2:169154514:C:T, 2:169154516:T:A, 2:169154517:C:G, 2:169154529:C:G, 2:169154533:GA:G, 2:169154534:A:T, 2:169154536:C:A, 2:169154550:G:C, 2:169154562:G:A, 2:169154565:T:A, 2:169154568:G:A, 2:169154572:A:C, 2:169154592:A:C, 2:169154601:T:G, 2:169154603:C:T, 2:169156277:C:G, 2:169156279:G:A, 2:169156288:C:T, 2:169156295:C:A, 2:169156301:G:A, 2:169156315:G:T, 2:169156315:G:A, 2:169156325:C:G, 2:169156325:C:A, 2:169156331:A:T, 2:169156334:C:G, 2:169156351:T:C, 2:169156354:G:T, 2:169156359:T:A, 2:169156372:G:C, 2:169156384:A:T, 2:169156395:T:G, 2:169156396:T:A, 2:169156402:A:G, 2:169156405:T:C, 2:169157376:C:T, 2:169157379:G:C, 2:169157380:A:T, 2:169157385:C:A, 2:169157404:C:T, 2:169157415:G:A, 2:169157416:T:C, 2:169157424:C:G, 2:169157426:GATAA:G, 2:169157431:A:T, 2:169157433:C:T, 2:169157434:C:CGTGG, 2:169157437:C:T, 2:169157449:G:T, 2:169157461:G:C, 2:169157464:C:G, 2:169157464:C:T, 2:169157465:G:C, 2:169157482:A:G, 2:169157487:C:G, 2:169157491:C:A, 2:169157496:C:T, 2:169157502:T:C, 2:169162480:A:T, 2:169162487:C:T, 2:169162508:C:A, 2:169162516:T:A, 2:169162522:A:T, 2:169162532:G:A, 2:169162538:T:G, 2:169162550:T:A, 2:169162552:C:A, 2:169162552:C:T, 2:169162562:A:G, 2:169162570:T:A, 2:169162573:G:A, 2:169162588:G:T, 2:169162589:T:C, 2:169162589:T:A, 2:169162592:G:A, 2:169162592:GT:G, 2:169165935:G:A, 2:169165938:C:T, 2:169165947:C:T, 2:169165952:G:T, 2:169165953:T:G, 2:169165956:C:T, 2:169165958:T:C, 2:169165961:C:T, 2:169165964:C:T, 2:169165965:A:C, 2:169165968:C:T, 2:169165977:C:A, 2:169165988:T:C, 2:169165990:A:C, 2:169165993:A:T, 2:169165994:C:G, 2:169165995:T:C, 2:169165997:T:C, 2:169166001:T:G, 2:169166001:T:C, 2:169166003:CA:C, 2:169166006:C:A, 2:169166007:G:T, 2:169166010:T:C, 2:169166012:T:C, 2:169166024:A:T, 2:169166028:G:T, 2:169166028:G:A, 2:169166040:T:C, 2:169166045:G:A, 2:169168546:G:C, 2:169168547:T:C, 2:169168572:C:T, 2:169168605:G:T, 2:169168610:A:C, 2:169168611:T:C, 2:169168619:T:C, 2:169168621:G:T, 2:169168626:T:G, 2:169168629:A:T, 2:169168633:GA:G, 2:169168652:T:C, 2:169168662:C:G, 2:169168671:G:T, 2:169168673:G:A, 2:169168674:T:G, 2:169169701:C:T, 2:169169701:C:G, 2:169169711:C:T, 2:169169726:CCAAA:C, 2:169169728:A:G, 2:169169732:A:G, 2:169169741:A:G, 2:169169760:AC:A, 2:169169761:C:T, 2:169169780:T:G, 2:169169782:G:T, 2:169169782:G:A, 2:169169794:T:C, 2:169169801:G:A, 2:169169809:G:A, 2:169169812:C:T, 2:169169816:T:C, 2:169170552:A:C, 2:169170553:C:T, 2:169170556:T:C, 2:169170559:C:A, 2:169170568:G:A, 2:169170574:T:C, 2:169170583:T:A, 2:169170584:C:T, 2:169170585:G:T, 2:169170617:T:G, 2:169170624:C:A, 2:169170628:T:C, 2:169170631:A:C, 2:169170646:C:T, 2:169170667:G:A, 2:169172011:TCA:T, 2:169172035:T:A, 2:169172038:T:A, 2:169172045:A:C, 2:169172054:G:T, 2:169172059:T:G, 2:169172060:C:T, 2:169172063:A:G, 2:169172069:A:G, 2:169172075:G:T, 2:169172075:G:C, 2:169172077:G:A, 2:169172087:G:A, 2:169172107:T:A, 2:169172107:T:G, 2:169172110:C:G, 2:169172113:A:G, 2:169172114:C:T, 2:169172116:G:A, 2:169172121:G:T, 2:169172122:C:A, 2:169172134:T:C, 2:169173105:G:C, 2:169173114:T:G, 2:169173116:T:C, 2:169173120:C:T, 2:169173129:C:T, 2:169173131:T:C, 2:169173137:C:T, 2:169173137:C:G, 2:169173137:C:A, 2:169173138:C:T, 2:169173141:T:G, 2:169173143:C:T, 2:169173152:C:T, 2:169173162:T:C, 2:169173167:C:A, 2:169173173:T:C, 2:169173177:T:C, 2:169173186:T:G, 2:169173194:G:A, 2:169173198:A:C, 2:169173199:G:T, 2:169173200:T:C, 2:169173206:C:T, 2:169173210:G:C, 2:169173212:T:C, 2:169173215:G:A, 2:169173928:C:T, 2:169173930:A:T, 2:169173940:C:A, 2:169173954:C:A, 2:169173958:C:T, 2:169173961:T:C, 2:169173964:C:A, 2:169173971:A:C, 2:169173972:C:T, 2:169173974:C:A, 2:169173975:T:G, 2:169173977:C:A, 2:169173978:C:T, 2:169173984:T:C, 2:169173985:G:T, 2:169173997:G:T, 2:169173997:G:A, 2:169174005:G:A, 2:169174013:A:T, 2:169174014:A:T, 2:169174020:C:A, 2:169174029:C:T, 2:169174037:G:T, 2:169174041:G:T, 2:169174045:A:G, 2:169174046:G:T, 2:169174047:T:C, 2:169174065:G:A, 2:169174076:AC:A, 2:169174081:C:G, 2:169174084:T:G, 2:169174087:A:T, 2:169174097:C:A, 2:169174099:G:C, 2:169174099:G:T, 2:169174104:G:A, 2:169174110:G:A, 2:169174112:G:C, 2:169174132:A:G, 2:169174141:C:T, 2:169174157:G:T, 2:169174158:T:G, 2:169174162:TC:T, 2:169175192:C:A, 2:169175201:A:C, 2:169175202:G:C, 2:169175217:A:T, 2:169175219:C:A, 2:169175237:T:C, 2:169175243:T:C, 2:169175247:A:T, 2:169175253:T:C, 2:169175258:G:T, 2:169175274:C:T, 2:169175277:C:T, 2:169175283:A:T, 2:169175294:C:A, 2:169175294:C:T, 2:169175310:G:C, 2:169175310:G:T, 2:169175316:G:A, 2:169175322:G:A, 2:169175324:G:C, 2:169175330:T:C, 2:169175331:C:G, 2:169175333:T:C, 2:169175342:T:A, 2:169175355:T:C, 2:169175366:C:T, 2:169175368:T:A, 2:169175374:C:G, 2:169175374:C:A, 2:169175379:T:C, 2:169175385:T:C, 2:169176411:T:C, 2:169176417:T:C, 2:169176424:C:A, 2:169176435:T:G, 2:169176438:G:A, 2:169176441:C:G, 2:169176444:G:T, 2:169176447:C:T, 2:169176449:C:A, 2:169176450:A:G, 2:169176451:T:C, 2:169176453:G:A, 2:169176454:G:T, 2:169176457:T:C, 2:169176459:C:G, 2:169176463:A:G, 2:169176465:G:A, 2:169176465:G:T, 2:169176467:G:C, 2:169176477:A:G, 2:169176489:C:A, 2:169176489:C:T, 2:169176497:A:C, 2:169176502:G:A, 2:169176511:A:T, 2:169176514:T:C, 2:169176516:A:C, 2:169176522:T:C, 2:169176533:C:G, 2:169176538:T:G, 2:169176544:A:G, 2:169176550:G:A, 2:169176552:G:C, 2:169176564:T:C, 2:169176571:T:C, 2:169176573:C:T, 2:169176577:A:G, 2:169176582:T:C, 2:169176588:A:G, 2:169177802:C:A, 2:169177806:T:C, 2:169177808:G:C, 2:169177811:TG:T, 2:169177814:C:T, 2:169177826:T:C, 2:169177827:A:G, 2:169177832:T:A, 2:169177841:A:T, 2:169177844:G:T, 2:169177846:T:G, 2:169177866:GTGTC:G, 2:169177878:T:C, 2:169177887:C:T, 2:169177889:T:C, 2:169177890:ATT:A, 2:169177890:A:G, 2:169177901:T:G, 2:169177907:A:G, 2:169177922:C:T, 2:169177934:T:TA, 2:169177934:T:A, 2:169177940:G:A, 2:169177953:T:C, 2:169177962:C:A, 2:169177980:C:A, 2:169177985:T:C, 2:169177986:C:T, 2:169177986:C:CG, 2:169177988:T:A, 2:169177998:G:A, 2:169178000:C:T, 2:169178001:G:A, 2:169178019:C:T, 2:169181448:T:C, 2:169181451:A:T, 2:169181455:A:G, 2:169181457:C:T, 2:169181462:G:C, 2:169181492:GGT:G, 2:169181505:G:T, 2:169181509:T:A, 2:169181511:C:T, 2:169181514:T:C, 2:169181523:T:C, 2:169181533:T:C, 2:169181541:A:G, 2:169181552:G:C, 2:169181556:G:T, 2:169181566:T:C, 2:169181569:C:T, 2:169181569:C:A, 2:169181572:C:G, 2:169181574:C:T, 2:169181577:C:G, 2:169181581:T:G, 2:169181592:T:C, 2:169181596:C:T, 2:169181604:G:T, 2:169181606:C:G, 2:169181615:G:T, 2:169181616:T:C, 2:169182177:G:A, 2:169182180:G:A, 2:169182183:G:C, 2:169182198:G:T, 2:169182198:G:A, 2:169182204:C:G, 2:169182218:T:G, 2:169182227:T:C, 2:169182231:C:T, 2:169182233:C:G, 2:169182233:C:T, 2:169182248:A:G, 2:169182249:T:G, 2:169182249:T:A, 2:169182258:G:A, 2:169182267:T:C, 2:169182282:A:T, 2:169182285:G:A, 2:169182290:T:C, 2:169182291:C:A, 2:169182293:A:C, 2:169182297:G:A, 2:169182299:G:A, 2:169185509:A:G, 2:169185510:C:T, 2:169185519:C:T, 2:169185522:C:A, 2:169185524:A:C, 2:169185531:C:T, 2:169185537:C:T, 2:169185543:G:T, 2:169185555:T:C, 2:169185558:T:C, 2:169185560:G:T, 2:169185574:C:A, 2:169185578:T:C, 2:169185586:C:T, 2:169185586:C:G, 2:169185588:T:G, 2:169185605:C:T, 2:169185611:G:T, 2:169185614:T:C, 2:169185615:C:T, 2:169185618:TC:T, 2:169185623:T:C, 2:169185629:C:T, 2:169185630:T:C, 2:169185639:C:T, 2:169185641:C:A, 2:169185642:G:C, 2:169185647:A:G, 2:169185649:A:C, 2:169185653:A:G, 2:169185654:A:C, 2:169185657:C:A, 2:169185668:T:C, 2:169185704:T:C, 2:169185706:T:C, 2:169185732:G:A, 2:169185737:C:T, 2:169185744:T:C, 2:169185753:G:T, 2:169185758:A:G, 2:169185761:T:A, 2:169185793:T:A, 2:169185797:C:G, 2:169185808:TG:T, 2:169185812:G:T, 2:169185819:T:G, 2:169185825:T:A, 2:169185827:T:C, 2:169185830:G:C, 2:169185830:G:A, 2:169185831:A:G, 2:169185833:C:T, 2:169185836:A:G, 2:169185837:T:C, 2:169185840:C:T, 2:169185841:A:C, 2:169185846:C:T, 2:169185850:C:G, 2:169185851:T:A, 2:169185852:T:C, 2:169185864:C:G, 2:169185864:C:T, 2:169185870:G:A, 2:169185872:A:T, 2:169185882:A:G, 2:169185888:C:CAATA, 2:169185893:T:C, 2:169185897:C:T, 2:169185900:A:C, 2:169185910:G:C, 2:169185914:G:A, 2:169185929:C:A, 2:169185932:G:T, 2:169185936:G:A, 2:169185958:T:G, 2:169185964:G:T, 2:169185966:C:T, 2:169185968:G:C, 2:169185974:T:A, 2:169185976:G:C, 2:169185980:T:C, 2:169185992:A:G, 2:169185993:T:C, 2:169185995:G:A, 2:169186004:T:C, 2:169186014:T:C, 2:169187969:C:T, 2:169187972:C:T, 2:169187984:T:G, 2:169187985:C:T, 2:169187986:G:C, 2:169187991:T:C, 2:169188006:C:T, 2:169188013:G:C, 2:169188032:T:C, 2:169188033:C:A, 2:169188034:G:C, 2:169188051:C:T, 2:169188055:C:G, 2:169188060:A:G, 2:169188063:C:T, 2:169188066:G:C, 2:169188072:G:A, 2:169188077:G:T, 2:169188077:G:C, 2:169188080:G:A, 2:169188087:G:A, 2:169188099:G:T, 2:169188100:G:T, 2:169188101:T:G, 2:169188102:G:A, 2:169188108:G:C, 2:169188114:C:T, 2:169188133:A:T, 2:169188137:T:A, 2:169188139:C:G, 2:169188150:C:A, 2:169188151:G:T, 2:169188173:C:T, 2:169188179:T:C, 2:169188185:G:T, 2:169188191:T:C, 2:169188192:G:T, 2:169188194:T:A, 2:169188203:C:T, 2:169188204:A:C, 2:169188206:G:C, 2:169188218:C:G, 2:169188219:A:C, 2:169188222:C:G, 2:169188223:C:A, 2:169188224:C:T, 2:169188229:G:C, 2:169188236:T:G, 2:169188236:T:C, 2:169188239:T:G, 2:169188245:C:G, 2:169188245:C:T, 2:169191848:T:C, 2:169191848:T:G, 2:169191859:T:C, 2:169191860:A:C, 2:169191865:C:T, 2:169191866:A:G, 2:169191869:T:C, 2:169191872:A:G, 2:169191873:T:A, 2:169191876:A:T, 2:169191883:G:A, 2:169191893:T:C, 2:169191896:T:C, 2:169191901:C:A, 2:169191906:C:A, 2:169191911:T:C, 2:169191916:T:A, 2:169191917:C:A, 2:169191919:T:C, 2:169191929:T:C, 2:169191935:C:G, 2:169191938:C:T, 2:169191959:A:G, 2:169191960:C:G, 2:169191968:T:C, 2:169191985:G:A, 2:169191986:G:A, 2:169191991:T:C, 2:169192003:A:G, 2:169192008:C:G, 2:169192013:A:G, 2:169192022:A:G, 2:169192023:G:C, 2:169193778:T:C, 2:169193791:T:A, 2:169193808:T:A, 2:169193817:C:T, 2:169193822:C:T, 2:169193829:C:T, 2:169193834:G:C, 2:169193854:A:G, 2:169193855:C:G, 2:169193856:T:C, 2:169193869:C:T, 2:169193881:G:A, 2:169196916:G:C, 2:169196919:G:C, 2:169196920:C:T, 2:169196934:G:T, 2:169196936:A:C, 2:169196938:C:A, 2:169196940:A:G, 2:169196953:C:G, 2:169196958:T:C, 2:169196961:C:T, 2:169196969:A:T, 2:169197009:C:T, 2:169197015:C:T, 2:169197024:T:C, 2:169198794:G:A, 2:169198797:G:C, 2:169198799:G:T, 2:169198804:C:A, 2:169198810:T:C, 2:169198813:T:G, 2:169198816:C:G, 2:169198816:C:T, 2:169198818:C:G, 2:169198825:C:T, 2:169198828:T:C, 2:169198830:T:A, 2:169198833:C:T, 2:169198834:C:T, 2:169198846:A:G, 2:169198846:A:T, 2:169198851:C:A, 2:169198856:A:C, 2:169198857:A:G, 2:169198860:C:T, 2:169198862:A:C, 2:169198865:A:T, 2:169198873:T:A, 2:169198875:T:C, 2:169198878:C:A, 2:169198905:C:A, 2:169198912:C:A, 2:169201641:A:C, 2:169201651:T:C, 2:169201654:T:G, 2:169201654:T:C, 2:169201660:T:C, 2:169201669:T:A, 2:169201675:C:G, 2:169201676:C:T, 2:169201679:T:C, 2:169201686:A:C, 2:169201693:C:A, 2:169201693:C:T, 2:169201696:G:A, 2:169201705:C:T, 2:169201714:T:C, 2:169201729:G:A, 2:169201739:T:G, 2:169201761:T:TG, 2:169201763:C:A, 2:169201766:C:T, 2:169201774:C:CATTGTAGT, 2:169201775:C:A, 2:169201779:AC:A, 2:169201794:G:C, 2:169201795:T:C, 2:169201804:C:T, 2:169201810:G:C, 2:169201813:T:A, 2:169201817:G:C, 2:169201822:C:T, 2:169201826:G:A, 2:169201828:C:A, 2:169201832:T:C, 2:169201834:G:T, 2:169201844:A:T, 2:169201850:T:C, 2:169201855:G:A, 2:169201856:A:G, 2:169201861:G:C, 2:169202755:C:T, 2:169202763:A:T, 2:169202764:C:G, 2:169202767:T:A, 2:169202769:C:G, 2:169202771:T:C, 2:169202776:T:C, 2:169202796:G:T, 2:169202807:C:A, 2:169202812:T:A, 2:169202816:A:G, 2:169202818:T:A, 2:169202820:T:A, 2:169202824:G:C, 2:169202825:A:T, 2:169202826:G:C, 2:169202827:A:C, 2:169202861:C:T, 2:169202869:C:A, 2:169202870:G:A, 2:169202873:C:G, 2:169202885:C:A, 2:169202888:T:C, 2:169202889:G:T, 2:169202890:C:T, 2:169202895:C:G, 2:169202902:T:C, 2:169202905:T:C, 2:169202908:G:A, 2:169202909:C:T, 2:169202914:T:C, 2:169202918:A:G, 2:169202921:T:G, 2:169202929:T:C, 2:169202930:G:T, 2:169202956:G:A, 2:169203984:G:A, 2:169203994:T:C, 2:169204011:T:C, 2:169204011:T:A, 2:169204017:T:C, 2:169204020:T:C, 2:169204021:C:T, 2:169204022:A:C, 2:169204033:T:A, 2:169204038:T:C, 2:169204039:G:C, 2:169204048:G:T, 2:169204050:T:A, 2:169204051:T:A, 2:169204052:C:A, 2:169204060:C:T, 2:169204080:T:A, 2:169204083:G:A, 2:169204083:G:T, 2:169204093:T:A, 2:169204105:C:T, 2:169204110:T:C, 2:169204116:G:C, 2:169204119:C:T, 2:169204120:C:T, 2:169204123:CAT:C, 2:169204123:C:T, 2:169204128:T:A, 2:169204138:G:A, 2:169204146:C:A, 2:169204158:A:G, 2:169204162:C:T, 2:169204176:T:C, 2:169204182:C:T, 2:169204183:C:T, 2:169204189:G:C, 2:169204191:G:C, 2:169204192:T:A, 2:169204207:G:C, 2:169204207:G:GGA, 2:169204215:G:A, 2:169204216:C:A, 2:169204224:A:G, 2:169204240:C:T, 2:169204242:C:A, 2:169204257:C:T, 2:169204261:C:G, 2:169204266:C:T, 2:169205486:C:T, 2:169205492:C:G, 2:169205510:C:T, 2:169205515:T:C, 2:169205518:T:C, 2:169205522:G:T, 2:169205534:TG:T, 2:169205564:G:C, 2:169205593:C:T, 2:169205597:C:T, 2:169205600:T:C, 2:169205635:T:G, 2:169205638:C:T, 2:169206022:C:T, 2:169206032:G:A, 2:169206035:T:A, 2:169206065:C:CG, 2:169206066:G:T, 2:169206072:T:C, 2:169206078:T:G, 2:169206080:C:A, 2:169206082:G:C, 2:169206084:T:C, 2:169206086:G:C, 2:169206089:C:A, 2:169206092:T:A, 2:169206102:T:C, 2:169206114:T:G, 2:169206123:G:C, 2:169206135:A:G, 2:169206143:C:A, 2:169206147:T:C, 2:169206154:C:A, 2:169206156:A:T, 2:169206164:G:A, 2:169206171:C:A, 2:169206174:C:T, 2:169206177:C:G, 2:169206333:A:C, 2:169206356:A:T, 2:169206372:T:C, 2:169206375:C:T, 2:169206389:C:T, 2:169206392:A:G, 2:169206398:G:A, 2:169206410:T:A, 2:169206414:T:A, 2:169206420:A:G, 2:169206437:C:T, 2:169206438:T:C, 2:169206441:C:T, 2:169206443:T:G, 2:169206446:T:C, 2:169206450:G:T, 2:169206456:T:A, 2:169206464:C:G, 2:169206473:T:C, 2:169206474:T:C, 2:169206479:G:A, 2:169206485:A:C, 2:169206486:A:C, 2:169206488:G:A, 2:169206506:G:C, 2:169206515:T:C, 2:169206518:A:G, 2:169206522:T:C, 2:169206552:G:A, 2:169206563:G:A, 2:169206575:C:G, 2:169206587:T:C, 2:169206588:C:T, 2:169206590:C:T, 2:169206590:C:A, 2:169206591:T:A, 2:169206592:T:G, 2:169206596:AG:A, 2:169206597:G:T, 2:169206599:G:A, 2:169206602:C:T, 2:169206605:A:C, 2:169206608:G:A, 2:169206617:C:T, 2:169206619:T:G, 2:169206624:G:A, 2:169206626:G:T, 2:169206651:A:G, 2:169206653:A:G, 2:169206657:G:A, 2:169206671:T:C, 2:169206686:G:A, 2:169206690:T:C, 2:169206692:T:C, 2:169206698:A:G, 2:169206713:C:G, 2:169206713:C:A, 2:169206716:G:T, 2:169206730:G:T, 2:169206734:A:C, 2:169206738:T:C, 2:169206740:G:A, 2:169206743:A:C, 2:169206744:C:G, 2:169206754:C:A, 2:169206757:G:T, 2:169206759:T:A, 2:169206766:G:C, 2:169206774:T:C, 2:169206774:T:A, 2:169206777:C:T, 2:169206786:G:A, 2:169206789:C:T, 2:169206793:G:T, 2:169206807:T:C, 2:169206809:C:A, 2:169206812:G:C, 2:169206813:C:T, 2:169206821:A:T, 2:169206823:C:A, 2:169206824:T:G, 2:169206828:T:C, 2:169206844:C:T, 2:169206857:T:C, 2:169206862:A:T, 2:169206878:T:C, 2:169206882:G:A, 2:169206888:G:A, 2:169206893:C:A, 2:169206894:G:A, 2:169206896:C:T, 2:169206905:C:A, 2:169206906:G:T, 2:169206908:A:C, 2:169206909:T:A, 2:169206913:T:A, 2:169206929:T:C, 2:169206933:T:C, 2:169206951:T:C, 2:169206954:C:T, 2:169206960:A:G, 2:169206961:A:C, 2:169206970:C:A, 2:169206972:A:G, 2:169206980:T:A, 2:169206980:T:C, 2:169206983:C:T, 2:169206983:C:A, 2:169207011:G:C, 2:169207011:G:A, 2:169207016:G:A, 2:169207025:C:T, 2:169207032:A:C, 2:169207043:G:A, 2:169207046:C:A, 2:169207047:G:A, 2:169207064:A:C, 2:169207094:T:C, 2:169207094:T:G, 2:169207110:G:A, 2:169207121:T:C, 2:169207125:G:A, 2:169207145:G:A, 2:169207147:C:T, 2:169207163:T:A, 2:169207164:T:G, 2:169207176:G:T, 2:169207181:T:C, 2:169207185:T:C, 2:169207188:T:G, 2:169207203:C:T, 2:169207208:A:G, 2:169207218:C:T, 2:169207220:G:A, 2:169207220:G:T, 2:169207227:A:G, 2:169207232:T:C, 2:169207246:A:T, 2:169209451:A:G, 2:169209456:C:A, 2:169209458:A:G, 2:169209459:C:T, 2:169209462:A:C, 2:169209472:A:C, 2:169209473:A:C, 2:169209473:A:G, 2:169209476:C:T, 2:169209477:C:A, 2:169209479:C:T, 2:169209480:G:A, 2:169209485:C:G, 2:169209494:C:T, 2:169209497:A:G, 2:169209501:C:T, 2:169209515:T:C, 2:169209516:T:A, 2:169209517:C:A, 2:169209527:G:T, 2:169209532:ATCTG:A, 2:169209533:T:C, 2:169209537:G:T, 2:169209545:C:G, 2:169209546:T:C, 2:169209549:G:C, 2:169209554:G:A, 2:169209554:G:C, 2:169209555:C:T, 2:169209563:G:T, 2:169209578:C:T, 2:169209591:A:G, 2:169209602:C:G, 2:169209605:G:A, 2:169209606:A:T, 2:169209614:T:C, 2:169209615:C:A, 2:169209620:T:C, 2:169209620:T:A, 2:169209623:A:G, 2:169209626:T:G, 2:169209629:A:G, 2:169209632:G:A, 2:169209633:C:G, 2:169209633:C:T, 2:169209635:T:C, 2:169211966:A:G, 2:169211968:C:A, 2:169211970:T:C, 2:169211974:C:T, 2:169211977:C:T, 2:169211979:A:G, 2:169211982:G:A, 2:169211988:A:G, 2:169211991:G:T, 2:169212008:C:A, 2:169212018:C:A, 2:169212022:A:G, 2:169212025:C:T, 2:169212037:A:C, 2:169212043:T:C, 2:169212045:G:A, 2:169212057:A:T, 2:169212079:A:T, 2:169212081:C:T, 2:169212085:T:C, 2:169212088:C:T, 2:169212094:T:C, 2:169212096:A:C, 2:169212097:G:C, 2:169212100:T:G, 2:169212114:C:G, 2:169212115:A:T, 2:169212118:C:A, 2:169212118:C:G, 2:169212132:C:A, 2:169212132:C:CCAAAAGCATT, 2:169212133:C:T, 2:169212136:C:T, 2:169212165:G:C, 2:169212169:T:G, 2:169212178:T:A, 2:169212180:C:G, 2:169212183:C:G, 2:169212184:A:C, 2:169212186:C:T, 2:169212198:T:C, 2:169212202:C:A, 2:169212204:G:A, 2:169213657:T:C, 2:169213664:G:T, 2:169213666:G:A, 2:169213669:A:G, 2:169213680:C:G, 2:169213690:T:A, 2:169213692:G:C, 2:169213711:C:G, 2:169213718:G:C, 2:169213722:G:A, 2:169213723:C:T, 2:169213725:C:T, 2:169213729:T:C, 2:169213735:T:A, 2:169213735:T:C, 2:169213737:T:A, 2:169213749:A:G, 2:169213750:T:C, 2:169213753:C:T, 2:169213765:C:G, 2:169213765:C:T, 2:169213768:C:T, 2:169213771:T:C, 2:169213781:G:T, 2:169213783:A:G, 2:169213785:G:A, 2:169213791:T:C, 2:169213792:G:A, 2:169213798:C:A, 2:169213813:G:A, 2:169213824:T:C, 2:169213825:G:A, 2:169213833:A:T, 2:169213840:G:A, 2:169213843:C:T, 2:169213851:T:A, 2:169213851:T:C, 2:169213863:C:T, 2:169213870:T:C, 2:169216252:C:G, 2:169216267:T:C, 2:169216273:C:T, 2:169216291:C:T, 2:169216296:A:T, 2:169216305:G:A, 2:169216311:C:T, 2:169216320:T:A, 2:169216324:C:T, 2:169216327:G:A, 2:169216328:G:C, 2:169216329:T:C, 2:169216335:G:A, 2:169216356:G:T, 2:169216360:CA:C, 2:169216365:A:C, 2:169216371:G:C, 2:169216375:T:C, 2:169216390:G:A, 2:169216392:A:G, 2:169216395:C:A, 2:169216411:G:A, 2:169216428:T:G, 2:169220455:C:G, 2:169220455:C:T, 2:169220457:C:G, 2:169220461:C:T, 2:169220467:C:T, 2:169220470:C:T, 2:169220474:T:C, 2:169220478:C:A, 2:169220482:T:C, 2:169220490:C:G, 2:169220491:C:T, 2:169220500:G:C, 2:169220505:G:T, 2:169220508:C:T, 2:169220512:C:G, 2:169220514:T:C, 2:169220518:C:A, 2:169220522:C:G, 2:169220541:A:C, 2:169220544:T:G, 2:169220547:C:T, 2:169220548:C:T, 2:169220550:T:G, 2:169220551:G:C, 2:169220551:G:A, 2:169220557:T:A, 2:169220558:C:G, 2:169225312:C:T, 2:169225315:T:C, 2:169225324:T:C, 2:169225332:T:C, 2:169225338:G:C, 2:169225356:A:C, 2:169225357:T:A, 2:169225365:A:G, 2:169225368:G:A, 2:169225378:T:C, 2:169225380:G:A, 2:169225390:C:A, 2:169225393:T:C, 2:169225398:A:G, 2:169225399:T:C, 2:169225405:C:T, 2:169225405:C:A, 2:169225411:C:T, 2:169225413:G:A, 2:169225413:G:T, 2:169225420:T:A, 2:169225428:T:A, 2:169225440:C:T, 2:169225450:C:T, 2:169226424:G:A, 2:169226424:G:T, 2:169226424:G:C, 2:169226434:C:T, 2:169226436:A:G, 2:169226437:AT:A, 2:169226441:A:G, 2:169226446:T:G, 2:169226447:T:C, 2:169226456:T:G, 2:169226472:C:T, 2:169226495:A:C, 2:169226498:G:A, 2:169226499:G:A, 2:169226503:C:T, 2:169226505:T:C, 2:169226507:G:A, 2:169226510:T:A, 2:169226510:T:C, 2:169226520:T:G, 2:169226524:C:A, 2:169226549:A:G, 2:169226552:A:G, 2:169226558:T:G, 2:169226563:TAC:T, 2:169226567:G:C, 2:169226571:T:C, 2:169226579:G:A, 2:169226580:G:C, 2:169226581:T:G, 2:169226584:A:T, 2:169226588:T:C, 2:169231715:T:A, 2:169231716:C:T, 2:169231728:A:G, 2:169231734:T:C, 2:169231735:C:A, 2:169231738:G:A, 2:169231743:A:G, 2:169231745:A:T, 2:169231780:G:T, 2:169231788:T:C, 2:169231804:A:C, 2:169231811:G:C, 2:169231818:C:A, 2:169231825:A:G, 2:169231827:G:A, 2:169231839:A:G, 2:169233416:G:T, 2:169233425:G:A, 2:169233431:T:A, 2:169233435:C:A, 2:169233438:C:T, 2:169233444:T:G, 2:169233455:C:A, 2:169233464:T:C, 2:169233470:A:G, 2:169233474:C:A, 2:169233483:G:C, 2:169233483:G:T, 2:169233493:A:T, 2:169233496:C:A, 2:169233499:C:G, 2:169233510:G:C, 2:169233516:C:T, 2:169233518:C:G, 2:169233522:G:T, 2:169233524:G:C, 2:169233524:G:A, 2:169233525:T:C, 2:169233530:C:G, 2:169233537:T:C, 2:169233542:T:G, 2:169233545:A:C, 2:169233557:A:C, 2:169233569:G:A, 2:169233570:C:T, 2:169233577:G:T, 2:169233578:T:C, 2:169233581:C:A, 2:169233588:T:C, 2:169235839:C:A, 2:169235848:T:C, 2:169235848:T:G, 2:169235850:G:T, 2:169235851:C:T, 2:169235854:T:G, 2:169235862:C:T, 2:169235867:A:C, 2:169235868:T:C, 2:169235868:T:A, 2:169235870:G:T, 2:169235877:T:C, 2:169235878:T:C, 2:169235884:C:T, 2:169235896:T:C, 2:169235898:T:A, 2:169235913:T:C, 2:169235918:G:C, 2:169235929:G:T, 2:169235934:T:C, 2:169235937:G:A, 2:169235942:G:C, 2:169235949:G:A, 2:169235950:T:C, 2:169235972:C:A, 2:169235974:T:C, 2:169235979:T:C, 2:169235982:A:C, 2:169235986:T:TG, 2:169235989:C:T, 2:169235991:G:A, 2:169235992:T:C, 2:169235995:G:A, 2:169236004:C:T, 2:169236010:T:C, 2:169236012:C:T, 2:169236018:C:T, 2:169236019:G:A, 2:169236023:G:C, 2:169236025:T:C, 2:169236026:G:GC, 2:169236027:C:T, 2:169236031:G:A, 2:169236033:T:G, 2:169236037:G:A, 2:169236040:C:T, 2:169236042:C:T, 2:169236046:C:T, 2:169236049:A:T, 2:169237107:T:C, 2:169237112:G:C, 2:169237112:G:A, 2:169237113:G:A, 2:169237122:C:T, 2:169237144:G:GT, 2:169237171:G:T, 2:169237178:T:C, 2:169237185:T:G, 2:169237187:G:C, 2:169237199:G:T, 2:169237202:T:C, 2:169237205:A:C, 2:169237208:G:A, 2:169237209:C:T, 2:169237210:A:C, 2:169237222:G:T, 2:169237229:T:C, 2:169237233:G:C, 2:169237238:A:G, 2:169237251:C:T, 2:169237252:A:C, 2:169237269:T:C, 2:169237275:T:C, 2:169237276:ACTGTCAAATAC:A, 2:169237279:G:C, 2:169237283:A:C, 2:169237284:A:G, 2:169238097:T:A, 2:169238098:C:T, 2:169238101:T:A, 2:169238105:T:A, 2:169238124:C:G, 2:169238128:G:T, 2:169238138:G:C, 2:169238144:C:A, 2:169238159:T:C, 2:169238164:C:T, 2:169238171:T:C, 2:169238183:C:G, 2:169238186:C:T, 2:169238189:C:A, 2:169238207:T:C, 2:169238210:C:T, 2:169238218:G:A, 2:169238228:T:C, 2:169238233:A:G, 2:169238236:T:G, 2:169238248:C:T, 2:169238248:C:A, 2:169238249:T:G, 2:169238252:C:A, 2:169238258:T:C, 2:169238265:G:T, 2:169238266:T:A, 2:169238267:T:C, 2:169238269:T:G, 2:169238271:A:T, 2:169238273:T:C, 2:169238276:C:T, 2:169238281:A:G, 2:169238290:A:G, 2:169238293:C:T, 2:169238300:A:T, 2:169239525:A:G, 2:169239529:G:A, 2:169239530:T:C, 2:169239541:G:C, 2:169239545:T:C, 2:169239551:C:A, 2:169239559:A:G, 2:169239562:A:T, 2:169239563:T:C, 2:169239564:G:C, 2:169239569:C:T, 2:169239571:G:C, 2:169239580:G:A, 2:169239589:A:G, 2:169239592:G:A, 2:169239592:G:T, 2:169239600:C:T, 2:169239613:T:C, 2:169239614:G:A, 2:169239625:G:C, 2:169239625:G:A, 2:169239633:A:C, 2:169239637:T:C, 2:169239643:T:C, 2:169239645:A:T, 2:169239646:T:C, 2:169239649:A:G, 2:169239661:G:T, 2:169239661:G:C, 2:169239677:C:T, 2:169239680:G:A, 2:169239681:TA:T, 2:169239682:A:C, 2:169239706:T:C, 2:169239718:G:C, 2:169239728:C:T, 2:169239748:C:T, 2:169239750:A:T, 2:169239752:T:C, 2:169239754:A:G, 2:169239755:A:T, 2:169239757:T:C, 2:169239758:C:A, 2:169239774:A:C, 2:169240946:A:G, 2:169240956:T:C, 2:169240958:T:C, 2:169240961:C:A, 2:169240969:C:T, 2:169240970:T:C, 2:169240973:G:A, 2:169240976:C:A, 2:169240985:T:C, 2:169240991:A:G, 2:169240993:A:C, 2:169241006:C:A, 2:169241009:T:C, 2:169241018:G:A, 2:169241029:A:G, 2:169241032:C:T, 2:169241032:C:G, 2:169241033:C:T, 2:169241036:C:G, 2:169241038:C:T, 2:169241047:C:T, 2:169241057:C:T, 2:169241062:A:G, 2:169241063:T:C, 2:169241072:C:T, 2:169241072:C:G, 2:169241089:C:A, 2:169241093:T:C, 2:169241096:G:C, 2:169241096:G:A, 2:169241097:A:C, 2:169241107:G:C, 2:169241114:T:C, 2:169241117:G:T, 2:169241126:T:A, 2:169241132:C:A, 2:169241134:C:T, 2:169241137:A:G, 2:169241144:C:G, 2:169241145:G:T, 2:169241159:G:C, 2:169241195:T:C, 2:169241201:A:G, 2:169241215:G:A, 2:169241216:A:G, 2:169241219:G:C, 2:169241221:C:A, 2:169241224:GT:G, 2:169241227:T:C, 2:169241236:C:G, 2:169241239:G:GCATTGTGC, 2:169241240:C:T, 2:169241257:C:A, 2:169241266:C:A, 2:169241269:T:C, 2:169241276:G:A, 2:169241278:C:T, 2:169241296:T:G, 2:169241311:C:T, 2:169241331:T:G, 2:169241334:A:T, 2:169241341:T:A, 2:169241348:T:C, 2:169241354:G:T, 2:169241354:G:A, 2:169241357:G:T, 2:169241363:T:C, 2:169242954:A:T, 2:169242962:C:T, 2:169242967:T:C, 2:169242969:A:T, 2:169242970:T:C, 2:169242979:T:C, 2:169242997:C:T, 2:169243000:T:A, 2:169243007:G:A, 2:169243020:A:C, 2:169243030:T:C, 2:169243033:C:G, 2:169243039:G:A, 2:169243043:A:C, 2:169243045:T:C, 2:169243063:C:T, 2:169243413:C:A, 2:169243416:A:C, 2:169243444:C:T, 2:169243460:AT:A, 2:169243463:G:C, 2:169243469:T:A, 2:169243469:T:C, 2:169243471:C:T, 2:169243473:TC:T, 2:169243474:C:G, 2:169243475:G:A, 2:169243495:T:A, 2:169243498:C:G, 2:169243498:C:T, 2:169243502:G:A, 2:169243505:G:T, 2:169243516:G:A, 2:169244692:C:T, 2:169244700:C:A, 2:169244701:T:C, 2:169244732:C:T, 2:169244734:G:A, 2:169244744:C:G, 2:169244748:G:T, 2:169244750:T:C, 2:169244761:C:T, 2:169244762:A:G, 2:169244780:T:C, 2:169244788:G:A, 2:169244803:G:A, 2:169244806:G:T, 2:169244807:G:T, 2:169244807:G:A, 2:169244813:G:A, 2:169244815:G:A, 2:169244816:T:C, 2:169244828:G:C, 2:169244839:C:T, 2:169244839:C:A, 2:169244840:C:T, 2:169244843:C:G, 2:169244857:C:G, 2:169244864:C:T, 2:169244870:G:A, 2:169244875:T:A, 2:169244875:T:C, 2:169244878:G:T, 2:169244881:G:C, 2:169244891:C:T, 2:169244894:C:T, 2:169244896:T:C, 2:169244905:G:A, 2:169244912:C:T, 2:169244915:A:T, 2:169244920:G:A, 2:169244920:GA:G, 2:169244926:G:T, 2:169244926:G:C, 2:169244929:T:G, 2:169244930:T:G, 2:169244930:T:C, 2:169244932:T:G, 2:169246707:A:C, 2:169246713:C:T, 2:169246714:C:T, 2:169246737:T:C, 2:169246740:T:C, 2:169246758:T:C, 2:169246765:G:C, 2:169246771:A:C, 2:169246772:A:C, 2:169246780:C:T, 2:169246788:C:A, 2:169246801:G:C, 2:169246818:C:A, 2:169246830:G:C, 2:169246830:G:A, 2:169246833:G:A, 2:169246848:TC:T, 2:169246849:C:G, 2:169246852:C:G, 2:169246864:AG:A, 2:169246881:C:G, 2:169246887:C:G, 2:169246889:A:C, 2:169246891:A:C, 2:169246894:G:A, 2:169246896:C:T, 2:169246908:C:T, 2:169246909:G:A, 2:169246912:G:A, 2:169246921:G:A, 2:169246938:T:C, 2:169246939:G:A, 2:169246950:C:T, 2:169246951:C:G, 2:169246959:T:C, 2:169246977:G:A, 2:169246979:G:T, 2:169246981:T:C, 2:169246986:C:T, 2:169246986:C:A, 2:169247378:C:G, 2:169247386:A:G, 2:169247392:A:G, 2:169247398:T:C, 2:169247404:T:C, 2:169247410:T:C, 2:169247413:A:G, 2:169247416:A:G, 2:169247417:T:G, 2:169247428:C:T, 2:169247431:C:T, 2:169247432:T:A, 2:169247434:C:T, 2:169247436:G:C, 2:169247443:G:A, 2:169247444:T:C, 2:169247452:C:T, 2:169247456:C:T, 2:169247461:G:T, 2:169247462:C:A, 2:169247474:G:A, 2:169247485:C:G, 2:169247496:G:C, 2:169247498:C:T, 2:169247498:C:A, 2:169247501:T:TA, 2:169247505:A:C, 2:169247507:A:C, 2:169247508:T:A, 2:169256106:C:G, 2:169256108:C:T, 2:169256109:C:T, 2:169256115:T:C, 2:169256121:G:A, 2:169256127:A:G, 2:169256131:A:T, 2:169256138:A:T, 2:169256141:T:C, 2:169256145:C:G, 2:169256154:C:T, 2:169256164:T:A, 2:169256165:C:T, 2:169256177:T:C, 2:169256178:C:A, 2:169256186:C:T, 2:169256196:T:C, 2:169256200:A:T, 2:169256206:A:T, 2:169256208:A:G, 2:169256213:T:A, 2:169256217:C:A, 2:169256217:C:T, 2:169256218:C:A, 2:169256220:A:G, 2:169256229:G:A, 2:169256234:G:T, 2:169257127:C:A, 2:169257128:A:C, 2:169257149:G:T, 2:169257154:C:G, 2:169257158:G:A, 2:169257167:T:G, 2:169257175:G:A, 2:169257177:C:A, 2:169257192:G:C, 2:169257197:T:C, 2:169257200:A:G, 2:169257203:C:T, 2:169257209:T:C, 2:169257220:G:A, 2:169257223:C:G, 2:169257228:C:G, 2:169257232:T:C, 2:169257242:A:G, 2:169257251:T:A, 2:169259024:C:A, 2:169259025:C:A, 2:169259034:G:C, 2:169259049:G:T, 2:169259056:G:T, 2:169259062:T:G, 2:169259064:A:C, 2:169259097:G:C, 2:169259105:C:T, 2:169259107:T:C, 2:169259107:T:G, 2:169259111:A:T, 2:169259115:A:G, 2:169259118:C:T, 2:169259127:T:C, 2:169259128:G:T, 2:169259142:T:C, 2:169259146:G:C, 2:169259146:G:T, 2:169259155:A:C, 2:169259160:C:G, 2:169259175:C:T, 2:169259175:C:G, 2:169259182:C:T, 2:169259184:T:C, 2:169259187:T:G, 2:169259188:C:A, 2:169259190:A:T, 2:169259193:C:G, 2:169259197:T:C, 2:169259203:C:A, 2:169270902:A:T, 2:169270902:A:C, 2:169270902:A:G, 2:169270907:T:C, 2:169270912:T:C, 2:169270917:C:A, 2:169270918:T:G, 2:169270940:T:C, 2:169270944:C:G, 2:169270946:T:C, 2:169270960:A:G, 2:169270961:T:C, 2:169270968:G:T, 2:169270973:G:A, 2:169270987:A:G, 2:169270993:A:G, 2:169270994:C:A, 2:169270995:A:T, 2:169270998:G:C, 2:169271000:A:G, 2:169271002:G:A, 2:169271006:G:A, 2:169271008:T:A, 2:169271008:T:C, 2:169271012:C:T, 2:169271012:C:G, 2:169271015:A:G, 2:169271018:C:G, 2:169271021:G:C, 2:169271027:T:C, 2:169271029:A:G, 2:169271030:C:T, 2:169271034:T:A, 2:169271044:G:T, 2:169271050:G:T, 2:169271057:G:A, 2:169271075:C:T, 2:169271077:T:C, 2:169271078:G:T, 2:169271086:A:C, 2:169271089:A:G, 2:169271101:T:C, 2:169272927:C:T, 2:169272935:T:C, 2:169272950:T:A, 2:169272957:G:T, 2:169272962:C:A, 2:169272962:C:T, 2:169272970:G:T, 2:169272971:C:T, 2:169272987:C:G, 2:169272997:A:T, 2:169273001:T:C, 2:169273004:G:A, 2:169273005:TTC:T, 2:169273007:C:T, 2:169273020:C:T, 2:169273020:C:A, 2:169273022:C:T, 2:169273028:T:G, 2:169273028:T:A, 2:169273029:G:C, 2:169273032:C:G, 2:169273037:C:A, 2:169273043:T:C, 2:169273046:T:C, 2:169273059:G:A, 2:169273060:A:T, 2:169273061:T:C, 2:169275042:G:A, 2:169275042:G:T, 2:169275044:T:A, 2:169275045:G:A, 2:169275046:T:A, 2:169275051:G:C, 2:169275066:T:C, 2:169275071:C:T, 2:169275079:C:A, 2:169275096:A:G, 2:169275096:A:C, 2:169275098:T:C, 2:169275101:A:C, 2:169275107:G:A, 2:169275108:G:T, 2:169275119:G:C, 2:169275128:T:C, 2:169275131:G:A, 2:169275140:A:G, 2:169275141:C:T, 2:169275143:G:A, 2:169275153:T:C, 2:169275154:C:T, 2:169275158:T:C, 2:169275177:C:T, 2:169275186:A:T, 2:169275189:T:C, 2:169275192:C:G, 2:169275194:C:T, 2:169275195:C:T, 2:169275197:A:C, 2:169275201:G:A, 2:169275204:G:A, 2:169275206:G:A, 2:169275215:G:A, 2:169275216:A:C, 2:169275222:C:T, 2:169275234:T:A, 2:169277749:G:C, 2:169277755:C:T, 2:169277760:T:A, 2:169277767:C:T, 2:169277776:T:C, 2:169277778:T:C, 2:169277787:C:T, 2:169277790:G:C, 2:169277791:A:T, 2:169277802:T:C, 2:169277810:C:A, 2:169277822:A:T, 2:169277829:G:A, 2:169277843:C:T, 2:169277847:C:T, 2:169277853:T:C, 2:169277857:T:C, 2:169277863:C:T, 2:169277874:C:T, 2:169277880:C:T, 2:169277888:C:T, 2:169277890:T:C, 2:169277917:C:A, 2:169277925:C:T, 2:169277925:C:G, 2:169277930:C:G, 2:169277934:T:C, 2:169277944:A:G, 2:169277949:T:C, 2:169279378:G:T, 2:169279381:G:A, 2:169279381:G:T, 2:169279385:C:T, 2:169279403:G:C, 2:169279408:C:T, 2:169279420:G:T, 2:169279422:T:C, 2:169279423:A:G, 2:169279429:G:T, 2:169279438:T:G, 2:169279454:T:G, 2:169279463:C:G, 2:169279464:T:C, 2:169279465:A:C, 2:169279471:T:C, 2:169279481:T:A, 2:169279490:G:C, 2:169279513:T:C, 2:169279514:C:CCA, 2:169279525:T:C, 2:169279528:T:C, 2:169279529:C:G, 2:169279529:C:T, 2:169279550:T:G, 2:169279553:G:C, 2:169279556:C:T, 2:169279561:T:C, 2:169279564:A:C, 2:169279568:T:G, 2:169279569:T:A, 2:169279573:C:G, 2:169279573:C:T, 2:169279578:A:C, 2:169279580:T:A, 2:169279580:T:C, 2:169279583:CA:C, 2:169279588:G:T, 2:169279592:A:C, 2:169279595:C:A, 2:169280351:T:C, 2:169280354:T:C, 2:169280360:A:T, 2:169280361:C:T, 2:169280361:C:A, 2:169280364:T:C, 2:169280370:T:C, 2:169280374:A:C, 2:169280378:A:G, 2:169280385:G:T, 2:169280393:T:C, 2:169280396:T:C, 2:169280414:G:A, 2:169280423:C:G, 2:169280424:G:A, 2:169280427:T:G, 2:169280439:C:T, 2:169280443:G:C, 2:169280447:C:T, 2:169280451:A:G, 2:169280487:G:C, 2:169280493:T:C, 2:169280505:T:A, 2:169280505:T:C, 2:169280508:A:C, 2:169280510:G:T, 2:169280510:G:C, 2:169280511:C:A, 2:169280517:C:A, 2:169282881:T:C, 2:169282894:A:G, 2:169282899:C:T, 2:169282902:C:T, 2:169282902:C:A, 2:169282904:C:A, 2:169282906:C:G, 2:169282918:C:A, 2:169282921:C:G, 2:169282922:T:G, 2:169282924:C:T, 2:169282942:G:T, 2:169282944:C:T, 2:169282951:G:A, 2:169282953:C:G, 2:169282954:T:G, 2:169282959:C:T, 2:169282961:C:A, 2:169282981:A:G, 2:169282984:T:C, 2:169282984:T:A, 2:169282994:A:T, 2:169282995:T:C, 2:169282996:CA:C, 2:169282998:A:G, 2:169289025:C:A, 2:169289031:C:G, 2:169289039:G:T, 2:169289043:T:C, 2:169289046:T:A, 2:169289050:G:C, 2:169289056:T:C, 2:169289068:G:A, 2:169289070:G:A, 2:169289085:C:T, 2:169289095:G:T, 2:169289099:C:G, 2:169289109:T:C, 2:169289119:A:T, 2:169289125:A:T, 2:169289127:G:A, 2:169289146:C:A, 2:169290861:A:T, 2:169290870:G:C, 2:169290870:G:GT, 2:169290874:T:G, 2:169290878:C:A, 2:169290878:C:G, 2:169290895:T:G, 2:169290898:A:G, 2:169290904:C:T, 2:169290916:T:G, 2:169290934:C:G, 2:169290934:C:T, 2:169290935:G:A, 2:169290937:C:A, 2:169290946:G:A, 2:169290964:G:C, 2:169290989:G:A, 2:169290994:C:A, 2:169292255:C:T, 2:169292255:C:A, 2:169292259:C:T, 2:169292262:C:T, 2:169292265:C:T, 2:169292266:A:C, 2:169292267:T:G, 2:169292276:T:C, 2:169292280:T:C, 2:169292282:C:T, 2:169292286:C:T, 2:169292295:C:T, 2:169292298:C:T, 2:169292304:C:A, 2:169292306:C:G, 2:169292310:T:G, 2:169292314:A:T, 2:169292319:T:G, 2:169292319:T:C, 2:169292320:G:T, 2:169292322:A:G, 2:169292327:C:G, 2:169292355:C:G, 2:169292360:G:A, 2:169292360:G:T, 2:169292369:T:C, 2:169294147:C:A, 2:169294148:T:C, 2:169294159:TC:T, 2:169294160:C:T, 2:169294162:T:G, 2:169294163:C:T, 2:169294170:G:T, 2:169294178:A:G, 2:169294181:C:T, 2:169294186:T:C, 2:169294198:A:G, 2:169294199:C:T, 2:169294210:G:A, 2:169294214:T:A, 2:169294217:A:G, 2:169294228:C:T, 2:169294244:T:C, 2:169294247:G:A, 2:169294247:G:T, 2:169294249:A:G, 2:169294255:ATC:A, 2:169294255:A:T, 2:169294597:T:TAA, 2:169294601:G:T, 2:169294605:T:A, 2:169294606:T:A, 2:169294615:C:T, 2:169294632:T:C, 2:169294639:T:C, 2:169294649:C:A, 2:169294672:C:T, 2:169294674:C:T, 2:169294677:T:C, 2:169294683:C:A, 2:169294689:A:C, 2:169294690:G:T, 2:169294692:T:A, 2:169294692:T:C, 2:169294693:G:T, 2:169294695:T:G, 2:169294698:C:T, 2:169294702:T:C, 2:169294704:G:T, 2:169294705:G:T, 2:169294706:G:T, 2:169294707:T:C, 2:169294708:A:T, 2:169294710:T:C, 2:169294712:T:A, 2:169307279:A:C, 2:169307279:AC:A, 2:169307285:G:C, 2:169307294:A:C, 2:169307294:A:T, 2:169307303:A:C, 2:169307307:G:A, 2:169307307:G:C, 2:169307317:T:C, 2:169307320:C:T, 2:169307321:G:C, 2:169307323:G:C, 2:169307326:C:A, 2:169307330:C:A, 2:169307337:T:C, 2:169307339:A:C, 2:169307340:C:T, 2:169307341:T:C, 2:169307356:C:A, 2:169307356:C:T, 2:169307357:A:T, 2:169307371:T:C, 2:169307372:C:G, 2:169307373:T:A, 2:169307373:T:C, 2:169307379:C:T, 2:169307382:G:C, 2:169307384:G:C, 2:169307388:G:A, 2:169307388:G:T, 2:169307389:T:C, 2:169307394:T:C, 2:169307395:G:T, 2:169307397:G:T, 2:169318760:A:AC, 2:169318761:C:G, 2:169318761:C:T, 2:169318762:A:G, 2:169318770:T:C, 2:169318804:C:A, 2:169318825:T:A, 2:169318828:G:T, 2:169318839:C:T, 2:169318843:C:T, 2:169318845:C:T, 2:169318854:T:C, 2:169318875:G:C, 2:169320778:G:T, 2:169320782:C:A, 2:169320783:C:T, 2:169320786:T:A, 2:169320794:G:C, 2:169320798:C:A, 2:169320808:G:C, 2:169320834:C:G, 2:169320853:A:C, 2:169320864:G:T, 2:169320865:A:C, 2:169320869:T:C, 2:169320876:T:C, 2:169320884:T:C, 2:169362323:T:C, 2:169362326:C:T, 2:169362335:G:A, 2:169362336:G:A, 2:169362345:A:C, 2:169362347:G:A, 2:169362348:C:A, 2:169362363:G:A, 2:169362372:A:G, 2:169362374:G:C, 2:169362375:C:A, 2:169362378:C:T, 2:169362380:G:T, 2:169362381:C:G, 2:169362387:G:T, 2:169362392:C:G, 2:169128674:A:C, 2:169128677:C:T, 2:169128686:C:T, 2:169128691:T:C, 2:169128692:T:A, 2:169128694:G:A, 2:169128697:G:A, 2:169128700:T:G, 2:169128709:G:C, 2:169128719:T:C, 2:169128727:T:C, 2:169128743:C:G, 2:169128751:G:T, 2:169128778:G:T, 2:169128785:A:C, 2:169128797:T:G, 2:169128802:G:A, 2:169128820:T:C, 2:169128821:C:T, 2:169128829:A:T, 2:169128829:A:G, 2:169129011:A:T, 2:169129012:C:T, 2:169129015:G:T, 2:169129022:G:C, 2:169129023:A:G, 2:169129026:G:A, 2:169129033:C:A, 2:169129056:T:A, 2:169129073:C:A, 2:169129080:G:A, 2:169129085:C:T, 2:169132578:G:T, 2:169132599:G:A, 2:169132602:G:C, 2:169132617:A:T, 2:169132618:C:A, 2:169132630:G:T, 2:169132655:AT:A, 2:169132657:T:A, 2:169132663:C:T, 2:169137399:G:A, 2:169137399:G:C, 2:169137403:G:A, 2:169137411:T:C, 2:169137424:C:A, 2:169137439:T:C, 2:169137442:G:A, 2:169137450:A:T, 2:169137453:A:T, 2:169137459:G:T, 2:169137463:G:C, 2:169137469:C:A, 2:169137469:C:T, 2:169137475:C:T, 2:169137476:C:T, 2:169137489:T:C, 2:169137493:T:A, 2:169138576:C:T, 2:169138582:C:G, 2:169138584:A:G, 2:169138624:C:T, 2:169138624:C:G, 2:169138630:T:C, 2:169138653:G:A, 2:169138660:A:C, 2:169138665:A:G, 2:169138666:C:A, 2:169138681:C:T, 2:169138688:C:G, 2:169138693:C:A, 2:169138696:G:C, 2:169138698:C:T, 2:169138701:C:G, 2:169138702:T:C, 2:169138705:A:T, 2:169139249:A:T, 2:169139251:C:T, 2:169139255:G:A, 2:169139260:T:C, 2:169139263:G:A, 2:169139269:G:A, 2:169139281:G:A, 2:169139285:C:T, 2:169139290:C:G, 2:169139299:T:C, 2:169139318:C:A, 2:169139326:C:G, 2:169139327:C:T, 2:169139329:A:G, 2:169139332:A:T, 2:169139371:G:C, 2:169139538:CTTACTTG:C, 2:169139543:T:C, 2:169139548:C:T, 2:169139573:C:T, 2:169139587:T:A, 2:169139591:C:T, 2:169139597:A:T, 2:169139609:A:G, 2:169140456:T:C, 2:169140461:T:G, 2:169140461:T:C, 2:169140468:G:C, 2:169140474:T:G, 2:169140475:C:A, 2:169140476:T:C, 2:169140479:T:A, 2:169140485:T:C, 2:169140489:A:T, 2:169140499:G:T, 2:169140503:A:T, 2:169140503:A:C, 2:169140513:A:AT, 2:169140513:A:T, 2:169140515:G:T, 2:169140516:G:T, 2:169140521:G:C, 2:169140521:G:T, 2:169140522:G:A, 2:169140525:G:C, 2:169140533:G:A, 2:169140539:T:C, 2:169140540:C:T, 2:169140545:G:C, 2:169142679:T:A, 2:169142683:A:C, 2:169142715:C:T, 2:169142727:G:T, 2:169142729:A:T, 2:169142735:G:T, 2:169142743:C:T, 2:169142757:CAG:C, 2:169142772:A:T, 2:169142778:T:C, 2:169142779:T:C, 2:169142781:C:T, 2:169142784:AG:A, 2:169142785:G:A, 2:169142786:G:T, 2:169142787:T:C, 2:169142790:G:A, 2:169142793:A:G, 2:169145755:T:A, 2:169145758:T:C, 2:169145759:A:G, 2:169145760:T:G, 2:169145779:C:T, 2:169145780:G:A, 2:169145786:G:C, 2:169145812:G:T, 2:169145829:T:A, 2:169145851:C:T, 2:169145855:C:T, 2:169145861:C:T, 2:169145863:T:G, 2:169145872:G:T, 2:169145876:T:C, 2:169145888:G:T, 2:169145890:T:C, 2:169145908:C:T, 2:169145914:G:C, 2:169145915:G:T, 2:169146737:A:C, 2:169146739:C:T, 2:169146741:T:A, 2:169146741:T:C, 2:169146742:C:T, 2:169146750:A:G, 2:169146774:T:C, 2:169146777:T:G, 2:169146790:T:C, 2:169146796:C:T, 2:169146808:C:T, 2:169146819:A:C, 2:169146819:A:G, 2:169146822:C:T, 2:169146823:G:A, 2:169146838:A:G, 2:169146843:A:G, 2:169146852:C:G, 2:169146855:G:A, 2:169146869:G:C, 2:169146871:C:T, 2:169146891:C:T, 2:169146891:C:A, 2:169146892:G:A, 2:169146892:G:T, 2:169146897:T:C, 2:169146905:C:T, 2:169146915:G:A, 2:169146919:C:G, 2:169146937:C:G, 2:169146938:C:G, 2:169146945:G:A, 2:169146952:A:T, 2:169150898:C:G, 2:169150920:T:C, 2:169150923:C:T, 2:169150925:G:A, 2:169150937:A:G, 2:169150941:C:T, 2:169150967:C:T, 2:169150976:A:G, 2:169150977:G:C, 2:169150982:G:A, 2:169150983:C:A, 2:169150986:C:A, 2:169150986:C:G, 2:169150988:T:G, 2:169152798:C:A, 2:169152800:T:C, 2:169152805:A:G, 2:169152835:A:C, 2:169152838:A:G, 2:169152839:C:A, 2:169152845:T:G, 2:169152855:G:T, 2:169152901:A:T, 2:169152902:T:C, 2:169152917:T:G, 2:169152923:C:A, 2:169152944:G:A, 2:169154466:C:T, 2:169154487:C:A, 2:169154501:T:C, 2:169154511:C:CAA, 2:169154511:C:G, 2:169154513:TC:T, 2:169154514:C:T, 2:169154516:T:A, 2:169154517:C:G, 2:169154529:C:G, 2:169154533:GA:G, 2:169154534:A:T, 2:169154536:C:A, 2:169154550:G:C, 2:169154562:G:A, 2:169154565:T:A, 2:169154568:G:A, 2:169154572:A:C, 2:169154592:A:C, 2:169154603:C:T, 2:169156277:C:G, 2:169156279:G:A, 2:169156288:C:T, 2:169156295:C:A, 2:169156301:G:A, 2:169156315:G:A, 2:169156325:C:G, 2:169156325:C:A, 2:169156331:A:T, 2:169156351:T:C, 2:169156354:G:T, 2:169156372:G:C, 2:169156384:A:T, 2:169156395:T:G, 2:169156396:T:A, 2:169156402:A:G, 2:169156405:T:C, 2:169157376:C:T, 2:169157379:G:C, 2:169157380:A:T, 2:169157385:C:A, 2:169157416:T:C, 2:169157424:C:G, 2:169157426:GATAA:G, 2:169157431:A:T, 2:169157433:C:T, 2:169157434:C:CGTGG, 2:169157437:C:T, 2:169157449:G:T, 2:169157461:G:C, 2:169157464:C:G, 2:169157464:C:T, 2:169157465:G:C, 2:169157482:A:G, 2:169157487:C:G, 2:169157491:C:A, 2:169157496:C:T, 2:169157502:T:C, 2:169162480:A:T, 2:169162487:C:T, 2:169162508:C:A, 2:169162516:T:A, 2:169162522:A:T, 2:169162532:G:A, 2:169162538:T:G, 2:169162550:T:A, 2:169162552:C:T, 2:169162552:C:A, 2:169162562:A:G, 2:169162570:T:A, 2:169162573:G:A, 2:169162588:G:T, 2:169162589:T:C, 2:169162589:T:A, 2:169162592:GT:G, 2:169162592:G:A, 2:169165938:C:T, 2:169165947:C:T, 2:169165952:G:T, 2:169165953:T:G, 2:169165956:C:T, 2:169165958:T:C, 2:169165961:C:T, 2:169165964:C:T, 2:169165965:A:C, 2:169165968:C:T, 2:169165977:C:A, 2:169165988:T:C, 2:169165990:A:C, 2:169165997:T:C, 2:169166001:T:G, 2:169166001:T:C, 2:169166003:CA:C, 2:169166006:C:A, 2:169166007:G:T, 2:169166010:T:C, 2:169166012:T:C, 2:169166024:A:T, 2:169166028:G:A, 2:169166028:G:T, 2:169166045:G:A, 2:169168546:G:C, 2:169168547:T:C, 2:169168572:C:T, 2:169168610:A:C, 2:169168611:T:C, 2:169168619:T:C, 2:169168621:G:T, 2:169168626:T:G, 2:169168629:A:T, 2:169168633:GA:G, 2:169168652:T:C, 2:169168662:C:G, 2:169168671:G:T, 2:169168673:G:A, 2:169168674:T:G, 2:169169701:C:T, 2:169169701:C:G, 2:169169726:CCAAA:C, 2:169169728:A:G, 2:169169732:A:G, 2:169169760:AC:A, 2:169169780:T:G, 2:169169782:G:A, 2:169169794:T:C, 2:169169801:G:A, 2:169169809:G:A, 2:169169812:C:T, 2:169170552:A:C, 2:169170553:C:T, 2:169170556:T:C, 2:169170559:C:A, 2:169170568:G:A, 2:169170574:T:C, 2:169170583:T:A, 2:169170584:C:T, 2:169170585:G:T, 2:169170617:T:G, 2:169170628:T:C, 2:169170646:C:T, 2:169172011:TCA:T, 2:169172035:T:A, 2:169172038:T:A, 2:169172045:A:C, 2:169172059:T:G, 2:169172060:C:T, 2:169172063:A:G, 2:169172069:A:G, 2:169172075:G:T, 2:169172075:G:C, 2:169172077:G:A, 2:169172087:G:A, 2:169172107:T:G, 2:169172107:T:A, 2:169172110:C:G, 2:169172114:C:T, 2:169172116:G:A, 2:169172121:G:T, 2:169172122:C:A, 2:169172134:T:C, 2:169173105:G:C, 2:169173114:T:G, 2:169173116:T:C, 2:169173120:C:T, 2:169173129:C:T, 2:169173131:T:C, 2:169173137:C:A, 2:169173137:C:T, 2:169173137:C:G, 2:169173138:C:T, 2:169173141:T:G, 2:169173143:C:T, 2:169173152:C:T, 2:169173167:C:A, 2:169173173:T:C, 2:169173177:T:C, 2:169173186:T:G, 2:169173194:G:A, 2:169173199:G:T, 2:169173200:T:C, 2:169173206:C:T, 2:169173210:G:C, 2:169173212:T:C, 2:169173215:G:A, 2:169173930:A:T, 2:169173940:C:A, 2:169173954:C:A, 2:169173958:C:T, 2:169173961:T:C, 2:169173964:C:A, 2:169173971:A:C, 2:169173972:C:T, 2:169173974:C:A, 2:169173975:T:G, 2:169173977:C:A, 2:169173978:C:T, 2:169173984:T:C, 2:169173985:G:T, 2:169173997:G:T, 2:169173997:G:A, 2:169174005:G:A, 2:169174013:A:T, 2:169174014:A:T, 2:169174020:C:A, 2:169174029:C:T, 2:169174037:G:T, 2:169174041:G:T, 2:169174045:A:G, 2:169174046:G:T, 2:169174047:T:C, 2:169174065:G:A, 2:169174076:AC:A, 2:169174081:C:G, 2:169174084:T:G, 2:169174087:A:T, 2:169174097:C:A, 2:169174099:G:C, 2:169174099:G:T, 2:169174104:G:A, 2:169174110:G:A, 2:169174112:G:C, 2:169174132:A:G, 2:169174141:C:T, 2:169174157:G:T, 2:169174158:T:G, 2:169174162:TC:T, 2:169175192:C:A, 2:169175201:A:C, 2:169175217:A:T, 2:169175219:C:A, 2:169175243:T:C, 2:169175247:A:T, 2:169175258:G:T, 2:169175274:C:T, 2:169175277:C:T, 2:169175283:A:T, 2:169175294:C:A, 2:169175294:C:T, 2:169175310:G:C, 2:169175310:G:T, 2:169175316:G:A, 2:169175322:G:A, 2:169175330:T:C, 2:169175331:C:G, 2:169175333:T:C, 2:169175342:T:A, 2:169175355:T:C, 2:169175366:C:T, 2:169175368:T:A, 2:169175374:C:A, 2:169175374:C:G, 2:169175379:T:C, 2:169175385:T:C, 2:169176411:T:C, 2:169176417:T:C, 2:169176424:C:A, 2:169176435:T:G, 2:169176438:G:A, 2:169176441:C:G, 2:169176444:G:T, 2:169176447:C:T, 2:169176449:C:A, 2:169176450:A:G, 2:169176451:T:C, 2:169176453:G:A, 2:169176454:G:T, 2:169176459:C:G, 2:169176465:G:A, 2:169176477:A:G, 2:169176497:A:C, 2:169176502:G:A, 2:169176511:A:T, 2:169176514:T:C, 2:169176516:A:C, 2:169176533:C:G, 2:169176538:T:G, 2:169176544:A:G, 2:169176550:G:A, 2:169176552:G:C, 2:169176564:T:C, 2:169176573:C:T, 2:169176577:A:G, 2:169176582:T:C, 2:169176588:A:G, 2:169177802:C:A, 2:169177808:G:C, 2:169177811:TG:T, 2:169177814:C:T, 2:169177826:T:C, 2:169177827:A:G, 2:169177832:T:A, 2:169177841:A:T, 2:169177844:G:T, 2:169177846:T:G, 2:169177866:GTGTC:G, 2:169177887:C:T, 2:169177889:T:C, 2:169177890:ATT:A, 2:169177890:A:G, 2:169177901:T:G, 2:169177907:A:G, 2:169177922:C:T, 2:169177934:T:A, 2:169177934:T:TA, 2:169177940:G:A, 2:169177953:T:C, 2:169177962:C:A, 2:169177985:T:C, 2:169177986:C:CG, 2:169177986:C:T, 2:169177988:T:A, 2:169177998:G:A, 2:169178000:C:T, 2:169178001:G:A, 2:169178019:C:T, 2:169181448:T:C, 2:169181451:A:T, 2:169181455:A:G, 2:169181457:C:T, 2:169181462:G:C, 2:169181492:GGT:G, 2:169181505:G:T, 2:169181509:T:A, 2:169181511:C:T, 2:169181514:T:C, 2:169181523:T:C, 2:169181533:T:C, 2:169181541:A:G, 2:169181552:G:C, 2:169181566:T:C, 2:169181569:C:T, 2:169181569:C:A, 2:169181572:C:G, 2:169181574:C:T, 2:169181577:C:G, 2:169181581:T:G, 2:169181596:C:T, 2:169181604:G:T, 2:169181606:C:G, 2:169181615:G:T, 2:169181616:T:C, 2:169182177:G:A, 2:169182180:G:A, 2:169182183:G:C, 2:169182198:G:T, 2:169182198:G:A, 2:169182204:C:G, 2:169182218:T:G, 2:169182227:T:C, 2:169182231:C:T, 2:169182233:C:G, 2:169182233:C:T, 2:169182282:A:T, 2:169182285:G:A, 2:169182290:T:C, 2:169182291:C:A, 2:169182293:A:C, 2:169182299:G:A, 2:169185509:A:G, 2:169185510:C:T, 2:169185519:C:T, 2:169185522:C:A, 2:169185524:A:C, 2:169185531:C:T, 2:169185543:G:T, 2:169185555:T:C, 2:169185558:T:C, 2:169185560:G:T, 2:169185574:C:A, 2:169185578:T:C, 2:169185586:C:T, 2:169185586:C:G, 2:169185588:T:G, 2:169185605:C:T, 2:169185611:G:T, 2:169185614:T:C, 2:169185615:C:T, 2:169185618:TC:T, 2:169185623:T:C, 2:169185629:C:T, 2:169185630:T:C, 2:169185639:C:T, 2:169185641:C:A, 2:169185642:G:C, 2:169185647:A:G, 2:169185649:A:C, 2:169185653:A:G, 2:169185654:A:C, 2:169185657:C:A, 2:169185704:T:C, 2:169185732:G:A, 2:169185737:C:T, 2:169185744:T:C, 2:169185753:G:T, 2:169185758:A:G, 2:169185761:T:A, 2:169185793:T:A, 2:169185797:C:G, 2:169185808:TG:T, 2:169185812:G:T, 2:169185819:T:G, 2:169185825:T:A, 2:169185827:T:C, 2:169185830:G:C, 2:169185830:G:A, 2:169185831:A:G, 2:169185833:C:T, 2:169185840:C:T, 2:169185841:A:C, 2:169185846:C:T, 2:169185850:C:G, 2:169185851:T:A, 2:169185852:T:C, 2:169185864:C:G, 2:169185864:C:T, 2:169185870:G:A, 2:169185872:A:T, 2:169185882:A:G, 2:169185888:C:CAATA, 2:169185893:T:C, 2:169185897:C:T, 2:169185900:A:C, 2:169185910:G:C, 2:169185914:G:A, 2:169185929:C:A, 2:169185932:G:T, 2:169185936:G:A, 2:169185958:T:G, 2:169185964:G:T, 2:169185966:C:T, 2:169185968:G:C, 2:169185974:T:A, 2:169185976:G:C, 2:169185980:T:C, 2:169185992:A:G, 2:169185993:T:C, 2:169185995:G:A, 2:169186014:T:C, 2:169187969:C:T, 2:169187972:C:T, 2:169187984:T:G, 2:169187985:C:T, 2:169187986:G:C, 2:169187991:T:C, 2:169188006:C:T, 2:169188013:G:C, 2:169188032:T:C, 2:169188033:C:A, 2:169188034:G:C, 2:169188051:C:T, 2:169188055:C:G, 2:169188060:A:G, 2:169188063:C:T, 2:169188066:G:C, 2:169188072:G:A, 2:169188077:G:T, 2:169188077:G:C, 2:169188080:G:A, 2:169188087:G:A, 2:169188099:G:T, 2:169188100:G:T, 2:169188101:T:G, 2:169188102:G:A, 2:169188108:G:C, 2:169188114:C:T, 2:169188133:A:T, 2:169188137:T:A, 2:169188150:C:A, 2:169188151:G:T, 2:169188173:C:T, 2:169188179:T:C, 2:169188185:G:T, 2:169188191:T:C, 2:169188192:G:T, 2:169188203:C:T, 2:169188204:A:C, 2:169188206:G:C, 2:169188218:C:G, 2:169188219:A:C, 2:169188222:C:G, 2:169188223:C:A, 2:169188224:C:T, 2:169188229:G:C, 2:169188236:T:G, 2:169188236:T:C, 2:169188239:T:G, 2:169188245:C:G, 2:169188245:C:T, 2:169191848:T:C, 2:169191848:T:G, 2:169191859:T:C, 2:169191860:A:C, 2:169191865:C:T, 2:169191866:A:G, 2:169191869:T:C, 2:169191872:A:G, 2:169191873:T:A, 2:169191876:A:T, 2:169191883:G:A, 2:169191893:T:C, 2:169191896:T:C, 2:169191901:C:A, 2:169191906:C:A, 2:169191911:T:C, 2:169191916:T:A, 2:169191917:C:A, 2:169191919:T:C, 2:169191935:C:G, 2:169191938:C:T, 2:169191959:A:G, 2:169191960:C:G, 2:169191968:T:C, 2:169191985:G:A, 2:169191986:G:A, 2:169192008:C:G, 2:169192022:A:G, 2:169192023:G:C, 2:169193778:T:C, 2:169193791:T:A, 2:169193808:T:A, 2:169193817:C:T, 2:169193822:C:T, 2:169193834:G:C, 2:169193854:A:G, 2:169193855:C:G, 2:169193881:G:A, 2:169196916:G:C, 2:169196919:G:C, 2:169196920:C:T, 2:169196934:G:T, 2:169196936:A:C, 2:169196938:C:A, 2:169196953:C:G, 2:169196958:T:C, 2:169196961:C:T, 2:169196969:A:T, 2:169197015:C:T, 2:169198794:G:A, 2:169198797:G:C, 2:169198799:G:T, 2:169198804:C:A, 2:169198810:T:C, 2:169198813:T:G, 2:169198816:C:G, 2:169198816:C:T, 2:169198818:C:G, 2:169198825:C:T, 2:169198828:T:C, 2:169198830:T:A, 2:169198833:C:T, 2:169198834:C:T, 2:169198846:A:G, 2:169198846:A:T, 2:169198851:C:A, 2:169198856:A:C, 2:169198857:A:G, 2:169198860:C:T, 2:169198862:A:C, 2:169198865:A:T, 2:169198873:T:A, 2:169198878:C:A, 2:169198905:C:A, 2:169198912:C:A, 2:169201641:A:C, 2:169201654:T:G, 2:169201654:T:C, 2:169201669:T:A, 2:169201675:C:G, 2:169201676:C:T, 2:169201679:T:C, 2:169201693:C:T, 2:169201696:G:A, 2:169201705:C:T, 2:169201714:T:C, 2:169201729:G:A, 2:169201739:T:G, 2:169201761:T:TG, 2:169201766:C:T, 2:169201774:C:CATTGTAGT, 2:169201775:C:A, 2:169201779:AC:A, 2:169201794:G:C, 2:169201795:T:C, 2:169201804:C:T, 2:169201810:G:C, 2:169201813:T:A, 2:169201822:C:T, 2:169201826:G:A, 2:169201828:C:A, 2:169201832:T:C, 2:169201844:A:T, 2:169201850:T:C, 2:169201861:G:C, 2:169202755:C:T, 2:169202767:T:A, 2:169202776:T:C, 2:169202796:G:T, 2:169202807:C:A, 2:169202812:T:A, 2:169202816:A:G, 2:169202824:G:C, 2:169202826:G:C, 2:169202827:A:C, 2:169202869:C:A, 2:169202870:G:A, 2:169202888:T:C, 2:169202889:G:T, 2:169202890:C:T, 2:169202895:C:G, 2:169202905:T:C, 2:169202908:G:A, 2:169202909:C:T, 2:169202914:T:C, 2:169202918:A:G, 2:169202956:G:A, 2:169203984:G:A, 2:169203994:T:C, 2:169204011:T:A, 2:169204011:T:C, 2:169204017:T:C, 2:169204022:A:C, 2:169204033:T:A, 2:169204038:T:C, 2:169204039:G:C, 2:169204052:C:A, 2:169204093:T:A, 2:169204105:C:T, 2:169204110:T:C, 2:169204116:G:C, 2:169204119:C:T, 2:169204120:C:T, 2:169204123:C:T, 2:169204123:CAT:C, 2:169204128:T:A, 2:169204138:G:A, 2:169204146:C:A, 2:169204158:A:G, 2:169204162:C:T, 2:169204176:T:C, 2:169204182:C:T, 2:169204183:C:T, 2:169204191:G:C, 2:169204192:T:A, 2:169204207:G:C, 2:169204207:G:GGA, 2:169204215:G:A, 2:169204224:A:G, 2:169204242:C:A, 2:169204257:C:T, 2:169204261:C:G, 2:169205486:C:T, 2:169205492:C:G, 2:169205510:C:T, 2:169205515:T:C, 2:169205518:T:C, 2:169205522:G:T, 2:169205534:TG:T, 2:169205593:C:T, 2:169205597:C:T, 2:169205600:T:C, 2:169205635:T:G, 2:169205638:C:T, 2:169206022:C:T, 2:169206032:G:A, 2:169206035:T:A, 2:169206065:C:CG, 2:169206066:G:T, 2:169206072:T:C, 2:169206078:T:G, 2:169206080:C:A, 2:169206086:G:C, 2:169206089:C:A, 2:169206092:T:A, 2:169206102:T:C, 2:169206114:T:G, 2:169206135:A:G, 2:169206143:C:A, 2:169206147:T:C, 2:169206154:C:A, 2:169206156:A:T, 2:169206164:G:A, 2:169206171:C:A, 2:169206174:C:T, 2:169206333:A:C, 2:169206372:T:C, 2:169206389:C:T, 2:169206398:G:A, 2:169206410:T:A, 2:169206414:T:A, 2:169206420:A:G, 2:169206441:C:T, 2:169206443:T:G, 2:169206446:T:C, 2:169206450:G:T, 2:169206464:C:G, 2:169206479:G:A, 2:169206485:A:C, 2:169206486:A:C, 2:169206488:G:A, 2:169206506:G:C, 2:169206518:A:G, 2:169206522:T:C, 2:169206552:G:A, 2:169206563:G:A, 2:169206575:C:G, 2:169206587:T:C, 2:169206588:C:T, 2:169206590:C:A, 2:169206591:T:A, 2:169206592:T:G, 2:169206596:AG:A, 2:169206597:G:T, 2:169206602:C:T, 2:169206605:A:C, 2:169206608:G:A, 2:169206617:C:T, 2:169206619:T:G, 2:169206624:G:A, 2:169206651:A:G, 2:169206653:A:G, 2:169206657:G:A, 2:169206671:T:C, 2:169206686:G:A, 2:169206690:T:C, 2:169206692:T:C, 2:169206698:A:G, 2:169206713:C:G, 2:169206716:G:T, 2:169206730:G:T, 2:169206734:A:C, 2:169206738:T:C, 2:169206740:G:A, 2:169206743:A:C, 2:169206744:C:G, 2:169206754:C:A, 2:169206757:G:T, 2:169206766:G:C, 2:169206774:T:C, 2:169206774:T:A, 2:169206777:C:T, 2:169206786:G:A, 2:169206789:C:T, 2:169206793:G:T, 2:169206807:T:C, 2:169206809:C:A, 2:169206812:G:C, 2:169206813:C:T, 2:169206821:A:T, 2:169206823:C:A, 2:169206824:T:G, 2:169206828:T:C, 2:169206844:C:T, 2:169206878:T:C, 2:169206882:G:A, 2:169206888:G:A, 2:169206893:C:A, 2:169206894:G:A, 2:169206896:C:T, 2:169206905:C:A, 2:169206906:G:T, 2:169206908:A:C, 2:169206909:T:A, 2:169206913:T:A, 2:169206954:C:T, 2:169206960:A:G, 2:169206961:A:C, 2:169206970:C:A, 2:169206972:A:G, 2:169206980:T:C, 2:169206980:T:A, 2:169206983:C:T, 2:169206983:C:A, 2:169207011:G:C, 2:169207011:G:A, 2:169207016:G:A, 2:169207025:C:T, 2:169207032:A:C, 2:169207043:G:A, 2:169207046:C:A, 2:169207047:G:A, 2:169207064:A:C, 2:169207094:T:C, 2:169207094:T:G, 2:169207110:G:A, 2:169207121:T:C, 2:169207125:G:A, 2:169207145:G:A, 2:169207147:C:T, 2:169207163:T:A, 2:169207164:T:G, 2:169207176:G:T, 2:169207181:T:C, 2:169207185:T:C, 2:169207188:T:G, 2:169207203:C:T, 2:169207208:A:G, 2:169207218:C:T, 2:169207220:G:A, 2:169207227:A:G, 2:169207232:T:C, 2:169207246:A:T, 2:169209451:A:G, 2:169209456:C:A, 2:169209458:A:G, 2:169209459:C:T, 2:169209462:A:C, 2:169209472:A:C, 2:169209473:A:C, 2:169209473:A:G, 2:169209476:C:T, 2:169209477:C:A, 2:169209479:C:T, 2:169209480:G:A, 2:169209485:C:G, 2:169209494:C:T, 2:169209497:A:G, 2:169209501:C:T, 2:169209515:T:C, 2:169209516:T:A, 2:169209527:G:T, 2:169209532:ATCTG:A, 2:169209533:T:C, 2:169209537:G:T, 2:169209545:C:G, 2:169209546:T:C, 2:169209549:G:C, 2:169209554:G:A, 2:169209554:G:C, 2:169209555:C:T, 2:169209563:G:T, 2:169209578:C:T, 2:169209591:A:G, 2:169209602:C:G, 2:169209605:G:A, 2:169209606:A:T, 2:169209614:T:C, 2:169209615:C:A, 2:169209620:T:A, 2:169209620:T:C, 2:169209623:A:G, 2:169209626:T:G, 2:169209629:A:G, 2:169209632:G:A, 2:169209633:C:G, 2:169209633:C:T, 2:169209635:T:C, 2:169211966:A:G, 2:169211968:C:A, 2:169211970:T:C, 2:169211974:C:T, 2:169211977:C:T, 2:169211979:A:G, 2:169211982:G:A, 2:169211988:A:G, 2:169211991:G:T, 2:169212018:C:A, 2:169212022:A:G, 2:169212025:C:T, 2:169212043:T:C, 2:169212045:G:A, 2:169212057:A:T, 2:169212079:A:T, 2:169212081:C:T, 2:169212085:T:C, 2:169212094:T:C, 2:169212096:A:C, 2:169212097:G:C, 2:169212100:T:G, 2:169212114:C:G, 2:169212115:A:T, 2:169212118:C:A, 2:169212118:C:G, 2:169212132:C:A, 2:169212132:C:CCAAAAGCATT, 2:169212133:C:T, 2:169212136:C:T, 2:169212165:G:C, 2:169212169:T:G, 2:169212178:T:A, 2:169212180:C:G, 2:169212183:C:G, 2:169212184:A:C, 2:169212186:C:T, 2:169212202:C:A, 2:169213657:T:C, 2:169213669:A:G, 2:169213680:C:G, 2:169213692:G:C, 2:169213711:C:G, 2:169213718:G:C, 2:169213722:G:A, 2:169213725:C:T, 2:169213729:T:C, 2:169213735:T:C, 2:169213735:T:A, 2:169213737:T:A, 2:169213749:A:G, 2:169213750:T:C, 2:169213753:C:T, 2:169213768:C:T, 2:169213781:G:T, 2:169213783:A:G, 2:169213785:G:A, 2:169213791:T:C, 2:169213798:C:A, 2:169213813:G:A, 2:169213833:A:T, 2:169213840:G:A, 2:169213851:T:A, 2:169213851:T:C, 2:169213863:C:T, 2:169213870:T:C, 2:169216252:C:G, 2:169216267:T:C, 2:169216273:C:T, 2:169216291:C:T, 2:169216296:A:T, 2:169216305:G:A, 2:169216311:C:T, 2:169216320:T:A, 2:169216324:C:T, 2:169216327:G:A, 2:169216328:G:C, 2:169216329:T:C, 2:169216335:G:A, 2:169216356:G:T, 2:169216360:CA:C, 2:169216365:A:C, 2:169216371:G:C, 2:169216375:T:C, 2:169216390:G:A, 2:169216395:C:A, 2:169216411:G:A, 2:169216428:T:G, 2:169220455:C:G, 2:169220455:C:T, 2:169220457:C:G, 2:169220461:C:T, 2:169220467:C:T, 2:169220470:C:T, 2:169220474:T:C, 2:169220478:C:A, 2:169220490:C:G, 2:169220491:C:T, 2:169220505:G:T, 2:169220508:C:T, 2:169220512:C:G, 2:169220514:T:C, 2:169220522:C:G, 2:169220547:C:T, 2:169220548:C:T, 2:169220551:G:A, 2:169220557:T:A, 2:169220558:C:G, 2:169225312:C:T, 2:169225315:T:C, 2:169225324:T:C, 2:169225332:T:C, 2:169225338:G:C, 2:169225356:A:C, 2:169225357:T:A, 2:169225365:A:G, 2:169225368:G:A, 2:169225380:G:A, 2:169225405:C:T, 2:169225405:C:A, 2:169225413:G:A, 2:169225413:G:T, 2:169225420:T:A, 2:169225428:T:A, 2:169225440:C:T, 2:169225450:C:T, 2:169226424:G:A, 2:169226424:G:T, 2:169226424:G:C, 2:169226434:C:T, 2:169226436:A:G, 2:169226437:AT:A, 2:169226441:A:G, 2:169226446:T:G, 2:169226447:T:C, 2:169226456:T:G, 2:169226472:C:T, 2:169226495:A:C, 2:169226498:G:A, 2:169226499:G:A, 2:169226503:C:T, 2:169226505:T:C, 2:169226507:G:A, 2:169226510:T:A, 2:169226510:T:C, 2:169226520:T:G, 2:169226549:A:G, 2:169226552:A:G, 2:169226563:TAC:T, 2:169226571:T:C, 2:169226579:G:A, 2:169226580:G:C, 2:169226588:T:C, 2:169231715:T:A, 2:169231716:C:T, 2:169231728:A:G, 2:169231734:T:C, 2:169231735:C:A, 2:169231743:A:G, 2:169231804:A:C, 2:169231811:G:C, 2:169233416:G:T, 2:169233431:T:A, 2:169233435:C:A, 2:169233438:C:T, 2:169233455:C:A, 2:169233464:T:C, 2:169233470:A:G, 2:169233483:G:C, 2:169233483:G:T, 2:169233493:A:T, 2:169233496:C:A, 2:169233499:C:G, 2:169233510:G:C, 2:169233516:C:T, 2:169233518:C:G, 2:169233524:G:A, 2:169233525:T:C, 2:169233530:C:G, 2:169233537:T:C, 2:169233542:T:G, 2:169233545:A:C, 2:169233557:A:C, 2:169233569:G:A, 2:169233570:C:T, 2:169233577:G:T, 2:169233578:T:C, 2:169233581:C:A, 2:169235839:C:A, 2:169235848:T:G, 2:169235848:T:C, 2:169235850:G:T, 2:169235851:C:T, 2:169235867:A:C, 2:169235868:T:A, 2:169235868:T:C, 2:169235877:T:C, 2:169235884:C:T, 2:169235896:T:C, 2:169235898:T:A, 2:169235913:T:C, 2:169235918:G:C, 2:169235929:G:T, 2:169235934:T:C, 2:169235937:G:A, 2:169235942:G:C, 2:169235949:G:A, 2:169235972:C:A, 2:169235974:T:C, 2:169235982:A:C, 2:169235986:T:TG, 2:169235991:G:A, 2:169235992:T:C, 2:169235995:G:A, 2:169236004:C:T, 2:169236010:T:C, 2:169236012:C:T, 2:169236018:C:T, 2:169236019:G:A, 2:169236023:G:C, 2:169236025:T:C, 2:169236026:G:GC, 2:169236027:C:T, 2:169236031:G:A, 2:169236033:T:G, 2:169236037:G:A, 2:169236040:C:T, 2:169236042:C:T, 2:169236046:C:T, 2:169236049:A:T, 2:169237112:G:C, 2:169237112:G:A, 2:169237113:G:A, 2:169237122:C:T, 2:169237144:G:GT, 2:169237171:G:T, 2:169237178:T:C, 2:169237185:T:G, 2:169237187:G:C, 2:169237199:G:T, 2:169237202:T:C, 2:169237205:A:C, 2:169237208:G:A, 2:169237210:A:C, 2:169237222:G:T, 2:169237229:T:C, 2:169237233:G:C, 2:169237238:A:G, 2:169237251:C:T, 2:169237252:A:C, 2:169237276:ACTGTCAAATAC:A, 2:169237279:G:C, 2:169237283:A:C, 2:169237284:A:G, 2:169238097:T:A, 2:169238098:C:T, 2:169238101:T:A, 2:169238105:T:A, 2:169238124:C:G, 2:169238128:G:T, 2:169238138:G:C, 2:169238159:T:C, 2:169238183:C:G, 2:169238189:C:A, 2:169238218:G:A, 2:169238228:T:C, 2:169238236:T:G, 2:169238248:C:A, 2:169238249:T:G, 2:169238252:C:A, 2:169238258:T:C, 2:169238265:G:T, 2:169238266:T:A, 2:169238267:T:C, 2:169238269:T:G, 2:169238271:A:T, 2:169238273:T:C, 2:169238276:C:T, 2:169238281:A:G, 2:169238290:A:G, 2:169238300:A:T, 2:169239525:A:G, 2:169239529:G:A, 2:169239541:G:C, 2:169239545:T:C, 2:169239551:C:A, 2:169239559:A:G, 2:169239564:G:C, 2:169239569:C:T, 2:169239589:A:G, 2:169239592:G:A, 2:169239592:G:T, 2:169239613:T:C, 2:169239614:G:A, 2:169239625:G:C, 2:169239633:A:C, 2:169239643:T:C, 2:169239645:A:T, 2:169239646:T:C, 2:169239649:A:G, 2:169239661:G:C, 2:169239661:G:T, 2:169239680:G:A, 2:169239681:TA:T, 2:169239682:A:C, 2:169239718:G:C, 2:169239748:C:T, 2:169239750:A:T, 2:169239752:T:C, 2:169239757:T:C, 2:169239758:C:A, 2:169239774:A:C, 2:169240946:A:G, 2:169240956:T:C, 2:169240969:C:T, 2:169240970:T:C, 2:169240973:G:A, 2:169240976:C:A, 2:169240985:T:C, 2:169240991:A:G, 2:169240993:A:C, 2:169241006:C:A, 2:169241009:T:C, 2:169241018:G:A, 2:169241032:C:T, 2:169241032:C:G, 2:169241033:C:T, 2:169241036:C:G, 2:169241038:C:T, 2:169241047:C:T, 2:169241057:C:T, 2:169241062:A:G, 2:169241063:T:C, 2:169241072:C:T, 2:169241072:C:G, 2:169241089:C:A, 2:169241093:T:C, 2:169241096:G:C, 2:169241096:G:A, 2:169241097:A:C, 2:169241107:G:C, 2:169241114:T:C, 2:169241117:G:T, 2:169241126:T:A, 2:169241132:C:A, 2:169241134:C:T, 2:169241137:A:G, 2:169241145:G:T, 2:169241159:G:C, 2:169241195:T:C, 2:169241201:A:G, 2:169241215:G:A, 2:169241221:C:A, 2:169241224:GT:G, 2:169241236:C:G, 2:169241239:G:GCATTGTGC, 2:169241240:C:T, 2:169241257:C:A, 2:169241266:C:A, 2:169241269:T:C, 2:169241276:G:A, 2:169241278:C:T, 2:169241311:C:T, 2:169241331:T:G, 2:169241334:A:T, 2:169241341:T:A, 2:169241348:T:C, 2:169241354:G:T, 2:169241354:G:A, 2:169241357:G:T, 2:169241363:T:C, 2:169242954:A:T, 2:169242962:C:T, 2:169242967:T:C, 2:169242969:A:T, 2:169242970:T:C, 2:169242979:T:C, 2:169242997:C:T, 2:169243000:T:A, 2:169243007:G:A, 2:169243020:A:C, 2:169243033:C:G, 2:169243039:G:A, 2:169243043:A:C, 2:169243045:T:C, 2:169243063:C:T, 2:169243413:C:A, 2:169243416:A:C, 2:169243444:C:T, 2:169243460:AT:A, 2:169243463:G:C, 2:169243469:T:A, 2:169243469:T:C, 2:169243471:C:T, 2:169243473:TC:T, 2:169243474:C:G, 2:169243475:G:A, 2:169243495:T:A, 2:169243502:G:A, 2:169244692:C:T, 2:169244701:T:C, 2:169244732:C:T, 2:169244734:G:A, 2:169244744:C:G, 2:169244761:C:T, 2:169244762:A:G, 2:169244780:T:C, 2:169244788:G:A, 2:169244806:G:T, 2:169244807:G:T, 2:169244807:G:A, 2:169244815:G:A, 2:169244816:T:C, 2:169244839:C:A, 2:169244839:C:T, 2:169244840:C:T, 2:169244843:C:G, 2:169244864:C:T, 2:169244870:G:A, 2:169244878:G:T, 2:169244881:G:C, 2:169244894:C:T, 2:169244905:G:A, 2:169244912:C:T, 2:169244920:G:A, 2:169244920:GA:G, 2:169244926:G:T, 2:169244926:G:C, 2:169244930:T:C, 2:169244930:T:G, 2:169244932:T:G, 2:169246713:C:T, 2:169246714:C:T, 2:169246737:T:C, 2:169246740:T:C, 2:169246758:T:C, 2:169246765:G:C, 2:169246771:A:C, 2:169246780:C:T, 2:169246788:C:A, 2:169246818:C:A, 2:169246830:G:A, 2:169246830:G:C, 2:169246833:G:A, 2:169246848:TC:T, 2:169246849:C:G, 2:169246852:C:G, 2:169246864:AG:A, 2:169246887:C:G, 2:169246889:A:C, 2:169246891:A:C, 2:169246894:G:A, 2:169246896:C:T, 2:169246908:C:T, 2:169246909:G:A, 2:169246912:G:A, 2:169246921:G:A, 2:169246938:T:C, 2:169246939:G:A, 2:169246950:C:T, 2:169246951:C:G, 2:169246959:T:C, 2:169246979:G:T, 2:169246981:T:C, 2:169246986:C:T, 2:169246986:C:A, 2:169247378:C:G, 2:169247398:T:C, 2:169247404:T:C, 2:169247410:T:C, 2:169247413:A:G, 2:169247416:A:G, 2:169247417:T:G, 2:169247428:C:T, 2:169247431:C:T, 2:169247432:T:A, 2:169247434:C:T, 2:169247436:G:C, 2:169247443:G:A, 2:169247444:T:C, 2:169247452:C:T, 2:169247456:C:T, 2:169247474:G:A, 2:169247485:C:G, 2:169247496:G:C, 2:169247498:C:T, 2:169247498:C:A, 2:169247501:T:TA, 2:169247505:A:C, 2:169247507:A:C, 2:169247508:T:A, 2:169256106:C:G, 2:169256108:C:T, 2:169256109:C:T, 2:169256115:T:C, 2:169256121:G:A, 2:169256127:A:G, 2:169256131:A:T, 2:169256138:A:T, 2:169256141:T:C, 2:169256164:T:A, 2:169256165:C:T, 2:169256177:T:C, 2:169256178:C:A, 2:169256186:C:T, 2:169256196:T:C, 2:169256200:A:T, 2:169256206:A:T, 2:169256208:A:G, 2:169256213:T:A, 2:169256217:C:A, 2:169256217:C:T, 2:169256218:C:A, 2:169256220:A:G, 2:169256229:G:A, 2:169256234:G:T, 2:169257127:C:A, 2:169257128:A:C, 2:169257149:G:T, 2:169257154:C:G, 2:169257158:G:A, 2:169257167:T:G, 2:169257175:G:A, 2:169257177:C:A, 2:169257192:G:C, 2:169257200:A:G, 2:169257203:C:T, 2:169257209:T:C, 2:169257220:G:A, 2:169257223:C:G, 2:169257228:C:G, 2:169257232:T:C, 2:169257242:A:G, 2:169257251:T:A, 2:169259024:C:A, 2:169259025:C:A, 2:169259034:G:C, 2:169259049:G:T, 2:169259056:G:T, 2:169259062:T:G, 2:169259064:A:C, 2:169259097:G:C, 2:169259105:C:T, 2:169259107:T:C, 2:169259107:T:G, 2:169259111:A:T, 2:169259115:A:G, 2:169259118:C:T, 2:169259142:T:C, 2:169259146:G:C, 2:169259146:G:T, 2:169259155:A:C, 2:169259160:C:G, 2:169259175:C:T, 2:169259175:C:G, 2:169259182:C:T, 2:169259187:T:G, 2:169259188:C:A, 2:169259190:A:T, 2:169259193:C:G, 2:169259197:T:C, 2:169259203:C:A, 2:169270902:A:G, 2:169270902:A:T, 2:169270902:A:C, 2:169270907:T:C, 2:169270912:T:C, 2:169270917:C:A, 2:169270918:T:G, 2:169270940:T:C, 2:169270946:T:C, 2:169270960:A:G, 2:169270961:T:C, 2:169270973:G:A, 2:169270987:A:G, 2:169270993:A:G, 2:169270994:C:A, 2:169270995:A:T, 2:169270998:G:C, 2:169271000:A:G, 2:169271002:G:A, 2:169271006:G:A, 2:169271012:C:T, 2:169271012:C:G, 2:169271015:A:G, 2:169271018:C:G, 2:169271021:G:C, 2:169271027:T:C, 2:169271029:A:G, 2:169271030:C:T, 2:169271034:T:A, 2:169271044:G:T, 2:169271057:G:A, 2:169271077:T:C, 2:169271086:A:C, 2:169271089:A:G, 2:169272927:C:T, 2:169272950:T:A, 2:169272962:C:A, 2:169272962:C:T, 2:169272970:G:T, 2:169272971:C:T, 2:169272987:C:G, 2:169272997:A:T, 2:169273001:T:C, 2:169273004:G:A, 2:169273005:TTC:T, 2:169273007:C:T, 2:169273020:C:T, 2:169273020:C:A, 2:169273022:C:T, 2:169273028:T:G, 2:169273028:T:A, 2:169273029:G:C, 2:169273032:C:G, 2:169273037:C:A, 2:169273043:T:C, 2:169273046:T:C, 2:169273059:G:A, 2:169273060:A:T, 2:169273061:T:C, 2:169275042:G:T, 2:169275042:G:A, 2:169275044:T:A, 2:169275045:G:A, 2:169275046:T:A, 2:169275051:G:C, 2:169275066:T:C, 2:169275071:C:T, 2:169275079:C:A, 2:169275096:A:G, 2:169275096:A:C, 2:169275098:T:C, 2:169275101:A:C, 2:169275107:G:A, 2:169275108:G:T, 2:169275119:G:C, 2:169275128:T:C, 2:169275131:G:A, 2:169275140:A:G, 2:169275141:C:T, 2:169275143:G:A, 2:169275153:T:C, 2:169275154:C:T, 2:169275158:T:C, 2:169275186:A:T, 2:169275189:T:C, 2:169275194:C:T, 2:169275195:C:T, 2:169275197:A:C, 2:169275201:G:A, 2:169275204:G:A, 2:169275206:G:A, 2:169275215:G:A, 2:169275216:A:C, 2:169275222:C:T, 2:169275234:T:A, 2:169277749:G:C, 2:169277755:C:T, 2:169277760:T:A, 2:169277767:C:T, 2:169277776:T:C, 2:169277778:T:C, 2:169277787:C:T, 2:169277790:G:C, 2:169277791:A:T, 2:169277802:T:C, 2:169277810:C:A, 2:169277822:A:T, 2:169277829:G:A, 2:169277843:C:T, 2:169277847:C:T, 2:169277853:T:C, 2:169277857:T:C, 2:169277863:C:T, 2:169277874:C:T, 2:169277880:C:T, 2:169277888:C:T, 2:169277890:T:C, 2:169277917:C:A, 2:169277925:C:T, 2:169277925:C:G, 2:169277930:C:G, 2:169277934:T:C, 2:169277944:A:G, 2:169277949:T:C, 2:169279378:G:T, 2:169279381:G:A, 2:169279381:G:T, 2:169279385:C:T, 2:169279403:G:C, 2:169279408:C:T, 2:169279420:G:T, 2:169279422:T:C, 2:169279423:A:G, 2:169279429:G:T, 2:169279454:T:G, 2:169279463:C:G, 2:169279464:T:C, 2:169279465:A:C, 2:169279471:T:C, 2:169279481:T:A, 2:169279513:T:C, 2:169279514:C:CCA, 2:169279525:T:C, 2:169279528:T:C, 2:169279529:C:G, 2:169279529:C:T, 2:169279550:T:G, 2:169279553:G:C, 2:169279556:C:T, 2:169279561:T:C, 2:169279564:A:C, 2:169279568:T:G, 2:169279573:C:G, 2:169279573:C:T, 2:169279578:A:C, 2:169279580:T:A, 2:169279580:T:C, 2:169279583:CA:C, 2:169279588:G:T, 2:169279592:A:C, 2:169279595:C:A, 2:169280351:T:C, 2:169280354:T:C, 2:169280360:A:T, 2:169280361:C:A, 2:169280361:C:T, 2:169280364:T:C, 2:169280370:T:C, 2:169280374:A:C, 2:169280378:A:G, 2:169280393:T:C, 2:169280396:T:C, 2:169280414:G:A, 2:169280423:C:G, 2:169280424:G:A, 2:169280427:T:G, 2:169280439:C:T, 2:169280487:G:C, 2:169280493:T:C, 2:169280505:T:A, 2:169280508:A:C, 2:169280510:G:C, 2:169280510:G:T, 2:169280511:C:A, 2:169280517:C:A, 2:169282902:C:A, 2:169282904:C:A, 2:169282906:C:G, 2:169282918:C:A, 2:169282921:C:G, 2:169282922:T:G, 2:169282944:C:T, 2:169282951:G:A, 2:169282953:C:G, 2:169282954:T:G, 2:169282959:C:T, 2:169282961:C:A, 2:169282981:A:G, 2:169282984:T:A, 2:169282994:A:T, 2:169282995:T:C, 2:169282996:CA:C, 2:169282998:A:G, 2:169289025:C:A, 2:169289031:C:G, 2:169289039:G:T, 2:169289043:T:C, 2:169289046:T:A, 2:169289056:T:C, 2:169289070:G:A, 2:169289085:C:T, 2:169289095:G:T, 2:169289119:A:T, 2:169289125:A:T, 2:169289146:C:A, 2:169290861:A:T, 2:169290870:G:C, 2:169290870:G:GT, 2:169290874:T:G, 2:169290878:C:A, 2:169290878:C:G, 2:169290895:T:G, 2:169290904:C:T, 2:169290916:T:G, 2:169290934:C:G, 2:169290935:G:A, 2:169290937:C:A, 2:169290946:G:A, 2:169290964:G:C, 2:169290994:C:A, 2:169292255:C:T, 2:169292255:C:A, 2:169292259:C:T, 2:169292265:C:T, 2:169292266:A:C, 2:169292267:T:G, 2:169292276:T:C, 2:169292282:C:T, 2:169292286:C:T, 2:169292295:C:T, 2:169292298:C:T, 2:169292304:C:A, 2:169292306:C:G, 2:169292310:T:G, 2:169292314:A:T, 2:169292319:T:G, 2:169292319:T:C, 2:169292320:G:T, 2:169292322:A:G, 2:169292327:C:G, 2:169292360:G:T, 2:169292360:G:A, 2:169294147:C:A, 2:169294148:T:C, 2:169294159:TC:T, 2:169294160:C:T, 2:169294162:T:G, 2:169294163:C:T, 2:169294170:G:T, 2:169294178:A:G, 2:169294181:C:T, 2:169294186:T:C, 2:169294198:A:G, 2:169294210:G:A, 2:169294214:T:A, 2:169294217:A:G, 2:169294247:G:A, 2:169294255:ATC:A, 2:169294597:T:TAA, 2:169294601:G:T, 2:169294605:T:A, 2:169294606:T:A, 2:169294615:C:T, 2:169294632:T:C, 2:169294639:T:C, 2:169294649:C:A, 2:169294672:C:T, 2:169294674:C:T, 2:169294677:T:C, 2:169294683:C:A, 2:169294689:A:C, 2:169294690:G:T, 2:169294692:T:A, 2:169294692:T:C, 2:169294693:G:T, 2:169294698:C:T, 2:169294704:G:T, 2:169294705:G:T, 2:169294706:G:T, 2:169294707:T:C, 2:169294708:A:T, 2:169294712:T:A, 2:169307279:A:C, 2:169307279:AC:A, 2:169307285:G:C, 2:169307294:A:C, 2:169307294:A:T, 2:169307303:A:C, 2:169307320:C:T, 2:169307321:G:C, 2:169307323:G:C, 2:169307326:C:A, 2:169307330:C:A, 2:169307356:C:A, 2:169307356:C:T, 2:169307357:A:T, 2:169307372:C:G, 2:169307373:T:A, 2:169307373:T:C, 2:169307379:C:T, 2:169307382:G:C, 2:169307384:G:C, 2:169307388:G:A, 2:169307388:G:T, 2:169307389:T:C, 2:169307394:T:C, 2:169307395:G:T, 2:169307397:G:T, 2:169318760:A:AC, 2:169318761:C:T, 2:169318761:C:G, 2:169318770:T:C, 2:169318804:C:A, 2:169318828:G:T, 2:169318839:C:T, 2:169318843:C:T, 2:169320778:G:T, 2:169320782:C:A, 2:169320783:C:T, 2:169320798:C:A, 2:169320808:G:C, 2:169320853:A:C, 2:169320864:G:T, 2:169320865:A:C, 2:169362326:C:T, 2:169362335:G:A, 2:169362363:G:A, 2:169362372:A:G, 2:169362374:G:C, 2:169362375:C:A, 2:169362378:C:T, 2:169362380:G:T, 2:169362381:C:G, and 2:169362392:C:G (according to the GRCh38/hg38 human genome assembly).

In some embodiments, the subject's aggregate gene burden of having any one or more LRP2 variant nucleic acid molecules encoding LRP2 predicted loss-of-function polypeptides represents a weighted sum of a plurality of any of the LRP2 variant nucleic acid molecules encoding LRP2 predicted loss-of-function polypeptides. In some embodiments, the aggregate gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the LRP2 gene where the gene burden is the number of alleles multiplied by the association estimate with kidney disease or related outcome for each allele (e.g., a weighted burden score). This can include any genetic variants, regardless of their genomic annotation, in proximity to the LRP2 gene (up to 10 Mb around the gene) that show a non-zero association with kidney disease-related traits in a genetic association analysis. In some embodiments, when the subject has an aggregate gene burden above a desired threshold score, the subject has a decreased risk of developing a kidney disease. In some embodiments, when the subject has an aggregate gene burden below a desired threshold score, the subject has an increased risk of developing a kidney disease.

In some embodiments, any of the methods described herein can further comprise determining the subject's aggregate gene burden of having RPL3L variant nucleic acid molecules encoding RPL3L predicted loss-of-function polypeptides associated with a decreased risk of developing a kidney disease. The aggregate gene burden is the sum of all variants in the RPL3L gene, which can be carried out in an association analysis with a kidney disease. In some embodiments, the subject is homozygous for one or more RPL3L variant nucleic acid molecules encoding RPL3L predicted loss-of-function polypeptides associated with a decreased risk of developing a kidney disease. In some embodiments, the subject is heterozygous for one or more RPL3L variant nucleic acid molecules encoding RPL3L predicted loss-of-function polypeptides associated with a decreased risk of developing a kidney disease. The result of the association analysis suggests that RPL3L variant nucleic acid molecules encoding RPL3L predicted loss-of-function polypeptides are associated with decreased risk of developing a kidney disease. When the subject has a lower aggregate gene burden, the subject is at a higher risk of developing a kidney disease and the subject is administered or continued to be administered a therapeutic agent that treats, prevents, or inhibits a kidney disease in an amount that is the same as or less than a standard dosage amount, and/or an RPL3L inhibitor. When the subject has a greater aggregate gene burden, the subject is at a lower risk of developing a kidney disease and the subject is administered or continued to be administered a therapeutic agent that treats, prevents, or inhibits a kidney disease in a standard dosage amount. The greater the aggregate gene burden, the lower the risk of developing a kidney disease.

RPL3L predicted loss-of-function variants include, but are not limited to: 16:1944844:T:TCTCC, 16:1945498:C:T, 16:1945498:C:A, 16:1945863:G:GT, 16:1945932:T:G, 16:1946629:GGT:G, 16:1946656:TAG:T, 16:1946695:TCC:T, 16:1946696:C:A, 16:1946706:GC:G, 16:1946937:C:A, 16:1946946:TG:T, 16:1946957:C:CG, 16:1947094:GA:G, 16:1947197:T:TG, 16:1947243:CTG:C, 16:1947294:C:T, 16:1947363:GA:G, 16:1952890:G:A, 16:1952950:G:A, 16:1953044:T:C, 16:1953954:AC:A, 16:1954011:CA:C, 16:1954631:T:C, 16:1945932:T:A, 16:1946728:T:G, 16:1946952:C:A, 16:1947100:T:C, 16:1950860:AC:A, 16:1950882:T:A, 16:1950906:G:A, 16:1954150:T:C, 16:1954629:C:A, 16:1944838:T:C, 16:1944846:TC:T, 16:1944850:G:T, 16:1945501:T:TG, 16:1945568:C:CT, 16:1945906:CT:C, 16:1945931:C:T, 16:1946624:C:T, 16:1946655:G:C, 16:1946701:T:TGCGG, 16:1946727:C:T, 16:1947017:C:T, 16:1947082:C:T, 16:1947098:CCT:C, 16:1947193:C:A, 16:1947193:C:T, 16:1947206:G:A, 16:1947212:T:A, 16:1947240:ACT:A, 16:1947272:G:A, 16:1947290:G:A, 16:1947309:G:GGCCACCGT, 16:1947332:G:A, 16:1950842:A:G, 16:1950842:A:C, 16:1950877:G:GTACT, 16:1950880:CT:C, 16:1950951:T:TA, 16:1950980:C:G, 16:1952889:C:CCAGTGATG, 16:1952893:GC:G, 16:1952975:G:GC, 16:1952988:A:AG, 16:1953024:TC:T, 16:1953973:AC:A, 16:1954037:G:A, 16:1954071:GC:G, and 16:1954628:C:T (according to the GRCh38/hg38 human genome assembly).

RPL3L pLOF or missense variants predicted to be deleterious by 5 out of 5 in silico prediction algorithms with AAF<5% include, but are not limited to: 16:1944844:T:TCTCC, 16:1945498:C:T, 16:1945498:C:A, 16:1945544:G:T, 16:1945563:A:G, 16:1945596:C:T, 16:1945597:G:A, 16:1945609:C:T, 16:1945845:G:A, 16:1945854:C:T, 16:1945855:G:A, 16:1945863:G:GT, 16:1945875:C:A, 16:1945888:T:C, 16:1945896:T:A, 16:1945897:C:T, 16:1945912:C:T, 16:1945912:C:A, 16:1945918:G:A, 16:1945926:C:T, 16:1945932:T:G, 16:1946629:GGT:G, 16:1946630:G:T, 16:1946633:T:C, 16:1946638:G:T, 16:1946647:G:T, 16:1946654:C:T, 16:1946656:TAG:T, 16:1946673:G:T, 16:1946690:C:T, 16:1946690:C:A, 16:1946695:TCC:T, 16:1946696:C:A, 16:1946706:GC:G, 16:1946714:C:T, 16:1946719:C:T, 16:1946724:G:C, 16:1946937:C:A, 16:1946946:TG:T, 16:1946954:G:A, 16:1946957:C:A, 16:1946957:C:CG, 16:1946957:C:T, 16:1946958:G:A, 16:1946962:G:T, 16:1946964:G:T, 16:1946966:T:C, 16:1946985:G:A, 16:1947002:A:G, 16:1947003:C:T, 16:1947006:G:A, 16:1947008:G:T, 16:1947021:C:T, 16:1947023:C:A, 16:1947030:A:G, 16:1947032:G:T, 16:1947035:A:T, 16:1947041:C:T, 16:1947053:T:C, 16:1947062:C:A, 16:1947062:C:T, 16:1947063:G:A, 16:1947065:G:A, 16:1947086:C:A, 16:1947086:C:T, 16:1947087:G:T, 16:1947094:GA:G, 16:1947197:T:TG, 16:1947210:C:G, 16:1947243:CTG:C, 16:1947244:T:C, 16:1947254:C:T, 16:1947263:C:T, 16:1947265:G:A, 16:1947294:C:T, 16:1947316:G:A, 16:1947323:C:T, 16:1947328:A:G, 16:1947329:G:T, 16:1947345:G:T, 16:1947347:G:T, 16:1947349:G:T, 16:1947352:T:G, 16:1947362:G:A, 16:1947363:GA:G, 16:1947367:G:A, 16:1947368:G:T, 16:1950864:G:A, 16:1950865:A:C, 16:1950876:A:G, 16:1950881:T:A, 16:1950921:C:T, 16:1950971:C:G, 16:1950975:T:C, 16:1952885:G:T, 16:1952889:C:T, 16:1952890:G:A, 16:1952892:C:T, 16:1952893:G:A, 16:1952898:C:T, 16:1952904:T:A, 16:1952941:G:A, 16:1952944:G:A, 16:1952950:G:A, 16:1952976:C:G, 16:1952994:G:T, 16:1952995:G:C, 16:1952995:G:A, 16:1952997:G:A, 16:1953015:G:A, 16:1953016:C:T, 16:1953031:G:A, 16:1953044:T:C, 16:1953954:AC:A, 16:1953968:G:A, 16:1953976:T:C, 16:1953980:G:A, 16:1953991:G:A, 16:1954001:C:T, 16:1954010:C:A, 16:1954011:CA:C, 16:1954021:G:A, 16:1954033:G:A, 16:1954048:T:A, 16:1954054:G:A, 16:1954054:G:C, 16:1954076:G:A, 16:1954082:G:A, 16:1954090:C:G, 16:1954118:C:T, 16:1954123:C:T, 16:1954124:G:A, 16:1954136:A:G, 16:1954142:G:A, 16:1954144:T:C, 16:1954631:T:C, 16:1945560:T:A, 16:1945570:T:C, 16:1945570:T:G, 16:1945617:G:A, 16:1945880:C:G, 16:1945881:T:G, 16:1945929:C:T, 16:1945932:T:A, 16:1946626:A:G, 16:1946630:G:A, 16:1946636:T:C, 16:1946641:T:C, 16:1946654:C:A, 16:1946707:C:A, 16:1946720:G:C, 16:1946728:T:G, 16:1946940:T:G, 16:1946952:C:A, 16:1946955:T:C, 16:1946979:C:T, 16:1946984:C:T, 16:1947009:C:G, 16:1947012:G:A, 16:1947014:T:A, 16:1947018:A:T, 16:1947041:C:A, 16:1947042:G:A, 16:1947049:C:A, 16:1947051:T:C, 16:1947074:T:A, 16:1947095:A:G, 16:1947100:T:C, 16:1947202:C:T, 16:1947223:A:G, 16:1947281:G:C, 16:1947313:A:C, 16:1947326:T:C, 16:1947349:G:A, 16:1947357:C:A, 16:1947357:C:G, 16:1950844:C:G, 16:1950860:AC:A, 16:1950882:T:A, 16:1950888:T:C, 16:1950906:G:A, 16:1950911:T:G, 16:1950916:T:G, 16:1950929:T:G, 16:1950949:C:A, 16:1952875:A:G, 16:1952896:G:A, 16:1952949:C:G, 16:1952976:C:T, 16:1952980:C:T, 16:1953987:G:C, 16:1953987:G:T, 16:1954000:G:T, 16:1954000:G:A, 16:1954009:C:A, 16:1954018:G:A, 16:1954021:G:T, 16:1954026:G:C, 16:1954031:C:T, 16:1954150:T:C, 16:1954629:C:A, 16:1944838:T:C, 16:1944846:TC:T, 16:1944850:G:T, 16:1945500:A:G, 16:1945501:T:TG, 16:1945512:T:C, 16:1945516:C:G, 16:1945524:G:C, 16:1945526:C:G, 16:1945530:A:C, 16:1945537:C:T, 16:1945542:C:A, 16:1945543:C:T, 16:1945549:T:C, 16:1945558:T:C, 16:1945568:C:CT, 16:1945596:C:G, 16:1945597:G:T, 16:1945845:G:C, 16:1945866:C:T, 16:1945875:C:T, 16:1945878:C:T, 16:1945887:A:G, 16:1945895:G:C, 16:1945906:CT:C, 16:1945914:T:C, 16:1945927:C:T, 16:1945930:C:T, 16:1945931:C:T, 16:1946624:C:T, 16:1946636:T:G, 16:1946638:G:A, 16:1946655:G:C, 16:1946668:G:A, 16:1946701:T:TGCGG, 16:1946707:C:T, 16:1946719:C:A, 16:1946727:C:T, 16:1946943:T:G, 16:1946943:T:C, 16:1946946:T:G, 16:1946951:T:G, 16:1946961:G:A, 16:1946964:G:C, 16:1946975:T:C, 16:1946982:C:T, 16:1946985:G:C, 16:1946993:G:A, 16:1947003:C:G, 16:1947013:G:T, 16:1947015:G:C, 16:1947016:C:G, 16:1947017:C:T, 16:1947021:C:G, 16:1947026:A:G, 16:1947027:T:A, 16:1947030:A:T, 16:1947032:G:A, 16:1947033:C:G, 16:1947039:T:C, 16:1947041:C:G, 16:1947054:G:A, 16:1947056:G:A, 16:1947082:C:T, 16:1947087:G:A, 16:1947098:CCT:C, 16:1947193:C:T, 16:1947193:C:A, 16:1947206:G:A, 16:1947209:C:T, 16:1947212:T:A, 16:1947217:A:T, 16:1947218:C:A, 16:1947230:C:A, 16:1947240:ACT:A, 16:1947254:C:G, 16:1947272:G:A, 16:1947290:G:A, 16:1947301:A:G, 16:1947303:C:G, 16:1947309:G:GGCCACCGT, 16:1947326:T:G, 16:1947332:G:A, 16:1947334:A:G, 16:1947353:T:C, 16:1947358:T:G, 16:1950842:A:G, 16:1950842:A:C, 16:1950852:G:T, 16:1950867:T:A, 16:1950877:G:GTACT, 16:1950880:CT:C, 16:1950884:T:C, 16:1950885:T:C, 16:1950917:T:A, 16:1950933:G:A, 16:1950936:A:T, 16:1950951:T:C, 16:1950951:T:TA, 16:1950954:T:C, 16:1950972:T:C, 16:1950980:C:G, 16:1952884:A:G, 16:1952889:C:CCAGTGATG, 16:1952893:GC:G, 16:1952893:G:T, 16:1952907:C:T, 16:1952914:G:A, 16:1952917:C:T, 16:1952919:G:A, 16:1952920:C:G, 16:1952929:T:G, 16:1952932:T:C, 16:1952940:C:G, 16:1952955:G:A, 16:1952967:C:A, 16:1952970:A:G, 16:1952971:C:T, 16:1952975:G:GC, 16:1952977:C:T, 16:1952979:A:G, 16:1952988:A:AG, 16:1953000:T:C, 16:1953006:A:G, 16:1953015:G:T, 16:1953018:T:C, 16:1953022:C:T, 16:1953024:TC:T, 16:1953026:C:A, 16:1953030:C:G, 16:1953967:C:G, 16:1953973:AC:A, 16:1953979:C:G, 16:1953988:T:G, 16:1953991:G:T, 16:1953995:T:C, 16:1953997:C:T, 16:1954004:T:C, 16:1954006:T:C, 16:1954010:C:T, 16:1954012:A:T, 16:1954018:G:T, 16:1954024:A:C, 16:1954024:A:G, 16:1954034:G:C, 16:1954034:G:A, 16:1954037:G:A, 16:1954049:C:T, 16:1954055:G:A, 16:1954071:GC:G, 16:1954072:C:T, 16:1954073:C:T, 16:1954087:C:T, 16:1954108:C:T, 16:1954109:C:A, 16:1954123:C:G, 16:1954130:C:T, 16:1954147:G:A, and 16:1954628:C:T (according to the GRCh38/hg38 human genome assembly).

In some embodiments, the subject's aggregate gene burden of having any one or more RPL3L variant nucleic acid molecules encoding RPL3L predicted loss-of-function polypeptides represents a weighted sum of a plurality of any of the RPL3L variant nucleic acid molecules encoding RPL3L predicted loss-of-function polypeptides. In some embodiments, the aggregate gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the RPL3L gene where the gene burden is the number of alleles multiplied by the association estimate with kidney disease or related outcome for each allele (e.g., a weighted burden score). This can include any genetic variants, regardless of their genomic annotation, in proximity to the RPL3L gene (up to 10 Mb around the gene) that show a non-zero association with kidney disease-related traits in a genetic association analysis. In some embodiments, when the subject has an aggregate gene burden above a desired threshold score, the subject has a decreased risk of developing a kidney disease. In some embodiments, when the subject has an aggregate gene burden below a desired threshold score, the subject has an increased risk of developing a kidney disease.

In some embodiments, any of the methods described herein can further comprise determining the subject's aggregate gene burden of having SLC25A45 variant nucleic acid molecules encoding SLC25A45 predicted loss-of-function polypeptides associated with a decreased risk of developing a kidney disease. The aggregate gene burden is the sum of all variants in the SLC25A45 gene, which can be carried out in an association analysis with a kidney disease. In some embodiments, the subject is homozygous for one or more SLC25A45 variant nucleic acid molecules encoding SLC25A45 predicted loss-of-function polypeptides associated with a decreased risk of developing a kidney disease. In some embodiments, the subject is heterozygous for one or more SLC25A45 variant nucleic acid molecules encoding SLC25A45 predicted loss-of-function polypeptides associated with a decreased risk of developing a kidney disease. The result of the association analysis suggests that SLC25A45 variant nucleic acid molecules encoding SLC25A45 predicted loss-of-function polypeptides are associated with decreased risk of developing a kidney disease. When the subject has a lower aggregate gene burden, the subject is at a higher risk of developing a kidney disease and the subject is administered or continued to be administered a therapeutic agent that treats, prevents, or inhibits a kidney disease in an amount that is the same as or less than a standard dosage amount, and/or an SLC25A45 inhibitor. When the subject has a greater aggregate gene burden, the subject is at a lower risk of developing a kidney disease and the subject is administered or continued to be administered a therapeutic agent that treats, prevents, or inhibits a kidney disease in a standard dosage amount. The greater the aggregate gene burden, the lower the risk of developing a kidney disease.

SLC25A45 pLOF variants include, but are not limited to: 11:65376410:TC:T, 11:65376416:C:T, 11:65376489:AC:A, 11:65376489:A:AC, 11:65376568:GTC:G, 11:65376608:TA:T, 11:65376921:TCG:T, 11:65376923:G:A, 11:65376929:G:GC, 11:65376993:T:TG, 11:65377077:C:A, 11:65379375:C:T, 11:65379384:AC:A, 11:65379394:G:T, 11:65379418:G:T, 11:65379473:AG:A, 11:65379922:TG:T, 11:65379923:G:A, 11:65380176:C:A, 11:65376409:A:T, 11:65376520:G:A, 11:65376830:C:CT, 11:65376918:TC:T, 11:65379374:A:G, 11:65379439:CCG:C, 11:65379866:C:T, 11:65379884:TCTTGACCATGCAATCAA:T, 11:65376433:C:A, 11:65376466:A:AG, 11:65376506:TC:T, 11:65376540:C:CT, 11:65376557:G:C, 11:65376558:T:TA, 11:65376609:A:C, 11:65376676:C:T, 11:65376677:T:C, 11:65376677:T:A, 11:65376815:T:TA, 11:65376817:C:G, 11:65376837:TG:T, 11:65376878:T:TCC, 11:65376887:TG:T, 11:65376910:GC:G, 11:65376912:C:T, 11:65376944:C:A, 11:65377029:G:GT, 11:65377078:T:C, 11:65379373:C:CA, 11:65379374:A:C, 11:65379384:A:AC, 11:65379432:G:A, 11:65379449:TG:T, 11:65379860:CACTCACGG:C, 11:65379866:C:G, 11:65379872:C:A, 11:65379939:C:G, 11:65380130:A:C, and 11:65381950:A:G, 11:65381950:A:T (according to the GRCh38/hg38 human genome assembly).

SLC25A45 pLOF or missense variants with AAF<5% include, but are not limited to: 11:65376410:TC:T, 11:65376416:C:T, 11:65376420:C:T, 11:65376421:G:A, 11:65376421:G:T, 11:65376433:C:T, 11:65376435:T:G, 11:65376438:C:T, 11:65376460:C:T, 11:65376472:G:T, 11:65376472:G:A, 11:65376489:AC:A, 11:65376489:A:AC, 11:65376493:C:A, 11:65376495:C:T, 11:65376495:C:A, 11:65376496:G:A, 11:65376518:C:G, 11:65376519:T:G, 11:65376520:G:T, 11:65376522:C:A, 11:65376522:C:T, 11:65376523:G:A, 11:65376525:A:C, 11:65376528:C:T, 11:65376538:T:G, 11:65376541:A:G, 11:65376552:C:A, 11:65376553:C:T, 11:65376556:G:T, 11:65376559:A:G, 11:65376567:C:T, 11:65376568:GTC:G, 11:65376568:G:A, 11:65376577:C:T, 11:65376584:C:G, 11:65376589:T:A, 11:65376591:C:T, 11:65376608:TA:T, 11:65376615:G:A, 11:65376615:G:T, 11:65376627:C:A, 11:65376633:G:T, 11:65376637:T:C, 11:65376648:C:A, 11:65376648:C:T, 11:65376655:C:T, 11:65376657:A:G, 11:65376664:C:A, 11:65376670:C:A, 11:65376670:C:T, 11:65376674:G:T, 11:65376829:C:G, 11:65376847:C:T, 11:65376860:C:T, 11:65376886:G:A, 11:65376893:T:C, 11:65376895:T:A, 11:65376904:G:A, 11:65376912:C:A, 11:65376921:TCG:T, 11:65376923:G:A, 11:65376929:G:GC, 11:65376935:G:A, 11:65376937:G:A, 11:65376949:C:T, 11:65376964:G:A, 11:65376986:A:T, 11:65376989:G:A, 11:65376992:G:T, 11:65376993:T:TG, 11:65376994:G:T, 11:65376994:G:C, 11:65376995:G:T, 11:65377001:T:C, 11:65377013:C:T, 11:65377020:C:G, 11:65377021:T:C, 11:65377029:G:C, 11:65377039:C:T, 11:65377054:T:C, 11:65377063:G:T, 11:65377077:C:A, 11:65379375:C:T, 11:65379382:G:T, 11:65379384:AC:A, 11:65379389:C:T, 11:65379390:C:T, 11:65379394:G:T, 11:65379401:G:A, 11:65379404:A:T, 11:65379406:G:T, 11:65379408:A:G, 11:65379416:A:G, 11:65379418:G:T, 11:65379428:G:A, 11:65379429:G:T, 11:65379437:C:T, 11:65379437:C:G, 11:65379440:C:T, 11:65379442:C:G, 11:65379443:T:C, 11:65379444:C:T, 11:65379450:G:T, 11:65379452:G:C, 11:65379458:G:T, 11:65379458:G:C, 11:65379461:G:A, 11:65379467:A:G, 11:65379468:C:T, 11:65379473:AG:A, 11:65379485:T:C, 11:65379512:A:G, 11:65379518:A:G, 11:65379530:G:A, 11:65379537:T:C, 11:65379542:C:T, 11:65379877:C:T, 11:65379902:C:T, 11:65379905:T:C, 11:65379910:C:T, 11:65379911:G:A, 11:65379922:TG:T, 11:65379923:G:A, 11:65379934:C:A, 11:65380148:G:C, 11:65380157:A:G, 11:65380164:A:C, 11:65380166:C:A, 11:65380176:C:A, 11:65381939:C:G, 11:65381944:A:G, 11:65381947:G:A, 11:65376409:A:T, 11:65376429:T:C, 11:65376448:T:C, 11:65376468:G:A, 11:65376471:C:T, 11:65376478:T:A, 11:65376480:T:C, 11:65376493:C:T, 11:65376510:A:G, 11:65376520:G:A, 11:65376568:G:T, 11:65376582:A:G, 11:65376643:C:T, 11:65376648:C:G, 11:65376675:C:G, 11:65376829:C:A, 11:65376830:C:CT, 11:65376832:T:C, 11:65376848:G:A, 11:65376851:A:C, 11:65376862:T:G, 11:65376887:T:C, 11:65376916:G:T, 11:65376917:C:T, 11:65376918:TC:T, 11:65376919:C:G, 11:65376922:C:T, 11:65376950:G:A, 11:65376961:G:A, 11:65376970:T:C, 11:65376977:G:T, 11:65376988:C:T, 11:65376997:G:A, 11:65376999:G:C, 11:65377009:T:C, 11:65377012:G:C, 11:65377034:G:C, 11:65377036:A:G, 11:65377040:G:A, 11:65377058:A:G, 11:65379374:A:G, 11:65379378:G:T, 11:65379410:A:T, 11:65379421:G:C, 11:65379432:G:T, 11:65379437:C:A, 11:65379438:G:A, 11:65379439:CCG:C, 11:65379441:G:A, 11:65379471:G:T, 11:65379476:G:A, 11:65379494:A:G, 11:65379527:A:G, 11:65379866:C:T, 11:65379878:G:A, 11:65379881:A:C, 11:65379884:TCTTGACCATGCAATCAA:T, 11:65379938:C:T, 11:65380137:C:T, 11:65380142:T:C, 11:65380151:T:A, 11:65376411:C:T, 11:65376420:C:A, 11:65376433:C:A, 11:65376445:A:C, 11:65376451:C:T, 11:65376453:G:A, 11:65376454:C:A, 11:65376462:G:A, 11:65376463:G:A, 11:65376466:A:AG, 11:65376469:C:T, 11:65376490:C:T, 11:65376490:C:A, 11:65376492:C:G, 11:65376492:C:T, 11:65376499:A:G, 11:65376500:G:C, 11:65376502:A:C, 11:65376506:TC:T, 11:65376508:C:T, 11:65376511:G:T, 11:65376515:T:G, 11:65376519:T:C, 11:65376526:T:C, 11:65376529:T:C, 11:65376535:C:T, 11:65376537:A:G, 11:65376540:C:A, 11:65376540:C:CT, 11:65376543:T:C, 11:65376546:A:G, 11:65376550:T:A, 11:65376557:G:C, 11:65376558:T:TA, 11:65376561:A:G, 11:65376564:C:T, 11:65376573:A:G, 11:65376579:T:C, 11:65376589:T:C, 11:65376592:G:A, 11:65376605:G:C, 11:65376609:A:C, 11:65376609:A:G, 11:65376618:G:A, 11:65376640:C:T, 11:65376642:G:A, 11:65376649:C:T, 11:65376655:C:A, 11:65376657:A:C, 11:65376660:A:G, 11:65376663:A:C, 11:65376666:G:A, 11:65376669:G:A, 11:65376672:G:A, 11:65376675:C:A, 11:65376676:C:T, 11:65376677:T:A, 11:65376677:T:C, 11:65376815:T:TA, 11:65376817:C:G, 11:65376823:T:A, 11:65376837:TG:T, 11:65376842:A:C, 11:65376844:T:C, 11:65376844:T:G, 11:65376847:C:A, 11:65376856:C:T, 11:65376857:C:G, 11:65376862:T:C, 11:65376865:G:T, 11:65376870:G:T, 11:65376872:A:C, 11:65376874:T:G, 11:65376875:A:G, 11:65376877:A:T, 11:65376878:T:TCC, 11:65376886:G:T, 11:65376887:TG:T, 11:65376892:G:T, 11:65376893:T:G, 11:65376910:G:A, 11:65376910:GC:G, 11:65376911:C:T, 11:65376912:C:T, 11:65376917:C:A, 11:65376919:C:T, 11:65376922:C:A, 11:65376922:C:G, 11:65376926:A:G, 11:65376928:A:C, 11:65376929:G:T, 11:65376931:C:T, 11:65376932:C:G, 11:65376934:C:T, 11:65376938:G:A, 11:65376944:C:T, 11:65376944:C:G, 11:65376944:C:A, 11:65376947:C:T, 11:65376955:A:G, 11:65376956:T:C, 11:65376974:C:T, 11:65376983:G:T, 11:65376994:G:A, 11:65376998:G:A, 11:65377004:C:G, 11:65377015:C:G, 11:65377022:C:T, 11:65377025:T:C, 11:65377025:T:A, 11:65377029:G:GT, 11:65377052:G:A, 11:65377064:C:T, 11:65377068:A:C, 11:65377070:A:G, 11:65377075:G:T, 11:65377078:T:C, 11:65379373:C:CA, 11:65379374:A:C, 11:65379384:A:AC, 11:65379386:C:A, 11:65379386:C:G, 11:65379387:C:T, 11:65379390:C:G, 11:65379393:T:C, 11:65379395:C:T, 11:65379398:C:T, 11:65379402:C:A, 11:65379411:T:C, 11:65379414:G:A, 11:65379415:C:T, 11:65379432:G:A, 11:65379435:C:T, 11:65379435:C:A, 11:65379449:TG:T, 11:65379464:A:G, 11:65379465:G:T, 11:65379474:G:C, 11:65379497:A:G, 11:65379509:A:G, 11:65379513:C:T, 11:65379518:A:T, 11:65379534:A:G, 11:65379551:A:T, 11:65379552:A:C, 11:65379554:C:T, 11:65379860:CACTCACGG:C, 11:65379866:C:G, 11:65379872:C:A, 11:65379872:C:G, 11:65379877:C:A, 11:65379885:C:A, 11:65379886:T:C, 11:65379901:A:G, 11:65379904:A:G, 11:65379908:C:G, 11:65379910:C:A, 11:65379922:T:G, 11:65379925:G:A, 11:65379938:C:A, 11:65379939:C:G, 11:65380130:A:C, 11:65380132:C:A, 11:65380148:G:A, 11:65380161:C:T, 11:65380167:C:T, 11:65380173:C:T, 11:65381921:T:C, 11:65381922:C:A, 11:65381927:C:A, 11:65381939:C:T, 11:65381945:C:G, 11:65381945:C:T, 11:65381948:G:A, 11:65381948:G:T, 11:65381950:A:T, and 11:65381950:A:G (according to the GRCh38/hg38 human genome assembly).

In some embodiments, the subject's aggregate gene burden of having any one or more SLC25A45 variant nucleic acid molecules encoding SLC25A45 predicted loss-of-function polypeptides represents a weighted sum of a plurality of any of the SLC25A45 variant nucleic acid molecules encoding SLC25A45 predicted loss-of-function polypeptides. In some embodiments, the aggregate gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the SLC25A45 gene where the gene burden is the number of alleles multiplied by the association estimate with kidney disease or related outcome for each allele (e.g., a weighted burden score). This can include any genetic variants, regardless of their genomic annotation, in proximity to the SLC25A45 gene (up to 10 Mb around the gene) that show a non-zero association with kidney disease-related traits in a genetic association analysis. In some embodiments, when the subject has an aggregate gene burden above a desired threshold score, the subject has a decreased risk of developing a kidney disease. In some embodiments, when the subject has an aggregate gene burden below a desired threshold score, the subject has an increased risk of developing a kidney disease.

In some embodiments, any of the methods described herein can further comprise determining the subject's aggregate gene burden of having SLC7A9 variant nucleic acid molecules encoding SLC7A9 predicted loss-of-function polypeptides associated with a decreased risk of developing a kidney disease. The aggregate gene burden is the sum of all variants in the SLC7A9 gene, which can be carried out in an association analysis with a kidney disease. In some embodiments, the subject is homozygous for one or more SLC7A9 variant nucleic acid molecules encoding SLC7A9 predicted loss-of-function polypeptides associated with a decreased risk of developing a kidney disease. In some embodiments, the subject is heterozygous for one or more SLC7A9 variant nucleic acid molecules encoding SLC7A9 predicted loss-of-function polypeptides associated with a decreased risk of developing a kidney disease. The result of the association analysis suggests that SLC7A9 variant nucleic acid molecules encoding SLC7A9 predicted loss-of-function polypeptides are associated with decreased risk of developing a kidney disease. When the subject has a lower aggregate gene burden, the subject is at a higher risk of developing a kidney disease and the subject is administered or continued to be administered a therapeutic agent that treats, prevents, or inhibits a kidney disease in an amount that is the same as or less than a standard dosage amount, and/or an SLC7A9 inhibitor. When the subject has a greater aggregate gene burden, the subject is at a lower risk of developing a kidney disease and the subject is administered or continued to be administered a therapeutic agent that treats, prevents, or inhibits a kidney disease in a standard dosage amount. The greater the aggregate gene burden, the lower the risk of developing a kidney disease.

SLC7A9 predicted loss-of-function variants include, but are not limited to: 19:32830686:T:C, 19:32833195:G:T, 19:32833214:A:C, 19:32833242:C:A, 19:32833244:C:T, 19:32833280:AAC:A, 19:32842318:C:G, 19:32858454:G:T, 19:32858458:GTC:G, 19:32858477:TGGTTGAAAATG:T, 19:32858482:GA:G, 19:32858533:TCA:T, 19:32860638:ATTGAG:A, 19:32862207:C:CT, 19:32862459:A:G, 19:32862561:C:CAGTG, 19:32864161:GCA:G, 19:32864174:C:CG, 19:32864204:ACG:A, 19:32864283:A:T, 19:32864293:CCT:C, 19:32864648:G:T, 19:32868560:C:A, 19:32868561:T:C, 19:32830626:A:AG, 19:32833161:G:A, 19:32843856:TA:T, 19:32843856:TAA:T, 19:32858533:TC:T, 19:32859899:AC:A, 19:32860605:C:G, 19:32862127:TA:T, 19:32862460:C:T, 19:32862587:C:T, 19:32864294:CT:C, 19:32868486:G:A, 19:32868534:T:C, 19:32868560:C:T, 19:32868561:T:A, 19:32830615:GCTTGTT:G, 19:32830624:TC:T, 19:32830685:C:G, 19:32833146:T:TA, 19:32833154:A:AT, 19:32833195:GTAAAA:G, 19:32833225:CA:C, 19:32833315:T:TACGG, 19:32842167:C:A, 19:32842191:T:A, 19:32842248:C:CG, 19:32842278:TTA:T, 19:32842315:AC:A, 19:32843853:A:G, 19:32843854:C:A, 19:32843878:GA:G, 19:32843942:G:T, 19:32843952:C:T, 19:32859848:AC:A, 19:32859917:CA:C, 19:32859928:CA:C, 19:32859961:GT:G, 19:32860604:A:T, 19:32860628:CTG:C, 19:32862126:A:T, 19:32862209:TTG:T, 19:32864120:TCA:T, 19:32864169:A:T, 19:32864277:G:T, 19:32864289:T:TC, 19:32864289:TCC:T, 19:32864627:A:T, 19:32864627:A:ACCCAG, 19:32864701:C:CAG, 19:32868455:TG:T, 19:32868505:TC:T, and 19:32868507:T:A (according to the GRCh38/hg38 human genome assembly).

SLC7A9 pLOF or missense variants with AAF<1% include, but are not limited to: 19:32830639:G:A, 19:32830640:G:A, 19:32830646:C:T, 19:32830670:G:C, 19:32830681:G:A, 19:32830686:T:C, 19:32833160:T:C, 19:32833179:A:G, 19:32833195:G:T, 19:32833206:G:C, 19:32833214:A:C, 19:32833236:G:A, 19:32833242:C:A, 19:32833244:C:T, 19:32833247:G:A, 19:32833247:G:T, 19:32833247:G:C, 19:32833251:G:A, 19:32833262:A:T, 19:32833280:AAC:A, 19:32833295:G:A, 19:32833317:C:T, 19:32833319:G:A, 19:32833322:A:G, 19:32842188:C:T, 19:32842190:T:C, 19:32842193:C:T, 19:32842196:G:C, 19:32842196:G:A, 19:32842200:A:G, 19:32842203:T:C, 19:32842209:C:T, 19:32842212:T:C, 19:32842214:A:G, 19:32842226:G:A, 19:32842245:A:G, 19:32842248:C:T, 19:32842255:G:C, 19:32842274:G:A, 19:32842301:A:G, 19:32842304:G:A, 19:32842308:C:T, 19:32842310:A:C, 19:32842316:C:A, 19:32842318:C:G, 19:32843869:C:T, 19:32843886:C:T, 19:32843886:C:A, 19:32843893:C:T, 19:32843899:T:C, 19:32843910:A:C, 19:32843925:C:A, 19:32843932:G:A, 19:32843941:C:T, 19:32858440:C:G, 19:32858446:G:A, 19:32858454:G:T, 19:32858455:C:G, 19:32858458:GTC:G, 19:32858462:C:T, 19:32858477:TGGTTGAAAATG:T, 19:32858482:GA:G, 19:32858489:C:T, 19:32858497:A:C, 19:32858497:A:G, 19:32858533:TCA:T, 19:32858534:C:T, 19:32859857:G:A, 19:32859870:C:T, 19:32859885:C:T, 19:32859887:G:A, 19:32859900:C:T, 19:32859902:T:C, 19:32859920:G:A, 19:32859932:G:A, 19:32859939:C:T, 19:32859940:G:C, 19:32860618:C:A, 19:32860638:ATTGAG:A, 19:32860648:T:G, 19:32862119:A:G, 19:32862127:T:C, 19:32862142:T:C, 19:32862151:G:A, 19:32862151:G:C, 19:32862182:C:T, 19:32862207:C:CT, 19:32862214:T:A, 19:32862459:A:G, 19:32862466:G:T, 19:32862478:A:G, 19:32862481:C:T, 19:32862503:C:T, 19:32862506:T:A, 19:32862518:C:A, 19:32862518:C:T, 19:32862520:G:A, 19:32862521:C:T, 19:32862524:T:C, 19:32862539:C:G, 19:32862539:C:T, 19:32862553:C:T, 19:32862554:G:A, 19:32862561:C:CAGTG, 19:32862583:A:G, 19:32862586:A:G, 19:32864102:C:T, 19:32864105:C:T, 19:32864108:C:T, 19:32864152:T:A, 19:32864155:A:G, 19:32864161:G:A, 19:32864161:GCA:G, 19:32864174:C:T, 19:32864174:C:CG, 19:32864179:A:T, 19:32864204:ACG:A, 19:32864206:G:A, 19:32864209:G:A, 19:32864219:C:T, 19:32864220:G:C, 19:32864242:A:G, 19:32864245:T:C, 19:32864249:C:T, 19:32864252:G:A, 19:32864261:C:T, 19:32864272:A:T, 19:32864279:A:G, 19:32864281:G:A, 19:32864283:A:T, 19:32864293:CCT:C, 19:32864294:C:G, 19:32864307:C:A, 19:32864326:A:C, 19:32864332:A:T, 19:32864336:C:A, 19:32864629:C:A, 19:32864634:G:A, 19:32864637:G:T, 19:32864641:G:A, 19:32864643:A:G, 19:32864646:C:A, 19:32864647:C:T, 19:32864647:C:G, 19:32864648:G:T, 19:32864653:C:T, 19:32864657:C:G, 19:32864660:T:C, 19:32864664:A:T, 19:32864682:G:C, 19:32864688:G:A, 19:32864695:T:G, 19:32864716:C:T, 19:32864724:C:G, 19:32864734:T:C, 19:32864735:G:C, 19:32864746:C:T, 19:32864752:T:C, 19:32868465:T:C, 19:32868479:T:C, 19:32868491:G:A, 19:32868491:G:C, 19:32868509:C:T, 19:32868512:T:C, 19:32868519:G:T, 19:32868560:C:A, 19:32868561:T:C, 19:32830624:T:A, 19:32830624:T:G, 19:32830626:A:AG, 19:32833149:T:C, 19:32833161:G:A, 19:32833241:T:C, 19:32833251:G:T, 19:32833254:T:G, 19:32833289:A:T, 19:32833296:T:G, 19:32833305:C:T, 19:32833316:A:G, 19:32842205:A:C, 19:32843856:TAA:T, 19:32843856:TA:T, 19:32843874:G:A, 19:32843880:G:A, 19:32843884:G:T, 19:32843887:G:A, 19:32843901:T:G, 19:32843937:G:A, 19:32843943:T:C, 19:32858507:T:G, 19:32858530:C:T, 19:32858533:TC:T, 19:32859851:G:A, 19:32859897:A:G, 19:32859899:AC:A, 19:32860605:C:G, 19:32860613:G:A, 19:32860633:A:G, 19:32860639:T:C, 19:32862127:TA:T, 19:32862131:C:T, 19:32862147:A:C, 19:32862188:C:T, 19:32862198:A:T, 19:32862200:C:G, 19:32862202:A:T, 19:32862460:C:T, 19:32862482:C:T, 19:32862485:T:C, 19:32862506:T:C, 19:32862523:G:A, 19:32862557:C:T, 19:32862563:G:C, 19:32862587:C:T, 19:32864161:G:T, 19:32864163:A:C, 19:32864164:C:A, 19:32864165:A:G, 19:32864198:C:T, 19:32864226:G:C, 19:32864288:C:G, 19:32864294:CT:C, 19:32864302:G:T, 19:32864323:G:A, 19:32864635:T:G, 19:32864638:C:T, 19:32864661:A:C, 19:32864662:T:C, 19:32864665:T:G, 19:32864698:G:T, 19:32864706:T:G, 19:32864710:G:C, 19:32864712:G:A, 19:32864763:C:T, 19:32864764:T:C, 19:32868451:C:G, 19:32868451:C:A, 19:32868485:T:G, 19:32868486:G:A, 19:32868492:A:T, 19:32868499:A:C, 19:32868518:A:C, 19:32868534:T:C, 19:32868560:C:T, 19:32868561:T:A, 19:32830615:GCTTGTT:G, 19:32830624:TC:T, 19:32830629:G:T, 19:32830634:C:T, 19:32830639:G:T, 19:32830653:C:T, 19:32830654:A:G, 19:32830669:T:C, 19:32830670:G:A, 19:32830670:G:T, 19:32830671:C:T, 19:32830672:A:G, 19:32830673:T:C, 19:32830685:C:G, 19:32833146:T:TA, 19:32833154:A:AT, 19:32833159:C:G, 19:32833167:A:G, 19:32833172:A:C, 19:32833181:T:C, 19:32833187:A:G, 19:32833195:GTAAAA:G, 19:32833208:C:T, 19:32833209:C:T, 19:32833217:A:G, 19:32833223:A:G, 19:32833225:CA:C, 19:32833226:A:G, 19:32833235:A:G, 19:32833242:C:G, 19:32833260:T:A, 19:32833280:A:G, 19:32833281:A:G, 19:32833281:A:C, 19:32833284:C:T, 19:32833302:A:T, 19:32833315:T:TACGG, 19:32842167:C:A, 19:32842169:T:C, 19:32842171:G:C, 19:32842175:G:A, 19:32842177:C:A, 19:32842179:T:A, 19:32842187:T:G, 19:32842191:T:A, 19:32842191:T:C, 19:32842198:A:T, 19:32842215:G:A, 19:32842218:C:T, 19:32842230:G:C, 19:32842232:C:A, 19:32842248:C:CG, 19:32842260:A:C, 19:32842270:T:A, 19:32842271:A:G, 19:32842277:T:A, 19:32842278:TTA:T, 19:32842278:T:C, 19:32842280:A:G, 19:32842296:T:C, 19:32842298:T:C, 19:32842299:A:C, 19:32842304:G:T, 19:32842311:T:C, 19:32842315:AC:A, 19:32843853:A:G, 19:32843854:C:A, 19:32843858:A:C, 19:32843868:G:T, 19:32843878:GA:G, 19:32843892:A:G, 19:32843913:T:G, 19:32843914:T:G, 19:32843920:T:C, 19:32843931:C:T, 19:32843942:G:T, 19:32843952:C:T, 19:32858440:C:T, 19:32858441:T:C, 19:32858452:A:G, 19:32858453:A:G, 19:32858459:T:A, 19:32858471:C:T, 19:32858474:C:T, 19:32858495:A:G, 19:32858504:C:T, 19:32858506:A:G, 19:32858510:A:C, 19:32858521:T:C, 19:32858521:T:G, 19:32858527:A:G, 19:32858531:G:A, 19:32858536:C:A, 19:32859846:C:T, 19:32859848:AC:A, 19:32859851:G:T, 19:32859852:C:T, 19:32859872:G:A, 19:32859873:T:C, 19:32859875:G:A, 19:32859878:G:A, 19:32859880:C:T, 19:32859888:T:C, 19:32859890:A:T, 19:32859896:G:C, 19:32859905:A:G, 19:32859908:A:T, 19:32859917:CA:C, 19:32859920:G:C, 19:32859923:G:A, 19:32859928:CA:C, 19:32859942:T:A, 19:32859945:T:C, 19:32859960:G:C, 19:32859961:GT:G, 19:32859963:T:G, 19:32860604:A:T, 19:32860606:C:T, 19:32860613:G:T, 19:32860628:CTG:C, 19:32860646:G:T, 19:32860648:T:C, 19:32862122:C:T, 19:32862123:A:T, 19:32862123:A:C, 19:32862124:T:C, 19:32862126:A:T, 19:32862128:A:C, 19:32862130:G:C, 19:32862136:A:G, 19:32862136:A:C, 19:32862139:C:T, 19:32862146:A:G, 19:32862172:G:A, 19:32862178:T:C, 19:32862185:C:T, 19:32862191:A:G, 19:32862193:G:T, 19:32862209:TTG:T, 19:32862472:A:G, 19:32862476:C:T, 19:32862483:G:T, 19:32862483:G:C, 19:32862487:A:G, 19:32862490:A:G, 19:32862493:A:T, 19:32862494:T:C, 19:32862496:A:G, 19:32862497:T:C, 19:32862503:C:A, 19:32862515:T:C, 19:32862531:G:T, 19:32862535:T:C, 19:32862536:G:T, 19:32862538:A:G, 19:32862547:C:T, 19:32862548:C:G, 19:32862554:G:C, 19:32862572:C:T, 19:32862574:G:C, 19:32862574:G:A, 19:32862577:G:A, 19:32862582:G:C, 19:32864099:T:C, 19:32864102:C:A, 19:32864120:TCA:T, 19:32864126:C:T, 19:32864131:T:G, 19:32864135:G:A, 19:32864138:G:C, 19:32864143:C:A, 19:32864150:C:T, 19:32864158:G:C, 19:32864159:G:A, 19:32864169:A:T, 19:32864173:T:C, 19:32864183:T:C, 19:32864183:T:G, 19:32864190:G:C, 19:32864206:G:T, 19:32864216:T:G, 19:32864219:C:A, 19:32864230:G:A, 19:32864234:A:C, 19:32864234:A:G, 19:32864240:A:G, 19:32864243:G:A, 19:32864245:T:G, 19:32864254:A:G, 19:32864255:T:G, 19:32864272:A:C, 19:32864277:G:T, 19:32864281:G:C, 19:32864282:G:T, 19:32864285:A:G, 19:32864289:T:TC, 19:32864289:TCC:T, 19:32864290:C:T, 19:32864299:T:C, 19:32864299:T:A, 19:32864299:T:G, 19:32864308:A:G, 19:32864311:G:A, 19:32864314:C:G, 19:32864317:A:C, 19:32864327:A:G, 19:32864332:A:C, 19:32864627:A:T, 19:32864627:A:ACCCAG, 19:32864637:G:A, 19:32864646:C:T, 19:32864652:G:A, 19:32864655:G:A, 19:32864655:G:T, 19:32864656:C:T, 19:32864659:A:G, 19:32864664:A:G, 19:32864667:A:T, 19:32864671:A:G, 19:32864677:C:T, 19:32864682:G:A, 19:32864700:A:T, 19:32864701:C:G, 19:32864701:C:CAG, 19:32864703:G:A, 19:32864705:C:G, 19:32864709:G:A, 19:32864710:G:A, 19:32864717:G:T, 19:32864733:A:G, 19:32864745:A:C, 19:32864748:A:G, 19:32864757:A:T, 19:32864758:T:C, 19:32864763:C:G, 19:32864765:G:C, 19:32864773:C:T, 19:32868455:TG:T, 19:32868462:T:C, 19:32868464:G:T, 19:32868476:T:C, 19:32868505:T:G, 19:32868505:TC:T, 19:32868507:T:A, 19:32868507:T:C, 19:32868510:G:A, 19:32868510:G:C, 19:32868522:C:T, and 19:32868530:C:T (according to the GRCh38/hg38 human genome assembly).

In some embodiments, the subject's aggregate gene burden of having any one or more SLC7A9 variant nucleic acid molecules encoding SLC7A9 predicted loss-of-function polypeptides represents a weighted sum of a plurality of any of the SLC7A9 variant nucleic acid molecules encoding SLC7A9 predicted loss-of-function polypeptides. In some embodiments, the aggregate gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the SLC7A9 gene where the gene burden is the number of alleles multiplied by the association estimate with kidney disease or related outcome for each allele (e.g., a weighted burden score). This can include any genetic variants, regardless of their genomic annotation, in proximity to the SLC7A9 gene (up to 10 Mb around the gene) that show a non-zero association with kidney disease-related traits in a genetic association analysis. In some embodiments, when the subject has an aggregate gene burden above a desired threshold score, the subject has a decreased risk of developing a kidney disease. In some embodiments, when the subject has an aggregate gene burden below a desired threshold score, the subject has an increased risk of developing a kidney disease.

In some embodiments, the aggregate gene burden may be divided into quintiles, e.g., top quintile, intermediate quintile, and bottom quintile, wherein the top quintile of aggregate gene burden corresponds to the lowest risk group and the bottom quintile of aggregate gene burden corresponds to the highest risk group. In some embodiments, a subject having a greater aggregate gene burden comprises the highest weighted aggregate burdens, including, but not limited to the top 10%, top 20%, top 30%, top 40%, or top 50% of aggregate gene burdens from a subject population. In some embodiments, the genetic variants comprise the genetic variants having association with a kidney disease in the top 10%, top 20%, top 30%, top 40%, or top 50% of p-value range for the association. In some embodiments, each of the identified genetic variants comprise the genetic variants having association with a kidney disease with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with a kidney disease with p-value of less than 5×10⁻⁸. In some embodiments, the identified genetic variants comprise genetic variants having association with a kidney disease in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate gene burdens in the bottom decile, quintile, or tertile in a reference population. The threshold of the aggregate gene burden is determined on the basis of the nature of the intended practical application and the risk difference that would be considered meaningful for that practical application.

In some embodiments, when a subject is identified as having an increased risk of developing a kidney disease, the subject is further administered a therapeutic agent that treats, prevents, or inhibits a kidney disease, and/or an ALDH1L1 inhibitor, as described herein. For example, when the subject is ALDH1L1 reference, and therefore has an increased risk of developing a kidney disease, the subject is administered an ALDH1L1 inhibitor. In some embodiments, such a subject is also administered a therapeutic agent that treats, prevents, or inhibits a kidney disease. In some embodiments, when the subject is heterozygous for an ALDH1L1 variant nucleic acid molecule encoding an ALDH1L1 predicted loss-of-function polypeptide, the subject is administered the therapeutic agent that treats, prevents, or inhibits a kidney disease in a dosage amount that is the same as or less than a standard dosage amount, and is also administered an ALDH1L1 inhibitor. In some embodiments, the subject is ALDH1L1 reference. In some embodiments, the subject is heterozygous for an ALDH1L1 variant nucleic acid molecule encoding an ALDH1L1 predicted loss-of-function polypeptide. Furthermore, when the subject has a lower aggregate gene burden for having an ALDH1L1 variant nucleic acid molecule encoding an ALDH1L1 predicted loss-of-function polypeptide, and therefore has an increased risk of developing a kidney disease, the subject is administered a therapeutic agent that treats, prevents, or inhibits a kidney disease. In some embodiments, when the subject has a lower aggregate gene burden for having an ALDH1L1 variant nucleic acid molecule encoding an ALDH1L1 predicted loss-of-function polypeptide, the subject is administered the therapeutic agent that treats, prevents, or inhibits a kidney disease in a dosage amount that is the same as or less than the standard dosage amount administered to a subject who has a greater aggregate gene burden for having an ALDH1L1 variant nucleic acid molecule encoding an ALDH1L1 predicted loss-of-function polypeptide.

In some embodiments, when a subject is identified as having an increased risk of developing a kidney disease, the subject is further administered a therapeutic agent that treats, prevents, or inhibits a kidney disease, and/or an ALDOB inhibitor, as described herein. For example, when the subject is ALDOB reference, and therefore has an increased risk of developing a kidney disease, the subject is administered an ALDOB inhibitor. In some embodiments, such a subject is also administered a therapeutic agent that treats, prevents, or inhibits a kidney disease. In some embodiments, when the subject is heterozygous for an ALDOB variant nucleic acid molecule encoding an ALDOB predicted loss-of-function polypeptide, the subject is administered the therapeutic agent that treats, prevents, or inhibits a kidney disease in a dosage amount that is the same as or less than a standard dosage amount, and is also administered an ALDOB inhibitor. In some embodiments, the subject is ALDOB reference. In some embodiments, the subject is heterozygous for an ALDOB variant nucleic acid molecule encoding an ALDOB predicted loss-of-function polypeptide. Furthermore, when the subject has a lower aggregate gene burden for having an ALDOB variant nucleic acid molecule encoding an ALDOB predicted loss-of-function polypeptide, and therefore has an increased risk of developing a kidney disease, the subject is administered a therapeutic agent that treats, prevents, or inhibits a kidney disease. In some embodiments, when the subject has a lower aggregate gene burden for having an ALDOB variant nucleic acid molecule encoding an ALDOB predicted loss-of-function polypeptide, the subject is administered the therapeutic agent that treats, prevents, or inhibits a kidney disease in a dosage amount that is the same as or less than the standard dosage amount administered to a subject who has a greater aggregate gene burden for having an ALDOB variant nucleic acid molecule encoding an ALDOB predicted loss-of-function polypeptide.

In some embodiments, when a subject is identified as having an increased risk of developing a kidney disease, the subject is further administered a therapeutic agent that treats, prevents, or inhibits a kidney disease, and/or a G6PC inhibitor, as described herein. For example, when the subject is G6PC reference, and therefore has an increased risk of developing a kidney disease, the subject is administered a G6PC inhibitor. In some embodiments, such a subject is also administered a therapeutic agent that treats, prevents, or inhibits a kidney disease. In some embodiments, when the subject is heterozygous for a G6PC variant nucleic acid molecule encoding a G6PC predicted loss-of-function polypeptide, the subject is administered the therapeutic agent that treats, prevents, or inhibits a kidney disease in a dosage amount that is the same as or less than a standard dosage amount, and is also administered a G6PC inhibitor. In some embodiments, the subject is G6PC reference. In some embodiments, the subject is heterozygous for a G6PC variant nucleic acid molecule encoding a G6PC predicted loss-of-function polypeptide. Furthermore, when the subject has a lower aggregate gene burden for having a G6PC variant nucleic acid molecule encoding a G6PC predicted loss-of-function polypeptide, and therefore has an increased risk of developing a kidney disease, the subject is administered a therapeutic agent that treats, prevents, or inhibits a kidney disease. In some embodiments, when the subject has a lower aggregate gene burden for having a G6PC variant nucleic acid molecule encoding a G6PC predicted loss-of-function polypeptide, the subject is administered the therapeutic agent that treats, prevents, or inhibits a kidney disease in a dosage amount that is the same as or less than the standard dosage amount administered to a subject who has a greater aggregate gene burden for having a G6PC variant nucleic acid molecule encoding a G6PC predicted loss-of-function polypeptide.

In some embodiments, when a subject is identified as having an increased risk of developing a kidney disease, the subject is further administered a therapeutic agent that treats, prevents, or inhibits a kidney disease, and/or an LRP2 inhibitor, as described herein. For example, when the subject is LRP2 reference, and therefore has an increased risk of developing a kidney disease, the subject is administered an LRP2 inhibitor. In some embodiments, such a subject is also administered a therapeutic agent that treats, prevents, or inhibits a kidney disease. In some embodiments, when the subject is heterozygous for an LRP2 variant nucleic acid molecule encoding an LRP2 predicted loss-of-function polypeptide, the subject is administered the therapeutic agent that treats, prevents, or inhibits a kidney disease in a dosage amount that is the same as or less than a standard dosage amount, and is also administered an LRP2 inhibitor. In some embodiments, the subject is LRP2 reference. In some embodiments, the subject is heterozygous for an LRP2 variant nucleic acid molecule encoding an LRP2 predicted loss-of-function polypeptide. Furthermore, when the subject has a lower aggregate gene burden for having an LRP2 variant nucleic acid molecule encoding an LRP2 predicted loss-of-function polypeptide, and therefore has an increased risk of developing a kidney disease, the subject is administered a therapeutic agent that treats, prevents, or inhibits a kidney disease. In some embodiments, when the subject has a lower aggregate gene burden for having an LRP2 variant nucleic acid molecule encoding an LRP2 predicted loss-of-function polypeptide, the subject is administered the therapeutic agent that treats, prevents, or inhibits a kidney disease in a dosage amount that is the same as or less than the standard dosage amount administered to a subject who has a greater aggregate gene burden for having an LRP2 variant nucleic acid molecule encoding an LRP2 predicted loss-of-function polypeptide.

In some embodiments, when a subject is identified as having an increased risk of developing a kidney disease, the subject is further administered a therapeutic agent that treats, prevents, or inhibits a kidney disease, and/or an RPL3L inhibitor, as described herein. For example, when the subject is RPL3L reference, and therefore has an increased risk of developing a kidney disease, the subject is administered an RPL3L inhibitor. In some embodiments, such a subject is also administered a therapeutic agent that treats, prevents, or inhibits a kidney disease. In some embodiments, when the subject is heterozygous for an RPL3L variant nucleic acid molecule encoding an RPL3L predicted loss-of-function polypeptide, the subject is administered the therapeutic agent that treats, prevents, or inhibits a kidney disease in a dosage amount that is the same as or less than a standard dosage amount, and is also administered an RPL3L inhibitor. In some embodiments, the subject is RPL3L reference. In some embodiments, the subject is heterozygous for an RPL3L variant nucleic acid molecule encoding an RPL3L predicted loss-of-function polypeptide. Furthermore, when the subject has a lower aggregate gene burden for having an RPL3L variant nucleic acid molecule encoding an RPL3L predicted loss-of-function polypeptide, and therefore has an increased risk of developing a kidney disease, the subject is administered a therapeutic agent that treats, prevents, or inhibits a kidney disease. In some embodiments, when the subject has a lower aggregate gene burden for having an RPL3L variant nucleic acid molecule encoding an RPL3L predicted loss-of-function polypeptide, the subject is administered the therapeutic agent that treats, prevents, or inhibits a kidney disease in a dosage amount that is the same as or less than the standard dosage amount administered to a subject who has a greater aggregate gene burden for having an RPL3L variant nucleic acid molecule encoding an RPL3L predicted loss-of-function polypeptide.

In some embodiments, when a subject is identified as having an increased risk of developing a kidney disease, the subject is further administered a therapeutic agent that treats, prevents, or inhibits a kidney disease, and/or an SLC25A45 inhibitor, as described herein. For example, when the subject is SLC25A45 reference, and therefore has an increased risk of developing a kidney disease, the subject is administered an SLC25A45 inhibitor. In some embodiments, such a subject is also administered a therapeutic agent that treats, prevents, or inhibits a kidney disease. In some embodiments, when the subject is heterozygous for an SLC25A45 variant nucleic acid molecule encoding an SLC25A45 predicted loss-of-function polypeptide, the subject is administered the therapeutic agent that treats, prevents, or inhibits a kidney disease in a dosage amount that is the same as or less than a standard dosage amount, and is also administered an SLC25A45 inhibitor. In some embodiments, the subject is SLC25A45 reference. In some embodiments, the subject is heterozygous for an SLC25A45 variant nucleic acid molecule encoding an SLC25A45 predicted loss-of-function polypeptide. Furthermore, when the subject has a lower aggregate gene burden for having an SLC25A45 variant nucleic acid molecule encoding an SLC25A45 predicted loss-of-function polypeptide, and therefore has an increased risk of developing a kidney disease, the subject is administered a therapeutic agent that treats, prevents, or inhibits a kidney disease. In some embodiments, when the subject has a lower aggregate gene burden for having an SLC25A45 variant nucleic acid molecule encoding an SLC25A45 predicted loss-of-function polypeptide, the subject is administered the therapeutic agent that treats, prevents, or inhibits a kidney disease in a dosage amount that is the same as or less than the standard dosage amount administered to a subject who has a greater aggregate gene burden for having an SLC25A45 variant nucleic acid molecule encoding an SLC25A45 predicted loss-of-function polypeptide.

In some embodiments, when a subject is identified as having an increased risk of developing a kidney disease, the subject is further administered a therapeutic agent that treats, prevents, or inhibits a kidney disease, and/or an SLC7A9 inhibitor, as described herein. For example, when the subject is SLC7A9 reference, and therefore has an increased risk of developing a kidney disease, the subject is administered an SLC7A9 inhibitor. In some embodiments, such a subject is also administered a therapeutic agent that treats, prevents, or inhibits a kidney disease. In some embodiments, when the subject is heterozygous for an SLC7A9 variant nucleic acid molecule encoding an SLC7A9 predicted loss-of-function polypeptide, the subject is administered the therapeutic agent that treats, prevents, or inhibits a kidney disease in a dosage amount that is the same as or less than a standard dosage amount, and is also administered an SLC7A9 inhibitor. In some embodiments, the subject is SLC7A9 reference. In some embodiments, the subject is heterozygous for an SLC7A9 variant nucleic acid molecule encoding an SLC7A9 predicted loss-of-function polypeptide. Furthermore, when the subject has a lower aggregate gene burden for having an SLC7A9 variant nucleic acid molecule encoding an SLC7A9 predicted loss-of-function polypeptide, and therefore has an increased risk of developing a kidney disease, the subject is administered a therapeutic agent that treats, prevents, or inhibits a kidney disease. In some embodiments, when the subject has a lower aggregate gene burden for having an SLC7A9 variant nucleic acid molecule encoding an SLC7A9 predicted loss-of-function polypeptide, the subject is administered the therapeutic agent that treats, prevents, or inhibits a kidney disease in a dosage amount that is the same as or less than the standard dosage amount administered to a subject who has a greater aggregate gene burden for having an SLC7A9 variant nucleic acid molecule encoding an SLC7A9 predicted loss-of-function polypeptide.

In some embodiments, when the subject has a lower aggregate gene burden for having one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides, and therefore has an increased risk of developing a kidney disease, the subject is administered a therapeutic agent that treats, prevents, or inhibits a kidney disease. In some embodiments, when the subject has a lower aggregate gene burden for having one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides, the therapeutic agent that prevents a kidney disease is administered or continued to be administered to the subject in an amount that is the same as or less than the standard dosage amount administered to a subject who has a greater aggregate gene burden for having one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides.

In addition, the subject may be administered a combination of any one or more ALDH1L1 inhibitors, ALDOB inhibitors, G6PC inhibitors, LRP2 inhibitors, RPL3L inhibitors, SLC25A45 inhibitors, and/or SLC7A9 inhibitors, wherein the combination comprises the inhibitors for the targets for which the the lower gene burden for having one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides. For example, when the subject is determined to have lower gene burden of ALDOB, G6PC, RPL3L, and SLC7A9 variant nucleic acid molecules encoding ALDOB, G6PC, RPL3L, and SLC7A9 predicted loss-of-function polypeptides, the subject is is administered or continued to be administered the therapeutic agent that treats, prevents, or inhibits a kidney disease in an amount that is the same as or less than a standard dosage amount and is administered a combination of an ALDOB inhibitor, a G6PC inhibitor, a RPL3L inhibitor, and an SLC7A9 inhibitor. In some embodiments, the subject is administered the combination of inhibitors that corresponds to any subset of ALDH1L1, ALDOB, G6PC, LRP2, RPL3L, SLC25A45, and/or SLC7A9 for which the subject was found to have a lower gene burden of variant nucleic acid molecule encoding corresponding loss-of-function polypeptides. The presence of a lower gene burden of any one or more ALDH1L1, ALDOB, G6PC, LRP2, RPL3L, SLC25A45, and/or SLC7A9 predicted loss-of-function polypeptides indicates the subject has a increased risk of developing a kidney disease. The presence of a greater gene burden of any one or more of ALDH1L1, ALDOB, G6PC, LRP2, RPL3L, SLC25A45, and/or SLC7A9 predicted loss-of-function polypeptides is considered more protective of kidney diseases. Thus, having a greater gene burden of any four of ALDH1L1, ALDOB, G6PC, LRP2, RPL3L, SLC25A45, and SLC7A9 predicted loss-of-function polypeptides is more protective than having a gene burden of any three of ALDH1L1, ALDOB, G6PC, LRP2, RPL3L, SLC25A45, and SLC7A9 predicted loss-of-function polypeptides, which is, in turn, more protective than having a gene burden of any two of ALDH1L1, ALDOB, G6PC, LRP2, RPL3L, SLC25A45, and SLC7A9 predicted loss-of-function polypeptides.

In any of the embodiments described herein the individual aggregate gene burdens for any combination of ALDH1L1, ALDOB, G6PC, LRP2, RPL3L, SLC25A45, and SLC7A9 can be combined into a single aggregate gene burden.

The present disclosure also provides methods of detecting the presence or absence of any one or more of ALDH1L1, ALDOB, G6PC, LRP2, RPL3L, SLC25A45, and/or SLC7A9 variant nucleic acid molecules (i.e., genomic nucleic acid molecules, mRNA molecules, or cDNA molecules produced from an mRNA molecucles) encoding any one or more of ALDH1L1, ALDOB, G6PC, LRP2, RPL3L, SLC25A45, and/or SLC7A9 predicted loss-of-function polypeptides in a biological sample from a subject. It is understood that gene sequences within a population and mRNA molecules encoded by such genes can vary due to polymorphisms such as single-nucleotide polymorphisms. The sequences provided herein for the ALDH1L1, ALDOB, G6PC, LRP2, RPL3L, SLC25A45, and SLC7A9 variant genomic nucleic acid molecules, ALDH1L1, ALDOB, G6PC, LRP2, RPL3L, SLC25A45, and SLC7A9 variant mRNA molecules, and ALDH1L1, ALDOB, G6PC, LRP2, RPL3L, SLC25A45, and SLC7A9 variant cDNA molecules are only exemplary sequences. Other sequences for the ALDH1L1, ALDOB, G6PC, LRP2, RPL3L, SLC25A45, and SLC7A9 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any one or more of ALDH1L1, ALDOB, G6PC, LRP2, RPL3L, SLC25A45, and/or SLC7A9 variant nucleic acid molecules encoding one or more of ALDH1L1, ALDOB, G6PC, LRP2, RPL3L, SLC25A45, and/or SLC7A9 predicted loss-of-function polypeptides, preliminary processing designed to isolate or enrich the biological sample for the genomic DNA can be employed. A variety of techniques may be used for this purpose. When detecting the level of any one or more of ALDH1L1, ALDOB, G6PC, LRP2, RPL3L, SLC25A45, and/or SLC7A9 variant mRNA molecules, different techniques can be used enrich the biological sample with mRNA molecules. Various methods to detect the presence or level of an mRNA molecule or the presence of a particular variant genomic DNA locus can be used.

In some embodiments, detecting an ALDH1L1 variant nucleic acid molecule encoding an ALDH1L1 predicted loss-of-function polypeptide in a subject comprises performing a sequence analysis on a biological sample obtained from the subject to determine whether an ALDH1L1 genomic nucleic acid molecule in the biological sample, and/or an ALDH1L1 mRNA molecule in the biological sample, and/or an ALDH1L1 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an ALDH1L1 variant nucleic acid molecule encoding an ALDH1L1 predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an ALDH1L1 genomic nucleic acid molecule or mRNA molecule, and if mRNA, optionally reverse transcribing the mRNA into cDNA. Such assays can comprise, for example determining the identity of these positions of the particular ALDH1L1 nucleic acid molecule. In some embodiments, the method is an in vitro method.

In some embodiments, the determining step, detecting step, or sequence analysis comprises sequencing at least a portion of the nucleotide sequence of the ALDH1L1 genomic nucleic acid molecule, the ALDH1L1 mRNA molecule, or the ALDH1L1 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an ALDH1L1 genomic nucleic acid molecule is analyzed. In some embodiments, only an ALDH1L1 mRNA is analyzed. In some embodiments, only an ALDH1L1 cDNA obtained from ALDH1L1 mRNA is analyzed.

In some embodiments, detecting an ALDOB variant nucleic acid molecule encoding an ALDOB predicted loss-of-function polypeptide in a subject comprises performing a sequence analysis on a biological sample obtained from the subject to determine whether an ALDOB genomic nucleic acid molecule in the biological sample, and/or an ALDOB mRNA molecule in the biological sample, and/or an ALDOB cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an ALDOB variant nucleic acid molecule encoding an ALDOB predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an ALDOB genomic nucleic acid molecule or mRNA molecule, and if mRNA, optionally reverse transcribing the mRNA into cDNA. Such assays can comprise, for example determining the identity of these positions of the particular ALDOB nucleic acid molecule. In some embodiments, the method is an in vitro method.

In some embodiments, the determining step, detecting step, or sequence analysis comprises sequencing at least a portion of the nucleotide sequence of the ALDOB genomic nucleic acid molecule, the ALDOB mRNA molecule, or the ALDOB cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an ALDOB genomic nucleic acid molecule is analyzed. In some embodiments, only an ALDOB mRNA is analyzed. In some embodiments, only an ALDOB cDNA obtained from ALDOB mRNA is analyzed.

In some embodiments, detecting a G6PC variant nucleic acid molecule encoding a G6PC predicted loss-of-function polypeptide in a subject comprises performing a sequence analysis on a biological sample obtained from the subject to determine whether a G6PC genomic nucleic acid molecule in the biological sample, and/or a G6PC mRNA molecule in the biological sample, and/or a G6PC cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of a G6PC variant nucleic acid molecule encoding a G6PC predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a G6PC genomic nucleic acid molecule or mRNA molecule, and if mRNA, optionally reverse transcribing the mRNA into cDNA. Such assays can comprise, for example determining the identity of these positions of the particular G6PC nucleic acid molecule. In some embodiments, the method is an in vitro method.

In some embodiments, the determining step, detecting step, or sequence analysis comprises sequencing at least a portion of the nucleotide sequence of the G6PC genomic nucleic acid molecule, the G6PC mRNA molecule, or the G6PC cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only a G6PC genomic nucleic acid molecule is analyzed. In some embodiments, only a G6PC mRNA is analyzed. In some embodiments, only a G6PC cDNA obtained from G6PC mRNA is analyzed.

In some embodiments, detecting an LRP2 variant nucleic acid molecule encoding an LRP2 predicted loss-of-function polypeptide in a subject comprises performing a sequence analysis on a biological sample obtained from the subject to determine whether an LRP2 genomic nucleic acid molecule in the biological sample, and/or an LRP2 mRNA molecule in the biological sample, and/or an LRP2 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an LRP2 variant nucleic acid molecule encoding an LRP2 predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an LRP2 genomic nucleic acid molecule or mRNA molecule, and if mRNA, optionally reverse transcribing the mRNA into cDNA. Such assays can comprise, for example determining the identity of these positions of the particular LRP2 nucleic acid molecule. In some embodiments, the method is an in vitro method.

In some embodiments, the determining step, detecting step, or sequence analysis comprises sequencing at least a portion of the nucleotide sequence of the LRP2 genomic nucleic acid molecule, the LRP2 mRNA molecule, or the LRP2 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an LRP2 genomic nucleic acid molecule is analyzed. In some embodiments, only an LRP2 mRNA is analyzed. In some embodiments, only an LRP2 cDNA obtained from LRP2 mRNA is analyzed.

In some embodiments, detecting an RPL3L variant nucleic acid molecule encoding an RPL3L predicted loss-of-function polypeptide in a subject comprises performing a sequence analysis on a biological sample obtained from the subject to determine whether an RPL3L genomic nucleic acid molecule in the biological sample, and/or an RPL3L mRNA molecule in the biological sample, and/or an RPL3L cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an RPL3L variant nucleic acid molecule encoding an RPL3L predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an RPL3L genomic nucleic acid molecule or mRNA molecule, and if mRNA, optionally reverse transcribing the mRNA into cDNA. Such assays can comprise, for example determining the identity of these positions of the particular RPL3L nucleic acid molecule. In some embodiments, the method is an in vitro method.

In some embodiments, the determining step, detecting step, or sequence analysis comprises sequencing at least a portion of the nucleotide sequence of the RPL3L genomic nucleic acid molecule, the RPL3L mRNA molecule, or the RPL3L cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an RPL3L genomic nucleic acid molecule is analyzed. In some embodiments, only an RPL3L mRNA is analyzed. In some embodiments, only an RPL3L cDNA obtained from RPL3L mRNA is analyzed.

In some embodiments, detecting an SLC25A45 variant nucleic acid molecule encoding an SLC25A45 predicted loss-of-function polypeptide in a subject comprises performing a sequence analysis on a biological sample obtained from the subject to determine whether an SLC25A45 genomic nucleic acid molecule in the biological sample, and/or an SLC25A45 mRNA molecule in the biological sample, and/or an SLC25A45 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an SLC25A45 variant nucleic acid molecule encoding an SLC25A45 predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an SLC25A45 genomic nucleic acid molecule or mRNA molecule, and if mRNA, optionally reverse transcribing the mRNA into cDNA. Such assays can comprise, for example determining the identity of these positions of the particular SLC25A45 nucleic acid molecule. In some embodiments, the method is an in vitro method.

In some embodiments, the determining step, detecting step, or sequence analysis comprises sequencing at least a portion of the nucleotide sequence of the SLC25A45 genomic nucleic acid molecule, the SLC25A45 mRNA molecule, or the SLC25A45 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an SLC25A45 genomic nucleic acid molecule is analyzed. In some embodiments, only an SLC25A45 mRNA is analyzed. In some embodiments, only an SLC25A45 cDNA obtained from SLC25A45 mRNA is analyzed.

In some embodiments, detecting an SLC7A9 variant nucleic acid molecule encoding an SLC7A9 predicted loss-of-function polypeptide in a subject comprises performing a sequence analysis on a biological sample obtained from the subject to determine whether an SLC7A9 genomic nucleic acid molecule in the biological sample, and/or an SLC7A9 mRNA molecule in the biological sample, and/or an SLC7A9 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an SLC7A9 variant nucleic acid molecule encoding an SLC7A9 predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an SLC7A9 genomic nucleic acid molecule or mRNA molecule, and if mRNA, optionally reverse transcribing the mRNA into cDNA. Such assays can comprise, for example determining the identity of these positions of the particular SLC7A9 nucleic acid molecule. In some embodiments, the method is an in vitro method.

In some embodiments, the determining step, detecting step, or sequence analysis comprises sequencing at least a portion of the nucleotide sequence of the SLC7A9 genomic nucleic acid molecule, the SLC7A9 mRNA molecule, or the SLC7A9 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an SLC7A9 genomic nucleic acid molecule is analyzed. In some embodiments, only an SLC7A9 mRNA is analyzed. In some embodiments, only an SLC7A9 cDNA obtained from SLC7A9 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an ALDH1L1 variant genomic sequence, variant mRNA sequence, or variant cDNA sequence and not the corresponding ALDH1L1 reference sequence under stringent conditions, and determining whether hybridization has occurred.

In some embodiments, the determining step, detecting step, or sequence analysis comprises: a) amplifying at least a portion of the nucleic acid molecule that encodes the ALDH1L1 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises contacting the biological sample with a primer or probe, such as an alteration-specific primer or alteration-specific probe, that specifically hybridizes to an ALDOB variant genomic sequence, variant mRNA sequence, or variant cDNA sequence and not the corresponding ALDOB reference sequence under stringent conditions, and determining whether hybridization has occurred.

In some embodiments, the determining step, detecting step, or sequence analysis comprises: a) amplifying at least a portion of the nucleic acid molecule that encodes the ALDOB polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises contacting the biological sample with a primer or probe, such as an alteration-specific primer or alteration-specific probe, that specifically hybridizes to a G6PC variant genomic sequence, variant mRNA sequence, or variant cDNA sequence and not the corresponding G6PC reference sequence under stringent conditions, and determining whether hybridization has occurred.

In some embodiments, the determining step, detecting step, or sequence analysis comprises: a) amplifying at least a portion of the nucleic acid molecule that encodes the G6PC polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises contacting the biological sample with a primer or probe, such as an alteration-specific primer or alteration-specific probe, that specifically hybridizes to an LRP2 variant genomic sequence, variant mRNA sequence, or variant cDNA sequence and not the corresponding LRP2 reference sequence under stringent conditions, and determining whether hybridization has occurred.

In some embodiments, the determining step, detecting step, or sequence analysis comprises: a) amplifying at least a portion of the nucleic acid molecule that encodes the LRP2 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises contacting the biological sample with a primer or probe, such as an alteration-specific primer or alteration-specific probe, that specifically hybridizes to an RPL3L variant genomic sequence, variant mRNA sequence, or variant cDNA sequence and not the corresponding RPL3L reference sequence under stringent conditions, and determining whether hybridization has occurred.

In some embodiments, the determining step, detecting step, or sequence analysis comprises: a) amplifying at least a portion of the nucleic acid molecule that encodes the RPL3L polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises contacting the biological sample with a primer or probe, such as an alteration-specific primer or alteration-specific probe, that specifically hybridizes to an SLC25A45 variant genomic sequence, variant mRNA sequence, or variant cDNA sequence and not the corresponding SLC25A45 reference sequence under stringent conditions, and determining whether hybridization has occurred.

In some embodiments, the determining step, detecting step, or sequence analysis comprises: a) amplifying at least a portion of the nucleic acid molecule that encodes the SLC25A45 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises contacting the biological sample with a primer or probe, such as an alteration-specific primer or alteration-specific probe, that specifically hybridizes to an SLC7A9 variant genomic sequence, variant mRNA sequence, or variant cDNA sequence and not the corresponding SLC7A9 reference sequence under stringent conditions, and determining whether hybridization has occurred.

In some embodiments, the determining step, detecting step, or sequence analysis comprises: a) amplifying at least a portion of the nucleic acid molecule that encodes the SLC7A9 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an ALDH1L1 variant genomic nucleic acid molecule, variant mRNA molecule, or variant cDNA molecule, an ALDOB variant genomic nucleic acid molecule, variant mRNA molecule, or variant cDNA molecule, a G6PC variant genomic nucleic acid molecule, variant mRNA molecule, or variant cDNA molecule, an LRP2 variant genomic nucleic acid molecule, variant mRNA molecule, or variant cDNA molecule, an RPL3L variant genomic nucleic acid molecule, variant mRNA molecule, or variant cDNA molecule, an SLC25A45 variant genomic nucleic acid molecule, variant mRNA molecule, or variant cDNA molecule, an SLC7A9 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6×sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to ALDH1L1, ALDOB, G6PC, LRP2, RPL3L, SLC25A45, or SLC7A9 variant nucleic acid molecule (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to ALDH1L1, ALDOB, G6PC, LRP2, RPL3L, SLC25A45, or SLC7A9 variant genomic nucleic acid molecules, ALDH1L1, ALDOB, G6PC, LRP2, RPL3L, SLC25A45, or SLC7A9 variant mRNA molecules, and/or ALDH1L1, ALDOB, G6PC, LRP2, RPL3L, SLC25A45, or SLC7A9 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the ALDH1L1, ALDOB, G6PC, LRP2, RPL3L, SLC25A45, or SLC7A9 variant genomic nucleic acid molecules, ALDH1L1, ALDOB, G6PC, LRP2, RPL3L, SLC25A45, or SLC7A9 variant mRNA molecules, and/or ALDH1L1, ALDOB, G6PC, LRP2, RPL3L, SLC25A45, or SLC7A9 variant cDNA molecules disclosed herein. The primers described herein can be used to amplify ALDH1L1, ALDOB, G6PC, LRP2, RPL3L, SLC25A45, or SLC7A9 variant genomic nucleic acid molecules, ALDH1L1, ALDOB, G6PC, LRP2, RPL3L, SLC25A45, or SLC7A9 variant mRNA molecules, or ALDH1L1, ALDOB, G6PC, LRP2, RPL3L, SLC25A45, or SLC7A9 variant cDNA molecules, or a fragment thereof.

In the context of the disclosure “specifically hybridizes” means that the probe or primer (such as, for example, the alteration-specific probe or alteration-specific primer) does not hybridize to a nucleic acid sequence encoding an ALDH1L1 reference genomic nucleic acid molecule, an ALDH1L1 reference mRNA molecule, and/or an ALDH1L1 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of an ALDH1L1 reference genomic nucleic acid molecule is set forth in SEQ ID NO:1 (encompassing chr3:126,103,562-126,180,802 in the GRCh38/hg38 human genome assembly).

The nucleotide sequence of an ALDH1L1 reference mRNA molecule is set forth in SEQ ID NO:2. The nucleotide sequence of another ALDH1L1 reference mRNA molecule is set forth in SEQ ID NO:3. The nucleotide sequence of another ALDH1L1 reference mRNA molecule is set forth in SEQ ID NO:4. The nucleotide sequence of another ALDH1L1 reference mRNA molecule is set forth in SEQ ID NO:5. The nucleotide sequence of another ALDH1L1 reference mRNA molecule is set forth in SEQ ID NO:6. The nucleotide sequence of another ALDH1L1 reference mRNA molecule is set forth in SEQ ID NO:7. The nucleotide sequence of another ALDH1L1 reference mRNA molecule is set forth in SEQ ID NO:8. The nucleotide sequence of another ALDH1L1 reference mRNA molecule is set forth in SEQ ID NO:9. The nucleotide sequence of another ALDH1L1 reference mRNA molecule is set forth in SEQ ID NO:10. The nucleotide sequence of another ALDH1L1 reference mRNA molecule is set forth in SEQ ID NO:11. The nucleotide sequence of another ALDH1L1 reference mRNA molecule is set forth in SEQ ID NO:12. The nucleotide sequence of another ALDH1L1 reference mRNA molecule is set forth in SEQ ID NO:13. The nucleotide sequence of another ALDH1L1 reference mRNA molecule is set forth in SEQ ID NO:14. The nucleotide sequence of another ALDH1L1 reference mRNA molecule is set forth in SEQ ID NO:15. The nucleotide sequence of another ALDH1L1 reference mRNA molecule is set forth in SEQ ID NO:16. The nucleotide sequence of another ALDH1L1 reference mRNA molecule is set forth in SEQ ID NO:17. The nucleotide sequence of another ALDH1L1 reference mRNA molecule is set forth in SEQ ID NO:18. The nucleotide sequence of another ALDH1L1 reference mRNA molecule is set forth in SEQ ID NO:19. The nucleotide sequence of another ALDH1L1 reference mRNA molecule is set forth in SEQ ID NO:20. The nucleotide sequence of another ALDH1L1 reference mRNA molecule is set forth in SEQ ID NO:21. The nucleotide sequence of another ALDH1L1 reference mRNA molecule is set forth in SEQ ID NO:22. The nucleotide sequence of another ALDH1L1 reference mRNA molecule is set forth in SEQ ID NO:23. The nucleotide sequence of another ALDH1L1 reference mRNA molecule is set forth in SEQ ID NO:24. The nucleotide sequence of another ALDH1L1 reference mRNA molecule is set forth in SEQ ID NO:25. The nucleotide sequence of another ALDH1L1 reference mRNA molecule is set forth in SEQ ID NO:26. The nucleotide sequence of another ALDH1L1 reference mRNA molecule is set forth in SEQ ID NO:27. The nucleotide sequence of another ALDH1L1 reference mRNA molecule is set forth in SEQ ID NO:28.

The nucleotide sequence of an ALDH1L1 reference cDNA molecule is set forth in SEQ ID NO:29. The nucleotide sequence of another ALDH1L1 reference cDNA molecule is set forth in SEQ ID NO:30. The nucleotide sequence of another ALDH1L1 reference cDNA molecule is set forth in SEQ ID NO:31. The nucleotide sequence of another ALDH1L1 reference cDNA molecule is set forth in SEQ ID NO:32. The nucleotide sequence of another ALDH1L1 reference cDNA molecule is set forth in SEQ ID NO:33. The nucleotide sequence of another ALDH1L1 reference cDNA molecule is set forth in SEQ ID NO:34. The nucleotide sequence of another ALDH1L1 reference cDNA molecule is set forth in SEQ ID NO:35. The nucleotide sequence of another ALDH1L1 reference cDNA molecule is set forth in SEQ ID NO:36. The nucleotide sequence of another ALDH1L1 reference cDNA molecule is set forth in SEQ ID NO:37. The nucleotide sequence of another ALDH1L1 reference cDNA molecule is set forth in SEQ ID NO:38. The nucleotide sequence of another ALDH1L1 reference cDNA molecule is set forth in SEQ ID NO:39. The nucleotide sequence of another ALDH1L1 reference cDNA molecule is set forth in SEQ ID NO:40. The nucleotide sequence of another ALDH1L1 reference cDNA molecule is set forth in SEQ ID NO:41. The nucleotide sequence of another ALDH1L1 reference cDNA molecule is set forth in SEQ ID NO:42. The nucleotide sequence of another ALDH1L1 reference cDNA molecule is set forth in SEQ ID NO:43. The nucleotide sequence of another ALDH1L1 reference cDNA molecule is set forth in SEQ ID NO:44. The nucleotide sequence of another ALDH1L1 reference cDNA molecule is set forth in SEQ ID NO:45. The nucleotide sequence of another ALDH1L1 reference cDNA molecule is set forth in SEQ ID NO:46. The nucleotide sequence of another ALDH1L1 reference cDNA molecule is set forth in SEQ ID NO:47. The nucleotide sequence of another ALDH1L1 reference cDNA molecule is set forth in SEQ ID NO:48. The nucleotide sequence of another ALDH1L1 reference cDNA molecule is set forth in SEQ ID NO:49. The nucleotide sequence of another ALDH1L1 reference cDNA molecule is set forth in SEQ ID NO:51. The nucleotide sequence of another ALDH1L1 reference cDNA molecule is set forth in SEQ ID NO:52. The nucleotide sequence of another ALDH1L1 reference cDNA molecule is set forth in SEQ ID NO:53. The nucleotide sequence of another ALDH1L1 reference cDNA molecule is set forth in SEQ ID NO:54. The nucleotide sequence of another ALDH1L1 reference cDNA molecule is set forth in SEQ ID NO:55. The nucleotide sequence of another ALDH1L1 reference cDNA molecule is set forth in SEQ ID NO:56. The nucleotide sequence of another ALDH1L1 reference cDNA molecule is set forth in SEQ ID NO:57.

The amino acid sequence of an ALDH1L1 reference polypeptide is set forth in SEQ ID NO:58, and is 505 amino acids in length. The amino acid sequence of another ALDH1L1 reference polypeptide is set forth in SEQ ID NO:59, and is 912 amino acids in length. The amino acid sequence of another ALDH1L1 reference polypeptide is set forth in SEQ ID NO:60, and is 902 amino acids in length. The amino acid sequence of another ALDH1L1 reference polypeptide is set forth in SEQ ID NO:61, and is 801 amino acids in length. The amino acid sequence of another ALDH1L1 reference polypeptide is set forth in SEQ ID NO:62, and is 470 amino acids in length. The amino acid sequence of another ALDH1L1 reference polypeptide is set forth in SEQ ID NO:63, and is 954 amino acids in length. The amino acid sequence of another ALDH1L1 reference polypeptide is set forth in SEQ ID NO:64, and is 555 amino acids in length. The amino acid sequence of another ALDH1L1 reference polypeptide is set forth in SEQ ID NO:65, and is 240 amino acids in length. The amino acid sequence of another ALDH1L1 reference polypeptide is set forth in SEQ ID NO:66, and is 160 amino acids in length. The amino acid sequence of another ALDH1L1 reference polypeptide is set forth in SEQ ID NO:67, and is 210 amino acids in length. The amino acid sequence of another ALDH1L1 reference polypeptide is set forth in SEQ ID NO:68, and is 47 amino acids in length. The amino acid sequence of another ALDH1L1 reference polypeptide is set forth in SEQ ID NO:69, and is 177 amino acids in length. The amino acid sequence of another ALDH1L1 reference polypeptide is set forth in SEQ ID NO:70, and is 168 amino acids in length. The amino acid sequence of another ALDH1L1 reference polypeptide is set forth in SEQ ID NO:71, and is 157 amino acids in length. The amino acid sequence of another ALDH1L1 reference polypeptide is set forth in SEQ ID NO:72, and is 100 amino acids in length.

The nucleotide sequence of an ALDOB reference genomic nucleic acid molecule is set forth in SEQ ID NO:73 (encompassing chr9:101,421,439-101,449,664 in the GRCh38/hg38 human genome assembly).

The nucleotide sequence of an ALDOB reference mRNA molecule is set forth in SEQ ID NO:74. The nucleotide sequence of another ALDOB reference mRNA molecule is set forth in SEQ ID NO:75. The nucleotide sequence of another ALDOB reference mRNA molecule is set forth in SEQ ID NO:76. The nucleotide sequence of another ALDOB reference mRNA molecule is set forth in SEQ ID NO:77. The nucleotide sequence of another ALDOB reference mRNA molecule is set forth in SEQ ID NO:78. The nucleotide sequence of another ALDOB reference mRNA molecule is set forth in SEQ ID NO:79. The nucleotide sequence of another ALDOB reference mRNA molecule is set forth in SEQ ID NO:80. The nucleotide sequence of another ALDOB reference mRNA molecule is set forth in SEQ ID NO:81. The nucleotide sequence of another ALDOB reference mRNA molecule is set forth in SEQ ID NO:82. The nucleotide sequence of another ALDOB reference mRNA molecule is set forth in SEQ ID NO:83. The nucleotide sequence of another ALDOB reference mRNA molecule is set forth in SEQ ID NO:84. The nucleotide sequence of another ALDOB reference mRNA molecule is set forth in SEQ ID NO:85.

The nucleotide sequence of an ALDOB reference cDNA molecule is set forth in SEQ ID NO:86. The nucleotide sequence of another ALDOB reference cDNA molecule is set forth in SEQ ID NO:87. The nucleotide sequence of another ALDOB reference cDNA molecule is set forth in SEQ ID NO:88. The nucleotide sequence of another ALDOB reference cDNA molecule is set forth in SEQ ID NO:89. The nucleotide sequence of another ALDOB reference cDNA molecule is set forth in SEQ ID NO:90. The nucleotide sequence of another ALDOB reference cDNA molecule is set forth in SEQ ID NO:91. The nucleotide sequence of another ALDOB reference cDNA molecule is set forth in SEQ ID NO:92. The nucleotide sequence of another ALDOB reference cDNA molecule is set forth in SEQ ID NO:93. The nucleotide sequence of another ALDOB reference cDNA molecule is set forth in SEQ ID NO:94. The nucleotide sequence of another ALDOB reference cDNA molecule is set forth in SEQ ID NO:95. The nucleotide sequence of another ALDOB reference cDNA molecule is set forth in SEQ ID NO:96. The nucleotide sequence of another ALDOB reference cDNA molecule is set forth in SEQ ID NO:97.

The amino acid sequence of an ALDOB reference polypeptide is set forth in SEQ ID NO:98, and is 364 amino acids in length. The amino acid sequence of another ALDOB reference polypeptide is set forth in SEQ ID NO:99, and is 316 amino acids in length. The amino acid sequence of another ALDOB reference polypeptide is set forth in SEQ ID NO:100, and is 342 amino acids in length. The amino acid sequence of another ALDOB reference polypeptide is set forth in SEQ ID NO:101, and is 127 amino acids in length

The nucleotide sequence of a G6PC reference genomic nucleic acid molecule is set forth in SEQ ID NO:102 (encompassing chr17:42,900,799-42,914,438 in the GRCh38/hg38 human genome assembly).

The nucleotide sequence of a G6PC reference mRNA molecule is set forth in SEQ ID NO:103. The nucleotide sequence of another G6PC reference mRNA molecule is set forth in SEQ ID NO:104. The nucleotide sequence of another G6PC reference mRNA molecule is set forth in SEQ ID NO:105. The nucleotide sequence of another G6PC reference mRNA molecule is set forth in SEQ ID NO:106. The nucleotide sequence of another G6PC reference mRNA molecule is set forth in SEQ ID NO:107. The nucleotide sequence of another G6PC reference mRNA molecule is set forth in SEQ ID NO:108. The nucleotide sequence of another G6PC reference mRNA molecule is set forth in SEQ ID NO:109.

The nucleotide sequence of a G6PC reference cDNA molecule is set forth in SEQ ID NO:110. The nucleotide sequence of another G6PC reference cDNA molecule is set forth in SEQ ID NO:111. The nucleotide sequence of another G6PC reference cDNA molecule is set forth in SEQ ID NO:112. The nucleotide sequence of another G6PC reference cDNA molecule is set forth in SEQ ID NO:113. The nucleotide sequence of another G6PC reference cDNA molecule is set forth in SEQ ID NO:114. The nucleotide sequence of another G6PC reference cDNA molecule is set forth in SEQ ID NO:115. The nucleotide sequence of another G6PC reference cDNA molecule is set forth in SEQ ID NO:116.

The amino acid sequence of a G6PC reference polypeptide is set forth in SEQ ID NO:117, and is 357 amino acids in length. The amino acid sequence of another G6PC reference polypeptide is set forth in SEQ ID NO:118, and is 176 amino acids in length. The amino acid sequence of another G6PC reference polypeptide is set forth in SEQ ID NO:119, and is 163 amino acids in length.

The nucleotide sequence of an LRP2 reference genomic nucleic acid molecule is set forth in SEQ ID NO:120 (encompassing chr2:169,127,109-169,362,534 in the GRCh38/hg38 human genome assembly).

The nucleotide sequence of an LRP2 reference mRNA molecule is set forth in SEQ ID NO:121. The nucleotide sequence of another LRP2 reference mRNA molecule is set forth in SEQ ID NO:122. The nucleotide sequence of another LRP2 reference mRNA molecule is set forth in SEQ ID NO:123. The nucleotide sequence of another LRP2 reference mRNA molecule is set forth in SEQ ID NO:124. The nucleotide sequence of another LRP2 reference mRNA molecule is set forth in SEQ ID NO:125. The nucleotide sequence of another LRP2 reference mRNA molecule is set forth in SEQ ID NO:126. The nucleotide sequence of another LRP2 reference mRNA molecule is set forth in SEQ ID NO:127. The nucleotide sequence of another LRP2 reference mRNA molecule is set forth in SEQ ID NO:128. The nucleotide sequence of another LRP2 reference mRNA molecule is set forth in SEQ ID NO:129.

The nucleotide sequence of an LRP2 reference cDNA molecule is set forth in SEQ ID NO:130. The nucleotide sequence of another LRP2 reference cDNA molecule is set forth in SEQ ID NO:131. The nucleotide sequence of another LRP2 reference cDNA molecule is set forth in SEQ ID NO:132. The nucleotide sequence of another LRP2 reference cDNA molecule is set forth in SEQ ID NO:133. The nucleotide sequence of another LRP2 reference cDNA molecule is set forth in SEQ ID NO:134. The nucleotide sequence of another LRP2 reference cDNA molecule is set forth in SEQ ID NO:135. The nucleotide sequence of another LRP2 reference cDNA molecule is set forth in SEQ ID NO:136. The nucleotide sequence of another LRP2 reference cDNA molecule is set forth in SEQ ID NO:137. The nucleotide sequence of another LRP2 reference cDNA molecule is set forth in SEQ ID NO:138.

The amino acid sequence of an LRP2 reference polypeptide is set forth in SEQ ID NO:139, and is 4,655 amino acids in length. The amino acid sequence of another LRP2 reference polypeptide is set forth in SEQ ID NO:140, and is 1,255 amino acids in length. The amino acid sequence of another LRP2 reference polypeptide is set forth in SEQ ID NO:141, and is 1,751 amino acids in length. The amino acid sequence of another LRP2 reference polypeptide is set forth in SEQ ID NO:142, and is 268 amino acids in length. The amino acid sequence of another LRP2 reference polypeptide is set forth in SEQ ID NO:143, and is 646 amino acids in length.

The nucleotide sequence of an RPL3L reference genomic nucleic acid molecule is set forth in SEQ ID NO:144 (encompassing chr16:1,943,974-1,954,689 in the GRCh38/hg38 human genome assembly).

The nucleotide sequence of an RPL3L reference mRNA molecule is set forth in SEQ ID NO:145. The nucleotide sequence of another RPL3L reference mRNA molecule is set forth in SEQ ID NO:146. The nucleotide sequence of another RPL3L reference mRNA molecule is set forth in SEQ ID NO:147. The nucleotide sequence of another RPL3L reference mRNA molecule is set forth in SEQ ID NO:148. The nucleotide sequence of another RPL3L reference mRNA molecule is set forth in SEQ ID NO:149. The nucleotide sequence of another RPL3L reference mRNA molecule is set forth in SEQ ID NO:150.

The nucleotide sequence of an RPL3L reference cDNA molecule is set forth in SEQ ID NO:151. The nucleotide sequence of another RPL3L reference cDNA molecule is set forth in SEQ ID NO:152. The nucleotide sequence of another RPL3L reference cDNA molecule is set forth in SEQ ID NO:153. The nucleotide sequence of another RPL3L reference cDNA molecule is set forth in SEQ ID NO:154. The nucleotide sequence of another RPL3L reference cDNA molecule is set forth in SEQ ID NO:155. The nucleotide sequence of another RPL3L reference cDNA molecule is set forth in SEQ ID NO:156.

The amino acid sequence of an RPL3L reference polypeptide is set forth in SEQ ID NO:157, and is 407 amino acids in length. The amino acid sequence of another RPL3L reference polypeptide is set forth in SEQ ID NO:158, and is 47 amino acids in length.

The nucleotide sequence of an SLC25A45 reference genomic nucleic acid molecule is set forth in SEQ ID NO:159 (encompassing chr11:65,375,192-65,382,671 in the GRCh38/hg38 human genome assembly).

The nucleotide sequence of an SLC25A45 reference mRNA molecule is set forth in SEQ ID NO:160. The nucleotide sequence of another SLC25A45 reference mRNA molecule is set forth in SEQ ID NO:161. The nucleotide sequence of another SLC25A45 reference mRNA molecule is set forth in SEQ ID NO:162. The nucleotide sequence of another SLC25A45 reference mRNA molecule is set forth in SEQ ID NO:163. The nucleotide sequence of another SLC25A45 reference mRNA molecule is set forth in SEQ ID NO:164. The nucleotide sequence of another SLC25A45 reference mRNA molecule is set forth in SEQ ID NO:165. The nucleotide sequence of another SLC25A45 reference mRNA molecule is set forth in SEQ ID NO:166. The nucleotide sequence of another SLC25A45 reference mRNA molecule is set forth in SEQ ID NO:167. The nucleotide sequence of another SLC25A45 reference mRNA molecule is set forth in SEQ ID NO:168. The nucleotide sequence of another SLC25A45 reference mRNA molecule is set forth in SEQ ID NO:169. The nucleotide sequence of another SLC25A45 reference mRNA molecule is set forth in SEQ ID NO:170. The nucleotide sequence of another SLC25A45 reference mRNA molecule is set forth in SEQ ID NO:171. The nucleotide sequence of another SLC25A45 reference mRNA molecule is set forth in SEQ ID NO:172. The nucleotide sequence of another SLC25A45 reference mRNA molecule is set forth in SEQ ID NO:173. The nucleotide sequence of another SLC25A45 reference mRNA molecule is set forth in SEQ ID NO:174. The nucleotide sequence of another SLC25A45 reference mRNA molecule is set forth in SEQ ID NO:175. The nucleotide sequence of another SLC25A45 reference mRNA molecule is set forth in SEQ ID NO:176. The nucleotide sequence of another SLC25A45 reference mRNA molecule is set forth in SEQ ID NO:177. The nucleotide sequence of another SLC25A45 reference mRNA molecule is set forth in SEQ ID NO:178.

The nucleotide sequence of an SLC25A45 reference cDNA molecule is set forth in SEQ ID NO:179. The nucleotide sequence of another SLC25A45 reference cDNA molecule is set forth in SEQ ID NO:180. The nucleotide sequence of another SLC25A45 reference cDNA molecule is set forth in SEQ ID NO:181. The nucleotide sequence of another SLC25A45 reference cDNA molecule is set forth in SEQ ID NO:182. The nucleotide sequence of another SLC25A45 reference cDNA molecule is set forth in SEQ ID NO:183. The nucleotide sequence of another SLC25A45 reference cDNA molecule is set forth in SEQ ID NO:184. The nucleotide sequence of another SLC25A45 reference cDNA molecule is set forth in SEQ ID NO:185. The nucleotide sequence of another SLC25A45 reference cDNA molecule is set forth in SEQ ID NO:186. The nucleotide sequence of another SLC25A45 reference cDNA molecule is set forth in SEQ ID NO:187. The nucleotide sequence of another SLC25A45 reference cDNA molecule is set forth in SEQ ID NO:188. The nucleotide sequence of another SLC25A45 reference cDNA molecule is set forth in SEQ ID NO:189. The nucleotide sequence of another SLC25A45 reference cDNA molecule is set forth in SEQ ID NO:190. The nucleotide sequence of another SLC25A45 reference cDNA molecule is set forth in SEQ ID NO:191. The nucleotide sequence of another SLC25A45 reference cDNA molecule is set forth in SEQ ID NO:192. The nucleotide sequence of another SLC25A45 reference cDNA molecule is set forth in SEQ ID NO:193. The nucleotide sequence of another SLC25A45 reference cDNA molecule is set forth in SEQ ID NO:194. The nucleotide sequence of another SLC25A45 reference cDNA molecule is set forth in SEQ ID NO:195. The nucleotide sequence of another SLC25A45 reference cDNA molecule is set forth in SEQ ID NO:196. The nucleotide sequence of another SLC25A45 reference cDNA molecule is set forth in SEQ ID NO:197.

The amino acid sequence of an SLC25A45 reference polypeptide is set forth in SEQ ID NO:198, and is 288 amino acids in length. The amino acid sequence of another SLC25A45 reference polypeptide is set forth in SEQ ID NO:199, and is 226 amino acids in length. The amino acid sequence of another SLC25A45 reference polypeptide is set forth in SEQ ID NO:200, and is 264 amino acids in length. The amino acid sequence of another SLC25A45 reference polypeptide is set forth in SEQ ID NO:201, and is 246 amino acids in length. The amino acid sequence of another SLC25A45 reference polypeptide is set forth in SEQ ID NO:202, and is 184 amino acids in length. The amino acid sequence of another SLC25A45 reference polypeptide is set forth in SEQ ID NO:203, and is 194 amino acids in length. The amino acid sequence of another SLC25A45 reference polypeptide is set forth in SEQ ID NO:204, and is 118 amino acids in length.

The nucleotide sequence of an SLC7A9 reference genomic nucleic acid molecule is set forth in SEQ ID NO:205 (encompassing chr19:32,830,511-32,869,767 in the GRCh38/hg38 human genome assembly).

The nucleotide sequence of an SLC7A9 reference mRNA molecule is set forth in SEQ ID NO:206. The nucleotide sequence of another SLC7A9 reference mRNA molecule is set forth in SEQ ID NO:207. The nucleotide sequence of another SLC7A9 reference mRNA molecule is set forth in SEQ ID NO:208. The nucleotide sequence of another SLC7A9 reference mRNA molecule is set forth in SEQ ID NO:209. The nucleotide sequence of another SLC7A9 reference mRNA molecule is set forth in SEQ ID NO:210. The nucleotide sequence of another SLC7A9 reference mRNA molecule is set forth in SEQ ID NO:211. The nucleotide sequence of another SLC7A9 reference mRNA molecule is set forth in SEQ ID NO:212. The nucleotide sequence of another SLC7A9 reference mRNA molecule is set forth in SEQ ID NO:213. The nucleotide sequence of another SLC7A9 reference mRNA molecule is set forth in SEQ ID NO:214. The nucleotide sequence of another SLC7A9 reference mRNA molecule is set forth in SEQ ID NO:215. The nucleotide sequence of another SLC7A9 reference mRNA molecule is set forth in SEQ ID NO:216.

The nucleotide sequence of an SLC7A9 reference cDNA molecule is set forth in SEQ ID NO:217. The nucleotide sequence of another SLC7A9 reference cDNA molecule is set forth in SEQ ID NO:218. The nucleotide sequence of another SLC7A9 reference cDNA molecule is set forth in SEQ ID NO:219. The nucleotide sequence of another SLC7A9 reference cDNA molecule is set forth in SEQ ID NO:220. The nucleotide sequence of another SLC7A9 reference cDNA molecule is set forth in SEQ ID NO:221. The nucleotide sequence of another SLC7A9 reference cDNA molecule is set forth in SEQ ID NO:222. The nucleotide sequence of another SLC7A9 reference cDNA molecule is set forth in SEQ ID NO:223. The nucleotide sequence of another SLC7A9 reference cDNA molecule is set forth in SEQ ID NO:224. The nucleotide sequence of another SLC7A9 reference cDNA molecule is set forth in SEQ ID NO:225. The nucleotide sequence of another SLC7A9 reference cDNA molecule is set forth in SEQ ID NO:226. The nucleotide sequence of another SLC7A9 reference cDNA molecule is set forth in SEQ ID NO:227.

The amino acid sequence of an SLC7A9 reference polypeptide is set forth in SEQ ID NO:228, and is 487 amino acids in length.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat, prevent, or inhibit a kidney disease for use in the treatment and/or prevention of a kidney disease in a subject having: an ALDH1L1 variant genomic nucleic acid molecule encoding an ALDH1L1 predicted loss-of-function polypeptide; an ALDH1L1 variant mRNA molecule encoding an ALDH1L1 predicted loss-of-function polypeptide; or an ALDH1L1 variant cDNA molecule encoding an ALDH1L1 predicted loss-of-function polypeptide. Any of the therapeutic agents that treat, prevent, or inhibit a kidney disease described herein can be used in these methods.

The present disclosure also provides therapeutic agents that treat, prevent, or inhibit a kidney disease for use in the treatment and/or prevention of a kidney disease in a subject having: an ALDOB variant genomic nucleic acid molecule encoding an ALDOB predicted loss-of-function polypeptide; an ALDOB variant mRNA molecule encoding an ALDOB predicted loss-of-function polypeptide; or an ALDOB variant cDNA molecule encoding an ALDOB predicted loss-of-function polypeptide. Any of the therapeutic agents that treat, prevent, or inhibit a kidney disease described herein can be used in these methods.

The present disclosure also provides therapeutic agents that treat, prevent, or inhibit a kidney disease for use in the treatment and/or prevention of a kidney disease in a subject having: a G6PC variant genomic nucleic acid molecule encoding a G6PC predicted loss-of-function polypeptide; a G6PC variant mRNA molecule encoding a G6PC predicted loss-of-function polypeptide; or a G6PC variant cDNA molecule encoding a G6PC predicted loss-of-function polypeptide. Any of the therapeutic agents that treat, prevent, or inhibit a kidney disease described herein can be used in these methods.

The present disclosure also provides therapeutic agents that treat, prevent, or inhibit a kidney disease for use in the treatment and/or prevention of kidney disease in a subject having: an LRP2 variant genomic nucleic acid molecule encoding an LRP2 predicted loss-of-function polypeptide; an LRP2 variant mRNA molecule encoding an LRP2 predicted loss-of-function polypeptide; or an LRP2 variant cDNA molecule encoding an LRP2 predicted loss-of-function polypeptide. Any of the therapeutic agents that treat, prevent, or inhibit a kidney disease described herein can be used in these methods.

The present disclosure also provides therapeutic agents that treat, prevent, or inhibit a kidney disease for use in the treatment and/or prevention of a kidney disease in a subject having: an RPL3L variant genomic nucleic acid molecule encoding an RPL3L predicted loss-of-function polypeptide; an RPL3L variant mRNA molecule encoding an RPL3L predicted loss-of-function polypeptide; or an RPL3L variant cDNA molecule encoding an RPL3L predicted loss-of-function polypeptide. Any of the therapeutic agents that treat, prevent, or inhibit a kidney disease described herein can be used in these methods.

The present disclosure also provides therapeutic agents that treat, prevent, or inhibit a kidney disease for use in the treatment and/or prevention of a kidney disease in a subject having: an SLC25L45 variant genomic nucleic acid molecule encoding an SLC25L45 predicted loss-of-function polypeptide; an SLC25L45 variant mRNA molecule encoding an SLC25L45 predicted loss-of-function polypeptide; or an SLC25L45 variant cDNA molecule encoding an SLC25L45 predicted loss-of-function polypeptide. Any of the therapeutic agents that treat, prevent, or inhibit a kidney disease described herein can be used in these methods.

The present disclosure also provides therapeutic agents that treat, prevent, or inhibit a kidney disease for use in the treatment and/or prevention of a kidney disease in a subject having: an SLC7A9 variant genomic nucleic acid molecule encoding an SLC7A9 predicted loss-of-function polypeptide; an SLC7A9 variant mRNA molecule encoding an SLC7A9 predicted loss-of-function polypeptide; or an SLC7A9 variant cDNA molecule encoding an SLC7A9 predicted loss-of-function polypeptide. Any of the therapeutic agents that treat, prevent, or inhibit a kidney disease described herein can be used in these methods.

The present disclosure also provides uses of therapeutic agents that treat, prevent, or inhibit a kidney disease for use in the preparation of a medicament for treating and/or preventing a kidney disease in a subject having: an ALDH1L1 variant genomic nucleic acid molecule encoding an ALDH1L1 predicted loss-of-function polypeptide; an ALDH1L1 variant mRNA molecule encoding an ALDH1L1 predicted loss-of-function polypeptide; or an ALDH1L1 variant cDNA molecule encoding an ALDH1L1 predicted loss-of-function polypeptide. Any of the therapeutic agents that treat, prevent, or inhibit a kidney disease described herein can be used in these methods.

The present disclosure also provides uses of therapeutic agents that treat, prevent, or inhibit a kidney disease for use in the preparation of a medicament for treating and/or preventing a kidney disease in a subject having: an ALDOB variant genomic nucleic acid molecule encoding an ALDOB predicted loss-of-function polypeptide; an ALDOB variant mRNA molecule encoding an ALDOB predicted loss-of-function polypeptide; or an ALDOB variant cDNA molecule encoding an ALDOB predicted loss-of-function polypeptide. Any of the therapeutic agents that treat, prevent, or inhibit a kidney disease described herein can be used in these methods.

The present disclosure also provides uses of therapeutic agents that treat, prevent, or inhibit a kidney disease for use in the preparation of a medicament for treating and/or preventing a kidney disease in a subject having: a G6PC variant genomic nucleic acid molecule encoding a G6PC predicted loss-of-function polypeptide; a G6PC variant mRNA molecule encoding a G6PC predicted loss-of-function polypeptide; or a G6PC variant cDNA molecule encoding a G6PC predicted loss-of-function polypeptide. Any of the therapeutic agents that treat, prevent, or inhibit a kidney disease described herein can be used in these methods.

The present disclosure also provides uses of therapeutic agents that treat, prevent, or inhibit a kidney disease for use in the preparation of a medicament for treating and/or preventing a kidney disease in a subject having: an LRP2 variant genomic nucleic acid molecule encoding an LRP2 predicted loss-of-function polypeptide; an LRP2 variant mRNA molecule encoding an LRP2 predicted loss-of-function polypeptide; or an LRP2 variant cDNA molecule encoding an LRP2 predicted loss-of-function polypeptide. Any of the therapeutic agents that treat, prevent, or inhibit a kidney disease described herein can be used in these methods.

The present disclosure also provides uses of therapeutic agents that treat, prevent, or inhibit a kidney disease for use in the preparation of a medicament for treating and/or preventing a kidney disease in a subject having: an RPL3L variant genomic nucleic acid molecule encoding an RPL3L predicted loss-of-function polypeptide; an RPL3L variant mRNA molecule encoding an RPL3L predicted loss-of-function polypeptide; or an RPL3L variant cDNA molecule encoding an RPL3L predicted loss-of-function polypeptide. Any of the therapeutic agents that treat, prevent, or inhibit a kidney disease described herein can be used in these methods.

The present disclosure also provides uses of therapeutic agents that treat, prevent, or inhibit a kidney disease for use in the preparation of a medicament for treating and/or preventing a kidney disease in a subject having: an SLC25L45 variant genomic nucleic acid molecule encoding an SLC25L45 predicted loss-of-function polypeptide; an SLC25L45 variant mRNA molecule encoding an SLC25L45 predicted loss-of-function polypeptide; or an SLC25L45 variant cDNA molecule encoding an SLC25L45 predicted loss-of-function polypeptide. Any of the therapeutic agents that treat, prevent, or inhibit a kidney disease described herein can be used in these methods.

The present disclosure also provides uses of therapeutic agents that treat, prevent, or inhibit a kidney disease for use in the preparation of a medicament for treating and/or preventing a kidney disease in a subject having: an SLC7A9 variant genomic nucleic acid molecule encoding an SLC7A9 predicted loss-of-function polypeptide; an SLC7A9 variant mRNA molecule encoding an SLC7A9 predicted loss-of-function polypeptide; or an SLC7A9 variant cDNA molecule encoding an SLC7A9 predicted loss-of-function polypeptide. Any of the therapeutic agents that treat, prevent, or inhibit a kidney disease described herein can be used in these methods.

The present disclosure also provides ALDH1L1 inhibitors for use in the treatment and/or prevention of a kidney disease in a subject that is: ALDH1L1 reference, or has: an ALDH1L1 variant genomic nucleic acid molecule encoding an ALDH1L1 predicted loss-of-function polypeptide; an ALDH1L1 variant mRNA molecule encoding an ALDH1L1 predicted loss-of-function polypeptide; or an ALDH1L1 variant cDNA molecule encoding an ALDH1L1 predicted loss-of-function polypeptide. Any of the ALDH1L1 inhibitors described herein can be used in these methods.

The present disclosure also provides ALDOB inhibitors for use in the treatment and/or prevention of a kidney disease in a subject that is: ALDOB reference, or has: an ALDOB variant genomic nucleic acid molecule encoding an ALDOB predicted loss-of-function polypeptide; an ALDOB variant mRNA molecule encoding an ALDOB predicted loss-of-function polypeptide; or an ALDOB variant cDNA molecule encoding an ALDOB predicted loss-of-function polypeptide. Any of the ALDOB inhibitors described herein can be used in these methods.

The present disclosure also provides G6PC inhibitors for use in the treatment and/or prevention of a kidney disease in a subject that is: G6PC reference, or has: a G6PC variant genomic nucleic acid molecule encoding a G6PC predicted loss-of-function polypeptide; a G6PC variant mRNA molecule encoding a G6PC predicted loss-of-function polypeptide; or a G6PC variant cDNA molecule encoding a G6PC predicted loss-of-function polypeptide. Any of the G6PC inhibitors described herein can be used in these methods.

The present disclosure also provides LRP2 inhibitors for use in the treatment and/or prevention of a kidney disease in a subject that is: LRP2 reference, or has: an LRP2 variant genomic nucleic acid molecule encoding an LRP2 predicted loss-of-function polypeptide; an LRP2 variant mRNA molecule encoding an LRP2 predicted loss-of-function polypeptide; or an LRP2 variant cDNA molecule encoding an LRP2 predicted loss-of-function polypeptide. Any of the LRP2 inhibitors described herein can be used in these methods.

The present disclosure also provides RPL3L inhibitors for use in the treatment and/or prevention of a kidney disease in a subject that is: RPL3L reference, or has: an RPL3L variant genomic nucleic acid molecule encoding an RPL3L predicted loss-of-function polypeptide; an RPL3L variant mRNA molecule encoding an RPL3L predicted loss-of-function polypeptide; or an RPL3L variant cDNA molecule encoding an RPL3L predicted loss-of-function polypeptide. Any of the RPL3L inhibitors described herein can be used in these methods.

The present disclosure also provides SLC25A45 inhibitors for use in the treatment and/or prevention of a kidney disease in a subject that is: SLC25A45 reference, or has: an SLC25A45 variant genomic nucleic acid molecule encoding an SLC25A45 predicted loss-of-function polypeptide; an SLC25A45 variant mRNA molecule encoding an SLC25A45 predicted loss-of-function polypeptide; or an SLC25A45 variant cDNA molecule encoding an SLC25A45 predicted loss-of-function polypeptide. Any of the SLC25A45 inhibitors described herein can be used in these methods.

The present disclosure also provides SLC7A9 inhibitors for use in the treatment and/or prevention of a kidney disease in a subject that is: SLC7A9 reference, or has: an SLC7A9 variant genomic nucleic acid molecule encoding an SLC7A9 predicted loss-of-function polypeptide; an SLC7A9 variant mRNA molecule encoding an SLC7A9 predicted loss-of-function polypeptide; or an SLC7A9 variant cDNA molecule encoding an SLC7A9 predicted loss-of-function polypeptide. Any of the SLC7A9 inhibitors described herein can be used in these methods.

The present disclosure also provides uses of ALDH1L1 inhibitors in the preparation of a medicament for treating and/or preventing a kidney disease in a subject that is: ALDH1L1 reference, or has: an ALDH1L1 variant genomic nucleic acid molecule encoding an ALDH1L1 predicted loss-of-function polypeptide; an ALDH1L1 variant mRNA molecule encoding an ALDH1L1 predicted loss-of-function polypeptide; or an ALDH1L1 variant cDNA molecule encoding an ALDH1L1 predicted loss-of-function polypeptide. Any of the ALDH1L1 inhibitors described herein can be used in these methods.

The present disclosure also provides uses of ALDOB inhibitors in the preparation of a medicament for treating and/or preventing a kidney disease in a subject that is: ALDOB reference, or has: an ALDOB variant genomic nucleic acid molecule encoding an ALDOB predicted loss-of-function polypeptide; an ALDOB variant mRNA molecule encoding an ALDOB predicted loss-of-function polypeptide; or an ALDOB variant cDNA molecule encoding an ALDOB predicted loss-of-function polypeptide. Any of the ALDOB inhibitors described herein can be used in these methods.

The present disclosure also provides G6PC inhibitors for use in the preparation of a medicament for treating and/or preventing a kidney disease in a subject that is: a G6PC variant genomic nucleic acid molecule encoding a G6PC predicted loss-of-function polypeptide; a G6PC variant mRNA molecule encoding a G6PC predicted loss-of-function polypeptide; or a G6PC variant cDNA molecule encoding a G6PC predicted loss-of-function polypeptide. Any of the G6PC inhibitors described herein can be used in these methods.

The present disclosure also provides LRP2 inhibitors for use in the preparation of a medicament for treating and/or preventing a kidney disease in a subject that is: an LRP2 variant genomic nucleic acid molecule encoding an LRP2 predicted loss-of-function polypeptide; an LRP2 variant mRNA molecule encoding an LRP2 predicted loss-of-function polypeptide; or an LRP2 variant cDNA molecule encoding an LRP2 predicted loss-of-function polypeptide. Any of the LRP2 inhibitors described herein can be used in these methods.

The present disclosure also provides RPL3L inhibitors for use in the preparation of a medicament for treating and/or preventing a kidney disease in a subject that is: an RPL3L variant genomic nucleic acid molecule encoding an RPL3L predicted loss-of-function polypeptide; an RPL3L variant mRNA molecule encoding an RPL3L predicted loss-of-function polypeptide; or an RPL3L variant cDNA molecule encoding an RPL3L predicted loss-of-function polypeptide. Any of the RPL3L inhibitors described herein can be used in these methods.

The present disclosure also provides SLC25A45 inhibitors for use in the preparation of a medicament for treating and/or preventing a kidney disease in a subject that is: an SLC25A45 variant genomic nucleic acid molecule encoding an SLC25A45 predicted loss-of-function polypeptide; an SLC25A45 variant mRNA molecule encoding an SLC25A45 predicted loss-of-function polypeptide; or an SLC25A45 variant cDNA molecule encoding an SLC25A45 predicted loss-of-function polypeptide. Any of the SLC25A45 inhibitors described herein can be used in these methods.

The present disclosure also provides SLC7A9 inhibitors for use in the preparation of a medicament for treating and/or preventing a kidney disease in a subject that is: an SLC7A9 variant genomic nucleic acid molecule encoding an SLC7A9 predicted loss-of-function polypeptide; an SLC7A9 variant mRNA molecule encoding an SLC7A9 predicted loss-of-function polypeptide; or an SLC7A9 variant cDNA molecule encoding an SLC7A9 predicted loss-of-function polypeptide. Any of the SLC7A9 inhibitors described herein can be used in these methods.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Identification of Genes in which Loss-of-Function or Missense Variants are Associated with Increased eGFR

To identify genetic factors contributing to predisposition for or protection against chronic liver disease, exome sequencing was performed in 545,740 participants from the UK Biobank cohort (UKB) and Geisinger Health System DiscovEHR study (GHS). For each gene in the genome, associations with estimated glomerular filtration rate (eGFR), a widely used biomarker of kidney function in which higher levels indicate better function, were estimated for the burden of rare loss-of-function and missense variants identified by exome sequencing. Statistically significant findings were subsequently evaluated for their association with clinical diagnoses of kidney disease in UKB and GHS.

In the exome-wide analysis, the burden of rare (alternative allele frequency (AAF)<1%) predicted loss-of-function (pLOF) or missense genetic variants in seven genes was strongly associated with increased eGFR at the exome-wide level of statistical significance (p<3.6×10⁻⁷, a Bonferroni correction for 20,000 genes and seven variant selection models, Table 8).

TABLE 8
Rare pLOF or missense variants in genes associated with increased eGFR levels

| | | Per allele beta | Per allele beta | | Genotype counts, | AAF, |
|---|---|---|---|---|---|---|
| | Genetic | (95% Cl), SD | (95% Cl), mL/min | | RR\|RA\|AA | fraction |
| Gene | exposure | units | units | P-value | genotypes | of 1 |
| ALDH1L1 | pLOF or | 0.05 | 0.89 | 1.39E−16 | 529024\|16679\|37 | 0.0153 |
| | missense (5/5) | (0.04, 0.07) | (0.68, 1.11) | | | |
| | variants with | | | | | |
| | AAF <1% | | | | | |
| ALDOB | pLOF or | 0.06 | 1.03 | 1.85E−14 | 534968\|10752\|20 | 0.0099 |
| | missense (1+/5) | (0.05, 0.08) | (0.77, 1.29) | | | |
| | variants with | | | | | |
| | AAF <1% | | | | | |
| G6PC | pLOF or | 0.12 | 2.03 | 4.07E−12 | 543603\|2137\|0 | 0.0020 |
| | missense (5/5) | (0.09, 0.15) | (1.46, 2.61) | | | |
| | variants with | | | | | |
| | AAF <1% | | | | | |
| LRP2 | pLOF or | 0.05 | 0.8 | 5.15E−30 | 501161\|44510\|69 | 0.0409 |
| | missense | (0.04, 0.06) | (0.66, 0.94) | | | |
| | variants with | | | | | |
| | AAF <0.1% | | | | | |
| LRP2 | pLOF or | 0.04 | 0.69 | 2.30E−38 | 466144\|79393\|203 | 0.0731 |
| | missense (1+/5) | (0.03, 0.05) | (0.59, 0.79) | | | |
| | variants with | | | | | |
| | AAF <1% | | | | | |
| RPL3L | pLOF or | 0.04 | 0.64 | 1.34E−37 | 460204\|84156\|1380 | 0.0796 |
| | missense (5/5) | (0.03, 0.04) | (0.54, 0.74) | | | |
| | variants with | | | | | |
| | AAF <5% | | | | | |
| SLC25A45 | pLOF or | 0.05 | 0.86 | 1.33E−39 | 499245\|45844\|651 | 0.0432 |
| | missense | (0.04, 0.06) | (0.73, 0.99) | | | |
| | variants with | | | | | |
| | AAF <5% | | | | | |
| SLC7A9 | pLOF or | 0.08 | 1.33 | 9.22E−28 | 532725\|12997\|18 | 0.0119 |
| | missense | (0.06, 0.09) | (1.1, 1.57) | | | |
| | variants with | | | | | |
| | AAF <1% | | | | | |

RR indicates the number of individuals in the population studies carrying no missense variants in the specified gene; RA indicates the number of individuals carrying one or more missense variants of a single allele of the specified gene; AA indicates the number of individuals carrying one or more missense variants in both alleles of the specified gene; pLOF indicates predicted loss of function; missense (1/5) and missense (5/5) indicate missense variant predicted to be deleterious by at least 1 out of 5 or 5 out of 5, respectively, in silico prediction algorithms; AAF indicates alternate allele frequency.

The top 1-3 specific genetic variants in seven genes strongly associated with increased eGFR are listed in Table 9.

TABLE 9
Rare pLOF or missense variants in genes associated with increased eGFR levels

| | Top variant ID | | Per allele | | | | |
|---|---|---|---|---|---|---|---|
| | (Chromosome: | Per allele | beta | | Genotype | | % of |
| | Position: | beta (95% | (95% Cl), | | counts, RR\| | AAF, | individuals |
| Genetic | RA:AA) | Cl), SD | min/mL | | RA\|AA | fraction | carrying |
| exposure | Top variant RSID | units | units | P-value | genotypes | of 1 | variant |
| ALDH1L1 | 3:126160912:C:T | 0.06 | 0.98 | 1.3E−09 | 538,354\| | 0.00679 | 1.35% |
| burden of | rs143122118 | [0.04, 0.08] | [0.66, 1.29] | | 7,351\|28 | | |
| pLOF or | 3:126110055:G:C | 0.24 | 4.05 | 1.3E−05 | 525,051\| | 0.00020 | 0.04% |
| missense | rs775766256 | [0.13, 0.35] | [2.23, 5.87] | | 213\|0 | | |
| variants | | | | | | | |
| with 5 | | | | | | | |
| deleterious | | | | | | | |
| predictions | | | | | | | |
| with | | | | | | | |
| AAF <1% | | | | | | | |
| ALDOB | 9:101427574:C:G | 0.07 | 1.15 | 4.7E−10 | 537,930\| | 0.00520 | 1.04% |
| burden of | rs1800546 | [0.05, 0.09] | [0.79, 1.51] | | 5,629\|13 | | |
| pLOF or | 9:101426570:C:A | −3.28 | −55.5 | 1.1E−04 | 411,090\| | 1.2E−06 | 0.0002% |
| missense | NA | [−4.94, −1.62] | [−83.5, −27.4] | | 1/0 | | |
| variants | 9:101429913:G:A | 0.16 | 2.7 | 0.0023 | 525,019\| | 0.00023 | 0.05% |
| with 1+ | rs201397971 | [0.06, 0.26] | [0.96, 4.44] | | 243\|0 | | |
| deleterious | | | | | | | |
| prediction | | | | | | | |
| with | | | | | | | |
| AAF <1% | | | | | | | |
| G6PC | 17:42900952:TC:T | 0.15 | 2.59 | 0.0021 | 524,994\| | 0.00025 | 0.05% |
| burden of | rs1251265849 | [0.06, 0.25] | [0.94, 4.24] | | 265\|0 | | |
| pLOF or | 17:42911417:G:T | −2.59 | −43.7 | 0.0023 | 411,090\| | 1.2E−06 | 0.0002% |
| missense | rs746978011 | [−4.25, −0.93] | [−71.8, −15.7] | | 1\|0 | | |
| variants | 17:42903947:C:T | 0.11 | 1.89 | 0.0077 | 545,379\| | 0.00033 | 0.07% |
| with 5 | rs1801175 | [0.03, 0.19] | [0.5, 3.27] | | 358\|0 | | |
| deleterious | 17:42900910:G:C | 0.96 | 16.3 | 0.017 | 525,261\| | 3.8E−06 | 0.0008% |
| predictions | NA | [0.17, 1.75] | [2.9, 29.7] | | 4\|0 | | |
| with | 17:42903998:C:T | −2.02 | −34.2 | 0.017 | 411,090\| | 1.2E−06 | 0.0002% |
| AAF <1% | rs1157674386 | [−3.68, −0.36] | [−62.2, −6.1] | | 1\|0 | | |
| | 17:42911391:C:T | 0.09 | 1.6 | 0.023 | 534,712\| | 0.00036 | 0.07% |
| | rs80356487 | [0.01, 0.18] | [0.22, 2.99] | | 382\|0 | | |
| LRP2 | 2:169174132:A:G | 0.70 | 11.8 | 1.0E−09 | 411,036\| | 0.00007 | 0.01% |
| burden of | NA | [0.47, 0.92] | [8.0, 15.6] | | 55\|0 | | |
| pLOF or | 2:169170643:T:A | 0.19 | 3.16 | 2.1E−08 | 533,193\| | 0.00057 | 0.11% |
| missense | rs200475391 | [0.12, 0.25] | [2.06, 4.27] | | 604\|0 | | |
| variants | | | | | | | |
| with | | | | | | | |
| AAF <0.1% | | | | | | | |
| LRP2 | 2:169173147:C:T | 0.10 | 1.66 | 5.8E−23 | 537,143\| | 0.00599 | 1.19% |
| burden of | rs34355135 | [0.08, 0.12] | [1.33, 1.99] | | 6,463\|24 | | |
| pLOF or | 2:169174132:A:G | 0.70 | 11.8 | 1.0E−09 | 411,036\| | 0.00007 | 0.01% |
| missense | NA | [0.47, 0.92] | [8.0, 15.6] | | 55\|0 | | |
| variants | 2:169170643:T:A | 0.19 | 3.2 | 2.1E−08 | 533,193\| | 0.00057 | 0.11% |
| with 1 | rs200475391 | [0.12, 0.25] | [2.1, 4.2] | | 604\|0 | | |
| deleterious | | | | | | | |
| prediction | | | | | | | |
| with | | | | | | | |
| AAF <1% | | | | | | | |
| RPL3L | 16:1947003:C:T | 0.04 | 0.76 | 8.2E−23 | 513,157\| | 0.03027 | 5.95% |
| burden of | rs113956264 | [0.04, 0.05] | [0.61, 0.9] | | 31,911\|563 | | |
| pLOF or | 16:1953015:G:A | 0.03 | 0.42 | 2.3E−09 | 507,991\| | 0.03523 | 6.91% |
| missense | rs140185678 | [0.02, 0.03] | [0.29, 0.56] | | 36,960\|744 | | |
| variants | | | | | | | |
| with 5 | | | | | | | |
| deleterious | | | | | | | |
| predictions | | | | | | | |
| with | | | | | | | |
| AAF <5% | | | | | | | |
| SLC25A45 | 11:65376421:G:A | 0.05 | 0.85 | 2.7E−30 | 508,523\| | 0.03282 | 6.45% |
| burden of | rs34400381 | [0.04, 0.06] | [0.7, 0.99] | | 34,461\|611 | | |
| pLOF or | 11:65379542:C:T | 0.06 | 0.93 | 2.3E−08 | 538,745\| | 0.00642 | 1.28% |
| missense | rs78829599 | [0.04, 0.07] | [0.61, 1.26] | | 6,949\|30 | | |
| variants | | | | | | | |
| with | | | | | | | |
| AAF <5% | | | | | | | |
| SLC7A9 | 19:32862521:C:T | 0.08 | 1.43 | 7.7E−14 | 538,325\| | 0.00487 | 0.97% |
| burden of | rs79389353 | [0.06, 0.11] | [1.05, 1.8] | | 5,268\|13 | | |
| pLOF or | 19:32864261:C:T | 0.15 | 2.47 | 1.8E−06 | 542,927\| | 0.00065 | 0.13% |
| missense | rs121908480 | [0.09, 0.21] | [1.46, 3.49] | | 700\|2 | | |
| variants | | | | | | | |
| with | | | | | | | |
| AAF <1% | | | | | | | |

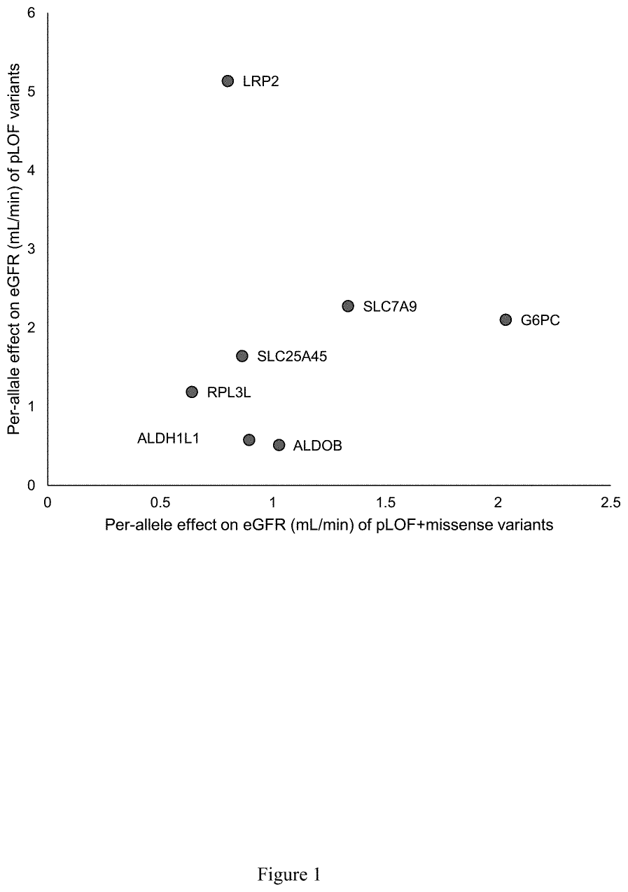

The effect estimate of the burden of pLOF variants in each of these seven genes was directionally consistent with the burden of pLOF or any missense variants (see, FIG. 1), suggesting that the majority of rare missense variants included in the analysis result in a loss-of-function. Hence, these associations are consistent with loss-of-function in these genes.

### Example 2: Identification of Genes in which Loss-of-Function or Missense Variant is Associated with Protection Against Kidney Disease

The association of rare pLOF or missense variants in these seven genes with kidney disease outcomes was estimated. The burdens of rare pLOF or missense variants were associated with protection against any kidney disease (a broadly-defined composite outcome including kidney diseases of different types and etiology), any chronic kidney disease including or excluding microalbuminuria, later stages of chronic kidney disease such as stage 3 or greater, or stage 5 or greater, and severe kidney disease including end-stage renal disease or renal replacement therapy (Table 10). Heterozygous carriers of these genetic variants had 7% to 47% lower odds of kidney disease compared to non-carriers. Therefore, loss-of-function or deleterious missense variation in these genes is associated with protection against various types of kidney disease in humans.

TABLE 10
Association of any missense, predicted deleterious missense variants or predicted loss of function
variants in protective genes with any kidney disease, any chronic kidney disease, chronic kidney
disease stage 3+ or 5+, renal replacement therapy or end-stage renal disease in UKB and GHS

| Genetic exposure | Outcome | Per allele odds ratio (95% CI) | | P-value |
|---|---|---|---|---|
| ALDH1L1 pLOF or | Any kidney disease | 0.93 | (0.88, 0.98) | 0.005 |
| missense (5/5) | CKD, any | 0.88 | (0.82, 0.96) | 0.002 |
| variants with | CKD, any including | 0.93 | (0.87, 0.99) | 0.017 |
| AAF < 1% | microalbuminuria | | | |
| | CKD, stage 3+ | 0.87 | (0.79, 0.95) | 0.002 |
| | Renal replacement therapy | 0.82 | (0.69, 0.97) | 0.024 |
| ALDOB pLOF or | CKD, any | 0.85 | (0.77, 0.94) | 0.001 |
| missense (1+/5) | CKD, stage 3+ | 0.88 | (0.78, 0.98) | 0.019 |
| variants with | | | | |
| AAF < 1% | | | | |
| G6PC pLOF or | Any kidney disease | 0.86 | (0.74, 0.99) | 0.031 |
| missense (5/5) | CKD, any | 0.74 | (0.6, 0.91) | 0.004 |
| variants with | CKD, any including | 0.82 | (0.69, 0.97) | 0.017 |
| AAF < 1% | microalbuminuria | | | |
| | CKD, stage 3+ | 0.74 | (0.59, 0.93) | 0.008 |
| | CKD, stage 5+ | 0.55 | (0.32, 0.94) | 0.027 |
| | Renal replacement therapy | 0.56 | (0.36, 0.87) | 0.009 |
| | End stage renal disease | 0.53 | (0.3, 0.91) | 0.023 |
| LRP2 pLOF or | Any kidney disease | 0.95 | (0.92, 0.97) | 6E−05 |
| missense (1+/5) | CKD, any | 0.89 | (0.86, 0.93) | 4E−09 |
| variants with | CKD, stage 3+ | 0.88 | (0.85, 0.92) | 1E−08 |
| AAF < 1% | | | | |
| LRP2 pLOF or | CKD, stage 5+ | 0.83 | (0.72, 0.96) | 0.010 |
| missense variants | Renal replacement therapy | 0.85 | (0.77, 0.95) | 0.003 |
| with AAF < 0.1% | End stage renal disease | 0.84 | (0.73, 0.97) | 0.015 |
| RPL3L pLOF or | Any kidney disease | 0.97 | (0.95, 0.99) | 0.011 |
| missense (5/5) | CKD, any | 0.95 | (0.92, 0.99) | 0.005 |
| variants with | CKD, stage 3+ | 0.93 | (0.89, 0.96) | 2E−04 |
| AAF < 5% | CKD, stage 5+ | 0.90 | (0.81, 0.99) | 0.039 |
| SLC25A45 pLOF or | CKD, any | 0.91 | (0.87, 0.95) | 6E−05 |
| missense variants | CKD, any including | 0.94 | (0.91, 0.98) | 0.002 |
| with AAF < 5% | microalbuminuria | | | |
| | CKD, stage 3+ | 0.89 | (0.84, 0.94) | 1E−05 |
| SLC7A9 pLOF or | Any kidney disease | 0.90 | (0.85, 0.96) | 5E−04 |
| missense variants | CKD, any | 0.88 | (0.81, 0.96) | 0.005 |
| with AAF < 1% | CKD, any including | 0.90 | (0.84, 0.96) | 0.002 |
| | microalbuminuria | | | |
| | CKD, stage 3+ | 0.84 | (0.76, 0.92) | 4E−04 |

| | | Genotype counts, | Genotype counts, | AAF, |
|---|---|---|---|---|
| | | RR \| RA \| AA | RR \| RA \| AA | Fraction |
| Genetic exposure | Outcome | genotypes in cases | genotypes in controls | of 1 |
| ALDH1L1 pLOF or | Any kidney disease | 82105 \| 2424 \| 4 | 211734 \| 6963 \| 20 | 0.0156 |
| missense (5/5) | CKD, any | 39790 \| 1106 \| 0 | 210814 \| 6941 \| 20 | 0.0156 |
| variants with | CKD, any including | 57948 \| 1700 \| 2 | 211734 \| 6963 \| 20 | 0.0156 |
| AAF < 1% | microalbuminuria | | | |
| | CKD, stage 3+ | 34106 \| 936 \| 0 | 213468 \| 7003 \| 20 | 0.0156 |
| | Renal replacement therapy | 4893 \| 132 \| 0 | 213345 \| 6997 \| 20 | 0.0159 |
| ALDOB pLOF or | CKD, any | 40165 \| 731 \| 0 | 213354 \| 4415 \| 6 | 0.0100 |
| missense (1+/5) | CKD, stage 3+ | 34418 \| 624 \| 0 | 215988 \| 4496 \| 7 | 0.0100 |
| variants with | | | | |
| AAF < 1% | | | | |
| G6PC pLOF or | Any kidney disease | 83952 \| 350 \| 0 | 216855 \| 920 \| 0 | 0.0021 |
| missense (5/5) | CKD, any | 40723 \| 173 \| 0 | 216855 \| 920 \| 0 | 0.0021 |
| variants with | CKD, any including | 59217 \| 241 \| 0 | 216855 \| 920 \| 0 | 0.0021 |
| AAF < 1% | microalbuminuria | | | |
| | CKD, stage 3+ | 34888 \| 154 \| 0 | 219564 \| 927 \| 0 | 0.0021 |
| | CKD, stage 5+ | 3135 \| 8 \| 0 | 219564 \| 927 \| 0 | 0.0021 |
| | Renal replacement therapy | 4999 \| 10 \| 0 | 217799 \| 915 \| 0 | 0.0021 |
| | End stage renal disease | 2859 \| 6 \| 0 | 217924 \| 919 \| 0 | 0.0021 |
| LRP2 pLOF or | Any kidney disease | 72228 \| 12262 \| 43 | 185697 \| 32917 \| 103 | 0.0750 |
| missense (1+/5) | CKD, any | 35073 \| 5805 \| 18 | 184858 \| 32814 \| 103 | 0.0751 |
| variants with | CKD, stage 3+ | 30073 \| 4954 \| 15 | 187145 \| 33239 \| 107 | 0.0752 |
| AAF < 1% | | | | |
| LRP2 pLOF or | CKD, stage 5+ | 2919 \| 224 \| 0 | 201616 \| 18837 \| 38 | 0.0428 |
| missense variants | Renal replacement | 4640 \| 368 \| 1 | 200187 \| 18491 \| 36 | 0.0423 |
| with AAF < 0.1% | therapy | | | |
| | End stage renal disease | 2663 \| 202 \| 0 | 200301 \| 18506 \| 36 | 0.0424 |
| RPL3L pLOF or | Any kidney disease | 71392 \| 12936 \| 205 | 183811 \| 34299 \| 607 | 0.0806 |
| missense (5/5) | CKD, any | 34590 \| 6208 \| 98 | 182960 \| 34208 \| 607 | 0.0808 |
| variants with | CKD, stage 3+ | 29718 \| 5239 \| 85 | 185288 \| 34588 \| 615 | 0.0807 |
| AAF < 5% | CKD, stage 5+ | 2694 \| 442 \| 7 | 185288 \| 34588 \| 615 | 0.0811 |
| SLC25A45 pLOF or | CKD, any | 37702 \| 3154 \| 40 | 198654 \| 18846 \| 275 | 0.0437 |
| missense variants | CKD, any including | 54791 \| 4786 \| 73 | 199557 \| 18884 \| 276 | 0.0438 |
| with AAF < 5% | microalbuminuria | | | |
| | CKD, stage 3+ | 32371 \| 2639 \| 32 | 201211 \| 19003 \| 277 | 0.0436 |
| SLC7A9 pLOF or | Any kidney disease | 82566 \| 1962 \| 5 | 213183 \| 5525 \| 9 | 0.0124 |
| missense variants | CKD, any | 39958 \| 935 \| 3 | 212284 \| 5482 \| 9 | 0.0125 |
| with AAF < 1% | CKD, any including | 58247 \| 1400 \| 3 | 213183 \| 5525 \| 9 | 0.0125 |
| | microalbuminuria | | | |
| | CKD, stage 3+ | 34269 \| 771 \| 2 | 214856 \| 5627 \| 8 | 0.0126 |

RR indicates the number of individuals in the population studies carrying no missense variants; RA indicates the number of individuals carrying one or more missense variants of a single allele of the specified gene; AA indicates the number of individuals carrying one or more missense variants in both alleles of the specified gene; pLOF indicates predicted loss of function; missense (1+/5) and missense (5/5) indicate missense variant predicted to be deleterious by at least 1 out of 5 or 5 out of 5, respectively, in silico prediction algorithms; AAF indicates alternate allele frequency.

### Example 3: General Methodology

### Participating Cohorts:

Genetic association studies were performed in the United Kingdom (UK) Biobank (UKB) cohort (Sudlow et al., PLoS Med, 2015, 12, e1001779) and the DiscoverEHR cohort from the Geisinger Health System (GHS) MyCode Community Health Initiative (Carey et al., Genet. Med., 2016, 18, 906-13). The UKB is a population-based cohort study of people aged between 40 and 69 years recruited through 22 testing centers in the UK between 2006-2010. Over 430,000 participants from UKB with available whole-exome sequencing and clinical phenotype data were included. The GHS MyCode study Community Health Initiative is a health system-based cohort of patients from Central and Eastern Pennsylvania (USA) recruited in 2007-2019. Over 130,000 European ancestry participants from GHS with available whole-exome sequencing and clinical phenotype data were included.

### Phenotype Definitions:

eGFR was calculated from clinical laboratory measurements for creatinine extracted from electronic health records (EHRs) of participants from GHS. Median values were calculated for all participants with two or more measurements. In UKB, eGFR was calculated from creatinine measured by IFCC (International Federation of Clinical Chemistry) analysis on a Beckman Coulter AU5800 at the baseline visit of the study. In UKB, urine albumin:creatinine ratio (UACR) was derived from creatinine measured by enzymatic analysis and albumin measured by immunoturbidimetric analysis on a Beckman Coulter AU5400 on spot urine measured at the baseline visit of the study. Prior to genetic association analysis, continuous phenotype values were transformed by the inverse standard normal function, applied within each ancestry group and separately in men and women. Kidney disease outcomes were defined according to the International Classification of Diseases Tenth Revision (ICD-10) and read codes stored in EHRs. Self-reported disease status was used when available. Office of Population Censuses and Surveys Classification of Interventions and Procedures version 4 (OPCS4) codes were used for medical procedures in UKB.

Individuals with kidney diseases were identified as described in Table 11, combining EHR records, self-reports and eGFR and UACR measurements.

TABLE 11
Definitions of kidney disease and outcomes in UKB and GHS cohort

| | | Control definition |
|---|---|---|
| Kidney | Case definition | Participants were excluded from the control |
| disease | Participants were included as a case if they met | population for all kidney disease outcomes |
| outcome | any of the criteria specific to each disease | if they met any of the following criteria |
| Any kidney | ICD10: | ICD10: |
| disease | N00, N01, N02, N03, N04, N05, N06, N07, N08, N10, N11, | N00, N01, N02, N03, N04, N05, N06, N07, N08, N10, |
| | N12, N13, N14, N15, N16, N17, N18, N19, N25, N26, N27, | N11, N12, N13, N14, N15, N16, N17, N18, N19, N20, |
| | N28, N29, Q60, Q61, Q63, C64, R944, I12, I13, T861, | N21, N22, N23, N25, N26, N27, N28, N29, Q60, Q61, |
| | Z940, O904, O084, T824, Y841, Z490, Z491, Z492, Z992 | Q63, C64, R944, I12, I13, T861, Z940, O904, O084, |
| | UKB.f.20002: | T824, Y841, Z490, Z491, Z492, Z992 |
| | 1192, 1193, 1194, 1519, 1520, 1607, 1608, 1609, 1427, | UKB.f.20002: |
| | 1405 | 1192, 1193, 1194, 1197, 1519, 1520, 1607, 1608, |
| | UKB.f.20004: | 1609, 1427, 1405 |
| | 1195, 1476, 1487, 1580, 1581, 1582, 1618 | UKB.f.20004: |
| | UKB.OPCS4: | 1195, 1197, 1476, 1487, 1580, 1581, 1582, 1618 |
| | M01, M02, M03, M04, M05, M068, M069, M078, M079, | UKB.OPCS4: |
| | M08, M10, M111, M112, M131, M132, M133, M137, | M01, M02, M03, M04, M05, M061, M068, M069, M07, |
| | M164, L746, M172, M174, M178, M 179, X401, X402, | M08, M09, M10, M111, M112, M131, M132, M133, |
| | X403, X404, X405, X406, X408, X409, X411, X412, | M137, M14, M164, L746, M172, M174, M178, M179, |
| | X418, X419, X421, X428, X429, M012, M013, M014, | X401, X402, X403, X404, X405, X406, X408, X409, |
| | M015, M018, M019, M084 | X411, X412, X418, X419, X421, X428, X429, M012, |
| | UKB.f.42027: 0, 1, 2 | M013, M014, M015, M018, M019, M084 |
| | GHS.EHR: kidney transplant or dialysis in billing | UKB.f.42027: 0, 1, 2 |
| | procedures or surgery | GHS.EHR: kidney transplant or dialysis in billing |
| | eGFR < 60 ml/min | procedures or surgery |
| | UKB.UACR > 30 mg/g | eGFR < 90 ml/min |
| | | UKB.UACR > 10 mg/g |
| Chronic kidney | ICD10: | |
| disease, any | N18, Q611, Q612, Q613, Q615, I12, I13, T861, Z940, | |
| | Z492 | |
| | UKB.f.20002: 1193, 1607, 1427 | |
| | UKB.f.20004: 1195, 1476, 1580, 1581, 1582 | |
| | UKB.OPSC4: | |
| | L746, M012, M013, M014, M015, M018, M019, M084, | |
| | M174, M178, M 179, X402, X405, X406, X411, X412 | |
| | UKB.f.42027: 0, 1, 2 | |
| | GHS.EHR: kidney transplant in billing procedures | |
| | or surgery | |
| | eGFR < 60 ml/min | |
| | UKB.UACR >300 mg/g | |
| Chronic kidney | ICD10: | |
| disease, any | N18, Q611, Q612, Q613, Q615, I12, I13, T861, Z940, | |
| including | Z492 | |
| microalbuminuria | UKB.f.20002: 1193, 1607, 1427 | |
| | UKB.f.20004: 1195, 1476, 1580, 1581, 1582 | |
| | UKB.OPSC4: | |
| | L746, M012, M013, M014, M015, M018, M019, M084, | |
| | M174, M178, M179, X402, X405, X406, X411, X412 | |
| | UKB.f.42027: 0, 1, 2 | |
| | GHS.EHR: kidney transplant in billing procedures | |
| | or surgery | |
| | eGFR < 60 ml/min | |
| | UKB.UACR > 30 mg/g | |
| CKD stage 3+ | ICD10: | |
| | N183, N184, N185, N180, N186, T861, Z492, Z940, | |
| | N165 | |
| | UKB.f.20002: 1193 | |
| | UKB.f.20004: 1476, 1195, 1580, 1581, 1582 | |
| | UKB.OPSC4: | |
| | M012, M013, M014, M015, M018, M019, M084, M174, | |
| | M178, M179, X402, X405, X406, X411, X412 | |
| | UKB.f.42027: 0, 1, 2 | |
| | GHS.EHR: kidney transplant in billing procedures | |
| | or surgery | |
| | eGFR < 60 ml/min | |
| CKD stage 5+ | ICD10: N185, N180, N186, T861, Z492, Z940, N165 | |
| | UKB.f.20002: 1193 | |
| | UKB.f.20004: 1476, 1195, 1580, 1581, 1582 | |
| | UKB.OPSC4: | |
| | M012, M013, M014, M015, M018, M019, M084, M174, | |
| | M178, M179, X402, X405, X406, X411, X412 | |
| | UKB.f.42027: 0, 1, 2 | |
| | GHS.EHR: kidney transplant in billing procedures | |
| | or surgery | |
| | eGFR < 15 ml/min | |
| Renal replacement | ICD10: T824, T861, Y841, Z490, Z491, Z492, Z940, | |
| therapy | Z992 | |
| | UKB.f.20002: 1193 | |
| | UKB.f.20004: 1195, 1476, 1580, 1581, 1582 | |
| | UKB.OPSC4: | |
| | L746, M012, M013, M014, M015, M018, M019, M084, | |
| | M174, M178, M179, X401, X402, X403, X404, X405, | |
| | X406, X408, X409, X411, X412, X418, X419, X421, | |
| | X428, X429 | |
| | UKB.f.42027: 0, 1, 2 | |
| | GHS.EHR: kidney transplant or dialysis in billing | |
| | procedures or surgery | |
| End stage | ICD10: N180, N186, T861, Z492, Z940, N165 | |
| renal disease | UKB.f.20002: 1193 | |
| | UKB.f.20004: 1476, 1195, 1580, 1581, 1582 | |
| | UKB.OPSC4: | |
| | M012, M013, M014, M015, M018, M019, M084, M174, | |
| | M178, M179, X402, X405, X406, X411, X412 | |
| | UKB.f.42027: 0, 1, 2 | |
| | GHS.EHR: kidney transplant in billing procedures | |
| | or surgery | |

Individuals were excluded from the control population for all kidney outcomes if they met any of the following criteria: ICD10: N00, N01, N02, N03, N04, N05, N06, N07, N08, N10, N11, N12, N13, N14, N15, N16, N17, N18, N19, N20, N21, N22, N23, N25, N26, N27, N28, N29, Q60, Q61, Q63, C64, R944, 112, 113, T861, Z940, O904, O084, T824, Y841, Z490, Z491, Z492, Z992; UKB.f.20002: 1192, 1193, 1194, 1197, 1519, 1520, 1607, 1608, 1609, 1427, 1405; UKB.f.20004: 1195, 1197, 1476, 1487, 1580, 1581, 1582, 1618; UKB.OPCS4 code: M01, M02, M03, M04, M05, M061, M068, M069, M07, M08, M09, M10, M111, M112, M131, M132, M133, M137, M14, M164, L746, M172, M174, M178, M179, X401, X402, X403, X404, X405, X406, X408, X409, X411, X412, X418, X419, X421, X428, X429, M012, M013, M014, M015, M018, M019, M084; df.42027 in UKB: 0,1,2; GHS EHR: kidney transplant or dialysis in billing procedures or surgery; eGFR<90 ml/min; UKB.UACR>10 mg/g. ICD10 indicates the 10th revision of the International Statistical Classification of Diseases and Related Health Problems; UKB.OPCS4 indicates Office of Population Censuses and Surveys (OPCS) Classification of Interventions and Procedures version 4 as used in the UK Biobank (UKB); UKB.f.20002 indicates self-reported non-cancer illness codes as used in UKB; UKB.f.20004 indicates self-reported medical procedures as used in UKB; UKB.f.42027 indicates end-stage renal disease as defined by central working group for UKB; GHS.EHR indicates billing procedures or surgeries in Geisinger Health System (GHS) relevant to disease definition; UKB.UACR indicates urine albumin:creatinine ratio measured in UKB.

### Genotype Data:

High coverage whole exome sequencing was performed as previously described (Science, 2016, 354:aaf6814; and Nature, 2020, 586, 749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20×coverage over 85% of targeted bases in 96% of VCRome samples and 20×coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into these 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%.

Association Analysis of Gene Burden of Rare pLOF and Missense Variations:

Association between the burden of rare predicted loss-of-function or missense variants in a given gene and phenotype was examined by fitting a linear (for quantitative traits) or firth bias-corrected logistic (for binary traits) regression model adjusted for a polygenic score that approximates a genomic kinship matrix using REGENIE (Mbatchou et al., Nat. Genetics, 2020, 53, 1097-1103). Analyses were stratified by ancestry and adjusted for age, age², sex, age-by-sex and age²-by-sex interaction terms, experimental batch-related covariates, 10 common variant-derived principal components, and 20 rare variant-derived principal components. Results across cohorts for each variant-phenotype association were combined using fixed effects inverse variance weighted meta-analysis. In gene burden tests, all individuals are labeled as heterozygotes if they carry one or more qualifying rare variant (as described above based on frequency and functional annotation) and as homozygotes if they carry any qualifying variant in the homozygous state. This “composite genotype” is then used to test for association.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having a kidney disease, chronic kidney disease, a kidney stone, chronic glomerulonephritis, nephrosis, nephronophthisis, chronic interstitial nephritis, or nephrosclerosis, or preventing a subject from developing a kidney disease, chronic kidney disease, a kidney stone, chronic glomerulonephritis, nephrosis, nephronophthisis, chronic interstitial nephritis, or nephrosclerosis, the method comprising administering one or more Aldehyde Dehydrogenase 1 Family Member L1 (ALDH1L1) inhibitors, Fructose-Bisphosphate Aldolase B (ALDOB) inhibitors, Glucose-6-Phosphatase Catalytic Subunit 1 (G6PC) inhibitors, LDL Receptor Related Protein 2 (LRP2) inhibitors, Ribosomal Protein L3 Like (RPL3L) inhibitors, Solute Carrier Family 25, Member 4 (SLC25A45) inhibitors, or Solute Carrier Family 7 Member 9 (SLC7A9) inhibitors, or any combination thereof, to the subject.

2-8. (canceled)

9. The method according to claim 1, wherein i) the ALDH1L1 inhibitor comprises an inhibitory nucleic acid molecule that hybridizes to an ALDH1L1 nucleic acid molecule: ii) the ALDOB inhibitor comprises an inhibitory nucleic acid molecule that hybridizes to an ALDOB nucleic acid molecule: iii) the G6PC inhibitor comprises an inhibitory nucleic acid molecule that hybridizes to a G6PC nucleic acid molecule: iv) the LRP2 inhibitor comprises an inhibitory nucleic acid molecule that hybridizes to an LRP2 nucleic acid molecule: v) the RPL3L inhibitor comprises an inhibitory nucleic acid molecule that hybridizes to an RPL3L nucleic acid molecule: vi) the SLC25A45 inhibitor comprises an inhibitory nucleic acid molecule that hybridizes to an SLC25A45 nucleic acid molecule; and/or vii) the SLC7A9 inhibitor comprises an inhibitory nucleic acid molecule that hybridizes to an SLC7A9 nucleic acid molecule.

10-15. (canceled)

16. The method according to claim 9, wherein the inhibitory nucleic acid molecule comprises an antisense nucleic acid molecule, a small interfering RNA (siRNA), or a short hairpin RNA (shRNA) that hybridizes to an ALDH1L1 mRNA, an ALDOB mRNA, a G6PC mRNA, an LRP2 mRNA, an RPL3L mRNA, an SLC25A45 mRNA, or an SLC7A9 mRNA.

17-55. (canceled)

56. The method according to claim 1, further comprising administering a therapeutic agent that treats, prevents, or inhibits a kidney disease, chronic kidney disease, a kidney stone, chronic glomerulonephritis, nephrosis, nephronophthisis, chronic interstitial nephritis, and/or nephrosclerosis in an amount that is the same as or less than a standard dosage amount to a subject who does not have one or more of the ALDH1L1, ALDOB, G6PC, LRP2, RPL3L, SLC25A45, or SLC7A9 variant nucleic acid molecules.

57. The method according to claim 1, further comprising administering a therapeutic agent that treats, prevents, or inhibits a kidney disease, chronic kidney disease, a kidney stone, chronic glomerulonephritis, nephrosis, nephronophthisis, chronic interstitial nephritis, and/or nephrosclerosis in a dosage amount that is the same as or less than a standard dosage amount to a subject that is heterozygous for one or more of the ALDH1L1, ALDOB, G6PC, LRP2, RPL3L, SLC25A45, or SLC7A9 variant nucleic acid molecules.

58. The method according to claim 1, wherein any one or more of the ALDH1L1, ALDOB, G6PC, LRP2, RPL3L, SLC25A45, or SLC7A9 variant nucleic acid molecules is a missense variant, a splice-site variant, a stop-gain variant, a start-loss variant, a stop-loss variant, a frameshift variant, or an in-frame indel variant, or a variant that encodes a truncated predicted loss-of-function polypeptide.

59. The method according to claim 58, wherein any one or more of the ALDH1L1, ALDOB, G6PC, LRP2, RPL3L, SLC25A45, or SLC7A9 variant nucleic acid molecules encodes a truncated predicted loss-of-function polypeptide.

60. A method of treating a subject with a therapeutic agent that treats or inhibits a kidney disease wherein the subject has a kidney disease, or preventing a subject from developing a kidney disease by administering a therapeutic agent that prevents a kidney disease, the method comprising the steps of:
determining whether the subject has any one or more variant nucleic acid molecules encoding an Aldehyde Dehydrogenase 1 Family Member L1 (ALDH1L1), a Fructose-Bisphosphate Aldolase B (ALDOB), a Glucose-6-Phosphatase Catalytic Subunit 1 (G6PC), an LDL Receptor Related Protein 2 (LRP2), a Ribosomal Protein L3 Like (RPL3L), a Solute Carrier Family 25, Member 4 (SLC25A45), or a Solute Carrier Family 7 Member 9 (SLC7A9) predicted loss-of-function polypeptide by:
obtaining or having obtained a biological sample from the subject; and
performing or having performed a sequence analysis on the biological sample to determine if the subject has a genotype comprising any one or more of ALDH1L1, ALDOB, G6PC, LRP2, RPL3L, SLC25A45, or SLC7A9 variant nucleic acid molecules encoding a predicted loss-of-function polypeptide; and
administering or continuing to administer the therapeutic agent that treats, prevents, or inhibits the kidney disease in an amount that is the same as or less than a standard dosage amount to a subject that is ALDH1L1, ALDOB, G6PC, LRP2, RPL3L, SLC25A45, and SLC7A9 reference, and/or administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject;
administering or continuing to administer the therapeutic agent that treats, prevents, or inhibits the kidney disease in an amount that is the same as or less than a standard dosage amount to a subject that is heterozygous for any one or more of ALDH1L1, ALDOB, G6PC, LRP2, RPL3L, SLC25A45, or SLC7A9 variant nucleic acid molecules, and/or administering one or more ALDH1L1 inhibitors, ALDOB inhibitors, G6PC inhibitors, LRP2 inhibitors, RPL3L inhibitors, SLC25A45 inhibitors, or SLC7A9 inhibitors, or any combination thereof, to the subject; or
administering or continuing to administer the therapeutic agent that treats, prevents, or inhibits the kidney disease in a standard dosage amount to a subject that is homozygous for any one or more of ALDH1L1, ALDOB, G6PC, LRP2, RPL3L, SLC25A45, or SLC7A9 variant nucleic acid molecules;
wherein the presence of a genotype having one or more of ALDH1L1, ALDOB, G6PC, LRP2, RPL3L, SLC25A45, or SLC7A9 variant nucleic acid molecules indicates the subject has a decreased risk of developing the kidney disease.

61. The method according to claim 60, wherein the subject is: i) ALDH1L1 reference, and the subject is administered or continued to be administered the therapeutic agent that treats, prevents, or inhibits the kidney disease in an amount that is the same as or less than a standard dosage amount, and is administered an ALDH1L1 inhibitor; ii) ALDOB reference, and the subject is administered or continued to be administered the therapeutic agent that treats, prevents, or inhibits the kidney disease in an amount that is the same as or less than a standard dosage amount, and is administered an ALDOB inhibitor; iii) G6PC reference, and the subject is administered or continued to be administered the therapeutic agent that treats, prevents, or inhibits the kidney disease in an amount that is the same as or less than a standard dosage amount, and is administered a G6PC inhibitor; iv) LRP2 reference, and the subject is administered or continued to be administered the therapeutic agent that treats, prevents, or inhibits the kidney disease in an amount that is the same as or less than a standard dosage amount, and is administered an LRP2 inhibitor; v) RPL3L reference, and the subject is administered or continued to be administered the therapeutic agent that treats, prevents, or inhibits the kidney disease in an amount that is the same as or less than a standard dosage amount, and is administered an RPL3L inhibitor; vi) SLC25A45 reference, and the subject is administered or continued to be administered the therapeutic agent that treats, prevents, or inhibits the kidney disease in an amount that is the same as or less than a standard dosage amount, and is administered an SLC25A45 inhibitor; and/or vii) SLC7A9 reference, and the subject is administered or continued to be administered the therapeutic agent that treats, prevents, or inhibits the kidney disease in an amount that is the same as or less than a standard dosage amount, and is administered an SLC7A9 inhibitor.

62. The method according to claim 60, wherein the subject is heterozygous for: i) an ALDH1L1 variant nucleic acid molecule, and the subject is administered or continued to be administered the therapeutic agent that treats, prevents, or inhibits the kidney disease in an amount that is the same as or less than a standard dosage amount, and is administered an ALDH1L1 inhibitor; ii) an ALDOB variant nucleic acid molecule, and the subject is administered or continued to be administered the therapeutic agent that treats, prevents, or inhibits the kidney disease in an amount that is the same as or less than a standard dosage amount, and is administered an ALDOB inhibitor; iii) a G6PC variant nucleic acid molecule, and the subject is administered or continued to be administered the therapeutic agent that treats, prevents, or inhibits the kidney disease in an amount that is the same as or less than a standard dosage amount, and is administered a G6PC inhibitor; iv) an LRP2 variant nucleic acid molecule, and the subject is administered or continued to be administered the therapeutic agent that treats, prevents, or inhibits the kidney disease in an amount that is the same as or less than a standard dosage amount, and is administered an LRP2 inhibitor; v) an RPL3L variant nucleic acid molecule, and the subject is administered or continued to be administered the therapeutic agent that treats, prevents, or inhibits the kidney disease in an amount that is the same as or less than a standard dosage amount, and is administered an RPL3L inhibitor; vi) an SLC25A45 variant nucleic acid molecule, and the subject is administered or continued to be administered the therapeutic agent that treats, prevents, or inhibits the kidney disease in an amount that is the same as or less than a standard dosage amount, and is administered an SLC25A45 inhibitor; and/or vii) an SLC7A9 variant nucleic acid molecule, and the subject is administered or continued to be administered the therapeutic agent that treats, prevents, or inhibits the kidney disease in an amount that is the same as or less than a standard dosage amount, and is administered an SLC7A9 inhibitor.

63. The method according to claim 60, wherein any one or more of the ALDH1L1, ALDOB, G6PC, LRP2, RPL3L, SLC25A45, or SLC7A9 variant nucleic acid molecules is a missense variant, a splice-site variant, a stop-gain variant, a start-loss variant, a stop-loss variant, a frameshift variant, or an in-frame indel variant, or a variant that encodes a truncated predicted loss-of-function polypeptide.

64. The method according to claim 60, wherein any one or more of the ALDH1L1, ALDOB, G6PC, LRP2, RPL3L, SLC25A45, or SLC7A9 variant nucleic acid molecules encodes a truncated predicted loss-of-function polypeptide.

65. The method according to claim 60, wherein: i) the ALDH1L1 inhibitor comprises an inhibitory nucleic acid molecule that hybridizes to an ALDH1L1 nucleic acid molecule; ii) the ALDOB inhibitor comprises an inhibitory nucleic acid molecule that hybridizes to an ALDOB nucleic acid molecule; iii) the G6PC inhibitor comprises an inhibitory nucleic acid molecule that hybridizes to a G6PC nucleic acid molecule; iv) the LRP2 inhibitor comprises an inhibitory nucleic acid molecule that hybridizes to an LRP2 nucleic acid molecule; v) the RPL3L inhibitor comprises an inhibitory nucleic acid molecule that hybridizes to an RPL3L nucleic acid molecule; vi) the SLC25A45 inhibitor comprises an inhibitory nucleic acid molecule that hybridizes to an SLC25A45 nucleic acid molecule; and vii) the SLC7A9 inhibitor comprises an inhibitory nucleic acid molecule that hybridizes to an SLC7A9 nucleic acid molecule.

66. The method according to claim 65, wherein the inhibitory nucleic acid molecule comprises an antisense nucleic acid molecule, a small interfering RNA (siRNA), or a short hairpin RNA (shRNA).

67-72. (canceled)

73. The method according to claim 60, wherein the kidney disease is chronic kidney disease, a kidney stone, chronic glomerulonephritis, nephrosis, nephronophthisis, chronic interstitial nephritis, or nephrosclerosis.

74-79. (canceled)

80. The method according to claim 60, wherein:
when the kidney disease is chronic kidney disease, the therapeutic agent is chosen from furosemide, bumetanide, ethacrynic acid, metolazone, hydrochlorothiazide, a blood pressure medication, a phosphate binder, sodium bicarbonate, and a cholesterol medication, or any combination thereof;
when the kidney disease is a kidney stone, the therapeutic agent is chosen from potassium citrate, furosemide, bumetanide, ethacrynic acid, metolazone, hydrochlorothiazide, allopurinol, acetohydroxamic acid, and mercaptopropionyl glycine, or any combination thereof;
when the kidney disease is chronic glomerulonephritis, the therapeutic agent is chosen from lisinopril, enalapril, captopril, benazepril, fosinopril, quinapril, furosemide, bumetanide, ethacrynic acid, metolazone, hydrochlorothiazide, amlodipine, nifedipine, felodipine, isradipine, verapamil, diltiazem, metoprolol, bisoprolol, esmolol, atenolol, propranolol, sotalol, labetalol, pindolol, penbutolol, clonidine, tizanidine, and dexmedetomidine, or any combination thereof;
when the kidney disease is nephrosis, the therapeutic agent is chosen from lisinopril, enalapril, captopril, benazepril, fosinopril, quinapril, furosemide, bumetanide, ethacrynic acid, metolazone, hydrochlorothiazide, atorvastatin, fluvastatin, lovastatin, pravastatin, rosuvastatin, simvastatin, losartan valsartan heparin, warfarin, dabigatran, apixaban, rivaroxaban, rituximab, cyclosporine, and cyclophosphamide, or any combination thereof;
when the kidney disease is nephronophthisis, the therapeutic agent is erythropoietin; and;
when the kidney disease is chronic interstitial nephritis, the therapeutic agent is succimer or edetate.

81-157. (canceled)
